(19) United States
(12) Patent Application Publication
(10) Pub. No.: US 2022/0135584 A1
(43) Pub. Date: May 5, 2022

(54) BICYCLIC HETEROARYL COMPOUNDS AND USES THEREOF

(71) Applicant: Revolution Medicines, Inc., Redwood City, CA (US)

(72) Inventors: Andreas BUCKL, Redwood City, CA (US); James Joseph CREGG, Redwood City, CA (US); Naing AAY, Redwood City, CA (US); Arlyn A. TAMBO-ONG, Redwood City, CA (US); Elena S. KOLTUN, Redwood City, CA (US); Adrian Liam GILL, Redwood City, CA (US); Severin THOMPSON, Redwood City, CA (US); Micah J Gliedt, Redwood City, CA (US)

(21) Appl. No.: 17/310,930

(22) PCT Filed: Mar. 2, 2020
(86) PCT No.: PCT/US2020/020602

§ 371 Date: Aug. 31, 2021

Related U.S. Application Data
(60) Provisional application No. 62/812,810, filed on Mar. 1, 2019.
(60) Provisional application No. 62/949,780, filed on Dec. 18, 2019.

(51) Int. Cl. C07D 495/04; C07D 487/04; C07D 471/04; C07D 473/34

(52) U.S. Cl. CPC C07D 495/04; C07D 473/34; C07D 471/04; C07D 487/04

(57) ABSTRACT

The present disclosure is directed to modulators of SOS1 and their use in the treatment of disease. Also disclosed are pharmaceutical compositions comprising the same.

### CROSS REFERENCE TO RELATED APPLICATION(S)

The present application claims the benefit of priority to U.S. provisional application Ser. No. 62/812,810, filed Mar. 1, 2019, the disclosure of which is hereby incorporated by reference as if set forth in its entirety. The present application claims the benefit of priority to U.S. provisional application Ser. No. 62/949,780, filed Dec. 18, 2019, the disclosure of which is hereby incorporated by reference as if set forth in its entirety.

### FIELD OF THE DISCLOSURE

The present disclosure relates to inhibitors of SOS1 useful in the treatment of diseases or disorders. Specifically, the present disclosure is concerned with compounds and compositions inhibiting SOS1, methods of treating diseases associated with SOS1, and methods of synthesizing these compounds.

### BACKGROUND OF THE DISCLOSURE

RAS-family proteins including KRAS (V-Ki-ras2 Kirsten rat sarcoma viral oncogene homolog), NRAS (neuroblastoma RAS viral oncogene homolog) and HRAS (Harvey murine sarcoma virus oncogene) and any mutants thereof are small GTPases that exist in cells in either GTP-bound or GDP-bound states (McCormick et al., J. Mol. Med. (Berl)., 2016, 94(3):253-8; Nimnual et al., Sci. STKE., 2002, 2002(145):p136). RAS-family proteins have a weak intrinsic GTPase activity and slow nucleotide exchange rates (Hunter et al., Mol. Cancer Res., 2015, 13(9): 1325-35). Binding of GTPase activating proteins (GAPs) such as NF1 increases the GTPase activity of RAS-family proteins. The binding of guanine nucleotide exchange factors (GEFs) such as SOS1 (Son of Sevenless 1) promote release of GDP from RAS-family proteins, enabling GTP binding (Chardin et al., Science, 1993, 260(5112):1338-43). When in the GTP-bound state, RAS-family proteins are active and engage effector proteins including RAF and phosphoinositide 3-kinase (PI3K) to promote the RAF/mitogen or extracellular signal-regulated kinases (MEK/ERK). Published data indicate a critical involvement of SOS1 in mutant KRAS activation and oncogenic signaling in cancer (Jeng et al., Nat. Commun., 2012, 3:1168). Depleting SOS1 levels decreased the proliferation rate and survival of tumor cells carrying a KRAS mutation whereas no effect was observed in KRAS wild type cell lines. The effect of loss of SOS1 could not be rescued by introduction of a catalytic site mutated SOS1, demonstrating the essential role of SOS1 GEF activity in KRAS mutant cancer cells.

SOS1 is critically involved in the activation of RAS-family protein signaling in cancer via mechanisms other than mutations in RAS-family proteins. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, PDGFR-A/B, FGFR1/2/3, IGF1 R, INSR, ALK, ROS, TrkA, TrkB, TrkC, RET, c-MET, VEGFR1/2/3, AXL) (Pierre et al., Biochem. Pharmacol., 2011, 82(9): 1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275(8):5966-75). This localization of SOS1 to the plasma membrane, proximal to RAS-family proteins, enables SOS1 to promote RAS-family protein activation. SOS1 activation of RAS-family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6(3):268-74). Furthermore, alterations in SOS1 have been implicated in cancer. SOS1 mutations are found in embryonal rhabdomyosarcomas, Sertoli cell testis tumors, granular cell tumors of the skin (Denayer et al., Genes Chromosomes Cancer, 2010, 49(3):242-52) and lung adenocarcinoma (Cancer Genome Atlas Research Network, Nature, 2014, 511 (7511):543-50). Meanwhile over-expression of SOS1 has been described in bladder cancer (Watanabe et al., IUBMB Life, 2000, 49(4):317-20) and prostate cancer (Timofeeva et al., Int. J. Oncol., 2009; 35(4):751-60). In addition to cancer, hereditary SOS1 mutations are implicated in the pathogenesis of RASopathies like e.g., Noonan syndrome (NS), cardio-facio-cutaneous syndrome (CFC) and hereditary gingival fibromatosis type 1 (Pierre et al., Biochem. Pharmacol., 2011, 82(9):1049-56).

SOS1 is also a GEF for the activation of the GTPases RAC1 (Ras-related C3 botulinum toxin substrate 1) (Innocenti et al., J. Cell Biol., 2002, 156(1):125-36). RAC1, like RAS-family proteins, is implicated in the pathogenesis of a variety of human cancers and other diseases (Bid et al., Mol. Cancer Ther. 2013, 12(10):1925-34).

Son of Sevenless 2 (SOS2), a homolog of SOS1 in mammalian cells, also acts as a GEF for the activation of RAS-family proteins (Pierre et al., Biochem. Pharmacol., 2011, 82(9): 1049-56; Buday et al., Biochim. Biophys. Acta., 2008, 1786(2):178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19(4):642-54), systemic conditional SOS1 knockout adult mice are viable (Baltanas et al., Mol. Cell. Biol., 2013, 33(22):4562−78). SOS2 gene targeting did not result in any overt phenotype in mice (Esteban et al., Mol. Cell. Biol., 2000, 20(17):6410-3). In contrast, double SOS1 and SOS2 knockout leads to rapid lethality in adult mice (Baltanas et al., Mol. Cell. Biol., 2013, 33(22):4562−78). These published data suggest that selective targeting of individual SOS isoforms (e.g., selective SOS1 targeting) may be adequately tolerated to achieve a therapeutic index between SOS1/RAS-family protein driven cancers (or other SOS1/RAS-family protein pathologies) and normal cells and tissues.

Selective pharmacological inhibition of the binding of the catalytic site of SOS1 to RAS-family proteins is expected to prevent SOS1-mediated activation of RAS-family proteins to the GTP-bound form. Such SOS1 inhibitor compounds are be expected to consequently inhibit signaling in cells downstream of RAS-family proteins (e.g., ERK phosphorylation). In cancer cells associated with dependence on RAS-family proteins (e.g., KRAS mutant cancer cell lines), SOS1 inhibitor compounds are be expected to deliver anti-cancer efficacy (e.g., inhibition of proliferation, survival, metastasis, etc.). High potency towards inhibition of SOS1:RAS-family protein binding (nanomolar level IC₅₀ values) and ERK phosphorylation in cells (nanomolar level IC₅₀ values) are desirable characteristics for a SOS1 inhibitor compound. Furthermore, a desirable characteristic of a SOS1 inhibitor compound would be the selective inhibition of SOS1 over SOS2. This conclusion is based on the viable phenotype of SOS1 knockout mice and lethality of SOS1/SOS2 double knockout mice, as described above.

These characteristics have not been achieved in previously described SOS1 inhibitor compounds. In the last decades, the RAS family proteins-SOS1 protein interaction has gained increasing recognition. Several efforts to identify and optimize binders, which target either the effector binding site of RAS or the catalytic binding site of SOS1 (for a selected review see: Lu et al., Chem Med Chem. 2016, 11(8):814-21), have been made with limited success.

Recently, small activating molecules have been identified, which bind to a lipophilic pocket of SOS1 in close proximity to the RAS binding site (Burns et al., Proc. Natl. Acad. Sci. 2014, 111(9):3401-6). However, binding of these molecules seems to lead to increased nucleotide exchange and thereby activation of RAS instead of deactivation.

In an effort to stabilize the protein-protein-interaction of RAS-family proteins with SOS1 and to prevent reloading of RAS-family proteins with GTP, several different fragments were subsequently identified (Winter et al., J. Med. Chem. 2015, 58(5):2265-74). However, reversible binding of fragments to SOS1 did not translate into a measurable effect on the nucleotide exchange and only a weak effect was observed for fragments covalently bound to RAS.

Also recently, studies have been conducted to combine rational design and screening platforms to identify small molecule inhibitors of SOS1 (Evelyn et al., Chem. Biol. 2014, 21 (12):1618-28; Evelyn et al., J. Biol. Chem. 2015, 290(20):12879-98; Zheng et al., WO 2016/077793), i.e., compounds which bind to SOS1 and inhibit protein-protein interaction with RAS-family proteins. Although compounds with a slight inhibitory effect on SOS1 have been identified, the effects on guanine nucleotide exchange and cellular signal transduction modulation (e.g., ERK phosphorylation) are weak.

### BRIEF SUMMARY

The present disclosure relates to compounds capable of inhibiting the activity of SOS1. The present disclosure further provides a process for the preparation of compounds, pharmaceutical preparations comprising such compounds and methods of using such compounds and compositions in the management of diseases or disorders associated with the aberrant activity of SOS1.

One aspect of the present disclosure relates to compounds of Formula (I):

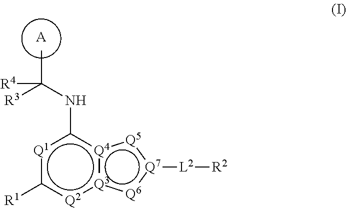

or a pharmaceutically acceptable salt, solvate, isomer, prodrug, or tautomer thereof, wherein:

Q¹ and Q² are independently CH or N;

Q³, Q⁴, and Q⁷ are independently C or N, wherein at least one of Q³ and Q⁴ is C and wherein Q³, Q⁴, and Q⁷ are not all N;

Q⁵ is CH, N, NH, O, or S;

Q⁶ is CH, N, NH, N—C₁₋₆ alkyl, N-C₁₋₆ heteroalkyl, N-(3-7 membered cycloalkyl), N-(3-7 membered heterocyclyl), O, or S;

wherein at least one of Q¹, Q², Q³, Q⁴, Q⁵, Q⁶ and Q⁷ is N, NH, O, or S;

R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}; wherein R^{1a} is H, C₁₋₆ alkyl, 3-6 membered heterocyclyl, or C₁₋₆ haloalkyl;

L² is selected from the group consisting of a bond, —C(O)—, —C(O)O—, —C(O)NH(CH₂)ₒ—, —S(O)₂—,

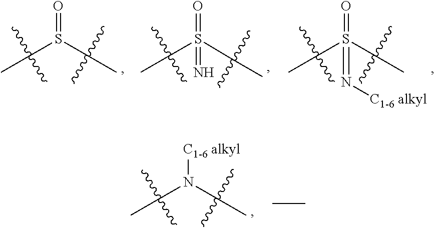

C(O)(CH₂)ₚ—, —(CH₂)ₚ—, and —O—; wherein o is 0, 1, or 2; and wherein p is a number from 1 to 6;

R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;

R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl.

Another aspect of the present disclosure relates to compounds of Formula (I-a):

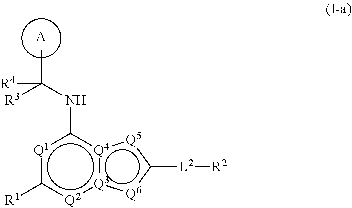

or a pharmaceutically acceptable salt, solvate, isomer, prodrug, or tautomer thereof, wherein:

Q¹, Q², Q5 and A are as defined in Formula (I);

Q³ and Q⁴ are independently C or N, wherein at least one of Q³ and Q⁴ is C;

Q⁶ is CH, N, NH, O, or S;

wherein at least one of Q¹, Q², Q³, Q⁴, Q⁵, and Q⁶ is N, NH, O, or S;

R¹ is selected from the group consisting of H, halogen, C₁₋₆ alkyl, cyclopropyl, —CN, and —OR^{1a}; wherein R^{1a} is H or C₁₋₆ alkyl;

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6;

R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl; and

R³ and R⁴ are independently H or C₁₋₆ alkyl; wherein at least one of R³ and R⁴ is not H; or R³ and R⁴ together with the atom to which they are attached combine to form a 3-6 membered cycloalkyl.

Yet another aspect of the present disclosure relates to compounds of Formula (II):

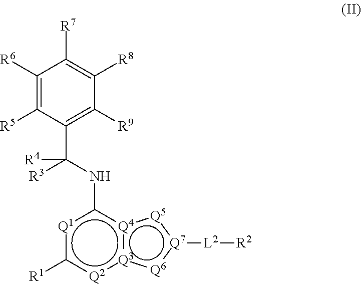

or a pharmaceutically acceptable salt, solvate, isomer, prodrug, or tautomer thereof, wherein:

L², Q¹, Q², Q³, Q⁴, Q⁵, Q⁶, Q⁷, R¹, R², R³ and R⁴ are as defined in Formula (I);

R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, 3-14 membered heterocyclyl, 6-10 membered aryl, or 5-10 membered heteroaryl;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR13R¹⁴, —NO₂, and CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

Yet another aspect of the present disclosure relates to compounds of Formula (II-a):

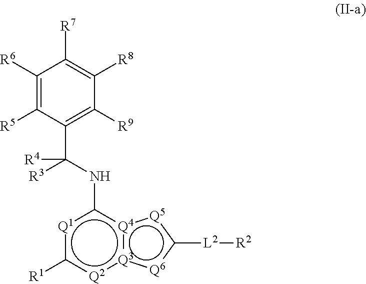

or a pharmaceutically acceptable salt, solvate, isomer, prodrug, or tautomer thereof, wherein:

Q¹, Q², Q⁵, R², R³, R⁴, R⁶, R⁶, R⁷, R⁸, R⁹, R¹⁰, R¹¹, R¹², R¹³ and R¹⁴ are as defined in Formula (II);

Q³ and Q⁴ are independently C or N, wherein at least one of Q³ and Q⁴ is C;

Q⁶ is CH, N, NH, O, or S;

wherein at least one of Q¹, Q², Q³, Q⁴, Q⁵, and Q⁶ is N, NH, O, or S;

R¹ is selected from the group consisting of H, halogen, C₁₋₆ alkyl, cyclopropyl, —CN, and —OR^{1a}; wherein R^{1a} is H or C₁₋₆ alkyl; and

L² is selected from the group consisting of a bond, —C(O)—, —C(O)O—, —C(O)NH(CH₂)ₒ—, —S(O)₂, —C(O)(CH₂)ₚ—, —(CH₂)ₚ, and —O—; wherein o is 0, 1, or 2; and wherein p is a number from 1 to 6.

Yet another aspect of the present disclosure relates to compounds of Formula (III):

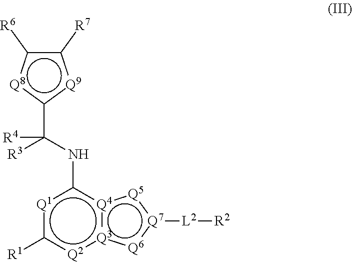

or a pharmaceutically acceptable salt, solvate, isomer, prodrug, or tautomer thereof, wherein:

L², Q¹, Q², Q³, Q⁴, Q⁵, Q⁶, Q⁷, R¹, R², R³ and R⁴ are as defined in Formula (I);

Q⁸ and Q⁹ are independently CH, N, NH, O, or S, provided at least one of Q⁸ and Q⁹ is N, NH, O, or S;

R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

Yet another aspect of the present disclosure relates to compounds of Formula (III-a):

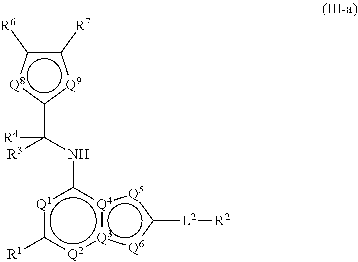

wherein L², Q¹, Q², Q³, Q⁴, Q⁵, Q⁶, Q⁸, Q⁹, R¹, R², R³, R⁴, R⁶, and R⁷ are as defined in Formula (III).

Another aspect of the present disclosure relates to a pharmaceutical composition comprising a compound, or a pharmaceutically acceptable salt, solvate, hydrate, tautomer, or isomer thereof, as set forth above and a pharmaceutically acceptable carrier.

Another aspect of the present disclosure relates to a method of inhibiting SOS1 in a subject, comprising administering to the subject a compound, or a pharmaceutically acceptable salt, solvate, hydrate, tautomer, or isomer thereof, or a pharmaceutical composition, as set forth above.

Another aspect of the present disclosure relates to a method of inhibiting the interaction of SOS1 and a RAS-family protein in a cell or inhibiting the interaction of SOS1 and RAC1 in a cell, comprising administering to the cell a compound, or a pharmaceutically acceptable salt, solvate, hydrate, tautomer, or isomer thereof, or a pharmaceutical composition, as set forth above.

Another aspect of the present disclosure relates to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof an effective amount of a compound, or a pharmaceutically acceptable salt, solvate, hydrate, tautomer, or isomer thereof, or a pharmaceutical composition, as set forth above.

Another aspect of the present disclosure relates to a method of treating or preventing cancer in a subject in need thereof, comprising administering to the subject an effective amount of a compound, or a pharmaceutically acceptable salt, solvate, hydrate, tautomer, or isomer thereof, or a pharmaceutical composition, as set forth above.

Another aspect of the present disclosure relates to a compound, or a pharmaceutically acceptable salt, solvate, hydrate, tautomer, or isomer thereof, as set forth above for use as a medicament.

Another aspect of the present disclosure relates to the use of the compound, or a pharmaceutically acceptable salt, solvate, hydrate, tautomer, or isomer thereof, or a pharmaceutical composition, as set forth above in the manufacture of a medicament for use in inhibiting the binding of hSOS1 to H— or N— or K-RAS including their clinically known mutations and which inhibits the nucleotide exchange reaction catalyzed by hSOS1 in the presence of a concentration of 20 μM or lower, but which are substantially inactive against EGFR-kinase at concentrations of 20 μM or lower.

Another aspect of the present disclosure relates to the use the compound, or a pharmaceutically acceptable salt, solvate, hydrate, tautomer, or isomer thereof, or a pharmaceutical composition, as set forth above in the manufacture of a medicament for use inhibiting the binding of hSOS1 specifically to K-RAS G12C protein and which inhibits the nucleotide exchange reaction catalyzed by hSOS1 in the presence of a concentration of 20 μM or lower, but which are substantially inactive against EGFR-kinase at concentrations of 20 μM or lower.

The present disclosure also provides a compound, or a pharmaceutically acceptable salt, prodrug, solvate, hydrate, tautomer, or isomer thereof, as set forth above that is useful in inhibiting SOS1.

### DETAILED DESCRIPTION OF THE DISCLOSURE

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which the present disclosure belongs. All patents and publications cited in this specification are incorporated herein by reference in their entireties.

### Terms

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment, an optionally substituted group has 2 substituents. In another embodiment, an optionally substituted group has 3 substituents. In another embodiment, an optionally substituted group has 4 substituents. In another embodiment, an optionally substituted group has 5 substituents. For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bonded to a halogen atom, a hydroxyl group, or any other substituent described herein. Thus the term “optionally substituted” means that a given chemical moiety has the potential to contain other functional groups, but does not necessarily have any further functional groups.

As used herein, “alkyl” may mean a straight chain or branched saturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-buty 1, 3-methyl-1-buty 1, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-buty1, 3 ,3-dimethyl-1 -butyl, 2-ethyl-1-buty 1, butyl, isobutyl, t-butyl, n-pentyl, isopentyl, neopentyl, n-hexyl and the like, and longer alkyl groups, such as heptyl, and octyl and the like. An alkyl group can be unsubstituted or substituted. Alkyl groups containing three or more carbon atoms may be straight or branched. As used herein, “lower alkyl” means an alkyl having from 1 to 6 carbon atoms.

As used herein, the term “heteroalkyl” refers to an “alkyl” group (as defined herein), in which at least one carbon atom has been replaced with a heteroatom (e.g., an 0, N, or S atom). The heteroatom may appear in the middle or at the end of the radical.

The term “alkenyl” means an aliphatic hydrocarbon group containing a carbon—carbon double bond and which may be straight or branched having about 2 to about 6 carbon atoms in the chain. Certain alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkynyl” means an aliphatic hydrocarbon group containing a carbon-carbon triple bond and which may be straight or branched having about 2 to about 6 carbon atoms in the chain. Certain alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

As used herein, the term “halo” or “halogen” means a fluoro, chloro, bromo, or iodo group.

The term “oxo” as used herein refers to an “═O” group. When an oxo group is bonded to a carbon atom, it can also be abbreviated herein as C(O) or as C═O. An oxo group can also be bonded to a sulfur atom (e.g., S═O and S(O)₂) or at phosphorous atom (e.g., P═O, PO₂, PO₃, PO₄, etc.).

The term “imine” as used herein refers to an “═N” group. When an imine is bonded to a carbon atom, it can also be abbreviated herein as C═N. Nitrogen can also be double bonded to sulfur, e.g., S═N, which is referred to as a thioimine.

The term “annular atoms” used in conjunction with terms relating to ring systems described herein (e.g., cycloalkyl, cycloalkenyl, aryl, heterocyclyl, and heteroaryl) refers to the total number of ring atoms present in the system. “Annular atoms” therefore does not include the atoms present in a substituent attached to the ring. Thus, the number of “annular atoms” includes all atoms present in a fused ring. For example, a 2-indolyl ring,

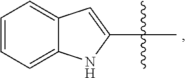 [SMILES: *c1cc2ccccc2[nH]1]

is considered a 5-membered heteroaryl, but is also a heteroaryl containing 9 annular atoms. In another example, pyridine is considered a 6-membered heteroaryl, and is a heteroaryl containing 6 annular atoms.

“Cycloalkyl” refers to a single saturated all carbon ring having 3 to 20 annular carbon atoms (i.e., C₃-C₂₀ cycloalkyl), for example from 3 to 15 annular atoms, for example, from 3 to 12 annular atoms. In certain embodiments, the cycloalkyl group is either monocyclic (“monocyclic cycloalkyl”) or contains a fused, bridged or spiro ring system such as a bicyclic system (“bicyclic cycloalkyl”) and can be saturated. “Cycloalkyl” includes ring systems where the cycloalkyl ring, as defined above, is fused with one or more cycloalkyl, cycloalkenyl, heterocyclyl, aryl or heteroaryl groups, wherein the point of attachment is on a cycloalkyl ring, and, in such instances, the number of carbon atoms recited continues to designate the number of carbons in the cycloalkyl ring containing the point of attachment. Examples of cycloalkyl groups include cyclohexyl, cycloheptyl, 2-adamantyl

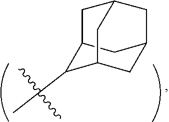

2-(2,3-dihydro-1H-indene)

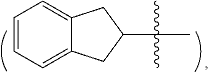

and 9-fluorenyl

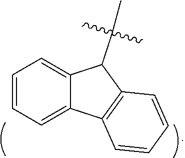

As noted above, cycloalkyl rings can be further characterized by the number of annular atoms. For example, a cyclohexyl ring is a C₆ cycloalkyl ring with 6 annular atoms, while 2-(2,3-dihydro-1H-indene) is a C₅ cycloalkyl ring with 9 annular atoms. Also, for example, 9-fluorenyl is a C₅ cycloalkyl ring with 13 annular atoms and 2-adamantyl is a C₆ cycloalkyl with 10 annular atoms.

As used herein, the term “cycloalkenyl” may refer to a partially saturated, monocyclic, fused or spiro polycyclic, all carbon ring having from 3 to 18 carbon atoms per ring and contains at least one double bond. “Cycloalkenyl” includes ring systems where the cycloalkenyl ring, as defined above, is fused with one or more cycloalkyl, cycloalkenyl, heterocyclyl, aryl or heteroaryl groups, wherein the point of attachment is on a cycloalkenyl ring, and, in such instances, the number of carbon atoms recited continues to designate the number of carbons in the cycloalkenyl ring containing the point of attachment. Cycloalkenyl rings can be further characterized by the number of annular atoms. Examples of cycloalkenyl include 1-cyclohex-1-enyl and cyclopent-1-enyl.

The term “aryl” as used herein refers to a single all carbon aromatic ring or a multiple condensed all carbon ring system wherein at least one of the rings is aromatic. For example, in certain embodiments, an aryl group has 5 to 20 annular carbon atoms, 5 to 14 annular carbon atoms, or 5 to 12 annular carbon atoms. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., cycloalkyl). “Aryl” includes ring systems where the aryl ring, as defined above, is fused with one or more cycloalkyl, cycloalkenyl, heterocyclyl, aryl or heteroaryl groups, and wherein the point of attachment is on an aryl ring, and, in such instances, the number of carbon atoms recited continues to designate the number of carbon atoms in the aryl ring containing the point of attachment. Examples of aryl groups include phenyl and 5-(2,3-dihydro-1H-indene):

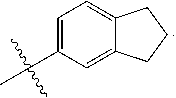 [SMILES: *c1ccc2c(c1)CCC2]

As noted above, aryl rings can be further characterized by the number of annular atoms. For example, phenyl is a C₆ aryl with 6 annular atoms, while 5-(2,3-dihydro-1H-indene) is a C₆ aryl with 9 annular atoms.

“Heterocyclyl” as used herein refers to a single saturated or partially unsaturated non-aromatic ring or a non-aromatic multiple ring system (including fused and spiro polycyclic) that has at least one heteroatom in the ring (at least one annular heteroatom selected from oxygen, nitrogen, phosphorus, and sulfur). Unless otherwise specified, a heterocyclyl group has from 5 to about 20 annular atoms, for example from 5 to 15 annular atoms, for example from 5 to 10 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen, phosphorus, and sulfur in the ring. The term also includes single saturated or partially unsaturated rings (e.g., 5, 6, 7, 8, 9, or 10-membered rings) having from about 4 to 9 annular carbon atoms and from about 1 to 3 annular heteroatoms selected from the group consisting of oxygen, nitrogen, phosphorus, and sulfur in the ring. “Heterocyclyl” includes ring systems where the heterocyclyl ring, as defined above, is fused with one or more cycloalkyl, cycloalkenyl, heterocyclyl, aryl or heteroaryl groups, wherein the point of attachment is on a heterocyclic ring, and, in such instances, the number of ring members recited continues to designate the number of annular atoms in the heterocyclic ring containing the point of attachment. Heterocyclic rings can be further characterized by the number of annular atoms. Examples of heterocyclic groups include piperidinyl (6-membered heterocycle with 6 annular atoms), azepanyl (7-membered heterocycle with 7 annular atoms), and 3-chromanyl (6-membered heterocycle with 10 annular atoms)

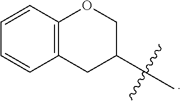 [SMILES: *C1COc2ccccc2C1]

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring. Thus, the term includes single heteroaryl rings of from about 1 to 10 annular carbon atoms and about 1-5 annular heteroatoms selected from the group consisting of oxygen, nitrogen and sulfur in the rings. The sulfur and nitrogen atoms may also be present in an oxidized form provided the ring is aromatic. “Heteroaryl” includes ring systems where the heteroaryl ring, as defined above, is fused with one or more cycloalkyl, cycloalkenyl, heterocyclyl, aryl or heteroaryl groups, wherein the point of attachment is on a heteroaryl ring, and, in such instances, the number of ring members continues to designate the number of ring members in the heteroaryl ring containing the point of attachment. Heteroaryl rings can be further characterized by the number of annular atoms. For example, pyridine is a 6-membered heteroaryl having 6 annular atoms.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

For example, compounds of the present disclosure can exist in tautomeric form. In some embodiments of compounds of the Formulae disclosed herein, R¹ can be —OH and tautomers of the compounds can exist in equilibrium, as shown below, depending on the identities of Q⁵ and Q⁶:

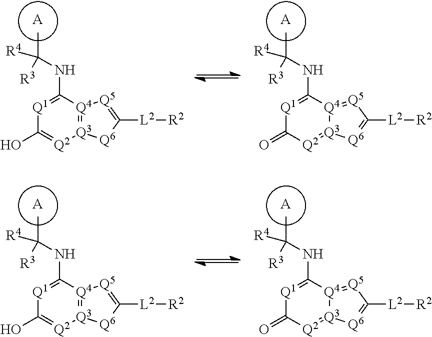

Compounds of the present disclosure can also include all isotopes of atoms occurring in the intermediates or final compounds. Isotopes include those atoms having the same atomic number but different mass numbers. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. One or more constituent atoms of the compounds of the present disclosure can be replaced or substituted with isotopes of the atoms in natural or non-natural abundance. In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms. Synthetic methods for including isotopes into organic compounds are known in the art.

The term “prodrug,” as used in this disclosure, means a compound which is convertible in vivo by metabolic means (e.g., by hydrolysis) to a disclosed compound. Furthermore, as used herein a prodrug is a drug which is inactive in the body, but is transformed in the body typically either during absorption or after absorption from the gastrointestinal tract into the active compound. The conversion of the prodrug into the active compound in the body may be done chemically or biologically (i.e., using an enzyme).

The term “solvate” refers to a complex of variable stoichiometry formed by a solute and solvent. Such solvents for the purpose of the present disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds herein may have one or more asymmetric carbon atom and may occur as racemates, racemic mixtures and as individual enantiomers or diastereomers.

The term “stereoisomers” refers to the set of compounds which have the same number and type of atoms and share the same bond connectivity between those atoms, but differ in three dimensional structure. The term “stereoisomer” refers to any member of this set of compounds. For instance, a stereoisomer may be an enantiomer or a diastereomer.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers.

An “effective amount” when used in connection with a compound is an amount effective for treating or preventing a disease in a subject as described herein.

The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more or any range derivable therein, reduction of activity (e.g., SOS1:ras-family protein binding activity) compared to normal.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds of Disclosed Formulae

In some embodiments, the present disclosure relates to compounds of the following formula:

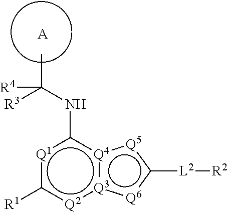

and pharmaceutically acceptable salts, prodrugs, solvates, hydrates, tautomers, and isomers thereof, wherein:

Q¹ and Q² are independently CH or N;

Q³ and Q⁴ are independently C or N, wherein at least one of Q³ and Q⁴ is C;

Q⁵ and Q⁶ are independently CH, N, NH, O, or S;

wherein at least one of Q, Q², Q³, Q⁴, Q⁵, and Q⁶ is N, NH, O, or S;

R¹ is H, halogen, C₁₋₆ alkyl, 3-membered cycloalkyl, CN, or OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R²c is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl.

In other embodiments, the present disclosure relates to compounds of the following formula:

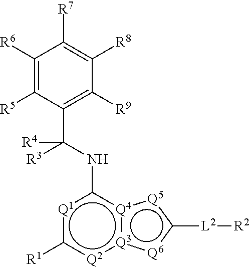

and pharmaceutically acceptable salts, prodrugs, solvates, hydrates, tautomers, and isomers thereof, wherein:

Q¹ and Q² are independently CH or N;

Q³ and Q⁴ are independently C or N, wherein at least one of Q³ and Q⁴ is C;

Q⁵ and Q⁶ are independently CH, N, NH, O, or S;

wherein at least one of Q¹, Q², Q³, Q⁴, Q⁵, and Q⁶ is N, NH, O, or S;

R¹ is halogen, C₁₋₆ alkyl, 3-membered cycloalkyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl;

R⁵, R⁶, R⁷, R⁸, and R⁹ are independently selected from the group consisting of H, D, C₁-C₆ alkyl, C₂-C₆ alkenyl, C₄-C₈ cycloalkenyl, C₂-C₆ alkynyl, C₃-C₈ cycloalkyl, —OH, halogen, —NO₂, —CN, —NR¹¹R¹², —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, or —CO₂R¹⁰, wherein each alkyl, alkenyl, cycloalkenyl, alkynyl, or cycloalkyl is optionally substituted with one or more —OH, halogen, —NO₂, oxo, —CN, —R¹⁰, —OR¹⁰, —NR¹¹R¹², —SR¹⁰, —S(O)₂NR¹¹R¹², —S(O)_{2R}¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, heterocycle, aryl, or heteroaryl;

R¹⁰, R¹¹, and R¹² are independently, at each occurrence, H, D, Cₗ-C₆ alkyl, C₂-C₆ alkenyl, C₄-C₈ cycloalkenyl, C₂-C₆ alkynyl, C₃-C₈ cycloalkyl, a monocyclic 3-12 membered heterocycle, a polycyclic 3-12 membered heterocycle, —OR¹³, —SR¹³, halogen, —NR¹³R¹⁴, —NO₂, or —CN; and

R¹³ and R¹⁴ are independently, at each occurrence, H, D, C₁-C₆ alkyl, C₂-C₆ alkenyl, C₄-C₈ cycloalkenyl, C₂-C₆ alkynyl, C₃-C₈ cycloalkyl, a monocyclic 3-12 membered heterocycle, or a polycyclic 3-12 membered heterocycle, wherein each alkyl, alkenyl, cycloalkenyl, alkynyl, cycloalkyl, or heterocycle is optionally substituted with one or more —OH, —SH, —NH₂, —NO₂, or —CN.

### Additional Compounds of Disclosed Formulae

The present disclosure additionally provides for compounds of Formula (I),

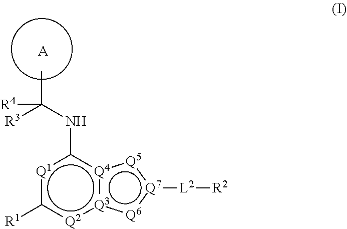

or a pharmaceutically acceptable salt, solvate, isomer, prodrug, or tautomer thereof, wherein:

Q¹ and Q² are independently CH or N;

Q³, Q⁴, and Q⁷ are independently C or N, wherein at least one of Q³ and Q⁴ is C and wherein Q³, Q⁴, and Q⁷ are not all N;

Q⁵ is CH, N, NH, O, or S;

Q⁶ is CH, N, NH, N—C₁₋₆ alkyl, N—C₁₋₆ heteroalkyl, N-(3-7 membered cycloalkyl), N-(3-7 membered heterocyclyl), O, or S;

wherein at least one of Q¹, Q², Q³, Q⁴, Q⁵, Q⁶, and Q⁷ is N, NH, O, or S;

R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}; wherein R^{1a} is H, C₁₋₆ alkyl, 3-6 membered heterocyclyl, or C₁₋₆haloalkyl;

L² is selected from the group consisting of a bond, —C(O)—, —C(O)O—, —C(O)NH(CH₂)ₒ—, —S(O)₂—,

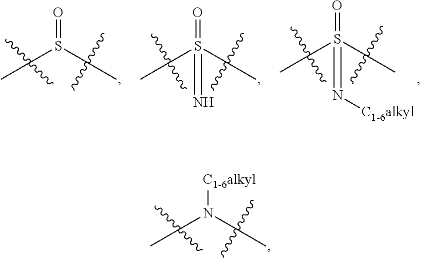

C(O)(CH₂)ₚ—, —(CH₂)ₚ—, and —O—; wherein o is 0, 1, or 2; and wherein p is a number from 1 to 6;

R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, —OH, —OR^{2a}, oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;

wherein R^{2a} is H, C₁₋₆ alkyl, C₁₋₆haloalkyl, 3-7 membered heterocyclyl, or —(CH₂)ᵣOCH₃, wherein r is 1, 2, or 3;

wherein R^{2b} is H or C₁₋₆ alkyl;

wherein R^{2c} is H or C₁₋₆ alkyl;

R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl.

In some embodiments of compounds of Formula (I), no more than four of Q¹, Q², Q₃, Q⁴, Q⁵, Q⁶, and Q⁷ is N, NH, NCH₃, O, or S. In some embodiments of compounds of Formula (I), no more than five of Q¹, Q², Q³, Q⁴, Q⁵, Q⁶, and Q⁷ is N, NH, NCH₃, O, or S.

The present disclosure also provides for compounds of Formula (I-a),

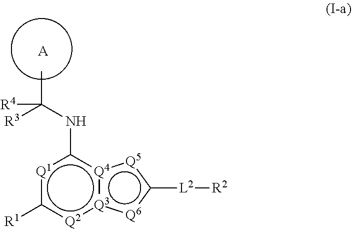

or a pharmaceutically acceptable salt, solvate, isomer, prodrug, or tautomer thereof, wherein:

Q¹, Q², Q⁵ and A are as defined above in Formula (I);

Q³ and Q⁴ are independently C or N, wherein at least one of Q³ and Q⁴ is C;

Q⁶ is CH, N, NH, O, or S;

wherein at least one of Q¹, Q², Q³, Q⁴, Q⁵, and Q⁶ is N, NH, O, or S;

R¹ is selected from the group consisting of H, halogen, C₁₋₆ alkyl, cyclopropyl, —CN, and —OR^{1a}; wherein R^{1a} is H or C₁₋₆ alkyl;

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6;

R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl; and

R³ and R⁴ are independently H or C₁₋₆ alkyl; wherein at least one of R³ and R⁴ is not H; or R³ and R⁴ together with the atom to which they are attached combine to form a 3-6 membered cycloalkyl.

As described herein for Formula (I) or (I-a), A is an optionally substituted 6-membered aryl or an optionally substituted 5-6 membered heteroaryl.

In certain embodiments of Formula (I) or (I-a), A is an optionally substituted 6-membered aryl. In certain embodiments, A is an optionally substituted 5-6 membered heteroaryl. In certain embodiments, A is an optionally substituted 5-membered heteroaryl. In certain embodiments, A is an optionally substituted 6-membered heteroaryl.

In certain embodiments of Formula (I) or (I-a), A is a 6-membered aryl. In certain embodiments of Formula I, A is a 6-membered aryl, which is substituted with R⁵, R⁶, R⁷, R⁸, and R⁹, as described herein and shown below:

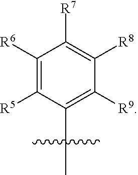

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰ , —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In the above, R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN.

In the above, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In certain embodiments of Formula (I) or (I-a), A is a 5-6 membered heteroaryl. In certain embodiments of Formula I, A is a 5-membered heteroaryl, which is substituted with R⁵, R⁶, R⁷, R⁸, and R⁹, as described herein and shown below:

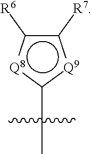

In some embodiments, Q⁸ and Q⁹ are independently CH, N, NH, O, or S, provided at least one of Q⁸ and Q⁹ is N, NH, O, or S.

In some embodiments, R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰D(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, S(O)₂NR¹¹R¹², —S(O)₂R¹⁰, —NR¹⁰S(O)₂NR¹¹R¹², —NR¹⁰ S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In the above, R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN.

In the above, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure also provides for compounds of Formula (II),

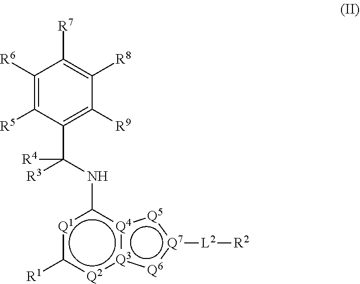

or a pharmaceutically acceptable salt, solvate, isomer, prodrug, or tautomer thereof, wherein:

L², Q¹, Q², Q³, Q⁴, Q⁵, Q⁶, Q⁷, R′, R², R³ and R⁴ are as defined above in Formula

R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, 3-14 membered heterocyclyl, 6-10 membered aryl, or 5-10 membered heteroaryl;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments of compounds of Formula (II), no more than four of Q¹, Q², Q³, Q⁴, Q⁵, and Q⁷ is N, NH, NCH₃, O, or S. In some embodiments of compounds of Formula (II), no more than five of Q¹, Q², Q³, Q⁴, Q⁵, Q⁶, and Q⁷ is N, NH, NCH₃, O, or S.

The present disclosure also provides for compounds of Formula (II-a),

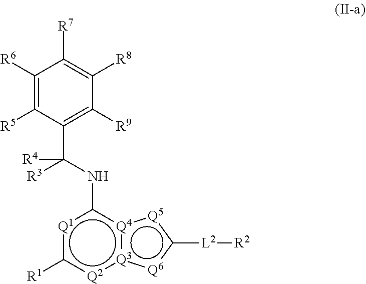

or a pharmaceutically acceptable salt, solvate, isomer, prodrug, or tautomer thereof, wherein:

Q¹, Q², Q⁵, R², R³, R⁴, R⁵, R⁶, R⁷, R⁸, R⁹, R¹⁰, R¹¹, R¹², R¹³ and R¹⁴ are as defined above in Formula (II);

Q³ and Q⁴ are independently C or N, wherein at least one of Q³ and Q⁴ is C;

Q⁶ is CH, N, NH, O, or S;

wherein at least one of Q¹, Q², Q³, Q⁴, Q⁵, and Q⁶ is N, NH, O, or S;

R¹ is selected from the group consisting of H, halogen, C₁₋₆ alkyl, cyclopropyl, —CN, and —OR^{1a}; wherein R^{1a} is H or C₁₋₆ alkyl; and

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6.

The present disclosure also provides for compounds of Formula (II-b),

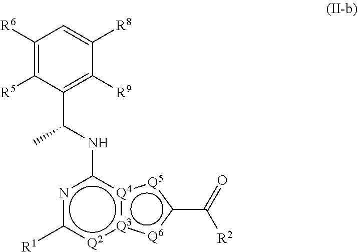

or a pharmaceutically acceptable salt, solvate, isomer, prodrug, or tautomer thereof, wherein:

Q² is CH or N;

Q³ and Q⁴ are independently C or N;

Q⁵ is CH, N, or NH;

Q⁶ is CH, N, NH, N—CH₃, or S;

R¹ is selected from the group consisting of —H, —CH₃, and —Cl;

R² is selected from the group consisting of 3-14 membered heterocyclyl optionally substituted with C₁₋₆ alkyl, C₁₋₆ haloalkyl, —OR^{2a}, —C(O)R^{2a}, 3-6 membered cycloalkyl, and 3-7 membered heterocyclyl, wherein R^{2a} is H or C₁₋₆ alkyl; and R⁵, R⁶, R⁷, R⁸, and R⁹ are independently selected from the group consisting of H, —F, —CHF₂, —CF₂CH₂OH, —CF₃, —NH₂.

The present disclosure also provides for compounds of Formula (III),

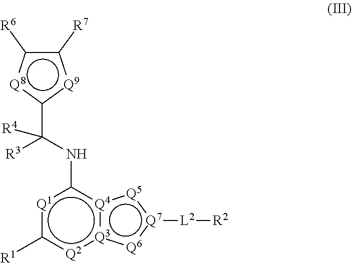

or a pharmaceutically acceptable salt, solvate, isomer, prodrug, or tautomer thereof, wherein:

L², Q¹, Q², Q³, Q⁴, Q⁵, Q⁶, Q⁷, R¹, R², R³ and R⁴ are as defined above in Formula (I);

Q⁸ and Q⁹ are independently CH, N, NH, O, or S, provided at least one of Q⁸ and Q⁹ is N, NH, O, or S;

R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, 3-14 membered heterocyclyl, 6-10 membered aryl, or 5-10 membered heteroaryl;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments of compounds of Formula (III), no more than five of Q¹, Q², Q³, Q⁴, Q⁵, Q⁶, and Q⁷ is N, NH, NCH₃, O, or S. In some embodiments of compounds of Formula (III), no more than four of Q¹, Q², Q³, Q⁴, Q⁵, Q⁶, and Q⁷ is N, NH, NCH₃, O, or S.

In some embodiments of compounds of Formula (III), one of Q⁸ and Q⁹ is CH and one of Q⁸ and Q⁹ is S.

The present disclosure also provides for compounds of Formula (III-a),

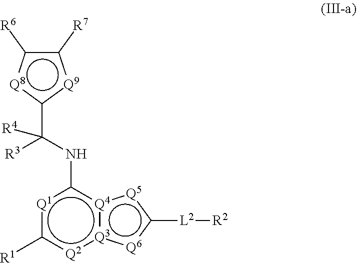

and pharmaceutically acceptable salts, prodrugs, solvates, hydrates, tautomers, or isomers thereof, wherein Q¹, Q², Q³, Q⁴, Q⁵, Q⁶, Q⁸, Q⁹, R¹, R², R³, R⁴, R⁵, R⁷, and L² are described as above.

As described above, Q¹ and Q² are independently CH or N. In certain embodiments, Q¹ is CH. In certain embodiments, Q¹ is N. In certain embodiments, Q² is CH. In certain embodiments, Q² is N.

As described above, Q³ and Q⁴ are independently C or N, wherein at least one of Q³ and Q⁴ is C. In certain embodiments, Q³ is C. In certain embodiments, Q³ is N. In certain embodiments, Q⁴ is C. In certain embodiments, Q⁴ is N.

As described above, Q⁵ and Q⁶ are independently CH, N, NH, O, or S. In certain embodiments, Q⁵ is CH. In certain embodiments, Q⁵ is N or NH. In certain embodiments, Q⁵ is N. In certain embodiments, Q⁵ is NH. In certain embodiments, Q⁵ is O or S. In certain embodiments, Q⁵ is O. In certain embodiments, Q⁵ is S. In certain embodiments, Q⁶ is CH. In certain embodiments, Q⁶ is N, NH, or N—CH₃. In certain embodiments, Q⁶ is N or NH. In certain embodiments, Q⁶ is N—CH₃. In certain embodiments, Q⁶ is N. In certain embodiments, Q⁶ is NH. In certain embodiments, Q⁶ is O or S. In certain embodiments, Q⁶ is O. In certain embodiments, Q⁶ is S.

In certain embodiments,

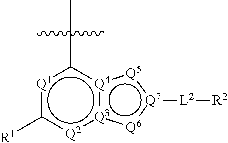

is selected from the group consisting of:

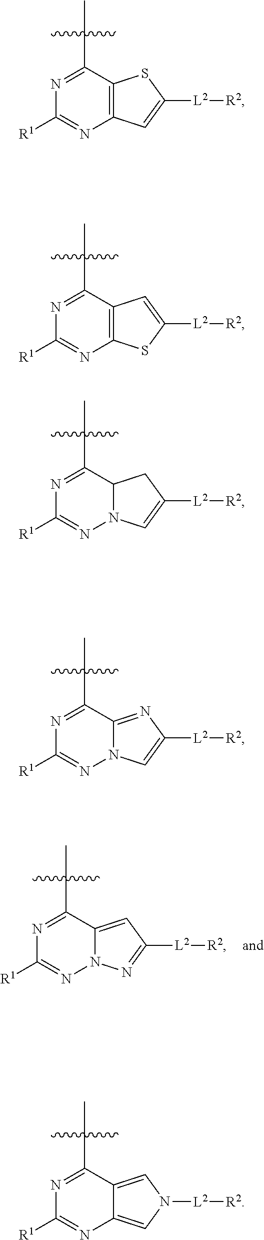

In certain embodiments,

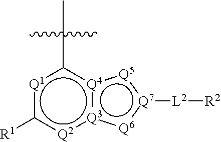

is selected from the group consisting of:

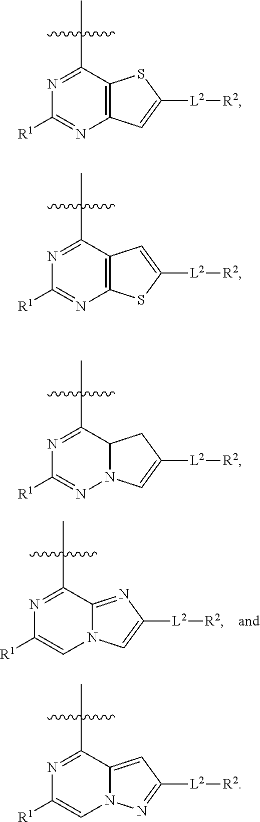

In certain embodiments,

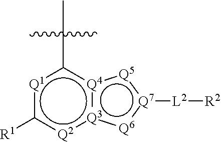

is selected from the group consisting of:

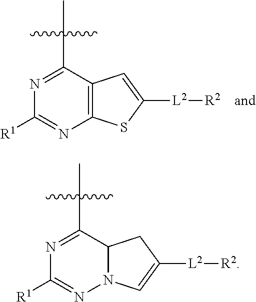

In certain embodiments,

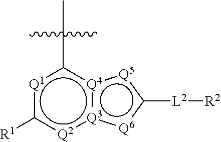

is selected from the group consisting of:

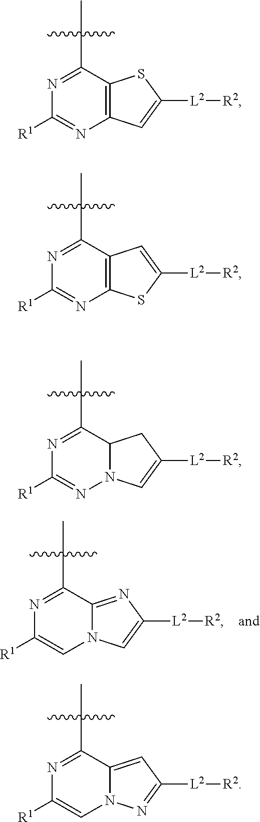

In certain embodiments,

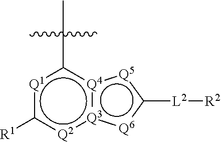

is selected from the group consisting of:

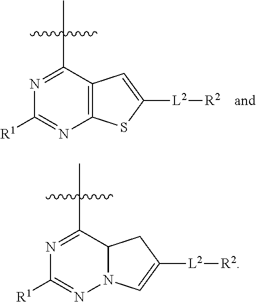

As described herein, R¹ is H, halogen, C₁₋₆ alkyl, cyclopropyl, —CN, or —OR^{1a}; wherein R^{1a} is H or C₁₋₆ alkyl. In certain embodiments, R¹ is halogen, C₁₋₆ alkyl, cyclopropyl, —CN, or —OR^{1a}; wherein R^{1a} is H or C₁₋₆ alkyl.

In certain embodiments, R¹ is H. In certain embodiments, R¹ is halogen. In certain embodiments, R¹ is C₁₋₆ alkyl. In certain embodiments, R¹ is C₁ alkyl, C₂ alkyl, C₃ alkyl, C₄ alkyl, C₅ alkyl, or C₆ alkyl. In certain embodiments, R¹ is cyclopropyl. In certain embodiments, R¹ is —CN. In certain embodiments, R¹ is —OR^{1a}; wherein R^{1a} is H or C₁₋₆ alkyl. In certain embodiments, R¹ is OH. In certain embodiments, R¹ is OR^{1a}; wherein R^{1a} is C₁₋₆ alkyl.

In certain embodiments, R¹ is selected from the group consisting of H, —CH₃, —Cl, cyclopropyl, and —OCH₃.

As described herein, L² is selected from the group consisting of a bond, —C(O)—, —C(O)O—, —C(O)NH(CH₂)ₒ—, —S(O)₂—,

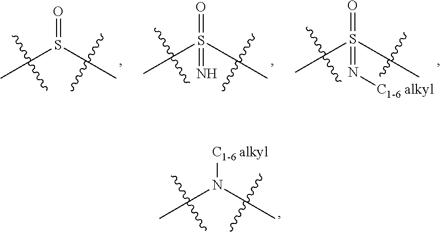

—C(O)(CH₂)ₚ—, —(CH₂)ₚ—, and —O—; wherein o is 0, 1, or 2; and wherein p is a number from 1 to 6. In some embodiments wherein L² comprises a carbonyl group, the carbon of the carbonyl group is bonded to Q⁷.

In some embodiments, L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6. In some embodiments wherein L² comprises a carbonyl group, the carbon of the carbonyl group is bonded to Q⁷.

In some embodiments, L² is selected from the group consisting of

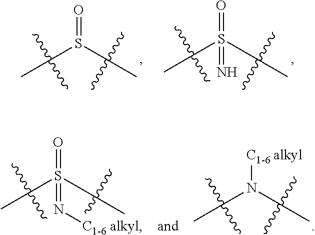

In certain embodiments, L² is a bond. In certain embodiments, L² is —C(O)—. In certain embodiments, L² is —C(O)O—, wherein the carbonyl carbon is bonded to Q⁷. In certain embodiments, L² is —C(O)NH(CH₂)ₒ—, wherein the carbonyl carbon is bonded to Q⁷. In certain embodiments, L² is —S(O)₂—. In certain embodiments, L² is —C(O)(CH₂)ₚ—. In certain embodiments, L² is —(CH₂)ₚ—. In certain embodiments, L² is —O—.

As described herein, o is 0, 1, or 2. In certain embodiments, o is 0. In certain embodiments, o is 1. In certain embodiments, o is 2.

As described above, p is a number from 1 to 6. In certain embodiments, p is 1. In certain embodiments, p is 2. In certain embodiments, p is 3. In certain embodiments, p is 4. In certain embodiments, p is 5. In certain embodiments, p is 6.

In some embodiments, R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, —OH, —OR^{2a}, oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl.

In some embodiments, R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, and 5-10 membered heteroaryl is independently optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is H. In some embodiments, R² is —CH₃. In certain embodiments, R² is —(CH₂)_{q}CH₃, wherein q is a number from 1 to 5. In certain embodiments, q is 1. In certain embodiments, q is 2. In certain embodiments, q is 3. In certain embodiments, q is 4. In certain embodiments, q is 5. In certain embodiments, R² is C₂₋₆ alkenyl, which is optionally substituted. In some embodiments, C₂ alkenyl, which is optionally substituted. In some embodiments, R² is —C═C—COOH.

In certain embodiments, R² is —NR^{2b}R^{2c}, wherein R^{2b} is H or C₁₋₆ alkyl; and wherein R^{2c} is H or C₁₋₆ alkyl. In some embodiments, R² is —NHCH₃.

In certain embodiments, R² is 3-14 membered heterocyclyl, wherein the 3-14 membered heterocyclyl is optionally substituted with C₁₋₆ alkyl, C₁₋₆haloalkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In some embodiments, R² is selected from among

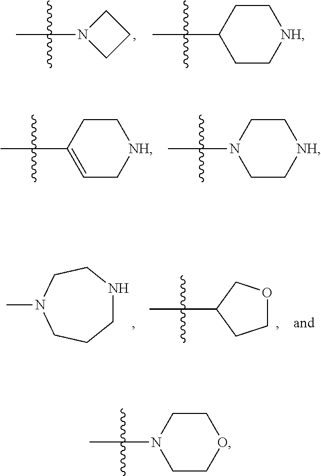

and each of which is optionally substituted at any of the carbon atoms or nitrogen atoms.

In some embodiments, R² is selected from among

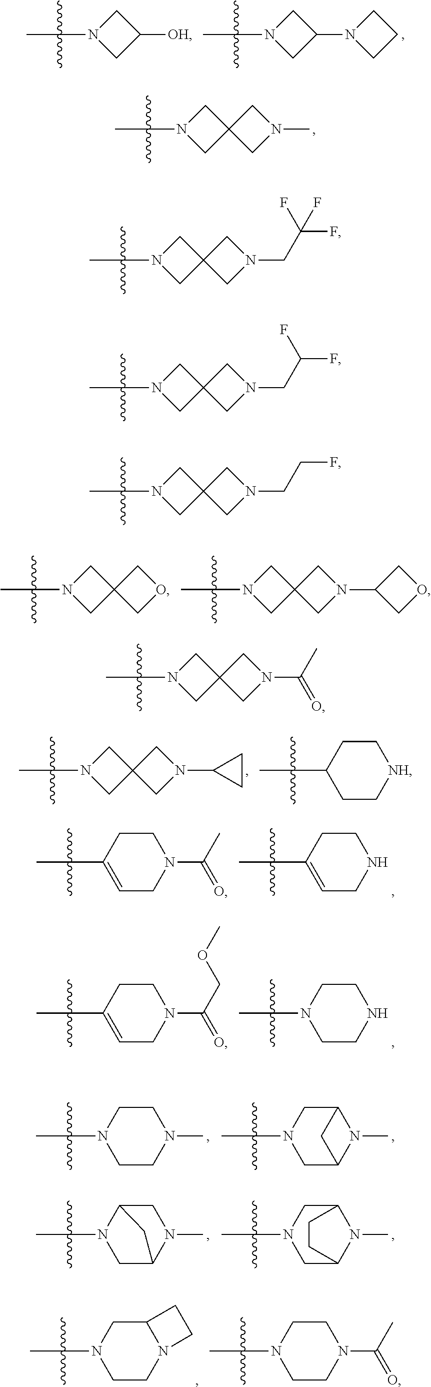

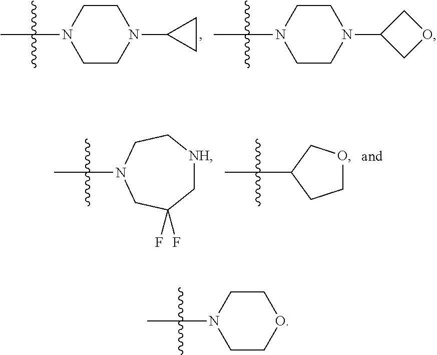

In certain embodiments, R² is 5-10 membered heteroaryl, wherein the 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein ^{R2c} is H or C₁₋₆ alkyl. In some embodiments, R² is

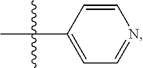 [SMILES: *c1ccncc1]

which is optionally substituted.

In some embodiments, R² is selected from among

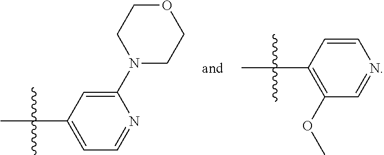

In certain embodiments, R² is 6-10 membered aryl, wherein the 6-10 membered aryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is 3-14 membered cycloalkyl, wherein the 3-14 membered cycloalkyl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl. In some embodiments, R² is selected from among cyclobutyl, cyclopentyl, or cyclohexyl, each of which is optionally substituted.

In some embodiments, R² is selected from among

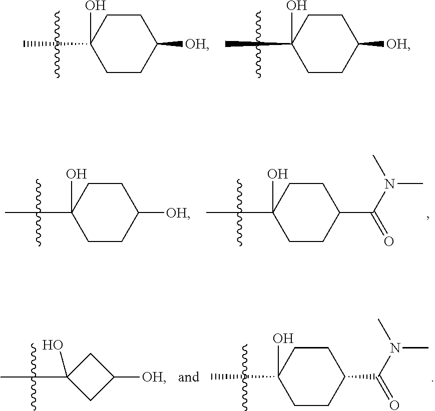

In certain embodiments, R² is 3-14 membered cycloalkenyl, wherein the 3-14 membered cycloalkenyl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

As described herein, R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl.

In certain embodiments, R³ is H. In certain embodiments, R³ is C₁₋₆ alkyl, such as C₁ alkyl, C₂ alkyl, C₃ alkyl, C₄ alkyl, C₅ alkyl, or C₆ alkyl.

In certain embodiments, R⁴ is H. In certain embodiments, R⁴ is C₁₋₆ alkyl, such as C₁ alkyl, C₂ alkyl, C₃ alkyl, C₄ alkyl, C₅ alkyl, or C₆ alkyl.

In certain embodiments, R³ is H and R⁴ is C₁₋₆ alkyl, such as C₁ alkyl, C₂ alkyl, C₃ alkyl, C₄ alkyl, C₅ alkyl, or C₆ alkyl.

In certain embodiments, R³ and R⁴ together with the atom to which they are attached combine to form a 3-6 membered cycloalkyl, such as 3, 4, 5 or 6-membered cycloalkyl.

As described herein, R⁵, R⁶, R⁷, R⁸, and R⁹ are independently selected from the group consisting of H, D, C₁-C₆ alkyl, C₂-C₆ alkenyl, C₄-C₈ cycloalkenyl, C₂-C₆ alkynyl, C₃-C₈ cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, or —CO₂R¹⁰, wherein each alkyl, alkenyl, cycloalkenyl, alkynyl, or cycloalkyl is optionally substituted with one or more —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, heterocycle, aryl, or heteroaryl.

As described herein, R¹⁰, R¹¹, and R¹² are at each occurrence independently selected from H, D, C₁-C₆ alkyl, C₂-C₆ alkenyl, C₄-C₈ cycloalkenyl, C₂-C₆ alkynyl, C₃-C₈ cycloalkyl, a monocyclic 3- to 12-membered heterocycle, a polycyclic 3- to 12-membered heterocycle, —OR¹³, —SR¹³, halogen, —NR¹³R¹⁴, —NO₂, or —CN.

As described herein, R¹³ and R¹⁴ are at each occurrence independently selected from H, D, C₁-C₆ alkyl, C₂-C₆ alkenyl, C₄-C₈ cycloalkenyl, C₂-C₆ alkynyl, C₃-C₈ cycloalkyl, a monocyclic 3- to 12-membered heterocycle, or a polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN.

In certain embodiments, one to three of R₅, R₆, R₇, R₈, and R₉ is C₁₋₆ alkyl optionally substituted with halogen. In certain embodiments, one to three of R⁵, R⁶, R⁷, R⁸, and R⁹ is CF₃. In certain embodiments, one to three of R⁵, R⁶, R⁷, R⁸, and R⁹ is CHF₂.

In certain embodiments, one to three of R₅, R₆, R₇, R₈, and R₉ is C₁₋₆ alkyl optionally substituted with halogen or —OH. In certain embodiments, one to three of R₅, R₆, R₇, R₈, and R₉ is C₁₋₆ alkyl optionally substituted with fluorine and —OH.

In certain embodiments, one to three of R₅, R₆, R₇, R₈, and R₉ is halogen, and one to three of R₅, R₆, R₇, R₈, and R₉ is C₁₋₆ alkyl optionally substituted with halogen. In certain embodiments, one to three of R₅, R₆, R₇, R₈, and R₉ is fluorine, and one to three of R₅, R₆, R₇, R₈, and R₉ is C₁₋₆ alkyl optionally substituted with fluorine.

In certain embodiments, one to three of R⁵, R⁶, R⁷, R⁸, and R⁹ is —NH₂.

In certain embodiments, one of R₅, R₆, R₇, R₈, and R₉ is —NH₂; and one of R₅, R₆, R₇, R₈, and R₉ is C₁₋₆ alkyl optionally substituted with halogen. In certain embodiments, one of R⁵, R⁶, R⁷, R⁸, and R⁹ is —NH₂; and one of R⁵, R⁶, R⁷, R⁸, and R⁹ is CF₃.

In some embodiments, A is selected from among:

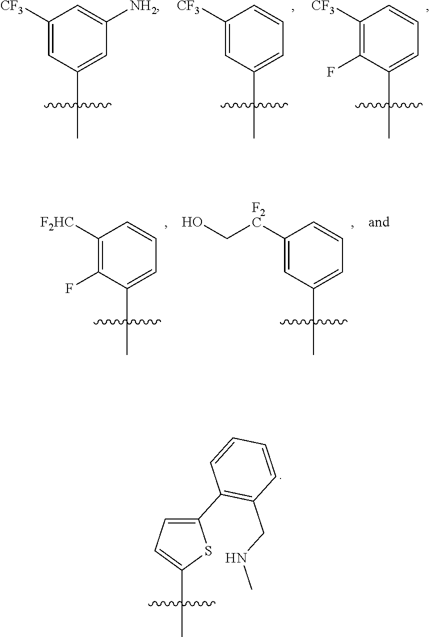

In some embodiments, the compound of Formula (I), (I-a), (II), (II-a), (III), or (III-a), or a pharmaceutically acceptable salt, solvate, stereoisomer, or tautomer thereof, has one, two, three or more of the following features:

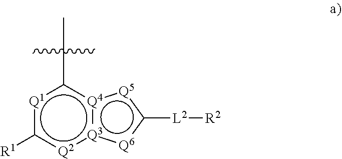

is selected from the group consisting of

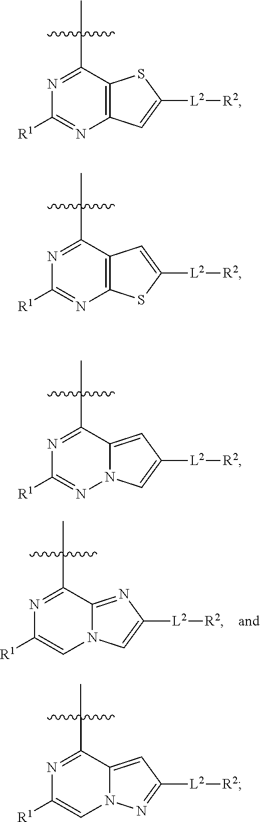

b) R³ is H and R⁴ is C₁₋₆ alkyl;
c) L² is a bond or —C(O)—; and
d) R² is optionally substituted cycloalkyl, optionally substituted cycloalkenyl, or optionally substituted heterocyclyl.

In some embodiments, the compound of formula II, or a pharmaceutically acceptable salt, solvate, stereoisomer, or tautomer thereof, has one, two, three or more of the following features:

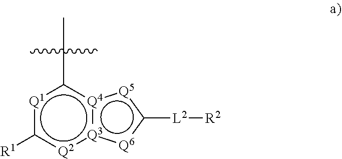

is selected from the group consisting of

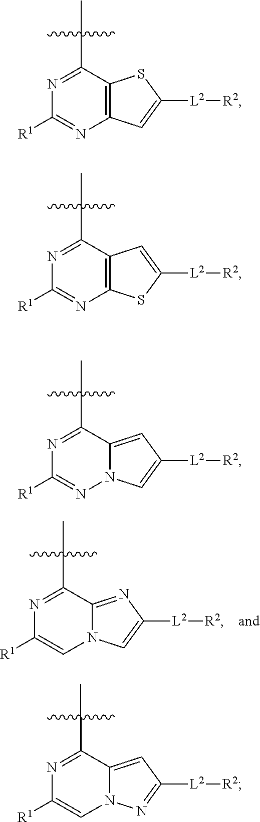

b) one to three of R⁵, R⁶, R⁷, R⁸, and R⁹ is C₁₋₆ alkyl, wherein the alkyl is optionally substituted with one or more halogen atoms;
c) R³ is H and R⁴ is C₁₋₆ alkyl;
d) L² is a bond or —C(O)—;
e) R² is optionally substituted cycloalkyl, optionally substituted cycloalkenyl, or optionally substituted heterocyclyl.

The present disclosure provides compound of formula I, or a pharmaceutically acceptable salt, solvate, isomer, prodrug, or tautomer thereof, which is

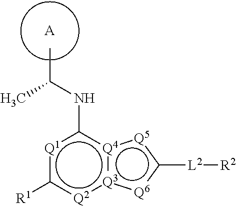

wherein A, L², Q¹, Q², Q³, Q⁴, Q⁵, Q⁶, R¹, R², m and n are as defined herein.

The present disclosure provides compound of formula II, or a pharmaceutically acceptable salt, solvate, isomer, prodrug, or tautomer thereof, which is

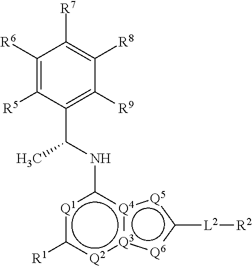

wherein L², Q¹, Q², Q³, Q⁴, Q⁵, Q⁶, R¹, R², R⁴, R⁵, R⁶, R⁷, R⁸, R⁹, m and n are as defined herein.

The present disclosure provides a compound, and pharmaceutically acceptable salts, solvates, stereoisomers, and tautomers thereof, selected from the group consisting of compounds of Table A:

TABLE A

| Example # | Structure | Example # | Structure |
|---|---|---|---|
| Example 1. | 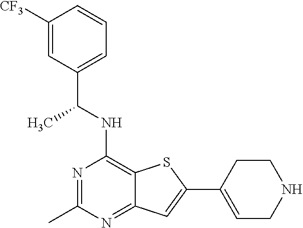 [SMILES: Cc1nc(N[C@H](C)c2cccc(C(F)(F)F)c2)c2sc(C3=CCNCC3)cc2n1] | Example 52. | 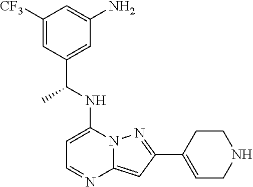 [SMILES: C[C@@H](Nc1ccnc2cc(C3=CCNCC3)nn12)c1cc(N)cc(C(F)(F)F)c1] |
| Example 2. | 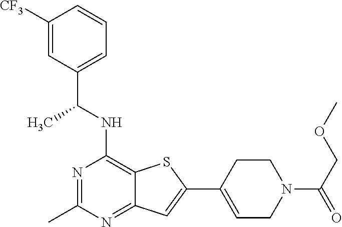 [SMILES: COCC(=O)N1CC=C(c2cc3nc(C)nc(N[C@H](C)c4cccc(C(F)(F)F)c4)c3s2)CC1] | Example 53. | 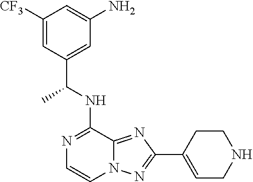 [SMILES: C[C@@H](Nc1nccn2nc(C3=CCNCC3)nc12)c1cc(N)cc(C(F)(F)F)c1] |
| Example 3. | 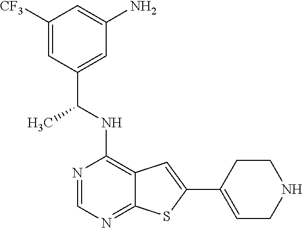 [SMILES: C[C@@H](Nc1ncnc2sc(C3=CCNCC3)cc12)c1cc(N)cc(C(F)(F)F)c1] | Example 54. | 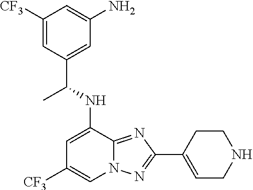 [SMILES: C[C@@H](Nc1cc(C(F)(F)F)cn2nc(C3=CCNCC3)nc12)c1cc(N)cc(C(F)(F)F)c1] |
| Example 4. | 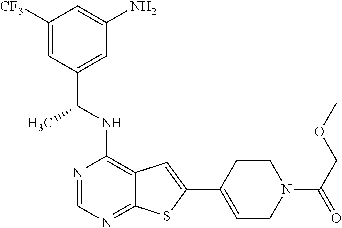 [SMILES: COCC(=O)N1CC=C(c2cc3c(N[C@H](C)c4cc(N)cc(C(F)(F)F)c4)ncnc3s2)CC1] | Example 55. | 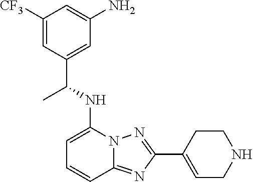 [SMILES: C[C@@H](Nc1cccc2nc(C3=CCNCC3)nn12)c1cc(N)cc(C(F)(F)F)c1] |
| Example 5. | 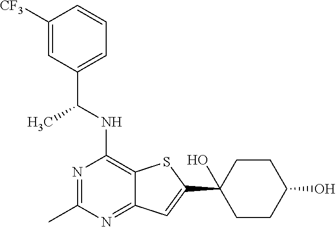 [SMILES: Cc1nc(N[C@H](C)c2cccc(C(F)(F)F)c2)c2sc([C@]3(O)CC[C@@H](O)CC3)cc2n1] | Example 56. | 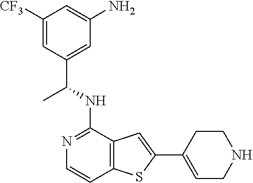 [SMILES: C[C@@H](Nc1nccc2sc(C3=CCNCC3)cc12)c1cc(N)cc(C(F)(F)F)c1] |
| Example 6. | 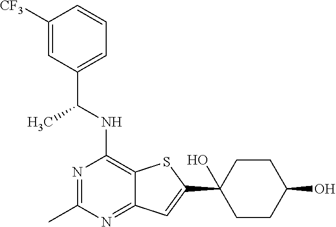 [SMILES: Cc1nc(N[C@H](C)c2cccc(C(F)(F)F)c2)c2sc([C@]3(O)CC[C@H](O)CC3)cc2n1] | Example 57. | 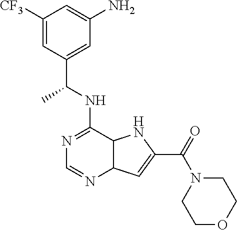 [SMILES: C[C@@H](NC1=NC=NC2C=C(C(=O)N3CCOCC3)NC12)c1cc(N)cc(C(F)(F)F)c1] |
| Example 7. | 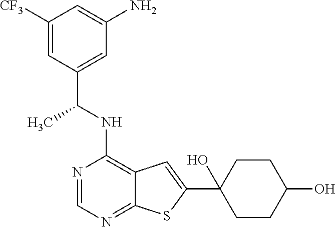 [SMILES: C[C@@H](Nc1ncnc2sc(C3(O)CCC(O)CC3)cc12)c1cc(N)cc(C(F)(F)F)c1] | Example 58., 60. | 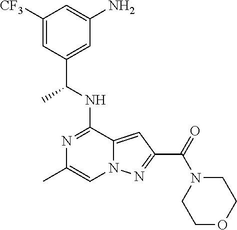 [SMILES: Cc1cn2nc(C(=O)N3CCOCC3)cc2c(N[C@H](C)c2cc(N)cc(C(F)(F)F)c2)n1] |
| Example 8. | 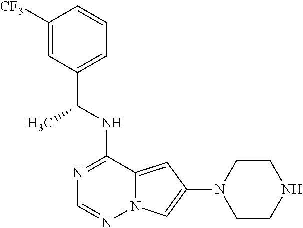 [SMILES: C[C@@H](Nc1ncnn2cc(N3CCNCC3)cc12)c1cccc(C(F)(F)F)c1] | Example 59. | 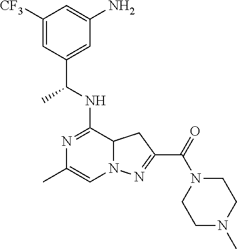 [SMILES: CC1=CN2N=C(C(=O)N3CCN(C)CC3)CC2C(N[C@H](C)c2cc(N)cc(C(F)(F)F)c2)=N1] |
| Example 9. | 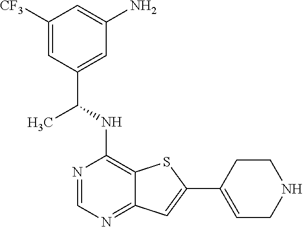 [SMILES: C[C@@H](Nc1ncnc2cc(C3=CCNCC3)sc12)c1cc(N)cc(C(F)(F)F)c1] | Example 61. | 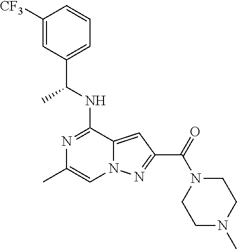 [SMILES: Cc1cn2nc(C(=O)N3CCN(C)CC3)cc2c(N[C@H](C)c2cccc(C(F)(F)F)c2)n1] |
| Example 10. | 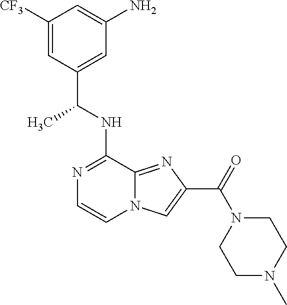 [SMILES: C[C@@H](Nc1nccn2cc(C(=O)N3CCN(C)CC3)nc12)c1cc(N)cc(C(F)(F)F)c1] | Example 62. | 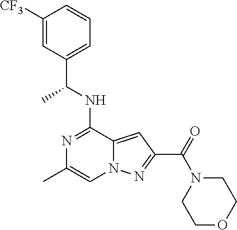 [SMILES: Cc1cn2nc(C(=O)N3CCOCC3)cc2c(N[C@H](C)c2cccc(C(F)(F)F)c2)n1] |
| Example 11. | 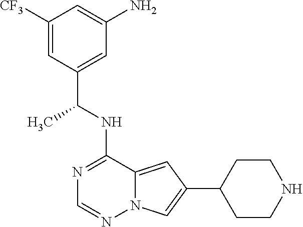 [SMILES: C[C@@H](Nc1ncnn2cc(C3CCNCC3)cc12)c1cc(N)cc(C(F)(F)F)c1] | Example 63. | 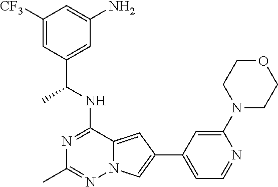 [SMILES: Cc1nc(N[C@H](C)c2cc(N)cc(C(F)(F)F)c2)c2cc(-c3ccnc(N4CCOCC4)c3)cn2n1] |
| Example 12. | 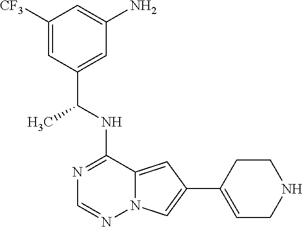 [SMILES: C[C@@H](Nc1ncnn2cc(C3=CCNCC3)cc12)c1cc(N)cc(C(F)(F)F)c1] | Example 64. | 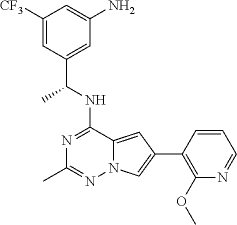 [SMILES: COc1ncccc1-c1cc2c(N[C@H](C)c3cc(N)cc(C(F)(F)F)c3)nc(C)nn2c1] |
| Example 13. | 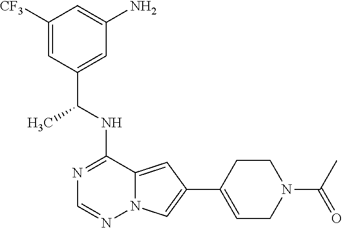 [SMILES: CC(=O)N1CC=C(c2cc3c(N[C@H](C)c4cc(N)cc(C(F)(F)F)c4)ncnn3c2)CC1] | Example 65. | 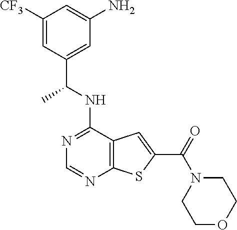 [SMILES: C[C@@H](Nc1ncnc2sc(C(=O)N3CCOCC3)cc12)c1cc(N)cc(C(F)(F)F)c1] |
| Example 14. | 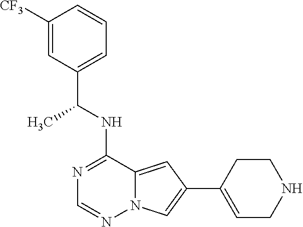 [SMILES: C[C@@H](Nc1ncnn2cc(C3=CCNCC3)cc12)c1cccc(C(F)(F)F)c1] | Example 66. | 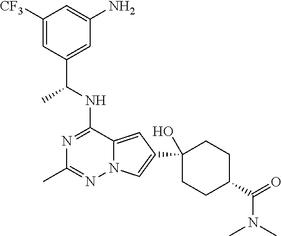 [SMILES: Cc1nc(N[C@H](C)c2cc(N)cc(C(F)(F)F)c2)c2cc([C@]3(O)CC[C@H](C(=O)N(C)C)CC3)cn2n1] |
| Example 15. | 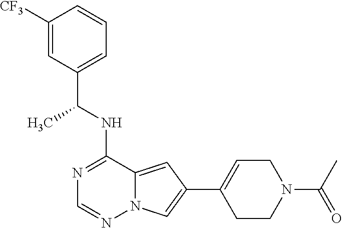 [SMILES: CC(=O)N1CC=C(c2cc3c(N[C@H](C)c4cccc(C(F)(F)F)c4)ncnn3c2)CC1] | Example 67. | 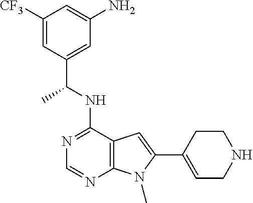 [SMILES: C[C@@H](Nc1ncnc2c1cc(C1=CCNCC1)n2C)c1cc(N)cc(C(F)(F)F)c1] |
| Example 16. | 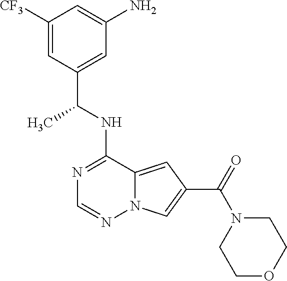 [SMILES: C[C@@H](Nc1ncnn2cc(C(=O)N3CCOCC3)cc12)c1cc(N)cc(C(F)(F)F)c1] | Example 68. | 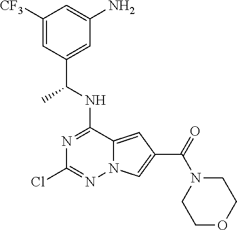 [SMILES: C[C@@H](Nc1nc(Cl)nn2cc(C(=O)N3CCOCC3)cc12)c1cc(N)cc(C(F)(F)F)c1] |
| Example 17. | 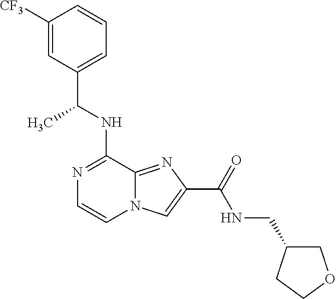 [SMILES: C[C@@H](Nc1nccn2cc(C(=O)NC[C@H]3CCOC3)nc12)c1cccc(C(F)(F)F)c1] | Example 69. | 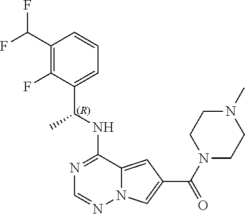 |
| Example 18. | 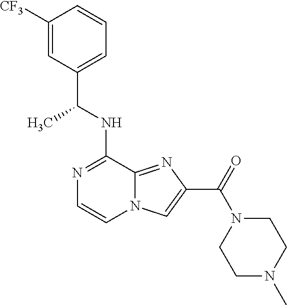 [SMILES: C[C@@H](Nc1nccn2cc(C(=O)N3CCN(C)CC3)nc12)c1cccc(C(F)(F)F)c1] | Example 70. | 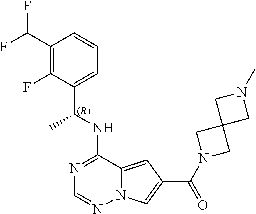 |
| Example 19. | 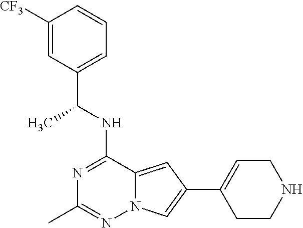 [SMILES: Cc1nc(N[C@H](C)c2cccc(C(F)(F)F)c2)c2cc(C3=CCNCC3)cn2n1] | Example 71. | 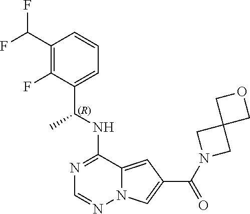 |
| Example 20. | 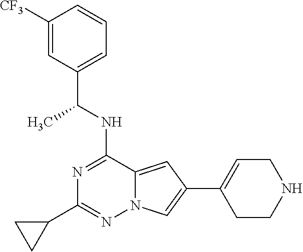 [SMILES: C[C@@H](Nc1nc(C2CC2)nn2cc(C3=CCNCC3)cc12)c1cccc(C(F)(F)F)c1] | Example 72. | 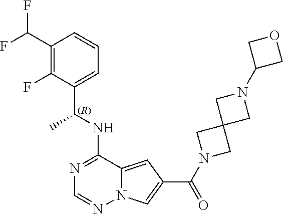 |
| Example 21. | 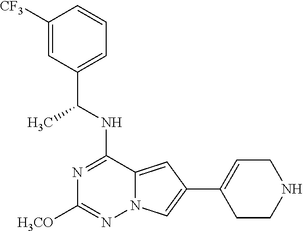 [SMILES: COc1nc(N[C@H](C)c2cccc(C(F)(F)F)c2)c2cc(C3=CCNCC3)cn2n1] | Example 73. | 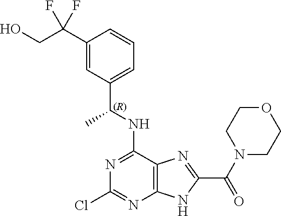 |
| Example 22. | 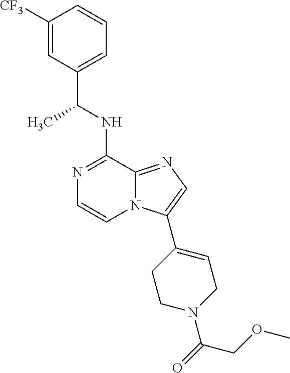 [SMILES: COCC(=O)N1CC=C(c2cnc3c(N[C@H](C)c4cccc(C(F)(F)F)c4)nccn23)CC1] | Example 74. | 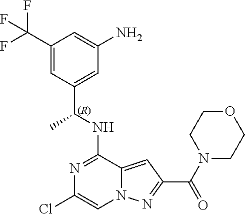 |
| Example 23. | 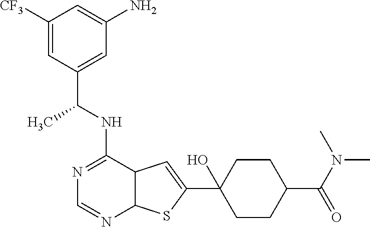 [SMILES: C[C@@H](NC1=NC=NC2SC(C3(O)CCC(C(=O)N(C)C)CC3)=CC12)c1cc(N)cc(C(F)(F)F)c1] | Example 75. | 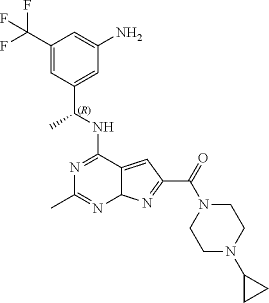 |
| Example 24. | 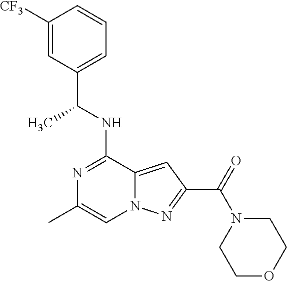 [SMILES: Cc1cn2nc(C(=O)N3CCOCC3)cc2c(N[C@H](C)c2cccc(C(F)(F)F)c2)n1] | Example 76. | 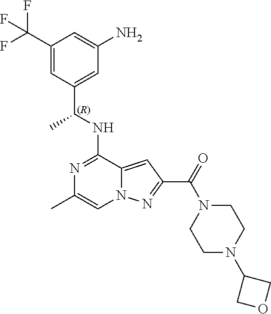 |
| Example 25. | 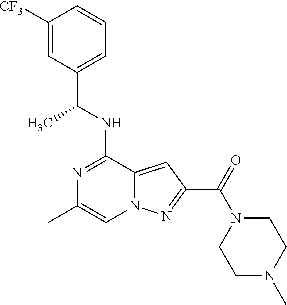 [SMILES: Cc1cn2nc(C(=O)N3CCN(C)CC3)cc2c(N[C@H](C)c2cccc(C(F)(F)F)c2)n1] | Example 77. | 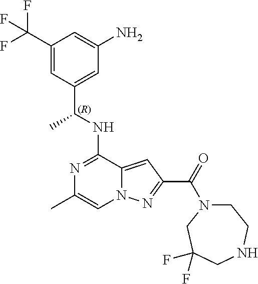 |
| Example 26. | 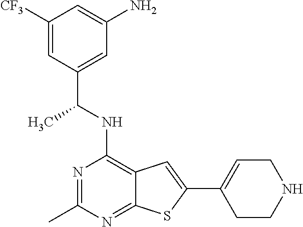 [SMILES: Cc1nc(N[C@H](C)c2cc(N)cc(C(F)(F)F)c2)c2cc(C3=CCNCC3)sc2n1] | Example 78. | 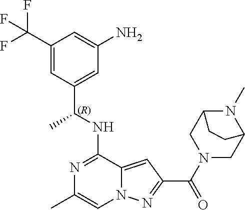 |
| Example 27. | 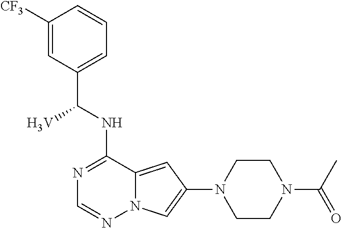 | Example 79. | 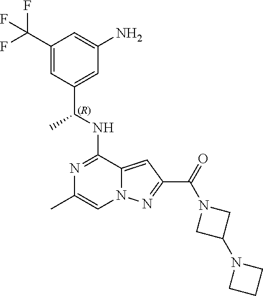 |
| Example 28. | 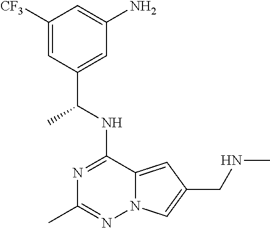 [SMILES: CNCc1cc2c(N[C@H](C)c3cc(N)cc(C(F)(F)F)c3)nc(C)nn2c1] | Example 80. | 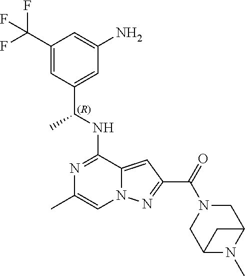 |
| Example 29. | 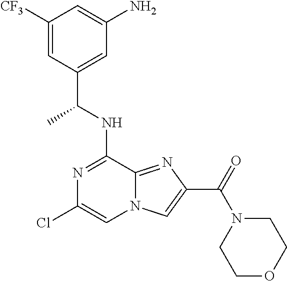 [SMILES: C[C@@H](Nc1nc(Cl)cn2cc(C(=O)N3CCOCC3)nc12)c1cc(N)cc(C(F)(F)F)c1] | Example 81. | 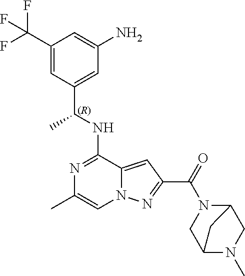 |
| Example 30. | 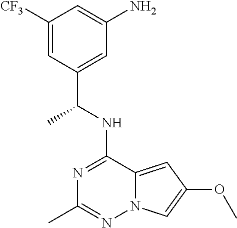 [SMILES: COc1cc2c(N[C@H](C)c3cc(N)cc(C(F)(F)F)c3)nc(C)nn2c1] | Example 82. | 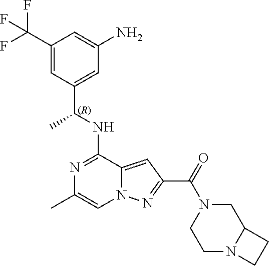 |
| Example 31. | 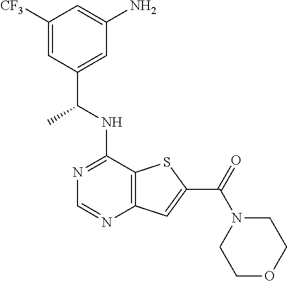 [SMILES: C[C@@H](Nc1ncnc2cc(C(=O)N3CCOCC3)sc12)c1cc(N)cc(C(F)(F)F)c1] | Example 83. | 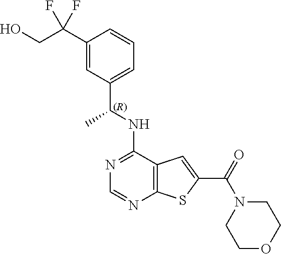 |
| Example 32. | 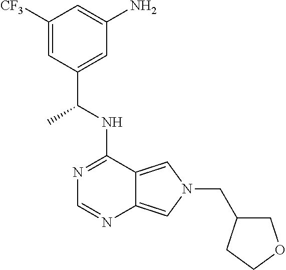 [SMILES: C[C@@H](Nc1ncnc2cn(CC3CCOC3)cc12)c1cc(N)cc(C(F)(F)F)c1] | Example 84. | 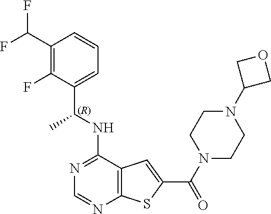 |
| Example 33. | 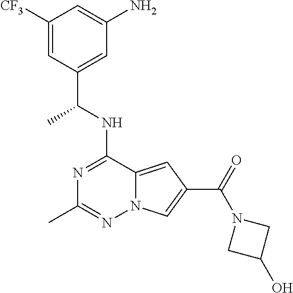 [SMILES: Cc1nc(N[C@H](C)c2cc(N)cc(C(F)(F)F)c2)c2cc(C(=O)N3CC(O)C3)cn2n1] | Example 85. | 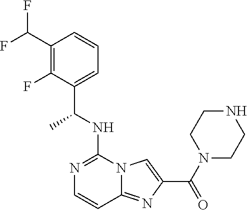 [SMILES: C[C@@H](Nc1nccc2nc(C(=O)N3CCNCC3)cn12)c1cccc(C(F)F)c1F] |
| Example 34 | 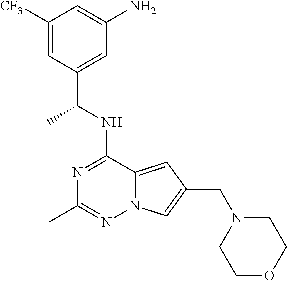 [SMILES: Cc1nc(N[C@H](C)c2cc(N)cc(C(F)(F)F)c2)c2cc(CN3CCOCC3)cn2n1] | Example 86. | 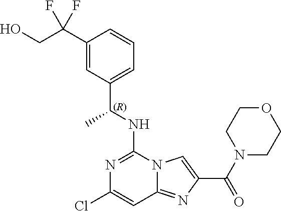 |
| Example 35. | 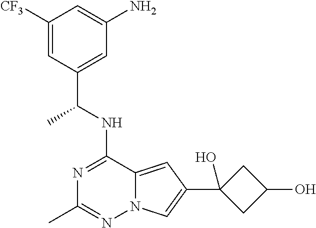 [SMILES: Cc1nc(N[C@H](C)c2cc(N)cc(C(F)(F)F)c2)c2cc(C3(O)CC(O)C3)cn2n1] | Example 87. | 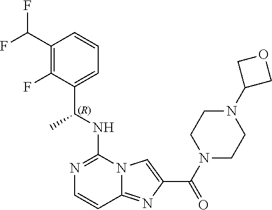 |
| Example 36. | 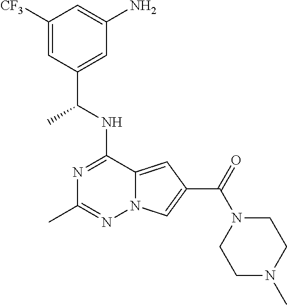 [SMILES: Cc1nc(N[C@H](C)c2cc(N)cc(C(F)(F)F)c2)c2cc(C(=O)N3CCN(C)CC3)cn2n1] | Example 88. | 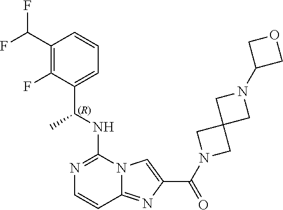 |
| Example 37. | 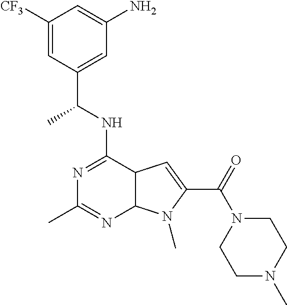 [SMILES: CC1=NC2C(C=C(C(=O)N3CCN(C)CC3)N2C)C(N[C@H](C)c2cc(N)cc(C(F)(F)F)c2)=N1] | Example 89. | 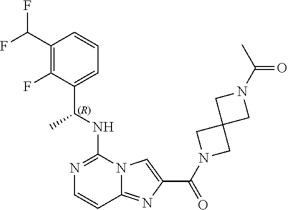 |
| Example 38. | 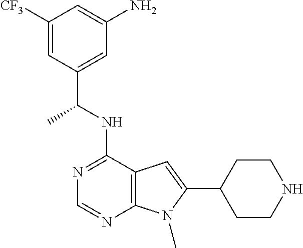 [SMILES: C[C@@H](Nc1ncnc2c1cc(C1CCNCC1)n2C)c1cc(N)cc(C(F)(F)F)c1] | Example 90. | 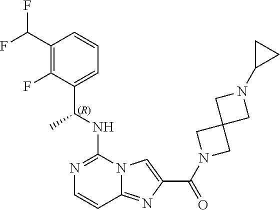 |
| Example 39. | 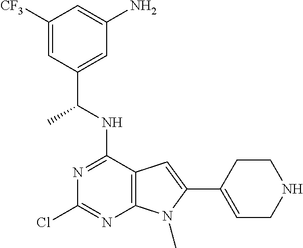 [SMILES: C[C@@H](Nc1nc(Cl)nc2c1cc(C1=CCNCC1)n2C)c1cc(N)cc(C(F)(F)F)c1] | Example 91. | 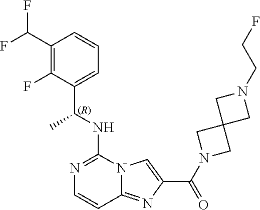 |
| Example 40. | 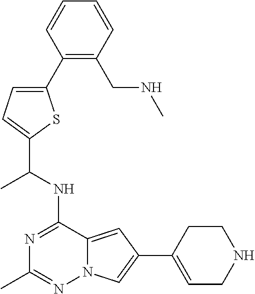 [SMILES: CNCc1ccccc1-c1ccc(C(C)Nc2nc(C)nn3cc(C4=CCNCC4)cc23)s1] | Example 92. | 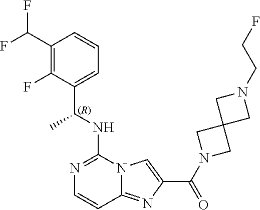 |
| Example 41. | 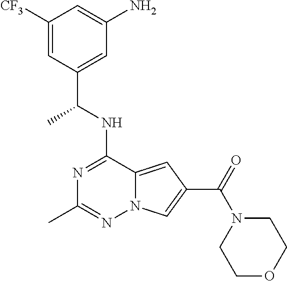 [SMILES: Cc1nc(N[C@H](C)c2cc(N)cc(C(F)(F)F)c2)c2cc(C(=O)N3CCOCC3)cn2n1] | Example 93. | 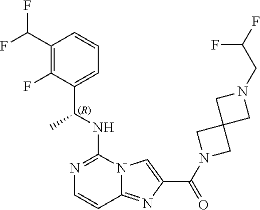 |
| Example 42. | 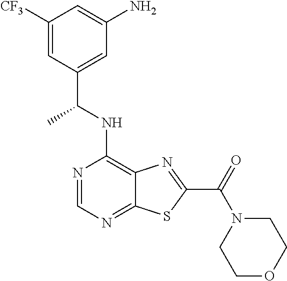 [SMILES: C[C@@H](Nc1ncnc2sc(C(=O)N3CCOCC3)nc12)c1cc(N)cc(C(F)(F)F)c1] | Example 94. | 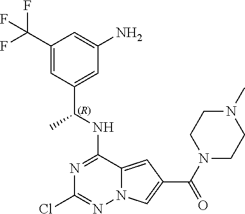 |
| Example 43. | 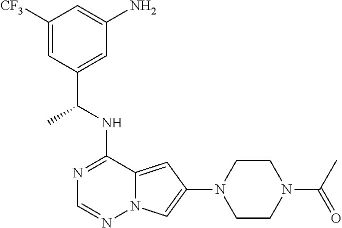 [SMILES: CC(=O)N1CCN(c2cc3c(N[C@H](C)c4cc(N)cc(C(F)(F)F)c4)ncnn3c2)CC1] | Example 95. | 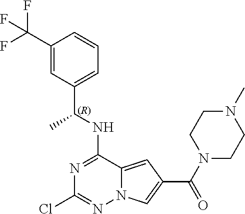 |
| Example 44. | 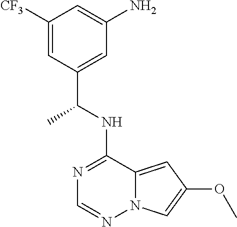 [SMILES: COc1cc2c(N[C@H](C)c3cc(N)cc(C(F)(F)F)c3)ncnn2c1] | Example 96. | 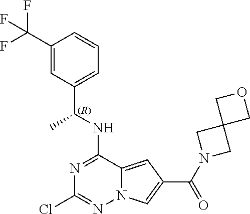 |
| Example 45. | 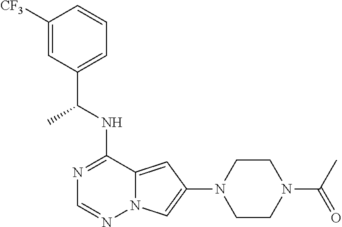 [SMILES: CC(=O)N1CCN(c2cc3c(N[C@H](C)c4cccc(C(F)(F)F)c4)ncnn3c2)CC1] | Example 97. | 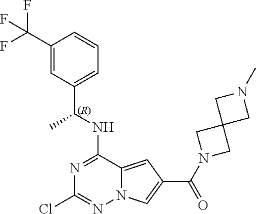 |
| Example 46. | 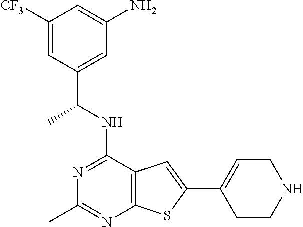 [SMILES: Cc1nc(N[C@H](C)c2cc(N)cc(C(F)(F)F)c2)c2cc(C3=CCNCC3)sc2n1] | Example 98. | 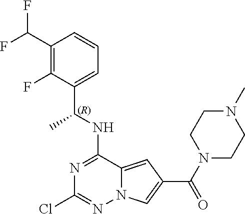 |
| Example 47. | 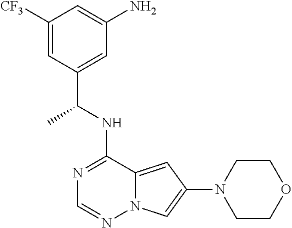 [SMILES: C[C@@H](Nc1ncnn2cc(N3CCOCC3)cc12)c1cc(N)cc(C(F)(F)F)c1] | Example 99. | 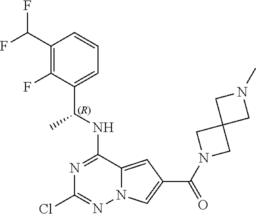 |
| Example 48. | 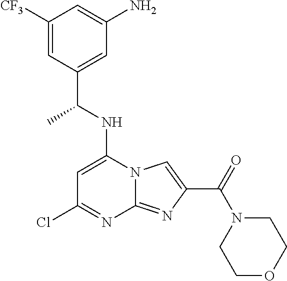 [SMILES: C[C@@H](Nc1cc(Cl)nc2nc(C(=O)N3CCOCC3)cn12)c1cc(N)cc(C(F)(F)F)c1] | Example 100. | 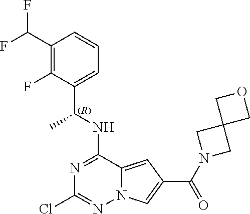 |
| Example 49. | 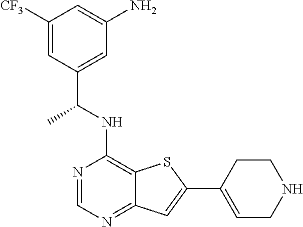 [SMILES: C[C@@H](Nc1ncnc2cc(C3=CCNCC3)sc12)c1cc(N)cc(C(F)(F)F)c1] | Example 101. | 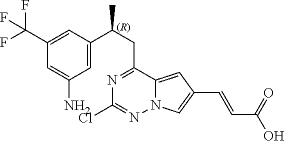 |
| Example 50. | 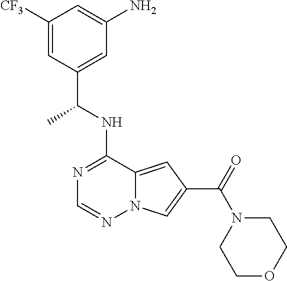 [SMILES: C[C@@H](Nc1ncnn2cc(C(=O)N3CCOCC3)cc12)c1cc(N)cc(C(F)(F)F)c1] | Example 102. | 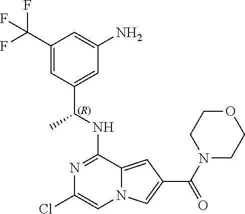 |
| Example 51. | 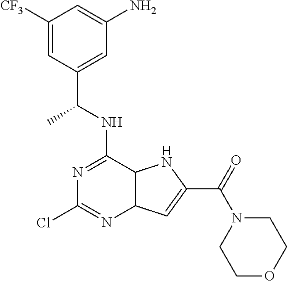 [SMILES: C[C@@H](NC1=NC(Cl)=NC2C=C(C(=O)N3CCOCC3)NC12)c1cc(N)cc(C(F)(F)F)c1] | Example 103. | 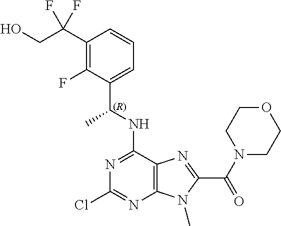 |

The present disclosure provides a compound, and pharmaceutically acceptable salts, solvates, stereoisomers, and tautomers thereof, selected from the group consisting of compounds of Collection 1:

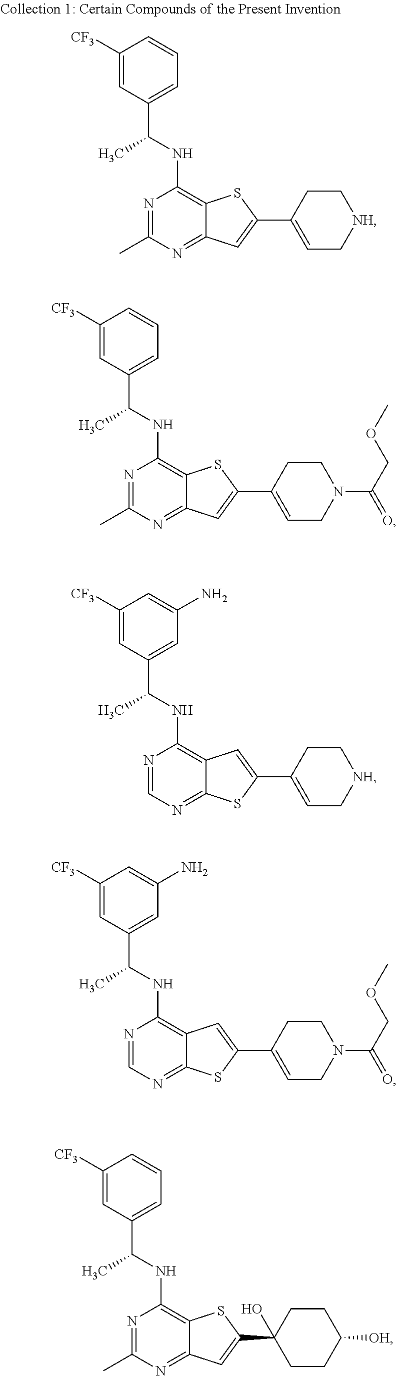 [SMILES: Cc1nc(N[C@H](C)c2cccc(C(F)(F)F)c2)c2sc(C3=CCNCC3)cc2n1 ; COCC(=O)N1CC=C(c2cc3nc(C)nc(N[C@H](C)c4cccc(C(F)(F)F)c4)c3s2)CC1 ; C[C@@H](Nc1ncnc2sc(C3=CCNCC3)cc12)c1cc(N)cc(C(F)(F)F)c1 ; COCC(=O)N1CC=C(c2cc3c(N[C@H](C)c4cc(N)cc(C(F)(F)F)c4)ncnc3s2)CC1 ; Cc1nc(N[C@H](C)c2cccc(C(F)(F)F)c2)c2sc([C@]3(O)CC[C@@H](O)CC3)cc2n1]

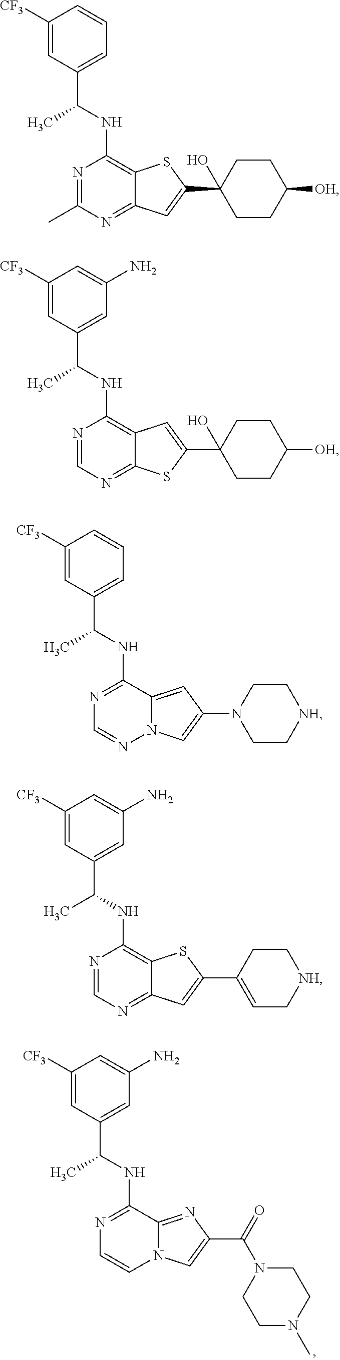 [SMILES: Cc1nc(N[C@H](C)c2cccc(C(F)(F)F)c2)c2sc([C@]3(O)CC[C@H](O)CC3)cc2n1 ; C[C@@H](Nc1ncnc2sc(C3(O)CCC(O)CC3)cc12)c1cc(N)cc(C(F)(F)F)c1 ; C[C@@H](Nc1ncnn2cc(N3CCNCC3)cc12)c1cccc(C(F)(F)F)c1 ; C[C@@H](Nc1ncnc2cc(C3=CCNCC3)sc12)c1cc(N)cc(C(F)(F)F)c1 ; C[C@@H](Nc1nccn2cc(C(=O)N3CCN(C)CC3)nc12)c1cc(N)cc(C(F)(F)F)c1]

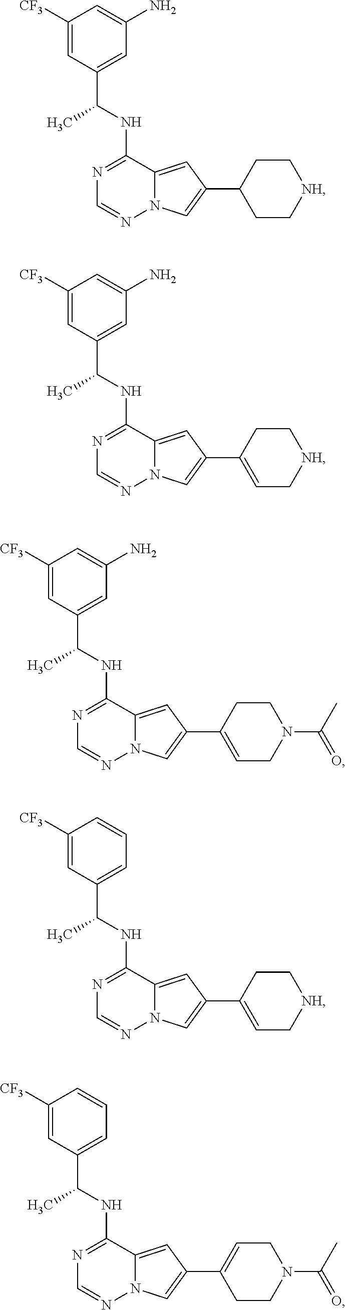 [SMILES: C[C@@H](Nc1ncnn2cc(C3CCNCC3)cc12)c1cc(N)cc(C(F)(F)F)c1 ; C[C@@H](Nc1ncnn2cc(C3=CCNCC3)cc12)c1cc(N)cc(C(F)(F)F)c1 ; CC(=O)N1CC=C(c2cc3c(N[C@H](C)c4cc(N)cc(C(F)(F)F)c4)ncnn3c2)CC1 ; C[C@@H](Nc1ncnn2cc(C3=CCNCC3)cc12)c1cccc(C(F)(F)F)c1 ; CC(=O)N1CC=C(c2cc3c(N[C@H](C)c4cccc(C(F)(F)F)c4)ncnn3c2)CC1]

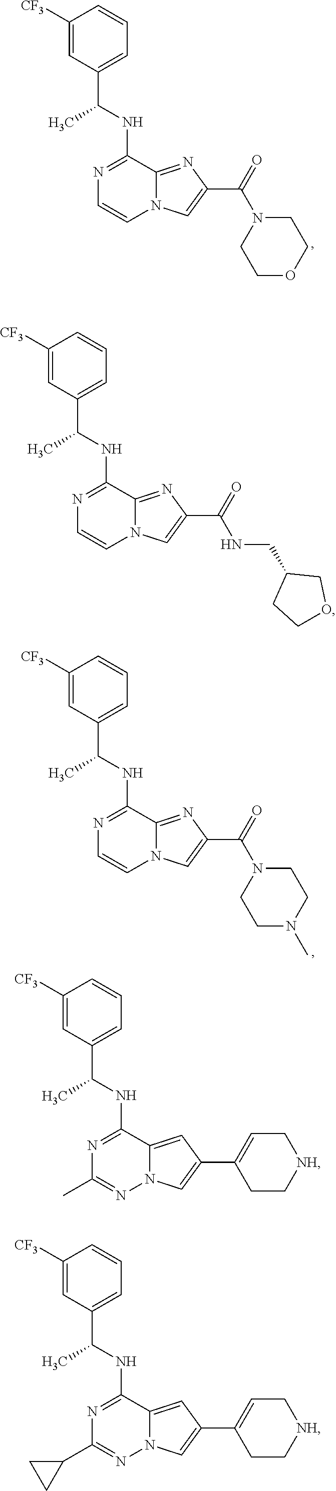 [SMILES: C[C@@H](Nc1nccn2cc(C(=O)N3CCOCC3)nc12)c1cccc(C(F)(F)F)c1 ; C[C@@H](Nc1nccn2cc(C(=O)NC[C@H]3CCOC3)nc12)c1cccc(C(F)(F)F)c1 ; C[C@@H](Nc1nccn2cc(C(=O)N3CCN(C)CC3)nc12)c1cccc(C(F)(F)F)c1 ; Cc1nc(N[C@H](C)c2cccc(C(F)(F)F)c2)c2cc(C3=CCNCC3)cn2n1 ; C[C@@H](Nc1nc(C2CC2)nn2cc(C3=CCNCC3)cc12)c1cccc(C(F)(F)F)c1]

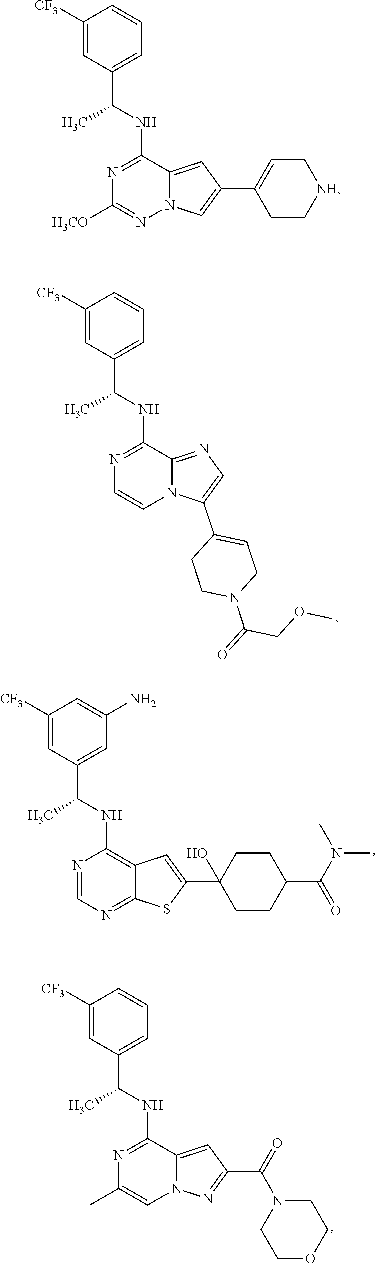 [SMILES: COc1nc(N[C@H](C)c2cccc(C(F)(F)F)c2)c2cc(C3=CCNCC3)cn2n1 ; COCC(=O)N1CC=C(c2cnc3c(N[C@H](C)c4cccc(C(F)(F)F)c4)nccn23)CC1 ; C[C@@H](Nc1ncnc2sc(C3(O)CCC(C(=O)N(C)C)CC3)cc12)c1cc(N)cc(C(F)(F)F)c1 ; Cc1cn2nc(C(=O)N3CCOCC3)cc2c(N[C@H](C)c2cccc(C(F)(F)F)c2)n1]

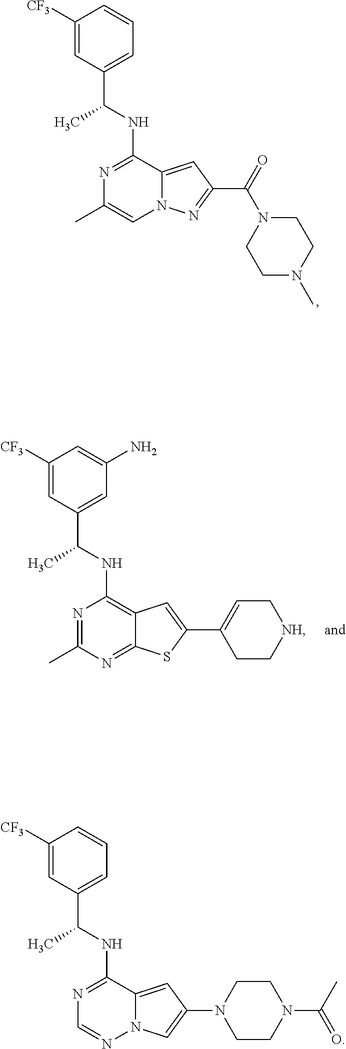

The present disclosure provides a compound, and pharmaceutically acceptable salts, solvates, stereoisomers, and tautomers thereof, selected from the group consisting of compounds of Collection 2:

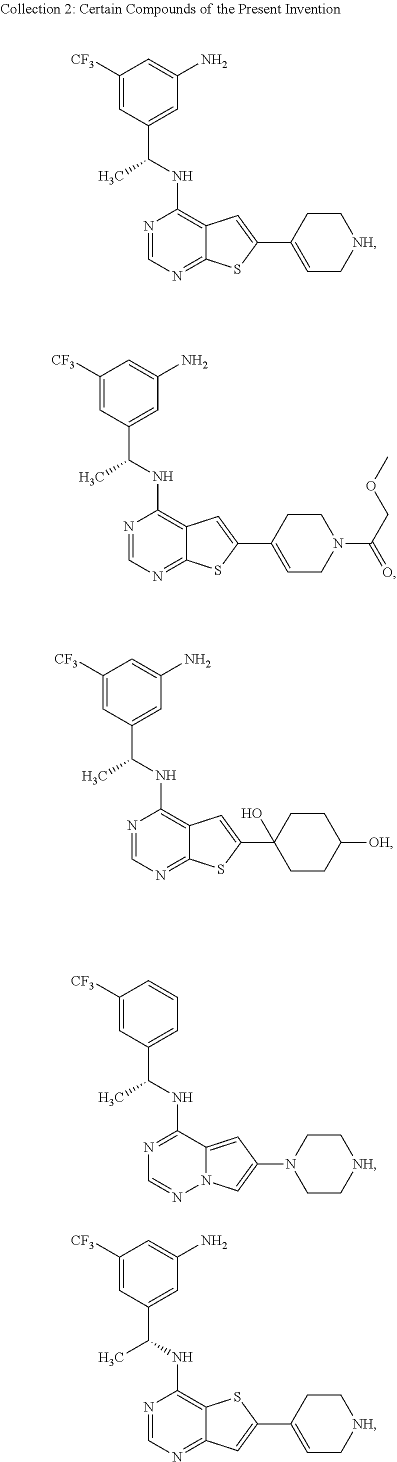 [SMILES: C[C@@H](Nc1ncnc2sc(C3=CCNCC3)cc12)c1cc(N)cc(C(F)(F)F)c1 ; COCC(=O)N1CC=C(c2cc3c(N[C@H](C)c4cc(N)cc(C(F)(F)F)c4)ncnc3s2)CC1 ; C[C@@H](Nc1ncnc2sc(C3(O)CCC(O)CC3)cc12)c1cc(N)cc(C(F)(F)F)c1 ; C[C@@H](Nc1ncnn2cc(N3CCNCC3)cc12)c1cccc(C(F)(F)F)c1 ; C[C@@H](Nc1ncnc2cc(C3=CCNCC3)sc12)c1cc(N)cc(C(F)(F)F)c1]

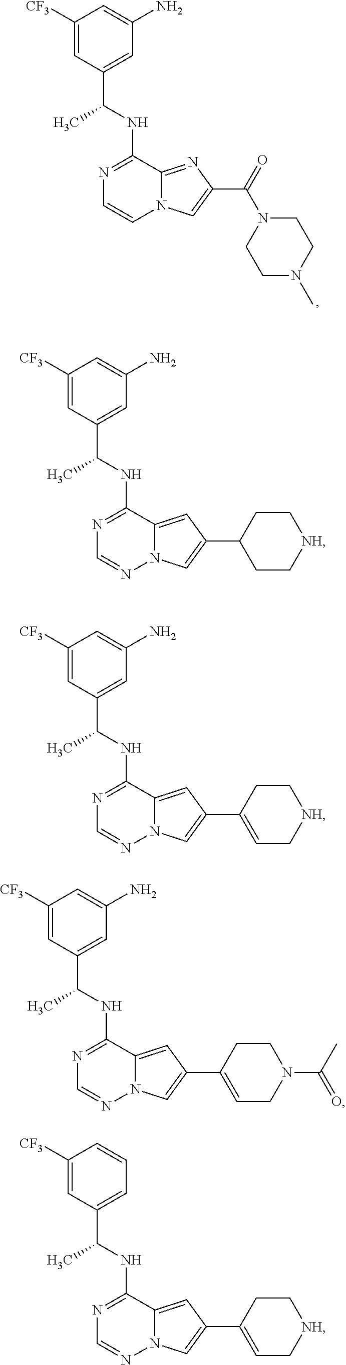

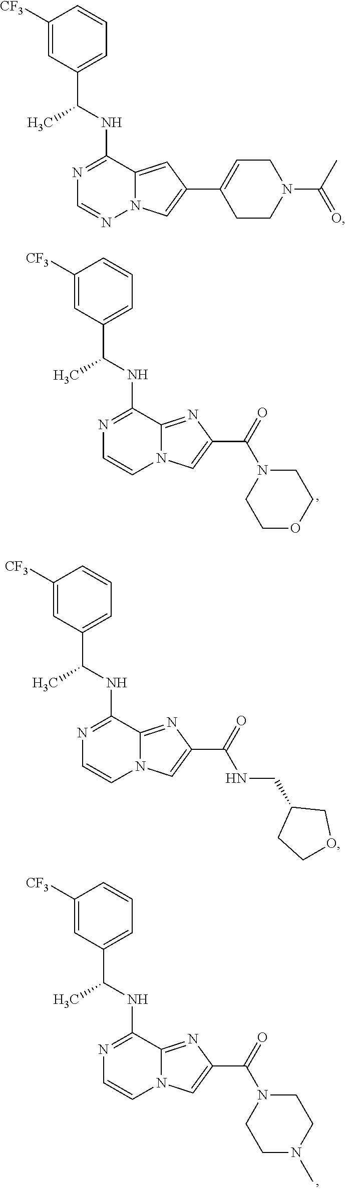

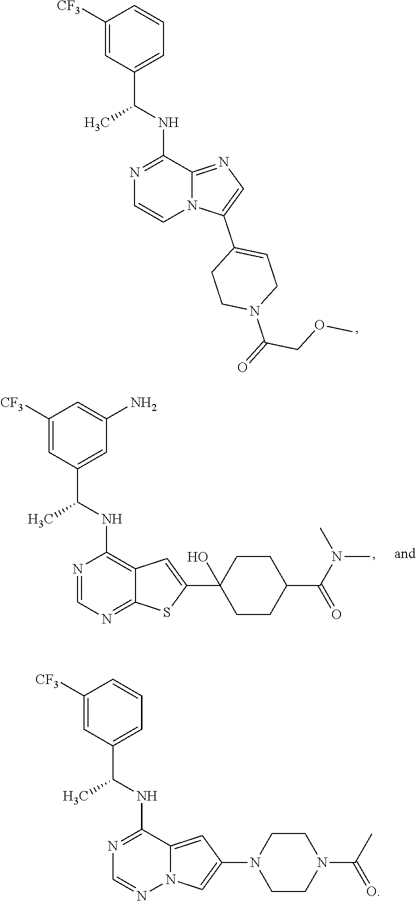 [SMILES: COCC(=O)N1CC=C(c2cnc3c(N[C@H](C)c4cccc(C(F)(F)F)c4)nccn23)CC1 ; C[C@@H](Nc1ncnc2sc(C3(O)CCC(C(=O)N(C)C)CC3)cc12)c1cc(N)cc(C(F)(F)F)c1 ; CC(=O)N1CCN(c2cc3c(N[C@H](C)c4cccc(C(F)(F)F)c4)ncnn3c2)CC1]

The present disclosure provides a compound, and pharmaceutically acceptable salts, solvates, stereoisomers, and tautomers thereof, selected from the group consisting of compounds of Collection 3:

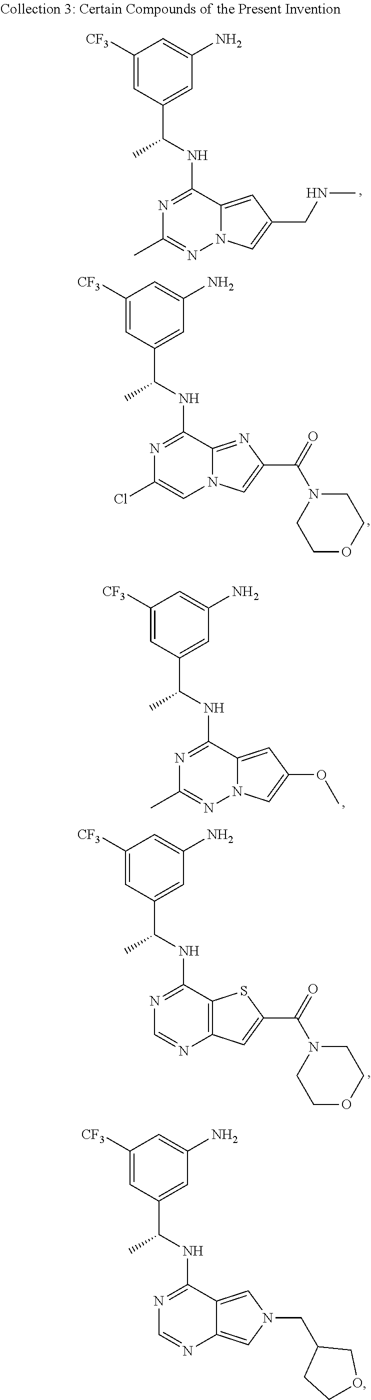

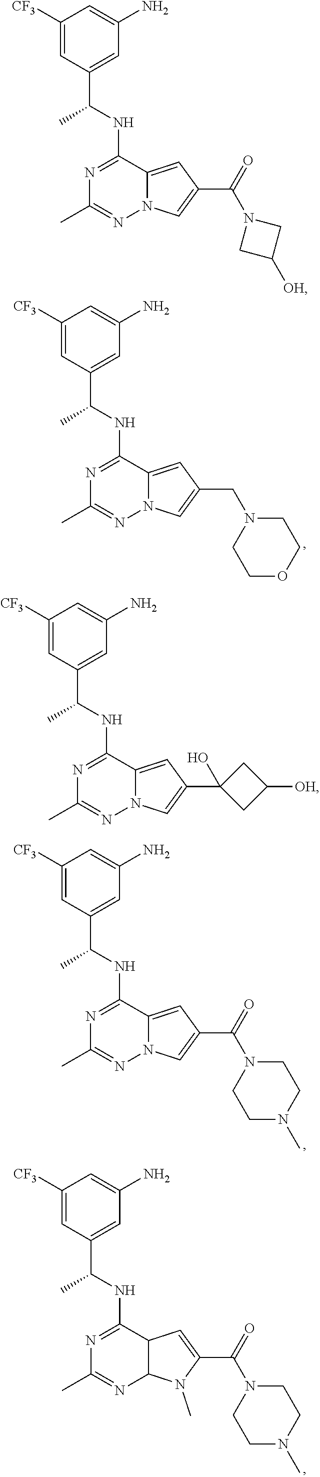

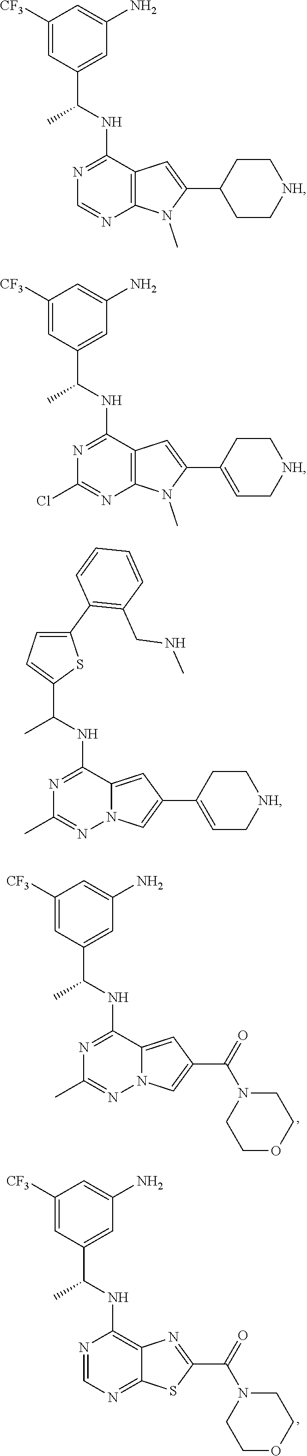 [SMILES: C[C@@H](Nc1ncnc2c1cc(C1CCNCC1)n2C)c1cc(N)cc(C(F)(F)F)c1 ; C[C@@H](Nc1nc(Cl)nc2c1cc(C1=CCNCC1)n2C)c1cc(N)cc(C(F)(F)F)c1 ; CNCc1ccccc1-c1ccc(C(C)Nc2nc(C)nn3cc(C4=CCNCC4)cc23)s1 ; Cc1nc(N[C@H](C)c2cc(N)cc(C(F)(F)F)c2)c2cc(C(=O)N3CCOCC3)cn2n1 ; C[C@@H](Nc1ncnc2sc(C(=O)N3CCOCC3)nc12)c1cc(N)cc(C(F)(F)F)c1]

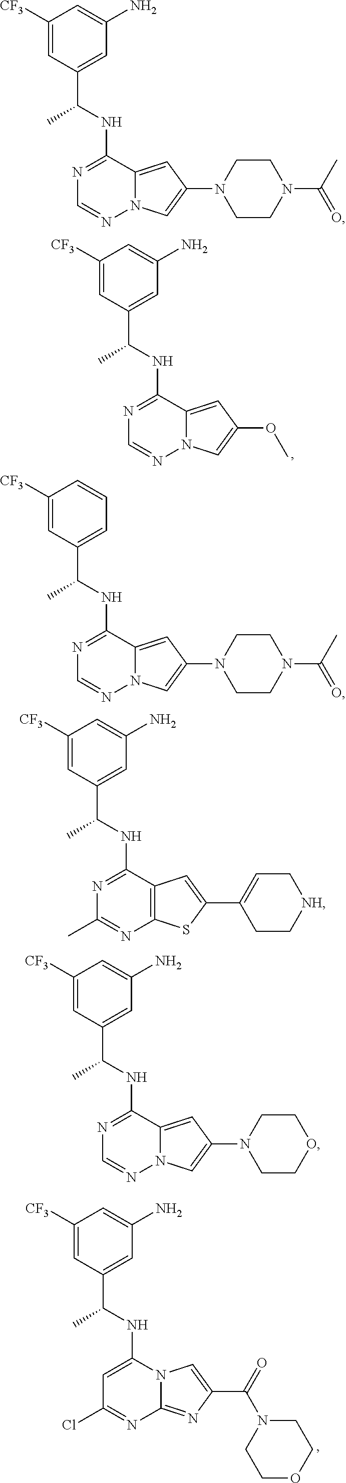

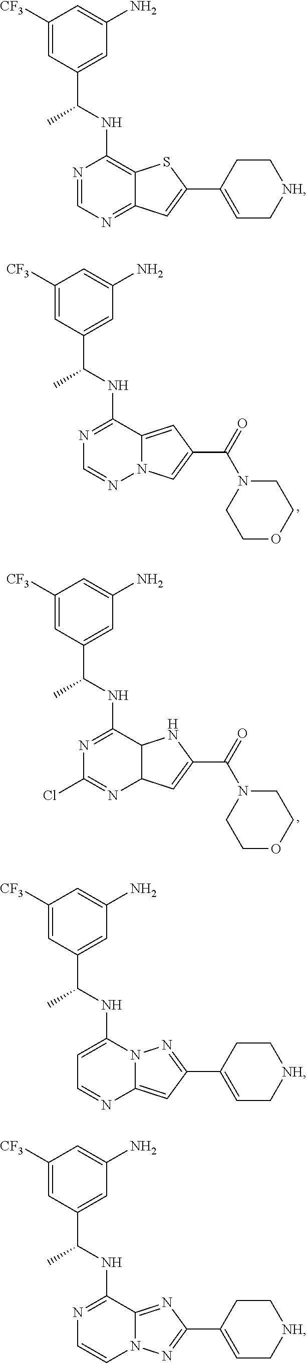 [SMILES: C[C@@H](Nc1ncnc2cc(C3=CCNCC3)sc12)c1cc(N)cc(C(F)(F)F)c1 ; C[C@@H](Nc1ncnn2cc(C(=O)N3CCOCC3)cc12)c1cc(N)cc(C(F)(F)F)c1 ; C[C@@H](NC1=NC(Cl)=NC2C=C(C(=O)N3CCOCC3)NC12)c1cc(N)cc(C(F)(F)F)c1 ; C[C@@H](Nc1ccnc2cc(C3=CCNCC3)nn12)c1cc(N)cc(C(F)(F)F)c1 ; C[C@@H](Nc1nccn2nc(C3=CCNCC3)nc12)c1cc(N)cc(C(F)(F)F)c1]

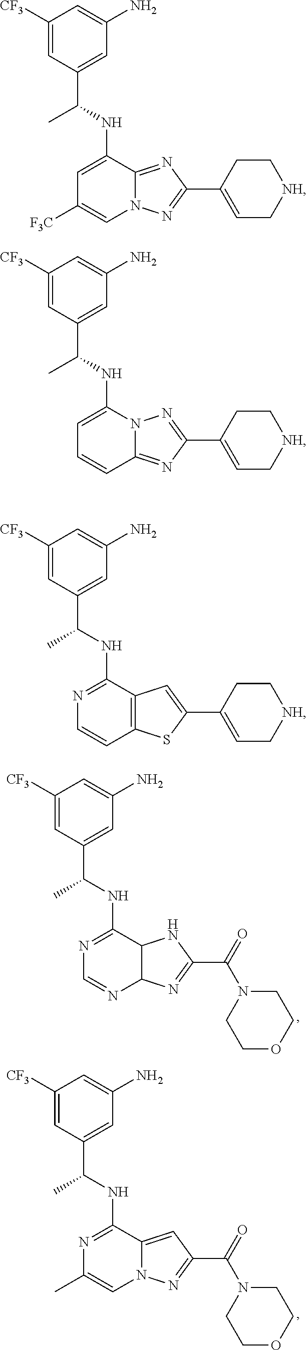

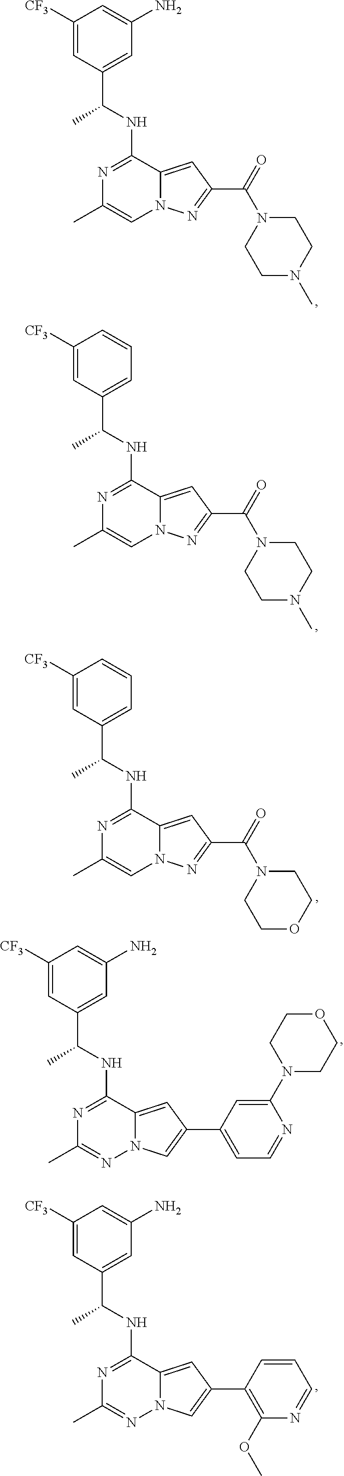

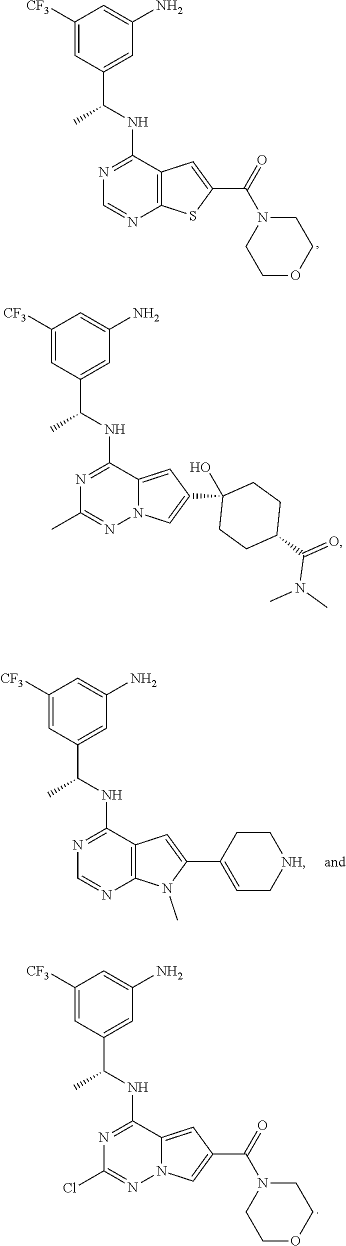

The present disclosure provides a compound, and pharmaceutically acceptable salts, solvates, stereoisomers, and tautomers thereof, selected from the group consisting of compounds of Collection 4:

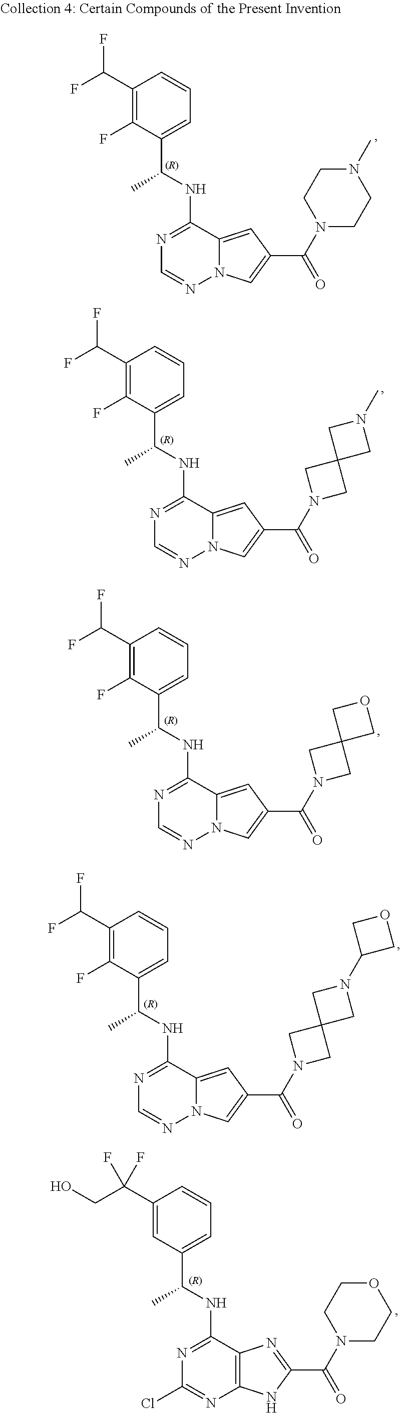

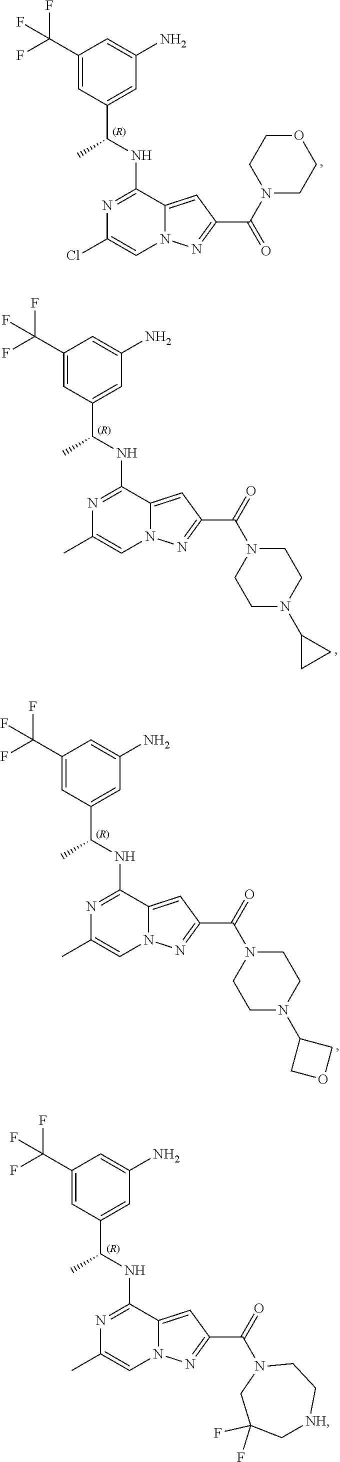

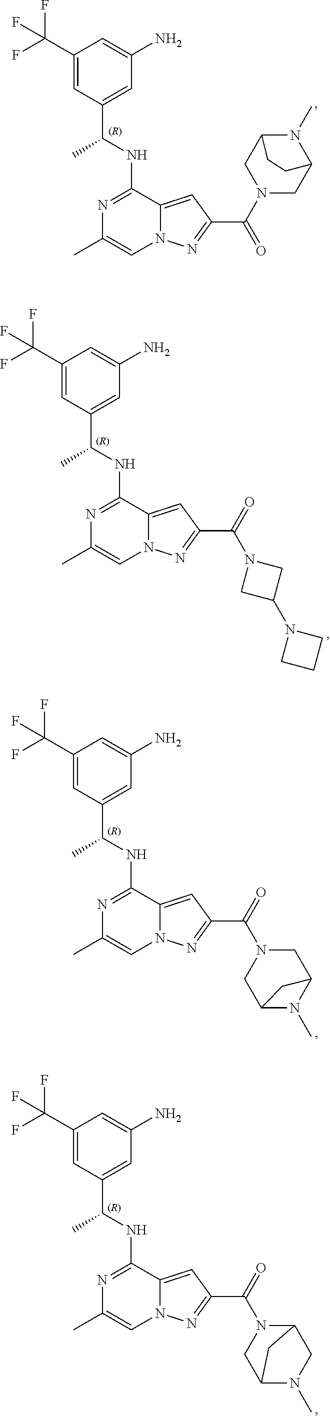

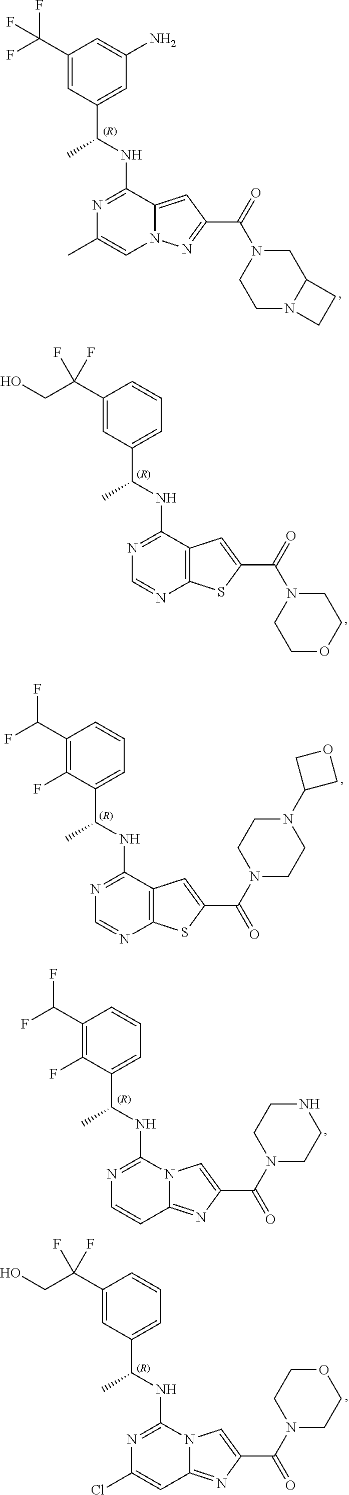

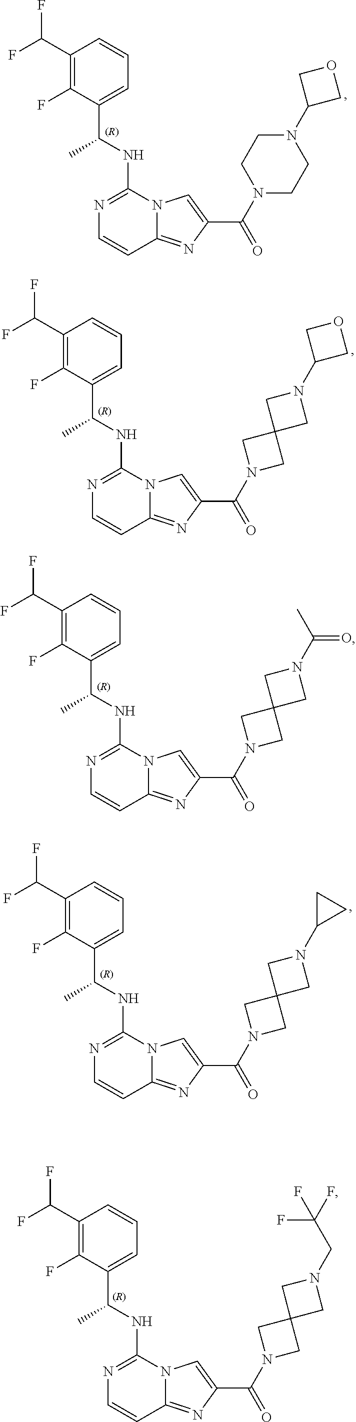

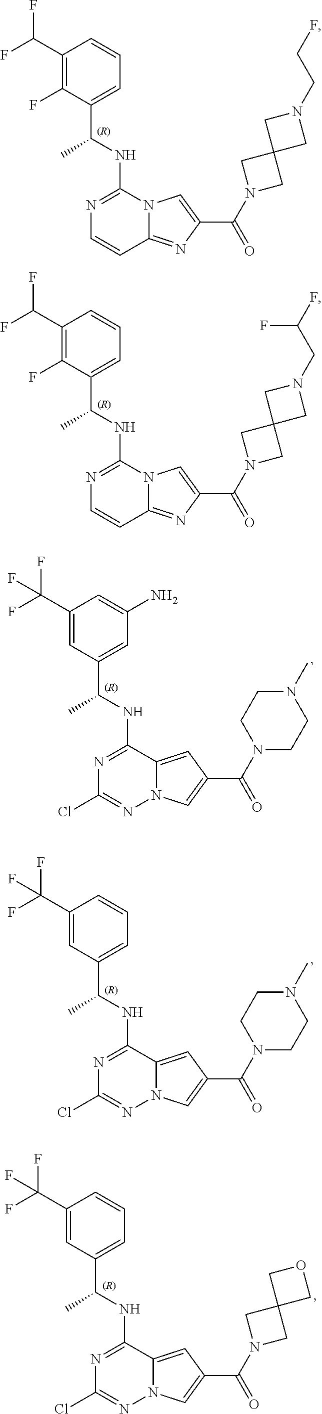

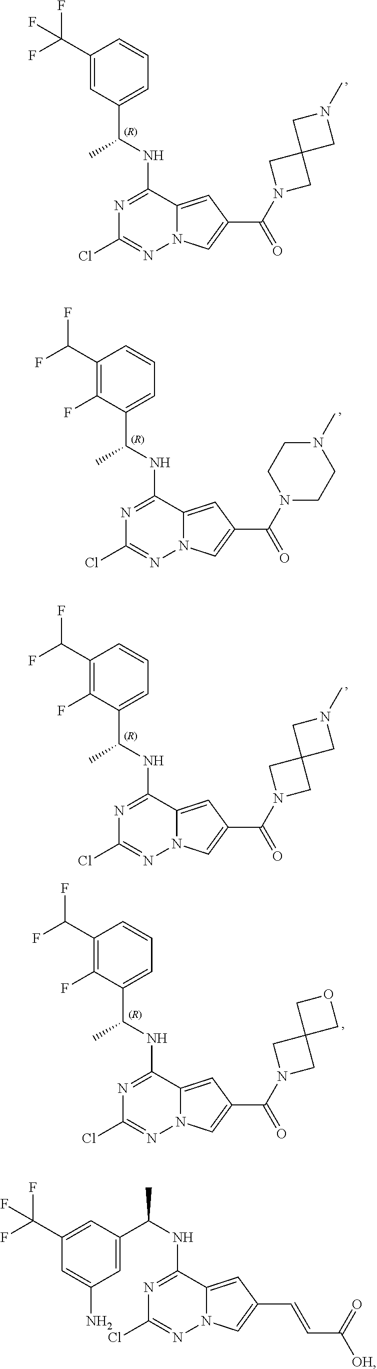

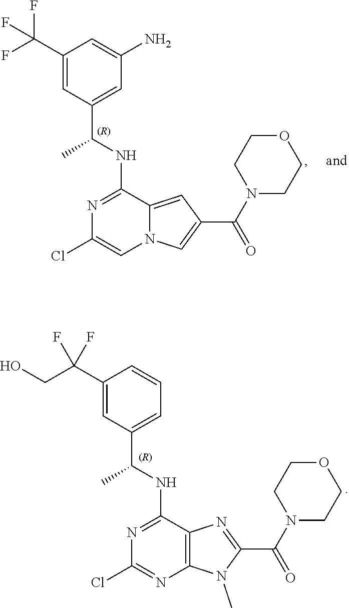

### Methods of Synthesizing the Disclosed Compounds

The compounds of the present invention may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of any Formula disclosed herein.

Those skilled in the art will recognize if a stereocenter exists in any of the compounds of the present disclosure. Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

### Preparation of Compounds

The compounds described herein may be made from commercially available starting materials or synthesized using known organic, inorganic, and/or enzymatic processes.

The compounds of the present invention can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods include but are not limited to those methods described below.

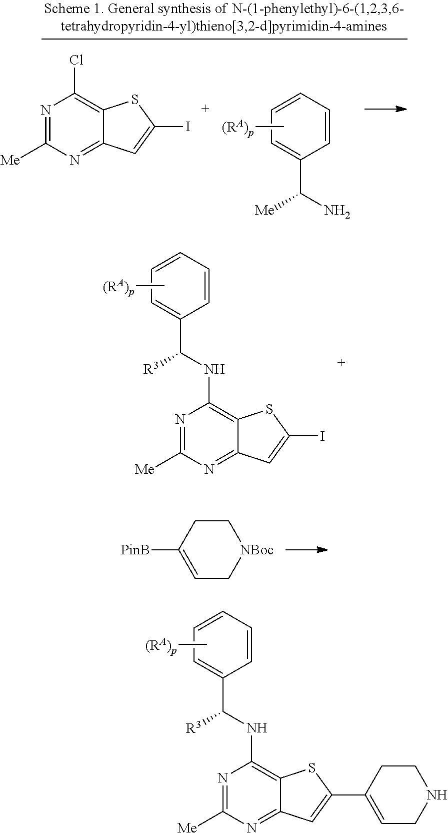

A general synthesis of N-(1-phenylethyl)-6-(1,2,3,6-tetrahydropyridin-4-yl)thieno[3,2-d]pyrimidin-4-amines or analogous heterocycle is outlined in Scheme 1. 4-Chloro-6-iodo-2-methylthieno[3,2-d]pyrimidine or analogous appropriately substituted double halogenated heterocyclic ring can undergo SNAr coupling with appropriately substituted benzyl amine in the presence of base. The resulting phenylethyl thienopyrimidine or analogous appropriately substituted heterocyclic ring can then be coupled to a substituted boronic acid derivative in the presence of Pd catalyst. Additional deprotection and/or functionalization steps can be required to produce the final compound.

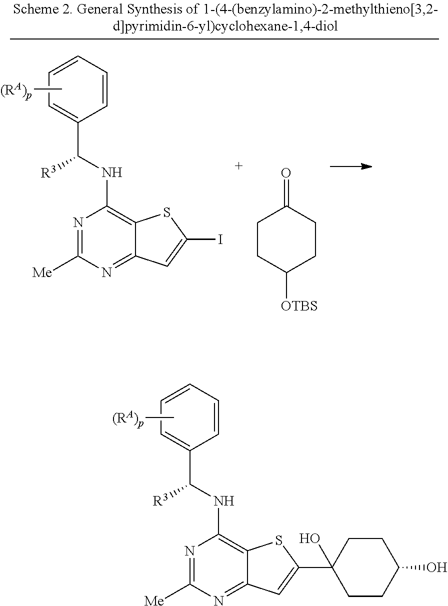

A general synthesis of 1-(4-(benzylamino)-2-methylthieno[3,2-d]pyrimidin-6-yl)cyclohexane-1,4-diol or analogous heterocycle is outlined in Scheme 2. 4-(Benzylamino)-2-methylthieno-[3,2-d]pyrimidines or analogous appropriately substituted heterocyclic ring (Scheme 1) can be coupled to appropriately protected (oxy)cyclohexan-1-ones via metal halogen exchange with LiHMDS or n-BuLi. Additional deprotection and/or functionalization steps can be required to produce the final compound.

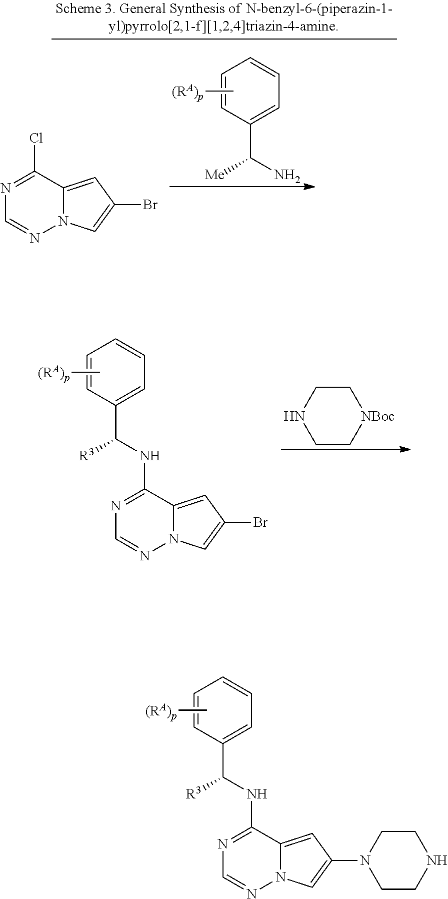

A general synthesis of N-benzyl-6-(piperazin-1-yl)pyrrolo[2,1-f][1,2,4]triazin-4-amine or analogous heterocycle is outlined in Scheme 3. 6-Bromo-4-chloropyrrolo[2,1-f][1,2,4]triazine or analogous appropriately substituted double halogenated heterocyclic ring can be coupled to a substituted benzyl amine. The resulting N-benzyl-pyrrolo[2,1-f][1,2,4]triazin can be coupled to a substituted primary or secondary amines in the presence of a palladium catalyst (e.g., t-BuXPhos). Additional deprotection and/or functionalization steps can be required to produce the final compound.

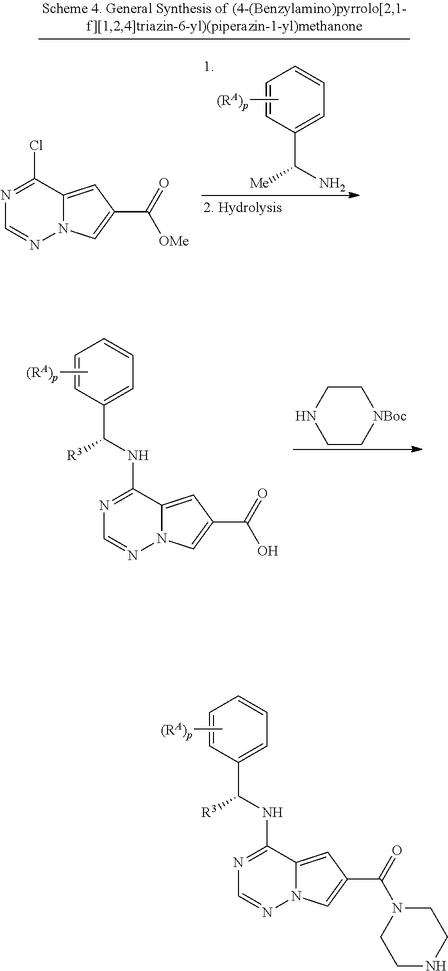

A general synthesis of (4-(Benzylamino)pyrrolo[2,1-f][1,2,4]triazin-6-yl)(piperazin-1-yl)methanone or analogous heterocycle is outlined in Scheme 4. Methyl 4-chloropyrrolo[2,1-f][1,2,4]triazine-6-carboxylate or analogous appropriately substituted halogenated heterocyclic ring can be coupled to a substituted benzyl amine. The resulting N-benzyl-pyrrolo[2,1-f][1,2,4]triazinyl methanone intermediate can be hydrolyzed and coupled to a substituted primary or secondary amines in the presence of a coupling agent. Additional deprotection and/or functionalization steps can be required to produce the final compound.

The present disclosure provides a compound of Formula Int-I:

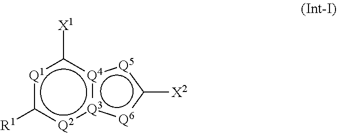

and salts, prodrugs, solvates, hydrates, tautomers, and isomers thereof, wherein:

X¹ is F, Cl, Br, or I;

X² is F, Cl, Br, or I.

Q¹ and Q² are independently CH or N;

Q³ and Q⁴ are independently C or N, wherein at least one of Q³ and Q⁴ is C;

Q⁵ and Q⁶ are independently CH, N, NH, O, or S;

wherein at least one of Q¹, Q², Q³, Q⁴, Q⁵, and Q⁶ is N, NH, O, or S;

R¹ is H, halogen, C₁₋₆ alkyl, 3-membered cycloalkyl, —CN, or —OR^{1a}; wherein R^{1a} is H or C₁₋₆ alkyl.

The present disclosure provides a compound of Formula Int-Ia:

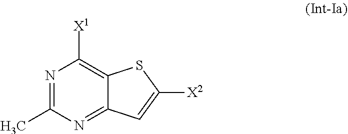

and salts, prodrugs, solvates, hydrates, tautomers, and isomers thereof, wherein X¹ is F, Cl, Br, or I; and X² is F, Cl, Br, or I.

The present disclosure provides a compound of Formula Int-II:

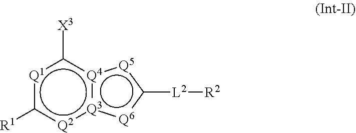

and salts, prodrugs, solvates, hydrates, tautomers, and isomers thereof, wherein:

X³ is F, Cl, Br, or I;

Q¹ and Q² are independently CH or N;

Q³ and Q⁴ are independently C or N, wherein at least one of Q³ and Q⁴ is C;

Q⁵ and Q⁶ are independently CH, N, NH, O, or S;

wherein at least one of Q¹, Q², Q³, Q⁴, Q⁵, and Q⁶ is N, NH, O, or S;

R¹ is H, halogen, C₁₋₆ alkyl, 3-membered cycloalkyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

The present disclosure provides a compound of Formula Int-IIa:

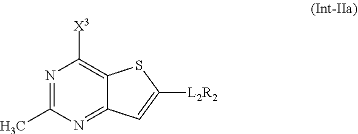

and salts, prodrugs, solvates, hydrates, tautomers, and isomers thereof, wherein:

X³ is F, Cl, Br, or I;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

The present disclosure provides a compound of Formula Int-III:

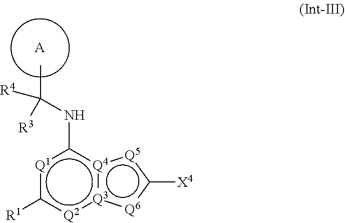

and salts, prodrugs, solvates, hydrates, tautomers, and isomers thereof, wherein:

X⁴ is F, Cl, Br, or I;

Q¹ and Q² are independently CH or N;

Q³ and Q⁴ are independently C or N, wherein at least one of Q³ and Q⁴ is C;

Q⁵ and Q⁶ are independently CH, N, NH, O, or S;

wherein at least one of Q¹, Q², Q³, Q⁴, Q⁵, and Q⁶ is N, NH, O, or S;

R¹ is H, halogen, C₁₋₆ alkyl, 3-membered cycloalkyl, —CN, or —OR^{1a}; wherein R^{1a} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl.

The present disclosure provides a compound of Formula Int-IIIa:

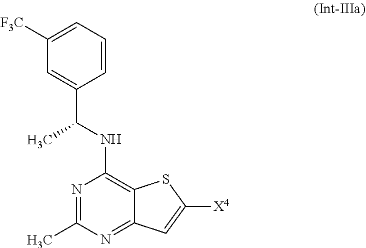

and salts, prodrugs, solvates, hydrates, tautomers, and isomers thereof, wherein X⁴ is F, Cl, Br, or I.

The present disclosure provides a compound, and salts, solvates, stereoisomers, and tautomers thereof, selected from the group consisting of:

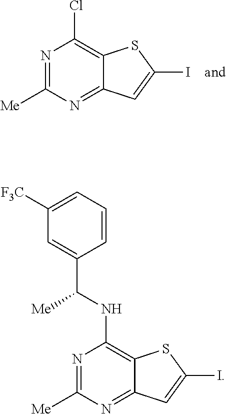 [SMILES: Cc1nc(Cl)c2sc(I)cc2n1 ; Cc1nc(N[C@H](C)c2cccc(C(F)(F)F)c2)c2sc(I)cc2n1]

### Therapeutic Use

Due to their biological properties the compounds of the present disclosure, their tautomers, racemates, enantiomers, diastereomers, mixtures thereof and the salts of all the above-mentioned forms may be suitable for treating diseases characterized by excessive or abnormal cell proliferation such as cancer.

For example, the following cancers, tumors and other proliferative diseases may be treated with compounds of the present disclosure, without being restricted thereto:

cancers/tumors/carcinomas of the head and neck: e.g., tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands); intraocular cancers (e.g., uveal melanoma), and orbital and adnexal cancers;

cancers/tumors/carcinomas of the lung: e.g., non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);

neoplasms of the mediastinum: e.g., neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma), astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors;

cancers/tumors/carcinomas of the gastrointestinal (GI) tract: e.g., tumors/carcinomas/cancers of the esophagus, stomach (gastric cancer), pancreas, liver and biliary tree (including hepatocellular carcinoma (HCC), e.g., childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g., renal pelvis, renal cell carcinoma (RCC), nephroblastoma (Wilms tumor), hypernephroma, Grawitz tumor; ureter; urinary bladder, e.g., urachal cancer, urothelial cancer; urethra, e.g., distal, bulbomembranous, prostatic; prostate (androgen dependent, androgen independent, castration resistant, hormone independent, hormone refractory), penis);

cancers/tumors/carcinomas of the testis: e.g., seminomas, non-seminomas;

gynecologic cancers/tumors/carcinomas: e.g., tumors/carcinomas/cancers of the ovary, fallopian tube, peritoneum, cervix, vulva, vagina, uterine body (including endometrium, fundus);

cancers/tumors/carcinomas of the breast: e.g., mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;

cancers/tumors/carcinomas of the endocrine system: e.g., tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VlPoma, glucagonoma, somatostatinoma, GRFoma, ACTHoma), carcinoid tumors;

sarcomas of the soft tissues: e.g., fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewings sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;

sarcomas of the bone: e.g., myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewings tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;

mesothelioma: e.g., pleural mesothelioma, peritoneal mesothelioma;

cancers of the skin: e.g., basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;

neoplasms of the peripheral and central nervous system and brain: e.g., astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors, neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);

lymphomas and leukemias: e.g., B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), Burkitt leukemia, T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkins disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML), JMML (juvenile myelomonocytic leukemia), acute leukemia of ambiguous lineage, myeloproliferative neoplasms, blastic plasmacytoid dendritic cell neoplasm, early T-cell precursor leukemia, natural killer cell leukemia/lymphoma, myeloid/lymphoid neoplasms with eosinophilia, myeloid sarcoma, transient abnormal myelopoiesis; and

cancers of unknown primary site (CUP).

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:

epithelial cancers, e.g., squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma; and

nonepithilial and mesenchymal cancers, e.g., sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

The compounds of the present disclosure may be used in therapeutic regimens in the context of first line, second line, or any further line treatments.

The compounds of the invention may be used for the prevention, short-term or long-term treatment of the above-mentioned diseases, optionally also in combination with radiotherapy and/or surgery and/or other compounds.

Of course, the above also includes the use of the compounds of the present disclosure in various methods of treating the above diseases by administering a therapeutically effective dose to a patient in need thereof, as well as the use of these compounds for the manufacture of medicaments for the treatment of such diseases, as well as pharmaceutical compositions including such compounds of the invention, as well as the preparation and/or manufacture of medicaments including such compounds of the invention, and the like.

### Additional Methods of Using the Disclosed Compounds

One aspect of the present disclosure relates to a method of inhibiting SOS1 in a subject in need thereof, comprising administering to the subject a SOS1 inhibitor of the present invention, or a pharmaceutically acceptable salt, solvate, hydrate, tautomer, or isomer thereof.

Another aspect of the present disclosure relates to a method of treating or preventing a disease that is effected or characterized by modification of the interaction of SOS1 and a RAS-family protein and/or RAC1 in a subject in need thereof. The method involves administering to a patient in need of treatment for diseases or disorders associated with SOS1 modulation an effective amount of a compound of any Formula disclosed herein, or a pharmaceutically acceptable salt, solvate, isomer, prodrug, or tautomer thereof.

In certain embodiments, a method is provided of inhibiting the interaction of SOS1 and a RAS-family protein in a cell or inhibiting the interaction of SOS1 and RAC1 in a cell, comprising administering to the cell a compound of any Formula disclosed herein, or a pharmaceutically acceptable salt, solvate, hydrate, tautomer, or isomer thereof, and a pharmaceutically acceptable carrier.

In certain embodiments, a method is provided of treating or preventing cancer in a subject in need thereof, comprising administering to the subject an effective amount of a compound of any Formula disclosed herein, or a pharmaceutically acceptable salt, solvate, hydrate, tautomer, or isomer thereof.

In certain embodiments, the disease can be, but is not limited to, cancer. In certain embodiments, the disease or cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, JMML (juvenile myelomonocytic leukemia), acute lymphoblastic leukemia/lymphoma, lymphomas, tumors of the central and peripheral nervous system, epithelial and nonepithelial tumors and mesenchymal tumor, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

In certain embodiments, the disease can be, but is not limited to, cancer. In certain embodiments, the disease or cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

In certain embodiments, the disease can be, but is not limited to, a RASopathy. In certain embodiments, the RASopathy is selected from the group consisting of Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

Another aspect of the present disclosure is directed to a method of inhibiting SOS1. The method involves administering to a patient in need thereof an effective amount of a compound of any Formula disclosed herein, or a pharmaceutically acceptable salt, solvate, isomer, prodrug, or tautomer thereof.

The present disclosure relates to compositions capable of modulating the activity of (e.g., inhibiting) SOS1. The present disclosure also relates to the therapeutic use of such compounds.

The disclosed compound can be administered in effective amounts to treat or prevent a disorder and/or prevent the development thereof in subjects.

Another aspect of the present disclosure relates to a compound of any Formula disclosed herein, or a pharmaceutically acceptable salt, hydrate, solvate, prodrug, stereoisomer, or tautomer thereof, for use in treating or preventing a disease that is affected by modification of the interaction of SOS1 and a RAS-family protein and/or RAC1. Another aspect of the present disclosure relates to a compound of any Formula disclosed herein, or a pharmaceutically acceptable salt, hydrate, solvate, prodrug, stereoisomer, or tautomer thereof, for use in treating or preventing a disease that is characterized by inhibition of the interaction of SOS1 with a RAS-family protein or the interaction of SOS1 with RAC1.

Another aspect of the present disclosure relates to a compound of any Formula disclosed herein, or a pharmaceutically acceptable salt, hydrate, solvate, prodrug, stereoisomer, or tautomer thereof, for use in treating or preventing a disease, wherein the treating or preventing is effected or characterized by inhibition of the interaction of SOS1 and a RAS-family protein or by inhibition of the interaction of SOS1 and RA.

Another aspect of the present disclosure relates to a compound of any Formula disclosed herein, or a pharmaceutically acceptable salt, hydrate, solvate, prodrug, stereoisomer, or tautomer thereof, for use inhibiting the binding of hSOS1 to H— or N— or K-RAS including their clinically known mutations and which inhibits the nucleotide exchange reaction catalyzed by hSOS1 in the presence of a concentration of 20 μM or lower, but which are substantially inactive against EGFR-kinase at concentrations of 20 μM or lower for the preparation of a medicament for the treatment or prophylaxis of a hyperproliferative disorder.

Another aspect of the present disclosure relates to a compound of any Formula disclosed herein, or a pharmaceutically acceptable salt, hydrate, solvate, prodrug, stereoisomer, or tautomer thereof, for the manufacture of a medicament for use inhibiting the binding of hSOS1 specifically to K-RAS G12C protein or another Ras mutant, as described herein, and which inhibits the nucleotide exchange reaction catalyzed by hSOS1 in the presence of a concentration of 20 μM or lower, but which are substantially inactive against EGFR-kinase at concentrations of 20 μM or lower for the preparation of a medicament for the treatment or prophylaxis of a hyperproliferative disorder.

In another aspect, the present disclosure relates to the use of a compound of any Formula disclosed herein, or a pharmaceutically acceptable salt, hydrate, solvate, prodrug, stereoisomer, or tautomer thereof, in the manufacture of a medicament for treating or preventing a disease.

Administration of the disclosed compounds can be accomplished via any mode of administration for therapeutic agents. These modes include systemic or local administration such as oral, nasal, parenteral, intravenous, transdermal, subcutaneous, vaginal, buccal, rectal or topical administration modes. Likewise, they can also be administered in intravenous (both bolus and infusion), intraperitoneal, subcutaneous or intramuscular form, and all using forms well known to those skilled in the pharmaceutical arts

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; 0 an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the present disclosure relates to a pharmaceutical composition comprising a compound of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### Combination Therapy

The methods of the invention may include a compound of the invention used alone or in combination with one or more additional therapies (e.g., non-drug treatments or therapeutic agents). Combination therapy may, for example, combine two therapies or may combine three therapies (e.g., a triple therapy of three therapeutic agents), or more. The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GALS, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-Ll antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as ribociclib, abemaciclib or palbociclib), seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TGO2 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TN0155, RMC-4550, RMC-4630, JAB-3068), another SOS1 inhibitor (e.g., BI-1701963), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312. In some embodiments, an anti-cancer agent is a Ras inhibitor (e.g., AMG 510, MRTX1257, MRTX849, LY349946, ARS-3248 (JNJ-74699157), or ARS-1620), or a Ras vaccine, or another therapeutic modality designed to directly or indirectly decrease the oncogenic activity of Ras.

In some embodiments, the Ras protein is wild-type. In some embodiments, the cancer comprises a Ras mutation. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-Ras mutants: Q61R, G13R, Q61K, G12S, Q61L, G12D,

G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R,

G13D, G12S, G12C, G12V, G12A, G13V, G12R, P185S, G13C, A146T, G60E, Q61P, A59D, E132K, E49K, T501, A146V, or A59T, and combinations thereof;

or a combination of any of the foregoing (e.g., both K-Ras G12C and K-Ras G13C). In some embodiments, the cancer comprises a Ras mutation selected from the group consisting of G12C, G13C, G12A, G12D, G13D, G12S, G13S, G12V and G13V.

In some embodiments, a therapeutic agent that may be combined with a compound of the present invention is an inhibitor of the MAP kinase (MAPK) pathway (or “MAPK inhibitor”). MAPK inhibitors include, but are not limited to, one or more MAPK inhibitor described in Cancers (Basel) 2015 Sep; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, R04987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); R05126766 (Roche, described in PLoS One. 2014 Nov 25;9(11)); and GSK1120212 (or JTP-74057, described in Clin Cancer Res. 2011 Mar 1;17(5):989-1000).

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 Sep; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-Ll antagonist.

In some embodiments, additional therapeutic agents include EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res.1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is osimertinib.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):34935-34985); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g., AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies, and may form the basis of a triple combination inhibitor with a SOS1 inhibitor.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO2015107493; WO2015107494; WO201507495; WO2016203404; WO2016203405; WO2016203406; WO2011022440; WO2017156397; WO2017079723; WO2017211303; WO2012041524; WO2017211303; WO2019051084; WO2017211303; US20160030594; US20110281942; WO2010011666; WO2014113584; WO2014176488; WO2017100279; WO2019051469; U.S. Pat. No. 8,637,684; WO2007117699; WO2015003094; WO2005094314; WO2008124815; WO2009049098; WO2009135000; WO2016191328; WO2016196591; WO2017078499; WO2017210134; WO2018013597; WO2018129402; WO2018130928; WO20181309928; WO2018136264; WO2018136265; WO2018160731; WO2018172984; and WO2010121212, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TN0155. In some embodiments, the SHP2 inhibitor is RMC-4550. In some embodiments, the SHP2 inhibitor is RCM-4630. In some embodiments, the SHP2 inhibitor is JAB-3068.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAG1, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 Al), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as VEGF-TRAPTM, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, US 5712291); ilomastat, (Arriva, USA, US5892112); emaxanib, (Pfizer, USA, US 5792783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™) bafilomycin Al, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATGS (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjetat); pertuzumab (Perjetat); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments, an additional compound used in combination therapy with a compound of the present invention is selected from the group consisting of a CDK4/6 inhibitor (e.g., abemaciclib, palbociclib, or ribociclib), a KRAS:GDP G12C inhibitor (e.g., AMG 510, MRTX 1257) or other mutant Ras:GDP inhibitor, a KRAS:GTP G12C inhibitor or other mutant Ras:GTP inhibitor, a MEK inhibitor (e.g., refametinib, selumetinib, trametinib, or cobimetinib), a SHP2 inhibitor (e.g., TN0155, RMC-4630), an ERK inhibitor, and an RTK inhibitor (e.g., an EGFR inhibitor).

In some embodiments, an additional compound used in combination therapy with a compound of the present invention is selected from the group consisting of ABT-737, AT-7519, carfilzomib, cobimetinib, danusertib, dasatinib, doxorubicin, GSK-343, JQ1, MLN-7243, NVP-ADW742, paclitaxel, palbociclib and volasertib. In some embodiments, an additional compound used in combination therapy with a compound of the present invention is selected from the group consisting of neratinib, acetinib and reversine.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments, a combination therapeutic regimen employs two therapeutic agents, one compound of the present invention and a second selected from the therapeutic agents described herein. In some embodiments, a combination therapeutic regimen employs three therapeutic agents, one compound of the present invention and two selected from the therapeutic agents described herein. In some embodiments, a combination therapeutic regimen employs four or more therapeutic agents, one compound of the present invention and three selected from the therapeutic agents described herein.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

In this Combination Therapy section, all references are incorporated by reference for the agents described, whether explicitly stated as such or not.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Definitions used in the following examples and elsewhere herein are:
AcCl Acetyl chloride
CH₂Cl₂, DCM Methylene chloride, Dichloromethane
CH₃CN, MeCN, Acetonitrile
and ACN
DIEA N,N-diisopropylethylamine
DIPEA Diisopropylethyl amine
DME Dimethylether
DMF N,N-Dimethylformamide
EDCI N-(3-Dimethylaminopropyl)-N′-ethyl-carbodiimide
EtOAc Ethyl acetate
h Hour
H₂O Water
HATU N-RDimethylamino)-1H-1,2,3-triazolo-[4,5-blpyridin-1-ylmethylene]-N-methylmethanaminium hexafluorophosphate N-oxide
HCl Hydrochloric acid
HOBt 1-Hy droxybenzotriazole
K₃PO₄ Potassium phosphate (tribasic)
LDA Lithium diisopropylamide
LiHDMS Lithium bis(trimethylsilyl)amide
MeOH Methanol
Na₂SO₄ Sodium sulfate
NMP N-methyl pyrrolidone
PMB p-Methoxybenzyl
PyBOP Benzotriazol-1-yl-oxytripyrrolidinophosphonium hexafluorophosphate
Rt Room temperature
T3P Propanephosphonic acid anhydride
TBAF Tetrabutylammonium fluoride
TEA Triethylamine
THF Tetrahydrofuran
TMSCl Trimethylsilyl chloride
Xantphos 4,5-Bis(diphenylphosphino)-9,9-dimethylxanthene

### Example 1. Synthesis of 2-methyl-6-(1,2,3,6-tetrahydropyridin-4-yl)-N-[(1R)-1-[3-(trifluoromethyl)phenyl]ethyl]thieno [3,2-d] pyrimidin-4-amine

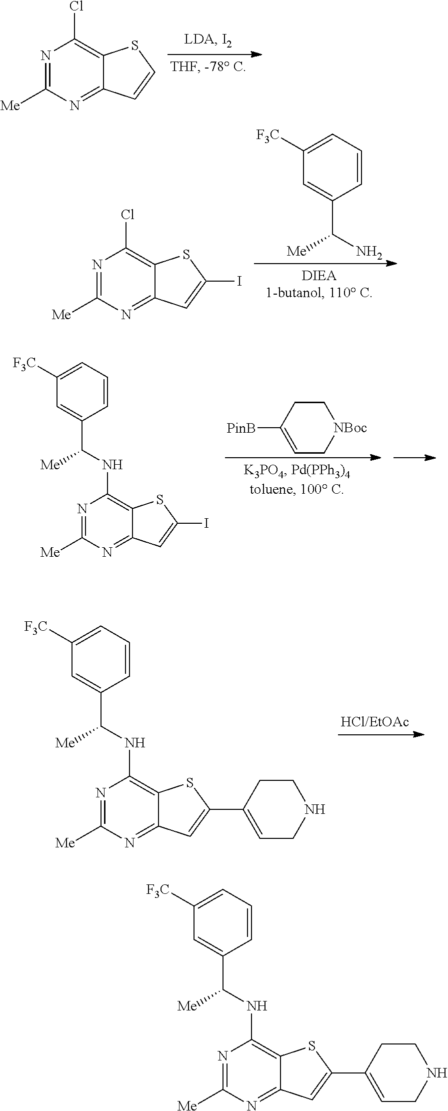

### Step 1.

To a mixture of 4-chloro-2-methyl-thieno[3,2-d]pyrimidine (400 mg, 2.17 mmol) in THF (12 mL) was added LDA (2 M, 1.30 mL) at −78° C. under N₂. The mixture was stirred at −78° C. for 30 min, then a solution of I₂ (567.28 μL, 2.82 mmol) in THF (6 mL) was added. The mixture was allowed to warm to rt and was left to stir for 2 h. The mixture was then poured into water extracted with DCM. The combined organic phases were washed with brine, dried over Na₂SO₄ and the solvent was removed under reduced pressure. The crude residue was triturated with EtOAc to afford 4-chloro-6-iodo-2-methyl-thieno[3,2-d]pyrimidine (540 mg, 80% yield). LCMS (ESI): m/z: [M+H] calculated for C₇H₅ClIN₂S: 310.9; found 311.0.

### Step 2.

To a mixture of 4-chloro-6-iodo-2-methyl-thieno[3,2-d]pyrimidine (400 mg, 1.29 mmol) and (1R)-1-[3-(trifluoromethyl)phenyl]ethanamine (292 mg, 1.55 mmol) in 1-butanol (10 mL) was added DIEA (448 pL, 2.58 mmol). The mixture was stirred at 110° C. for 18 h. After extraction with DCM the combined organic phases were washed with brine and dried over Na₂SO₄. The solvent was removed under reduced pressure and the crude residue was purified by column chromatography to give 6-iodo-2-methyl-N-[(1R)-1-[3-(trifluoromethyl)phenyl]ethyl]thieno[3,2-d]pyrimidin-4-amine (370 mg, 62% yield). ¹H NMR (400 MHz, METHANOL-d₄) δ=7.74 (s, 1H), 7.69-7.66 (m, 1H), 7.53-7.48 (m, 2H), 7.44 (s, 1H), 5.61 (q, J=7.1 Hz, 1H), 2.43 (s, 3H), 1.62 (d, J=7.1 Hz, 3H).

### Step 3.

To a mixture of 6-iodo-2-methyl-N-[(1R)-1-[3-(trifluoromethyl)phenyl]ethyl]thieno[3,2-d]pyrimidin-4-amine (200 mg, 431 μmol) and tert-butyl 4-(4,4,5,5-tetramethyl-1,3,2-dioxaborolan-2-yl)-3,6-dihydro-2H-pyridine-1-carboxylate (213 mg, 690 μmol) in toluene (9 mL) was added K₃PO₄ (366 mg, 1.73 mmol), Pd(PPh₃)₄ (Palladium-tetrakis(triphenylphosphine, 50 mg, 43 μmol). The mixture was stirred at 100° C. for 12 h under N₂. After cooling to rt the solvent was removed under reduced pressure and the crude residue was purified by column chromatography to give tert-butyl 4-[2-methyl-4-[[(1R)-1-[3-(trifluoromethyl)phenyl]ethyl]amino]thieno[3,2-d]pyrimidin-6-yl]-3,6-dihydro-2H-pyridine-1-carboxylate (50 mg, crude). LCMS (ESI): m/z: [M+H] calculated for C₂₆H₃₀F₃N₄O₂S: 519.2; found 519.3.

### Step 4.

tert-Butyl 4-[2-methyl-4-[[(1R)-1-[3-(trifluoromethyl)phenyl]ethyl]amino]thieno[3,2-d]pyrimidin-6-yl]-3,6-dihydro-2H-pyridine-1-carboxylate (50 mg, 96 μmol) was dissolved in HCl/EtOAc (6 mL). The mixture was stirred at 25° C. for 1 h, the solvent was removed under reduced pressure and the crude residue was purified by prep-HPLC to give 2-methyl-6-(1,2,3,6-tetrahydropyridin-4-yl)-N-[(1R)-1-[3-(trifluoromethyl)phenyl]ethyl]thieno[3,2-d]pyrimidin-4-amine monoformate (23 mg, 51% yield). LCMS (ESI): m/z: [M+H] calculated for C₂₁H₂₂F₃N₄S: 419.1; found 419.2; ¹H NMR (400 MHz, DMSO-d₆) δ ppm 9.23 (br s, 2H), 7.84 (s, 1H), 7.78 (d, J=7.5 Hz, 1H), 7.67-7.56 (m, 2H), 7.39 (s, 1H), 6.54 (s, 1H), 5.71 (s, 1H), 3.85 (s, 2H), 2.79 (s, 2H), 2.69-2.65 (m, 1H), 2.52 (d, J=1.8 Hz, 3H), 2.35-2.31 (m, 1H), 1.62 (d, J=7.0 Hz, 3H).

### Example 2. Synthesis of 2-methoxy-1-[4-(2-methyl-4-{[(1R)-1-[3-(trifluoromethyl)phenyl]ethyl]amino}thieno[3,2-d]pyrimidin-6-yl)-1,2,3,6-tetrahydropyridin-1-yl]ethan-1-one

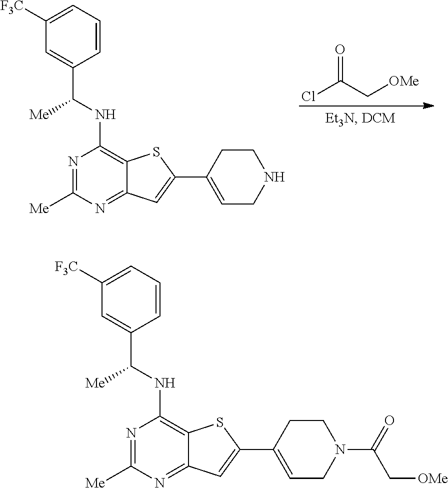 [SMILES: Cc1nc(N[C@H](C)c2cccc(C(F)(F)F)c2)c2sc(C3=CCNCC3)cc2n1 ; COCC(=O)Cl ; COCC(=O)N1CC=C(c2cc3nc(C)nc(N[C@H](C)c4cccc(C(F)(F)F)c4)c3s2)CC1]

### Step 1.

To a mixture of 2-methoxyacetyl chloride (2 μL, 20 μmol) and 2-methyl-6-(1,2,3,6-tetrahydropyridin-4-yl)-N-[(1R)-1-[3-(trifluoromethyl)phenyl]ethyl]thieno[3,2-d]pyrimidin-4-amine (11 mg, 26 μmol) in DCM (1 mL) was added Et₃N (15 μL, 105 μmol). The mixture was stirred at 25° C. for 30 min and then poured into water. The solvent was removed under reduced pressure and the crude residue was purified by prep-HPLC to give 2-methoxy-1-[4-[2-methyl-4-[[(1R)-1-[3-(trifluoromethyl)phenyl]ethyl]amino]thieno[3,2-d]pyrimidin-6-yl]-3,6-dihydro-2H-pyridin-1-yl]ethanone monoformate (3 mg, 23% yield). LCMS (ESI): m/z: [M+H] calculated for C₂₄H₂₆F₃N₄O₂S: 491.2; found: 491.3; ¹H NMR (400 MHz, METHANOL-d₄) δ ppm 7.76 (s, 1H), 7.72-7.65 (m, 1H), 7.55-7.47 (m, 2H), 7.18-7.11 (m, 1H), 6.49-6.39 (m, 1H), 5.64 (q, J=7.2 Hz, 1H), 4.29-4.17 (m, 4H), 3.85 (t, J=5.8 Hz, 1H), 3.73 (t, J=5.7 Hz, 1H), 3.45-3.39 (m, 3H), 2.74-2.61 (m, 2H), 2.44 (s, 3H), 1.64 (d, J=7.1 Hz, 3H).

### Example 3. Synthesis of N-[(1R)-1-[3-amino-5-(trifluoromethyl)phenyl]ethyl]-6-(1,2,3,6-tetrahydropyridin-4-yl)thieno[2,3-d]pyrimidin-4-amine

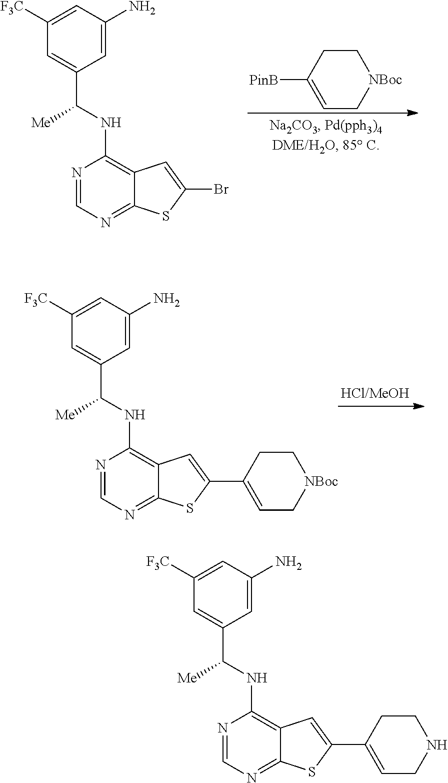

### Step 1.

To a solution of N-[(1R)-1-[3-amino-5-(trifluoromethyl)phenyl]ethyl]-6-bromo-thieno[2,3-d]pyrimidin-4-amine (30 mg, 72 μmol) and tert-butyl 4-(4,4,5,5-tetramethyl-1,3,2-dioxaborolan-2-yl)-3,6-dihydro-2H-pyridine-1-carboxylate (33 mg, 108 μmol) in DME (1 mL) and H₂O (0.2 mL) was added Na₂CO₃ (15 mg, 144 μmol) and Pd(PPh₃)₄ (Palladium-tetrakis(triphenylphosphine 8 mg, 7 μmol). The mixture was stirred at 85° C. for 16 h. After cooling to rt the reaction mixture was poured into water and the mixture was extracted with ethyl acetate. The combined organic phases were washed with brine and dried over Na₂SO₄. The solvent was removed under reduced pressure and the crude residue was purified by prep-TLC to give tert-butyl 4-[4-[[(1R)-1-[3-amino-5-(trifluoromethyl)phenyl]ethyl]amino]thieno[2,3-d]pyrimidin-6-yl]-3,6-dihydro-2H-pyridine-1-carboxylate (20 mg, 53% yield). LCMS (ESI): m/z: [M+H] calculated for C₂₅H₂₉F₃N₅O₂S: 520.2; found 520.3.

### Step 2.

A mixture of tert-butyl 4-[4-[[(1R)-1-[3-amino-5-(trifluoromethyl)phenyl]ethyl]amino]thieno[2,3-d]pyrimidin-6-yl]-3,6-dihydro-2H-pyridine-1-carboxylate (20 mg, 38 μmol) in HCl/MeOH (2 mL) was stirred at 25° C. for 2 h. The solvent was removed under reduced pressure and the residue was purified by prep-HPLC to give N-[(1R)-1-[3-amino-5-(trifluoromethyl)phenyl]ethyl]-6-(1,2,3,6-tetrahydropyridin-4-yl)thieno[2,3-d]pyrimidin-4-amine (6 mg, 39% yield). LCMS (ESI): m/z: [M+H] calculated for C₂₀H₂₁F₃N₅S: 420.1; found 420.2; ¹H NMR (400 MHz, METHANOL-d₄) δ ppm 8.27 (s, 1H) 7.65 (s, 1H) 6.94 (s, 2H) 6.80 (s, 1H) 6.26 (s, 1H) 5.47 (d, J=6.85 Hz, 1H) 3.79 (s, 2H) 3.41 (t, J=5.99 Hz, 2H) 2.83 (s, 2H) 1.60 (d, J=7.09 Hz, 3H).

### Example 4. Synthesis of 1-[4-(4-{[(1R)-1-[3-amino-5-(trifluoromethyl)phenyl]ethyl]amino}thieno[2,3-d]pyrimidin-6-yl)-1,2,3,6-tetrahydropyridin-1-yl]-2-methoxyethan-1-one

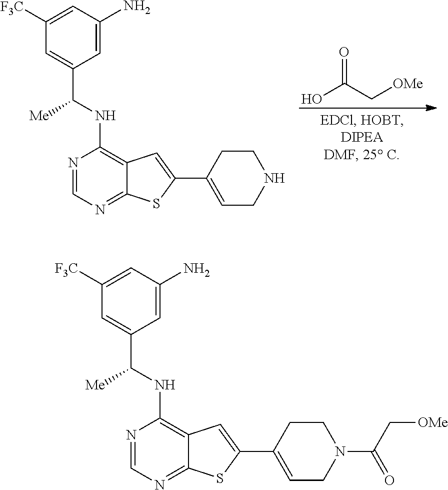 [SMILES: C[C@@H](Nc1ncnc2sc(C3=CCNCC3)cc12)c1cc(N)cc(C(F)(F)F)c1 ; COCC(=O)O ; COCC(=O)N1CC=C(c2cc3c(N[C@H](C)c4cc(N)cc(C(F)(F)F)c4)ncnc3s2)CC1]

### Step 1.

To a solution of 2-methoxyacetic acid (6 μL, 73 μmol) in DMF (2 mL) was added EDCI (18 mg, 92 μmol) and HOBt (10 mg, 77 μmol). Then DIPEA (80 μL, 462 μmol) and N-[(1R)-1-[3-amino-5-(trifluoromethyl)phenyl]ethyl]-6-(1,2,3,6-tetrahydropyridin-4-yl)thieno[2,3-d]pyrimidin-4-amine (32 mg, 77 μmol) were added to above mixture at 0° C. The reaction was stirred at 25° C. for 3 h. Aqueous NH₄Cl was added and the mixture was poured into water. The aqueous phase was extracted with EtOAc and the combined organic phases were washed with brine and dried over Na₂SO₄. The solvent was removed under reduced pressure and the crude residue was purified by prep-HPLC to give 1-[4-[4-[[(1R)-1-[3-amino-5-(trifluoromethyl)phenyl]ethyl]amino]thieno[2,3-d]pyrimidin-6-yl]-3,6-dihydro-2H-pyridin-1-yl]-2-methoxy-ethanone (7 mg, 18% yield). LCMS (ESI): m/z: [M+H] calculated for C₂₃H₂₅F₃N₅O₂S: 492.2; found 492.1; ¹H NMR (400 MHz, METHANOL-d₄) δ ppm 8.26 (s, 1H) 7.88-8.00 (m, 1H) 7.72 (s, 1H) 6.86 (s, 2H) 6.73 (s, 1H) 6.19 (s, 1H) 5.39-5.49 (m, 1H) 5.35 (s, 2H) 4.10-4.20 (m, 4H) 3.70 (s, 2H) 3.34 (s, 3H) 2.55-2.65 (m, 3H) 1.54 (d, J=6.84 Hz, 3H).

### Example 5. Synthesis of trans-1-(2-methyl-4-{[(1R)-1-[3-(trifluoromethyl)phenyl]ethyl]amino}thieno[3,2-d]pyrimidin-6-yl) cyclohexane-1,4-diol

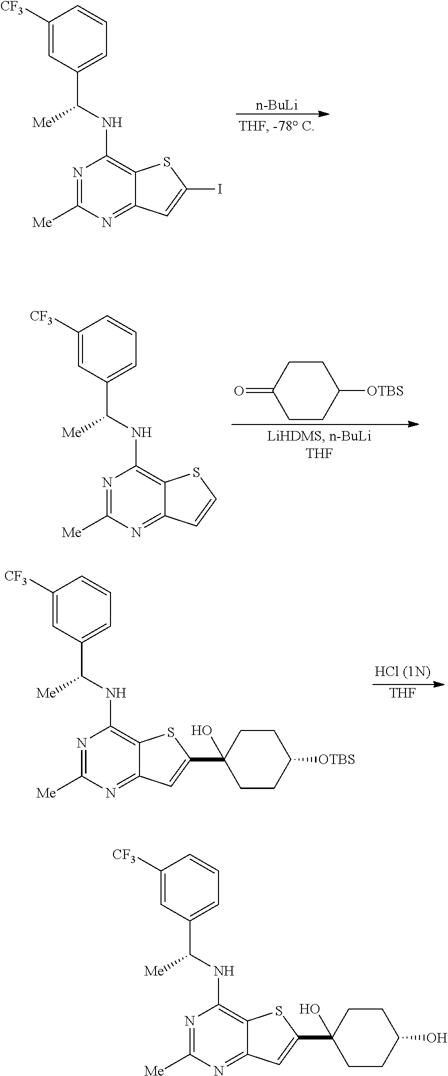

### Step 1.

To a mixture of 6-iodo-2-methyl-N-[(1R)-1-[3-(trifluoromethyl)phenyl]ethyl]thieno[3,2-d]pyrimidin-4-amine (520 mg, 1.12 mmol) in THF (5 mL) was added n-BuLi (2.5 M, 538 μL, 1.35 mmol) at −78° C. under N₂. The mixture was stirred at −78° C. for 30 min and then poured into water the solvent was removed under reduced pressure. The crude residue was purified by prep-TLC to give 2-methyl-N-[(1 R)-1-[3-(trifluoromethyl)phenyl]ethyl]thieno[3,2-d]pyrimidin-4-amine (390 mg, 99% yield). LCMS (ESI): m/z: [M+H] calculated for C16H15F3N3S: 338.09; found; 338.2. 1H NMR (400 MHz, METHANOL-d4) δ ppm 7.94-7.88 (m, 1H), 7.76 (s, 1H), 7.69 (d, J=6.2 Hz, 1H), 7.54-7.43 (m, 2H), 7.24 (d, J=5.4 Hz, 1H), 5.66 (q, J=7.0 Hz, 1H), 2.46 (s, 3H), 1.64 (d, J=7.1 Hz, 3H).

### Step 2.

To a solution of (R)-2-methyl-N-(1-(3-(trifluoromethyl)phenyl)ethyl)thieno[3,2-d]pyrimidin-4-amine (100 mg, 296 μmol) in THF (10 mL) was added LiHMDS (1 M, 1.19 mL, 1.19 mmol) at 0° C. The resulting solution was stirred for 30 min at 0° C. To the resulting mixture was then added n-BuLi (2.5 M, 1.19 mL, 3 mmol) at −78° C. A solution of 4-((tert-butyldimethylsilyl)oxy)cyclohexanone (744 μL, 2.96 mmol) in THF (5 mL) was added and the mixture was left to stir at −78° C. for 30 min and then poured into water. After extraction with EtOAc the combined organic phases were washed with brine and dried over Na₂SO₄. The solvent was removed under reduced pressure and the crude residue was purified by prep-HPLC to give cis-4-((tert-butyldimethylsilyl)oxy)-1-(2-methyl-4-(((R)-1-(3-(trifluoromethyl)phenyl)ethyl)amino)thieno[3,2-d]pyrimidin-6-yl)cyclohexanol (80 mg, 48% yield) and trans-4-((tert-butyldimethylsilyl)oxy)-1-(2-methyl-4-(((R)-1-(3-(trifluoromethyl)phenypl)ethyl)amino)thieno[3,2-d]pyrimidin-6-yl)cyclohexanol (30 mg, 18% yield). LCMS (ESI): m/z: [M+H] calculated for C₂₈H₃₉F₃N₃O₂SSi: 566.2; found 566.3.

### Step 3.

To a solution of cis-4-((tert-butyldimethylsilyl)oxy)-1-(2-methyl-4-(((R)-1-(3-(trifluoromethyl)phenyl)ethyl)amino)thieno[3,2-d]pyrimidin-6-yl)cyclohexanol (116 mg, 205.03 μmol, 1 eq) in THF (2 mL) was added HCl (1 M, 2.05 mL, 2.05 mmol) and the mixture was stirred at 25° C. for 1 h. The solvent was removed under reduced pressure and the residue was purified by prep-HPLC to give cis-1-(2-methyl-4-(((R)-1-(3-(trifluoromethyl)phenyl)ethyl)amino)thieno[3,2-d]pyrimidin-6-yl)cyclohexane-1,4-diol (33 mg, 36% yield). LCMS (ESI): m/z: [M+H] calculated for C₂₂H₂₅F₃N₃O₂S: 452.2; found 452.2; ¹H NMR (400 MHz, METHANOL-d4) δ ppm 7.75 (s, 1H) 7.69 (d, J=6.36 Hz, 1H) 7.46-7.53 (m, 2H) 7.07 (s, 1H) 5.64 (q, J=7.05 Hz, 1H) 3.63-3.74 (m, 1H) 2.43 (s, 3H) 1.90-2.08 (m, 4H) 1.76-1.88 (m, 4H) 1.63 (d, J=7.09 Hz, 3H).

### Example 6. Synthesis of cis-1-(2-methyl-4-{[(1R)-1-[3-(trifluoromethyl)phenyl]ethyl]amino}thieno[3,2-d]pyrimidin-6-yl)cyclohexane-1,4-diol

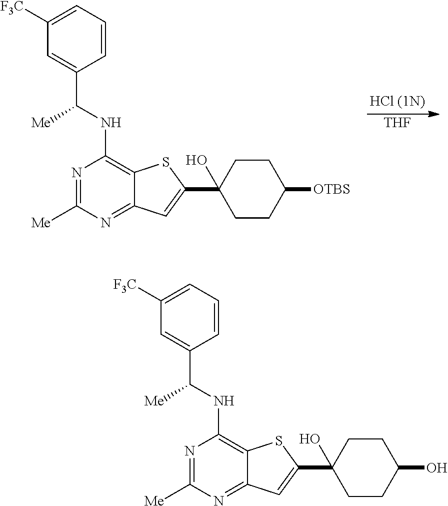

### Step 1.

To a solution of trans-4-((tert-butyldimethylsilypoxy)-1-(2-methyl-4-(((R)-1-(3-(trifluoromethyl)phenyl)ethyl)amino)thieno[3,2-d]pyrimidin-6-yl)cyclohexanol (50 mg, 88 μmol) in THF (2 mL) was added HCl (1 M, 884 μL, 884 μmol) and the mixture was stirred at 25° C. for 1 h. The solvent was removed under reduced pressure and the crude residue was purified by prep-HPLC to give trans-1-(2-methyl-4-(((R)-1-(3-(trifluoromethyl)phenyl)ethyl)amino)thieno[3,2-d]pyrimidin-6-yl)cyclohexane-1,4-diol (7 mg, 19% yield). LCMS (ESI): m/z: [M+H] calculated for C₂₂H₂₅F₃N₃O₂S: 452.15; found 452.3; ¹H NMR (400 MHz, METHANOL-d4) δ ppm 7.77 (s, 1H) 7.71 (br d, J=6.11 Hz, 1H) 7.48-7.55 (m, 2H) 7.14 (s, 1H) 5.66 (q, J=6.77 Hz, 1H) 4.01 (s, 1H) 2.45 (s, 3H) 2.27-2.36 (m, 2H) 1.98-2.09 (m, 2H) 1.81 (d, J=13.45 Hz, 2H) 1.63-1.74 (m, 5H).

### Example 7. Synthesis of 1-(4-{[(1R)-1-[3-amino-5-(trifluoromethyl)phenyl]ethyl]amino}thieno[2,3-d]pyrimidin-6-yl)cyclohexane-1,4-diol

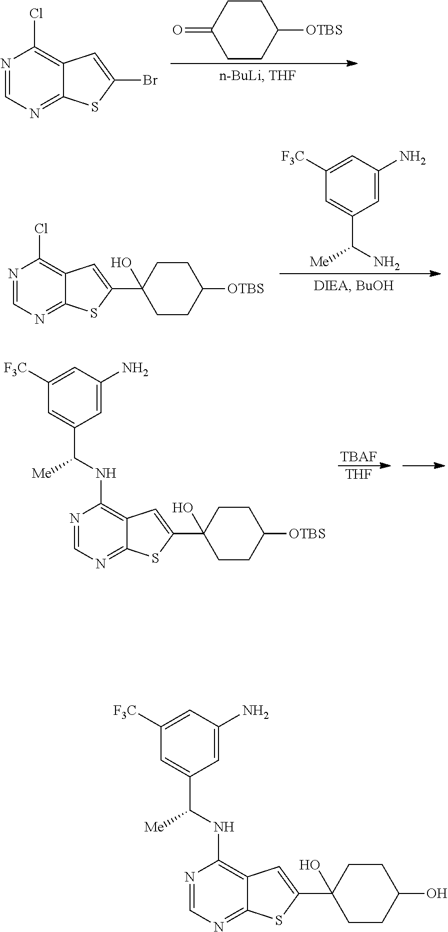

### Step 1.

A solution of 6-bromo-4-chloro-thieno[2,3-d]pyrimidine (300 mg, 1.20 mmol) in dry THF (3 mL) was cooled to −78° C. under N2. A solution of n-BuLi (2.5 M, 960 μL, 2.4 mmol) was then added, followed by a solution of 4-[tert-butyl(dimethyl)silyl]oxycyclohexanone (453 μL, 1.80 mmol) in dry THF (3 mL). This mixture was stirred at −78° C. for 2 h and then quenched by the addition of H₂O. The phases were separated and the solvent was removed under reduced pressure. The crude residue was purified by prep-HPLC to give 4-[tert-butyl(dimethyl)silyl]oxy-1-(4-chlorothieno[2,3-d]pyrimidin-6-yl)cyclohexanol (100 mg, 21% yield). ¹H NMR (400 MHz, METHANOL-d₄) δ ppm 8.76 (s, 1H) 7.35 (d, J=0.61 Hz, 1H) 3.82 (tt, J=9.61, 4.81 Hz, 1H) 1.95-2.09 (m, 4H) 1.75-1.91 (m, 4H) 0.93 (s, 9H) 0.11 (d, J=0.61 Hz, 6H).

### Step 2.

To a solution of 4-[tert-butyl(dimethyl)silyl]oxy-1-(4-chlorothieno[2,3-d]pyrimidin-6-yl)cyclohexanol (50 mg, 125 μmol) and 3-[(1R)-1-aminoethyl]-5-(trifluoromethyl)aniline (33 mg, 162 μmol) in EtOH (1 mL) was added DIEA (65 μL, 375 μmol). The mixture was stirred at 100° C. in a sealed tube for 12 h. After cooling to rt aqueous NaHCO₃ was added and the mixture was extracted with EtOAc. The combined organic phases were washed with brine and dried over Na₂SO₄. The solvent was removed under reduced pressure and the crude residue was purified by prep-TLC to give 1-[4-[[(1R)-1-[3-amino-5-(trifluoromethyl)phenyl]ethyl]amino]thieno[2,3-d]pyrimidin-6-yl]-4-[tert-butyl(dimethyl)silyl]oxy-cyclohexanol (50 mg, 69% yield). LCMS (ESI): m/z: [M+H] calculated for C₂₇H₃₈F₃N₄O₂SSi: 567.2; found 567.3;

### Step 3.

To a solution of 1-[4-[[(1R)-1-[3-amino-5-(trifluoromethyl)phenyl]ethyl]amino]thieno[2,3-d]pyrimidin-6-yl]-4-[tert-butyl(dimethyl)silyl]oxy-cyclohexanol (50 mg, 88 μmol) in THF (1 mL) was added TBAF (1 M, 176 μL, 176 μmol). The mixture was stirred at 70° C. for 2 h and the solvent was removed under reduced pressure. The crude residue was purified by prep-HPLC to give 1-[4-[[(1R)-1-[3-amino-5-(trifluoromethyl)phenyl]ethyl]amino]thieno[2,3-d]pyrimidin-6-yl]cyclohexane-1,4-diol (18 mg, 45% yield). LCMS (ESI): m/z: [M+H] calculated for C₂₁H₂₄F₃N₄O₂S: 453.1; found 453.1; ¹H NMR (400 MHz, METHANOL-d₄) δ ppm 8.22 (s, 1H) 7.49 (s, 1H) 6.94 (br s, 2H) 6.80 (s, 1H) 5.44 (q, J=6.85 Hz, 1H) 3.61-3.73 (m, 1H) 2.02-2.13 (m, 2H) 1.89-2.00 (m, 2H) 1.85 (dd, J=6.85, 2.93 Hz, 4H) 1.59 (d, J=6.97 Hz, 3H).

### Example 8. Synthesis of 6-(piperazin-1-yl)-N-[(1R)-1-[3-(trifluoromethyl)phenyl]ethyl]pyrrolo[2,14][1,2,4]triazin-4-amine

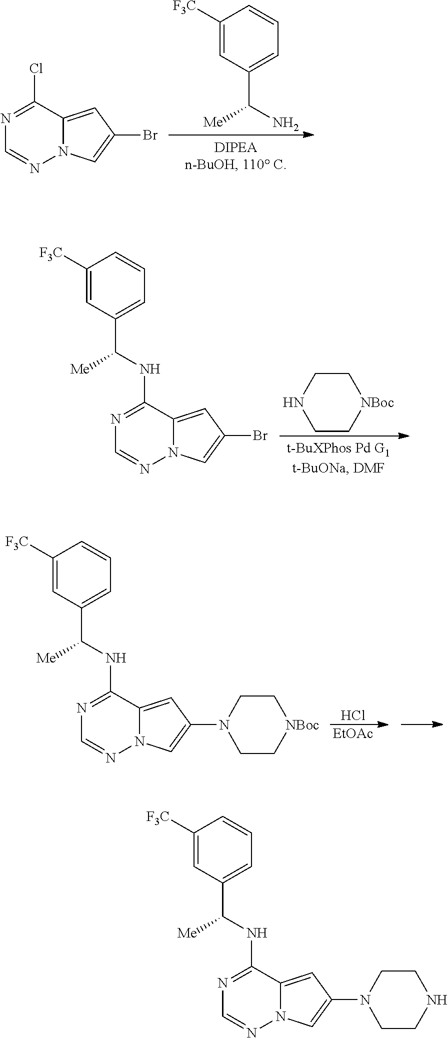

### Step 1.

To a solution of 6-bromo-4-chloro-pyrrolo[2,1-f][1,2,4]triazine (200 mg, 860 μmol) and (1R)-1-[3-(trifluoromethyl)phenyl]ethanamine (162 mg, 860 μmol) in n-BuOH (2 mL) was added DIEA (450 μL 2.58 mmol). The mixture was stirred at 130° C. for 1 h, cooled to rt and poured over ice-water (5 mL). After extraction with EtOAc the combined organic phases were washed with brine and dried over Na₂SO₄. The solvent was removed under reduced pressure and the crude residue was purified by column chromatography to give 6-bromo-N-[(1R)-1-[3-(trifluoromethyl)phenyl]ethyl]pyrrolo[2,1-f][1,2,4]triazin-4-amine (300 mg, 91% yield). ¹H NMR (400 MHz, CHLOROFORM-d) δ=7.91 (s, 1H), 7.65 (s, 1H), 7.61-7.54 (m, 3H), 7.52-7.46 (m, 1H), 6.63 (d, J=1.6 Hz, 1H), 5.59 (m, J=7.1 Hz, 1H), 5.36 (br d, J=7.0 Hz, 1H), 1.69 (d, J=6.8 Hz, 3H).

### Step 2.

To a solution of 6-bromo-N-[(1R)-1-[3-(trifluoromethyl)phenyl]ethyl]pyrrolo[2,1-f][1,2,4]triazin-4-amine (200 mg, 519 μmol) and tert-butyl piperazine-1-carboxylate (145 mg, 778 μmol) in DMF (1.5 mL) was added t-BuONa (99.80 mg, 1.04 mmol) and [2-(2-aminoethyl)phenyl]-chloro-palladium di tert-butyl-[2-(2,4,6-triisopropylphenyOphenyl]phosphane (36 mg, 52 μmol). The mixture was stirred at 110° C. for 10 h under N₂, cooled to rt and poured over ice-water. The mixture was extracted with ethyl acetate and the combined organic phases were washed with brine and dried over Na₂SO₄. The solvent was removed under reduced pressure and the residue was purified by column chromatography to give tert-butyl 4-[4-[[(1R)-1-[3-(trifluoromethyl)phenyl]ethyl]amino]pyrrolo[2,1-f][1,2,4]triazin-6-yl]piperazine-1-carboxylate (160 mg, 63% yield). ¹H NMR (400 MHz, CHLOROFORM-d) δ=7.89 (s, 1H), 7.66 (s, 1H), 7.60 (d, J=8.2 Hz, 1H), 7.57-7.53 (m, 1H), 7.50-7.45 (m, 1H), 7.19 (d, J=2.0 Hz, 1H), 6.11 (d, J=2.0 Hz, 1H), 5.63-5.55 (m, 1H), 5.20 (br d, J=7.5 Hz, 1H), 3.63-3.57 (m, 4H), 3.04 (s, 4H), 1.68 (d, J=6.8 Hz, 3H), 1.49 (s, 9H).

### Step 3.

A mixture of tert-butyl 4-[4-[[(1R)-1-[3-(trifluoromethyl)phenyl]ethyl]amino]pyrrolo[2,1-f][1,2,4]triazin-6-yl]piperazine-1-carboxylate (120 mg, 244 μmol) in HCl/EtOAc (5 mL, 4 N) was stirred at 25° C. for 30 min. The solvent was removed under reduced pressure and the crude residue was purified by prep HPLC to give 6-piperazin-1-yl-N-[(1R)-1-[3-(trifluoromethyl)phenyl]ethyl]pyrrolo[2,1-f][1,2,4]triazin-4-amine (85 mg, 78% yield). LCMS (ESI): m/z: [M+H] calculated for C₁₉H₂₂F₃N₆: 391.2; found 390.9; ¹H NMR (400 MHz, METHANOL-d4) δ=7.92 (s, 1H), 7.80-7.77 (m, 1H), 7.74 (d, J=7.8 Hz, 1H), 7.71 (s, 1H), 7.69-7.65 (m, 1H), 7.65-7.59 (m, 1H), 7.14 (s, 1H), 5.32 (s, 1H), 3.41 (s, 8H), 1.78 (d, J=6.8 Hz, 3H).

### Example 9. Synthesis of N-[(1 R) -1-[3-amino-5-(trifluoromethyl)phenyl]ethyl]-6-(1,2,3,6-tetrahydropyridin-4-yl)thieno[2,3-d]pyrimidin-4-amine

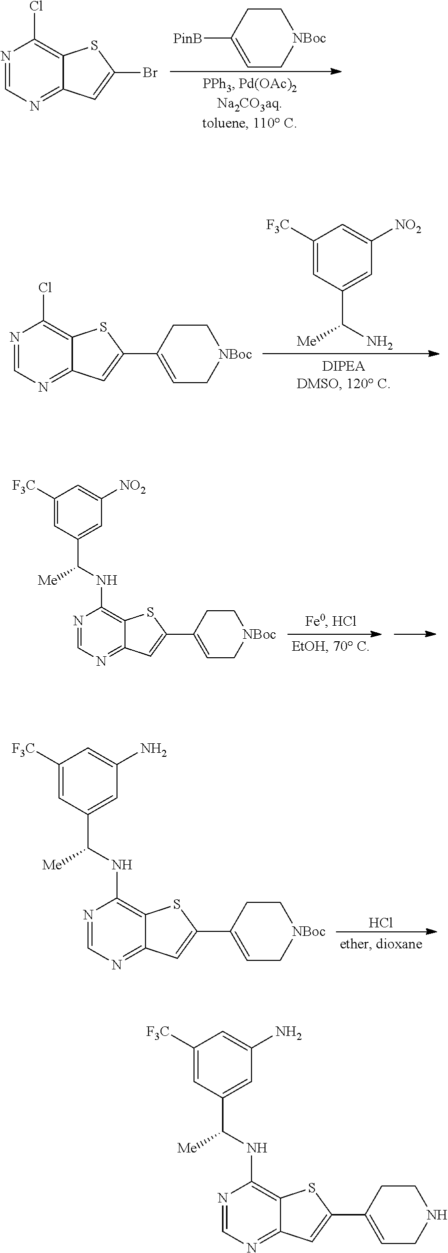

### Step 1.

6-Bromo-4-chlorothieno[3,2-d]pyrimidine (1.01 g, 4.1 mmol), (1-(tert-Butoxycarbonyl)-1,2,3,6-tetrahydropyridin-4-yl)boronic acid (1.06 g, 4.7 mmol), PPh₃ (373 mg, 1.4 mmol) and Pd(OAc)₂ (110 mg, 0.5 mmol) were dissolved in toluene (20 mL). A solution of Na₂CO₃ (1.47 g, 13.8 mmol) in water (5.0 mL) was added and the mixture was purged with Ar. The resulting solution was stirred for 12 h at 110° C. After cooling to rt solids were removed by filtration and the filtrate was washed with water and brine. The organic phase was dried over Na₂SO₄ and the solvent was removed under reduced pressure. The crude residue was purified by column chromatography to give tert-butyl 4-{4-chlorothieno[3,2-d]pyrimidin-6-yl}-1,2,3 ,6-tetrahydropyridine-1-carboxylate (1.14 g, 80% yield). ¹H NMR (300 MHz, Chloroform-d) δ 8.91 (s, 1H), 7.37 (s, 1H), 6.47 (s, 1H), 4.20-4.15 (m, 2H), 3.69 (t, J=5.7 Hz, 2H), 2.63 (s, 2H), 1.50 (s, 9H).

### Step 2.

To a solution of tert-butyl 4-{4-chlorothieno[3,2-d]pyrimidin-6-yl}-1,2,3,6-tetrahydropyridine-1-carboxylate (250 mg, 0.71 mmol) in DMSO (7.5 ml), (1R)-1-[3-nitro-5-(trifluoromethyl)phenyl]ethan-1-amine hydrochloride (212 mg, 0.78 mmol) and DIPEA (500 μl, 2.84 mmol) were added. The resulting solution was stirred for 6 h in a microwave reactor at 120° C. After cooling to rt the reaction mixture was poured into water and extracted with diethyl ether. The combined organic phases were washed witch water and dried over Na₂SO₄. The solvent was removed under reduced pressure to give tert-butyl 4-(4-{[(1R)-1-[3-nitro-5-(trifluoromethyl)phenyl]ethyl]amino}thieno[3,2-d]pyrimidin-6-yl)-1,2,3,6-tetrahydropyridine-1-carboxylate (408 mg, 105% yield, crude), which was used in the next step without further purification. LCMS (ESI): m/z: [M+H] calculated for C₂₅H₂₇F₃N₅O₄S: 550.2; found 550.0.

### Step 3.

tert-Butyl 4-(4-{[(1R)-1-[3-nitro-5-(trifluoromethyl)phenyl]ethyl]amino}thieno[3,2-d]pyrimidin-6-yl)-1,2,3,6-tetrahydropyridine-1-carboxylate (408 mg, 0.74 mmol) was dissolved in EtOH (8.2 ml) and aqueous HCl (1 M, 1.1 ml, 1.1 mmol) was added, followed by iron powder (228 mg, 4.08 mmol). The reaction mixture was stirred at 70° C. for 2 h. After cooling to rt the mixture was extracted with EtOAc and washed with sat. aq NaHCO₃. The solvent was removed under reduced pressure to give tert-butyl 4-(4-{[(1R)-1-[3-amino-5-(trifluoromethyl)phenyl]ethyl]amino}thieno[3,2-d]pyrimidin-6-yl)-1,2,3,6-tetrahydropyridine-1-carboxylate (444 mg, 119% yield crude), which was used without further purification. LCMS (ESI): m/z: [M+H] calculated for C₂₅H₂₉F₃N₅O₂S: 520.2; found 520.1.

### Step 4.

To a solution of tert-butyl 4-(4-{[(1R)-1-[3-amino-5-(trifluoromethyl)phenyl]ethyl]amino}thieno[3,2-d]pyrimidin-6-yl)-1,2,3,6-tetrahydropyridine-1-carboxylate (386 mg, 0.74 mmol) in ether (1.2 ml) HCl (4 M in dioxane, 0.93 ml, 3.7 mmol) was added and the mixture was stirred at rt for 12 h. The reaction was poured into the water and neutralized with NaHCO₃ aq. The mixture was extracted with DCM and the combined organic phases were dried over Na₂SO₄. The solvent was removed under reduced pressure and the crude product was purified by prep-HPLC to give N-[(1R)-1-[3-amino-5-(trifluoromethyl)phenyl]ethyl]-6-(1,2,3,6-tetrahydropyridin-4-yl)thieno[3,2-d]pyrimidin-4-amine (16.5 mg, 6% yield). LCMS (ESI): m/z: [M+H] calculated for C₂₀H₂₁F₃N₅S: 420.1; found 420.0; ¹H NMR (300 MHz, Methanol-d₄) δ 8.35 (s, 1H), 7.21 (s, 1H), 6.96 (d, J=5.9 Hz, 2H), 6.81 (s, 1H), 6.54 (s, 1H), 5.50 (q, J=7.0 Hz, 1H), 3.52 (d, J=3.2 Hz, 2H), 3.09 (t, J=5.7 Hz, 2H), 2.59 (s, 2H), 1.61 (d, J=7.1 Hz, 3H).

### Example 10. Synthesis of N-[(1R)-1-[3-amino-5-(trifluoromethyl)phenyl]ethyl]-2-(4-methylpiperazine-1-carbonyl)imidazo[1,2-a]pyrazin-8-amine

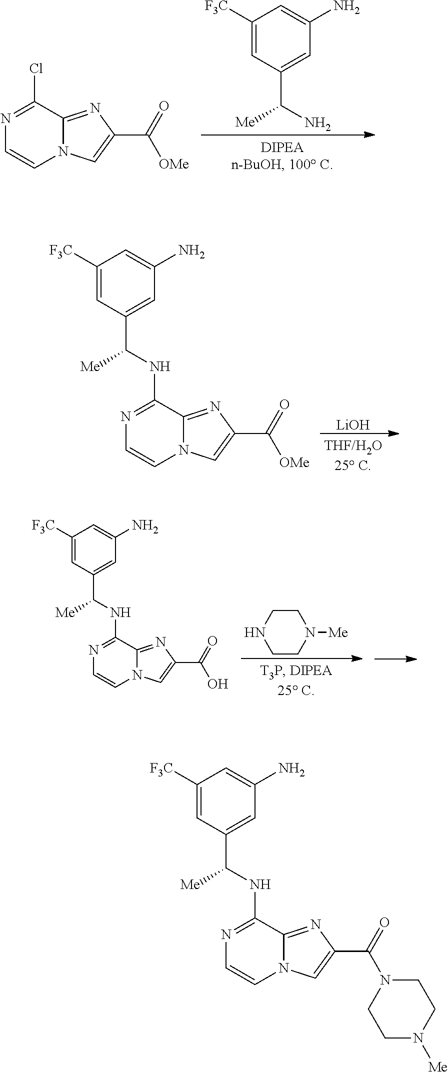

### Step 1.

To a solution of methyl 8-chloroimidazo[1,2-a]pyrazine-2-carboxylate (200 mg, 945 μmol) and (R)-3-(1-aminoethyl)-5-(trifluoromethyl)aniline (250 mg, 1.23 mmol) in n-BuOH (6 mL) was added DIPEA (1.65 mL, 9.45 mmol). The mixture was stirred at 100° C. for 12 h. After cooling to rt, H₂O was added and the mixture was extracted with EtOAc. The combined organic phases were dried with anhydrous Na₂SO₄ and the solvent was removed under reduced pressure. The residue was purified by prep-TLC to give (R)-methyl 8-((1-(3-amino-5-(trifluoromethyl)phenyl)ethyl)amino)imidazo[1,2-a]pyrazine-2-carboxylate (230 mg, 45% yield). LCMS (ESI): m/z: [M+H] calculated for C₁₇H₁₇F₃N₅O₂: 380.1; found; 380.2.

### Step 2.

To a solution of (R)-methyl 8-((1-(3-amino-5-(trifluoromethyl)phenyl)ethyl)amino)imidazo[1,2-a]pyrazine-2-carboxylate (230 mg, 606 μmol) in THF (4 mL) and H₂O (4 mL) was added LiOH monohydrate (38 mg, 909 μmol). The mixture was stirred at 25° C. for 1 h, aq. HCl (1N) was added until pH=3-4. The aqueous phase was extracted with DCM and the combined organic phases were dried over Na₂SO₄. The solvent was removed under reduced pressure to give (R)-8-((1-(3-amino-5-(trifluoromethyl)phenypethyl)amino)imidazo[1,2-a]pyrazine-2-carboxylic acid (220 mg, 78% yield). ¹H NMR (400 MHz, METHANOL-d₄) δ ppm 8.29 (s, 1H) 7.68 (d, J=4.77 Hz, 1H) 7.27 (d, J=4.77 Hz, 1H) 6.96 (d, J=6.48 Hz, 2H) 6.80 (s, 1H) 5.26 (q, J=7.17 Hz, 1H) 1.60 (d, J=6.97 Hz, 3H).

### Step 3.

To a solution of (R)-8-((1-(3-amino-5-(trifluoromethyl)phenyl)ethyl)amino)imidazo[1,2-a]pyrazine-2-carboxylic acid (100 mg, 273 μmol) and 1-methylpiperazine (45.55 μL, 410.61 μmol) in THF (5 mL) was added DIPEA (238 μL, 1.37 mmol) and T₃P (244 uL, 821 μmol). The mixture was stirred at 25° C. for 1 h, the solvent was removed under reduced pressure and the crude residue was purified by prep-HPLC to give (R)-(8-((1-(3-amino-5-(trifluoromethyl)phenyl)ethyl)amino)imidazo[1,2-a]pyrazin-2-yl)(4-methylpiperazin-1-yl)methanone (20 mg, 16% yield). LCMS (ESI): m/z: [M+H] calculated for C₂₁H₂₅F₃N₇O: 448.2; found 448.2; ¹H NMR (400 MHz, METHANOL-d₄) δ ppm 8.13 (s, 1H) 7.67 (d, J=4.65 Hz, 1H) 7.25 (d, J=4.77 Hz, 1H) 6.95 (d, J=5.14 Hz, 2H) 6.79 (s, 1H) 5.30 (q, J=6.89 Hz, 1H) 4.19 (s, 2H) 3.82 (s, 2H) 2.54 (s, 4H) 2.35 (s, 3H) 1.61 (d, J=6.97 Hz, 3H).

### Example 11. Synthesis of N-[(1R)-1-[3-amino-5-(trifluoromethyl)phenyl]ethyl]-6-(piperidin-4-yl)pyrrolo[2,1-f][1,2,4]triazin-4-amine

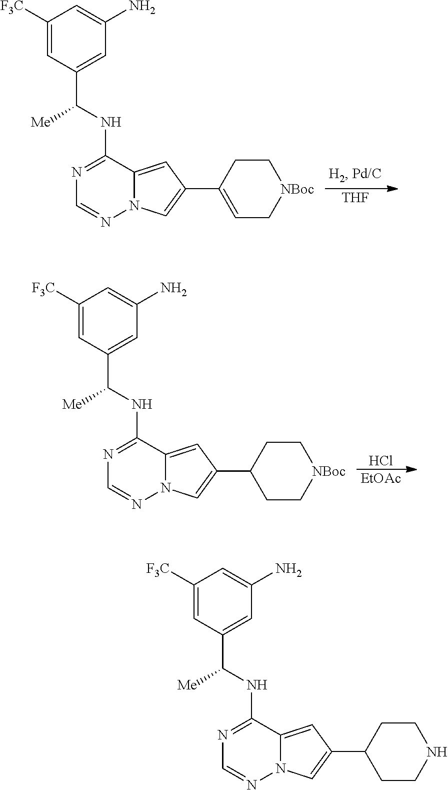 [SMILES: "EtOAc" ; C[C@@H](Nc1ncnn2cc(C3=CCN(C(=O)OC(C)(C)C)CC3)cc12)c1cc(N)cc(C(F)(F)F)c1 ; C[C@@H](Nc1ncnn2cc(C3CCN(C(=O)OC(C)(C)C)CC3)cc12)c1cc(N)cc(C(F)(F)F)c1 ; Cl ; C[C@@H](Nc1ncnn2cc(C3CCNCC3)cc12)c1cc(N)cc(C(F)(F)F)c1 ; "THF"]

### Step 1.

To a solution of tert-butyl 4-[4-[[(1R)-1-[3-amino-5-(trifluoromethyl)phenyl]ethyl]amino]pyrrolo[2,1-f][1,2,4]triazin-6-yl]-3,6-dihydro-2H-pyridine-1-carboxylate (90 mg, 179 μmol) in THF (2 mL) was added Pd/C (40 mg, 179 μmol, 10 wt %). The mixture was stirred under H₂ at 20° C. for 2 h, filtered and the solvent was removed under reduced pressure to give tert-butyl 4-[4-[[(1R)-1-[3-amino-5-(trifluoromethyl)phenyl]ethyl]amino]pyrrolo[2,1-f][1,2,4]triazin-6-yl]piperidine-1-carboxylate (70 mg, 77% yield), which was used in the next step without further purification. LCMS (ESI): m/z: [M+H] calculated for C₂₅H₃₂F₃N₆O₂: 505.2; found 505.1.

### Step 2.

To a solution of tert-butyl 4-[4-[[(1R)-1-[3-amino-5-(trifluoromethyl)phenyl]ethyl]amino]pyrrolo[2,1-f][1,2,4]triazin-6-yl]piperidine-1-carboxylate (65 mg, 128 μmol) in EtOAc (2 mL) was added HCl/EtOAc (4 M, 2 mL). The mixture was stirred at 25° C. for 1 h under N₂, the solvent was removed under reduced pressure and the crude residue was purified by prep-HPLC N-[(1R)-1-[3-amino-5-(trifluoromethyl)phenyl]ethyl]-6-(4-piperidyl)pyrrolo[2,1-f][1,2,4]triazin-4-amine (40 mg, 76% yield). LCMS (ESI): m/z: [M+H] calculated for C₂₀H₂₄F₃N₆: 405.2; found 405.3; ¹H NMR (400 MHz, METHANOL-d₄) δ ppm 7.93 (s, 1H), 7.84 (d, J=0.9 Hz, 1H), 7.54 (s, 1H), 7.43 (d, J=8.8 Hz, 2H), 7.29 (s, 1H), 5.39-5.24 (m, 1H), 3.57-3.45 (m, 2H), 3.25-3.06 (m, 3H), 2.27 (d, J=14.2 Hz, 2H), 1.99-1.84 (m, 2H), 1.78 (d, J=6.8 Hz, 3H).

### Example 12. Synthesis of N-[(1R)-1-[3-amino-5-(trifluoromethyl)phenyl]ethyl]-6-(1,2,3,6-tetrahydropyridin-4-yl)pyrrolo[2,1-f][1,2,4]triazin-4-amine

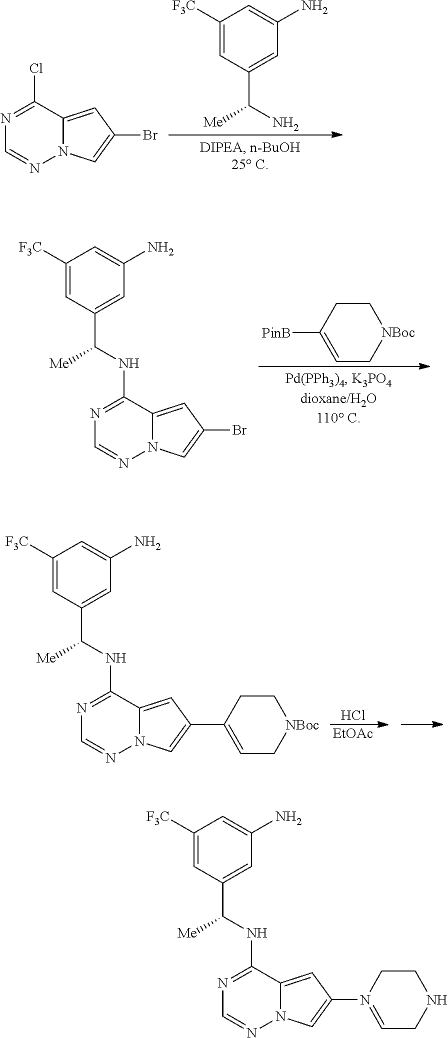

### Step 1.

To a solution of 6-bromo-4-chloro-pyrrolo[2,1-1][1,2,4]triazine (300 mg, 1.29 mmol) and DIPEA (450 uL, 2.58 mmol) in n-BuOH (2 mL) was added 3-[(1R)-1-aminoethyl]-5-(trifluoromethyl)aniline (289 mg, 1.42 mmol). The mixture was stirred at 25° C. for 3 h under N₂, the solvent was removed under reduced pressure and the crude residue was purified by silica gel chromatography to give N-[(1R)-1-[3-amino-5-(trifluoromethyl)phenyl]ethyl]-6-bromo-pyrrolo[2,1-f][1,2,4]triazin-4-amine (330 mg, 64% yield). ¹H NMR (400 MHz, METHANOL-d₄) δ ppm 7.77 (s, 1H), 7.55 (d, J=1.8 Hz, 1H), 7.02 (d, J=1.8 Hz, 1H), 6.91 (d, J=7.3 Hz, 2H), 6.80 (s, 1H), 5.42 (q, J=7.0 Hz, 1H), 1.59 (d, J=7.1 Hz, 3H).

### Step 2.

To a solution of N-[(1R)-1-[3-amino-5-(trifluoromethyl)phenyl]ethyl]-6-bromo-pyrrolo[2,1-f][1,2,4]triazin-4-amine (330 mg, 824 μmol) and tert-butyl 4-(4,4,5,5-tetramethyl-1,3,2-dioxaborolan-2-yl)-3,6-dihydro-2H-pyridine-1-carboxylate (382 mg, 1.24 mmol) in dioxane (5 mL) and H₂O (0.5 mL) was added K₃PO₄ (700 mg, 3.3 mmol) and Pd(PPh₃)₄ (Palladium-tetrakis(triphenylphosphine, 47 mg, 41 μmol) at 25° C. The mixture was stirred at 110° C. for 8 h under N₂, cooled to rt and filtered. The solvent was removed under reduced pressure and the crude residue was purified by silica gel chromatography to give tert-butyl 4-[4-[[(1R)-1-[3-amino-5-(trifluoromethyl)phenyl]ethyl]amino]pyrrolo[2,1-f][1,2,4]triazin-6-yl]-3,6-dihydro-2H-pyridine-1-carboxylate (260 mg, 62% yield). ¹H NMR (400 MHz, METHANOL-d₄) δ ppm 7.73 (s, 1H), 7.60 (d, J=1.3 Hz, 1H), 7.05 (d, J=1.3 Hz, 1H), 6.93 (d, J=7.7 Hz, 2H), 6.80 (s, 1H), 6.12 (s, 1H), 5.42 (q, J=6.8 Hz, 1H), 4.07 (d, J=5.7 Hz, 2H), 3.64 (s, 2H), 2.51 (s, 2H), 1.60 (d, J=7.1 Hz, 3H), 1.49 (s, 8H)

### Step 3.

A solution of tert-butyl 4-[4-[[(1R)-1-[3-amino-5-(trifluoromethyl)phenyl]ethyl]amino]pyrrolo[2,1-f][1,2,4]triazin-6-yl]-3,6-dihydro-2H-pyridine-1-carboxylate (40 mg, 79 μmol) in EtOAc (2 mL) was added HCl/EtOAc (4 M, 2 mL). The mixture was stirred at 25° C. for 1 h under N₂, the solvent was removed under reduced pressure and the crude residue was purified by prep-HPLC to give 3-(1,2,3,6-tetrahydropyridin-4-yl)-N-[(1S)-1-[3-(trifluoromethyl)phenyl]ethyl]imidazo[1,2-a]pyrazin-8-amine (80 mg, 92% yield). LCMS (ESI): m/z: [M+H] calculated for C₂₀H₂₂F₃N₆: 403.2, found 403.1; ¹H NMR (400 MHz, METHANOL-d₄) δ ppm 7.73 (s, 1H), 7.59 (d, J=1.6 Hz, 1H), 7.06 (d, J=1.3 Hz, 1H), 6.92 (d, J=7.5 Hz, 2H), 6.80 (s, 1H), 6.19 (s, 1H), 5.41 (q, J=6.9 Hz, 1H), 3.51 (d, J=2.8 Hz, 2H), 3.11 (t, J=5.9 Hz, 2H), 2.52 (d, J=1.7 Hz, 2H), 1.60 (d, J=7.1 Hz, 3H).

### Example 13. Synthesis of N-[(1R)-1-[3-amino-5-(trifluoromethyl)phenyl]ethyl]-6-(piperidin-4-yl)pyrrolo[2,1-f][1,2,4]triazin-4-amine

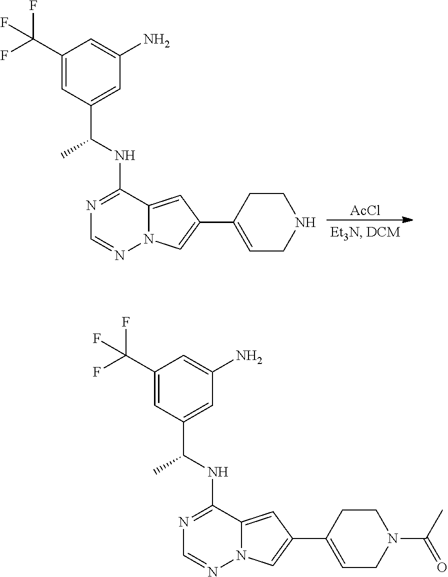 [SMILES: "AcCl" ; C[C@@H](Nc1ncnn2cc(C3=CCNCC3)cc12)c1cc(N)cc(C(F)(F)F)c1 ; CC(=O)N1CC=C(c2cc3c(N[C@H](C)c4cc(N)cc(C(F)(F)F)c4)ncnn3c2)CC1]

### Step 1.

To a solution of N-[(1R)-1-[3-amino-5-(trifluoromethyl)phenyl]ethyl]-6-(1,2,3,6-tetrahydropyridin-4-yl)pyrrolo[2,1-f][1,2,4]triazin-4-amine (65 mg, 161.52 μmol) in DCM (1 mL) was added Et₃N (67 μL, 484 μmol) and acetyl chloride (9 uL, 129 μmol). Then the mixture was stirred at 25° C. for 1 h under N₂ and then poured into water. After extraction with DCM, the combined organic phases were washed with brine and dried over Na₂SO₄. The solvent was removed under reduced pressure and the crude residue was purified by prep-HPLC to give 1-[4-[4-[[(1R)-1-[3-amino-5-(trifluoromethyl)phenyl]ethyl]amino]pyrrolo[2,1-f][1,2,4]triazin-6-yl]-3,6-dihydro-2H-pyridin-1-yl]ethanone (32 mg, 45% yield). LCMS (ESI): m/z: [M+H] calculated for C₂₂H₂₄F₃N₆O: 445.2; found 445.0; ¹H NMR (400 MHz, METHANOL-d₄) δ ppm 7.74 (s, 1H), 7.61 (dd, J=1.7, 7.4 Hz, 1H), 7.06 (s, 1H), 6.93 (d, J=7.7 Hz, 2H), 6.81 (s, 1H), 6.15 (dd, J=1.3, 3.1 Hz, 1H), 5.42 (q, J=6.9 Hz, 1H), 4.20 (dd, J=2.8, 5.0 Hz, 2H), 3.84-3.71 (m, 2H), 2.66-2.48 (m, 2H), 2.16 (d, J=14.8 Hz, 3H), 1.60 (d, J=7.1 Hz, 3H).

### Example 28. Synthesis of N-[(1R)-1-[3-amino-5-(trifluoromethyl)phenyl]ethyl]-2-methyl-6-(methylaminomethyl)pyrrolo[2,1-f][1,2,4]triazin-4-amine

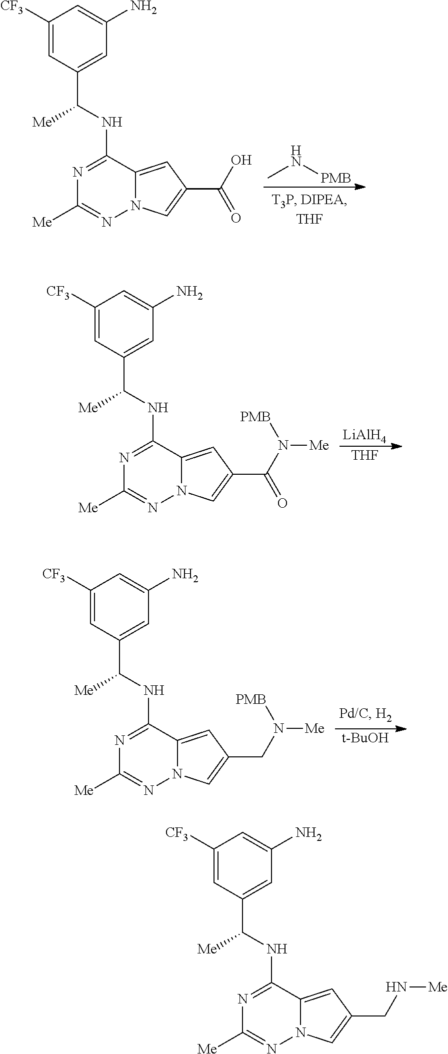

### Step 1.

To a mixture of 4-[[(1R)-1-[3-amino-5-(trifluoromethyl)phenyl]ethyl]amino]-2-methyl-pyrrolo[2,1-f][1,2,4]triazine-6-carboxylic acid (400 mg, 1.05 mmol) in THF (5 mL) was added T3P (468 μL, 1.58 mmol), 1-(4-methoxyphenyl)-N-methyl-methanamine (317 mg, 2.1 mmol) and DIPEA (732 μL, 4.2 mmol). The mixture was stirred at rt for 4 h, the solvent was removed under reduced pressure and the residue was purified by prep-TLC to give 4-[[(1R)-1-[3-amino-5-(trifluoromethyl)phenyl]ethyl]amino]-N-[(4-methoxyphenyl)methyl]-N2-dimethyl-pyrrolo[2,1-f][1,2,4]triazine-6-carboxamide (310 mg, 57% yield). ¹H NMR (400 MHz, METHANOL-d₄) δ ppm 7.80-7.58 (m, 1H), 7.31-7.13 (m, 3H), 6.93 (br d, J=7.8 Hz, 4H), 6.80 (s, 1H), 5.53 (d, J=6.8 Hz, 1H), 4.83-4.66 (m, 2H), 3.79 (s, 3H), 3.35 (s, 3H), 2.27 (br s, 3H), 1.58 (br d, J=6.8 Hz, 3H).

### Step 2.

To a mixture of 4-[[(1R)-1-[3-amino-5-(trifluoromethyl)phenyl]ethyl]amino]-N-[(4-methoxyphenyl)methyl]-N2-dimethyl-pyrrolo[2,1-f][1,2,4]triazine-6-carboxamide (100 mg, 0.2 mmol) in THF (2 mL) was added LiAlH₄ (22 mg, 0.59 mmol). The mixture was stirred at rt for 2 h, then diluted with H₂O (5 mL) and extracted with EtOAc (5 mL×3). The combined organic layers were washed with brine (5 mL), dried over Na₂SO₄ and filtered. The solvent was concentrated under reduced pressure and the crude residue was purified by prep-TLC to give N-[(1R)-1-[3-amino-5-(trifluoromethyl)phenyl]ethyl]-6-[[(4-methoxyphenyl)methyl-methyl-amino]methyl]-2-methyl-pyrrolo[2,1-f][1,2,4]triazin-4-amine (50 mg, 51% yield).

### Step 3.

To a mixture of N-[(1R)-1-[3-amino-5-(trifluoromethyl)phenyl]ethyl]-6-[[(4-methoxyphenyl) methyl-methyl-amino]methyl]-2-methyl-pyrrolo[2,1 -f][1,2,4]triazin-4-amine (25 mg, 50 μmol) in t-BuOH (1 mL) was added 10% Pd/C (0.5 g, 5.0 μmol). The suspension was degassed under vacuum and purged with H₂ several times. The mixture was stirred under H₂ (50 psi) at rt for 12 h, then MeOH (20 mL) was added and the mixture was filtered. The solvent was removed under reduced pressure and the residue was purified by prep-HPLC to give N-[(1R)-1-[3-amino-5-(trifluoromethyl)phenyl]ethyl]-2-methyl-6-(methylaminomethyl)pyrrolo[2,1-f][1,2,4]triazin-4-amine (4 mg, 20% yield). LCMS (ESI): m/z: [M+H] calculated for C₁₈H₂₂F₃N₆: 379.2; found 379.2; ¹H NMR (400 MHz, METHANOL-d₄) δ ppm 8.56 (br s, 1H), 7.58 (s, 1H), 7.01-6.94 (m, 3H), 6.82 (s, 1H), 5.56 (q, J=6.8 Hz, 1H), 4.20 (br s, 2H), 2.70 (s, 3H), 2.30 (s, 3H), 1.61 (d, J=6.8 Hz, 3H).

### Example 29. Synthesis of [8-[[(1R)-1-[3-amino-5-(trifluoromethyl)phenyl]ethyl]amino]-6-chloro-imidazo[1,2-a]pyrazin-2-yl]-morpholino-methanone

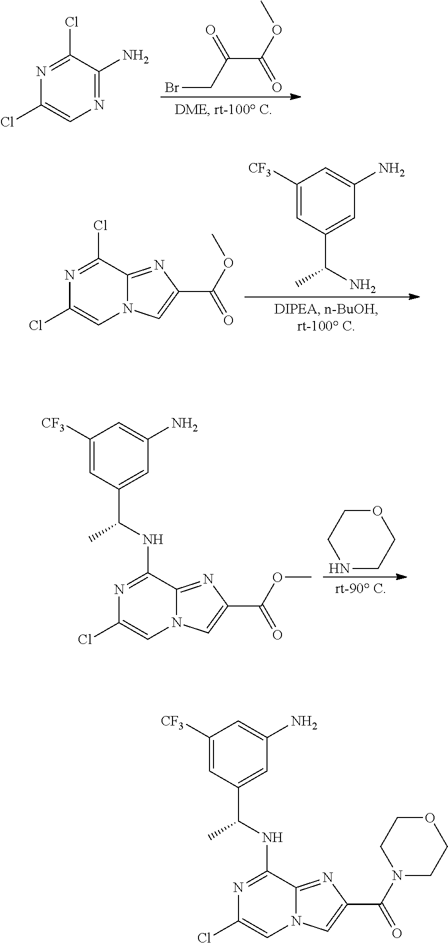

### Step 1.

To a mixture of 3,5-dichloropyrazin-2-amine (500 mg, 3.05 mmol) in DME (12 mL) was added methyl 3-bromo-2-oxo-propanoate (390 μL, 3.66 mmol) in one portion at rt under N₂. The mixture was heated to 100° C. and stirred for 14 h. The mixture was filtered and the filter cake was dried to afford methyl 6,8-dichloroimidazo[1,2-a]pyrazine-2-carboxylate HBr salt (350 mg, 35% yield). ¹H NMR (400 MHz, METHANOL-d₄) δ ppm 8.69-8.70 (m, 1H), 8.61-8.62 (m, 1H), 3.98 (s, 3H).

### Step 2.

To a mixture of methyl 6,8-dichloroimidazo[1,2-a]pyrazine-2-carboxylate HBr salt (340 mg, 1.04 mmol) and 3-[(1R)-1-aminoethyl]-5-(trifluoromethyl)aniline (212 mg, 1.04 mmol) in n-BuOH (7 mL) was added DIPEA (725 μL, 4.16 mmol). The mixture was heated to 100° C. and stirred for 1 h, cooled, H₂O (2 mL) added, and the mixture filtered. The solvent was concentrated under reduced pressure and the residue was purified by column chromatography to give 8-[[(1R)-1-[3-amino-5-(trifluoromethyl)phenyl]ethyl]amino]-6-chloro-imidazo[1,2-a]pyrazine-2-carboxylate (400 mg, 93% yield). ¹H NMR (400 MHz, DMSO-d₆) δ ppm 8.87 (br d, J=8.0 Hz, 1H), 8.43 (s, 1H), 7.92 (s, 1H), 6.97 (s, 1H), 6.83 (s, 1H), 6.69 (s, 1H), 5.54 (br s, 2H), 5.26 (t, J=6.8 Hz, 1H), 3.85 (s, 3H), 1.53 (d, J=7.2 Hz, 3H).

### Step 3.

A mixture of methyl 8-[[(1R)-1-[3-amino-5-(trifluoromethyl)phenyl]ethyl]amino]-6-chloro-imidazo[1,2-a]pyrazine-2-carboxylate (50 mg, 121 μmol) in morpholine (2 mL) was heated to 90° C. and stirred for 12 h. The mixture was filtered, the solvent was concentrated under reduced pressure and the residue was purified by prep-HPLC to give [8-[[(1R)-1-[3-amino-5-(trifluoromethyl)phenyl]ethyl]amino]-6-chloro-imidazo[1,2-a]pyrazin-2-yl]-morpholino-methanone (17 mg, 30% yield). LCMS (ESI): m/z: [M+H] calculated for C₂₀H₂₁ClF₃N₆O₂: 469.14; found: 469.1; ¹H NMR (400 MHz, DMSO-d₆) δ ppm 8.45 (br d, J=8.4 Hz, 1H), 8.20 (s, 1H), 7.94 (s, 1H), 6.92 (s, 1H), 6.83 (s, 1H), 6.70 (s, 1H), 5.55 (br s, 2H), 5.29 (s, 1H), 4.15 (s, 2H), 3.64 (s, 6H), 1.54 (d, J=6.8 Hz, 3H).

### Example 30. Synthesis of N-[(1R)-1-[3-amino-5-(trifluoromethyl)phenyl]ethyl]-6-methoxy-2-methyl-pyrrolo[2,14]-[1,2,4]triazin-4-amine

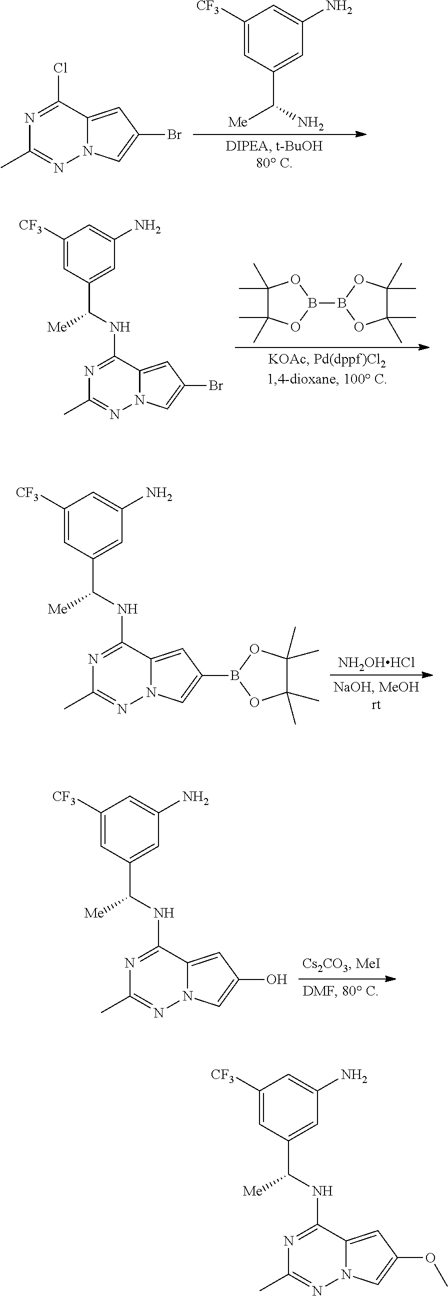

### Step 1.

To a mixture of 6-bromo-4-chloro-2-methyl-pyrrolo[2,1-f][1,2,4]triazine (1.0 g, 4.1 mmol) and 3-[(1R)-1-aminoethyl]-5-(trifluoromethyl)aniline (828 mg, 4.1 mmol) in t-BuOH (10 mL) was added DIPEA (1.41 mL, 8.1 mmol). The mixture was heated to 80° C. and stirred for 1.5 h, then cooled and poured into H₂O (10 mL). The mixture was extracted with EtOAc (10 mL×3), and the combined organic extracts were washed with brine (20 mL), dried with anhydrous Na₂SO₄, and filtered. The solvent was concentrated under reduced pressure and the residue was purified by column chromatography to give N-[(1R)-1-[3-amino-5-(trifluoromethyl)phenyl]ethyl]-6-bromo-2-methyl-pyrrolo[2,1-f][1,2,4]-triazin-4-amine (1.1 g, 66% yield). LCMS (ESI): m/z: [M+H] calculated for C₁₆H₁₆BrF₃N₅: 414.05; found 414.0; ¹H NMR (400 MHz, METHANOL-d₄) δ ppm 7.42 (d, J=2.0 Hz, 1H), 6.94 (s, 1H), 6.93-6.90 (m, 2H), 6.80 (s, 1H), 5.50 (q, J=6.8 Hz, 1H), 2.25 (s, 3H), 1.57 (d, J=6.8 Hz, 3H).

### Step 2.

To a mixture of N-[(1R)-1-[3-amino-5-(trifluoromethyl)phenyl]ethyl]-6-bromo-2-methyl-pyrrolo[2,1-f][1,2,4]triazin-4-amine (1.0 g, 2.4 mmol) and bis(pinacolato)diboron (613 mg, 2.4 mmol) in 1,4-dioxane (10 mL) under an atmosphere of N₂ was added AcOK (474 mg, 4.83 mmol) and Pd(dppf)Cl₂ ([1,1′-Bis(diphenylphosphino)ferrocene]dichloropalladium(II), 353 mg, 0.48 mmol). The mixture was heated to 100° C. and stirred for 1 h. The mixture was filtered, the solvent was concentrated under reduced pressure and the residue was purified by prep-HPLC to give N-[(1R)-1-[3-amino-5-(trifluoromethyl)phenyl]ethyl]-2-methyl-6-(4,4,5,5-tetramethyl-1,3,2-dioxaborolan-2-yl)pyrrolo[2,1-f][1,2,4]triazin-4-amine (150 mg, 14% yield). LCMS (ESI): m/z: [M+H] calculated for C₂₂H₂₈BF₃N₅O₂: 462.22; found 462.1.

### Step 3.

To a mixture of N-[(1R)-1-[3-amino-5-(trifluoromethyl)phenyl]ethyl]-2-methyl-6-(4,4,5,5-tetramethyl-1,3,2-dioxaborolan-2-yl)pyrrolo[2,1-f][1,2,4]triazin-4-amine (70 mg, 0.15 mmol) in MeOH (2 mL) was added NaOH (61 mg, 1.5 mmol) and hydroxylamine hydrochloride (53 mg, 0.76 mmol) under an atmosphere of N₂. The mixture was stirred at rt for 1 h, the solvent was concentrated under reduced pressure and the residue was purified by column chromatography to give 4-[[(1R)-1-[3-amino-5-(trifluoromethyl)phenyl]ethyl]amino]-2-methyl-pyrrolo[2,1-f][1,2,4]triazin-6-ol (40 mg, 75% yield). LCMS (ESI): m/z: [M+H] calculated for C₁₆H₁₇F₃N₅O: 352.13; found 352.0.

### Step 4.

To a mixture of 4-[[(1R)-1-[3-amino-5-(trifluoromethyl)phenyl]ethyl]amino]-2-methyl-pyrrolo[2,1-f][1,2,4]triazin-6-ol (30 mg, 85 μmol) in DMF (1 mL) at 0° C. was added Cs₂CO₃ (31 mg, 94 μmol), and the mixture was stirred at 0° C. for 12 min. MeI (5.3 μL, 85 μmol) was added slowly, and the mixture heated to 80° C. and stirred for 1 h. The mixture was filtered, the solvent was concentrated under reduced pressure and the residue was purified by prep-HPLC to give N-[(1R)-1-[3-amino-5-(trifluoromethyl)phenyl]ethyl]-6-methoxy-2-methyl-pyrrolo[2,1-f]-[1,2,4]triazin-4-amine (9 mg, 29% yield). LCMS (ESI): m/z: [M+H] calculated for C₁₇H₁₉F₃N₅O: 366.15; found 366.1; ¹H NMR (400 MHz, METHANOL-d₄) δ ppm 7.12 (d, J=2.0 Hz, 1H), 6.94 (s, 1H), 6.92 (s, 1H), 6.79 (s, 1H), 6.49 (d, J=2.0 Hz, 1H), 5.53-5.46 (m, 1H), 3.79 (s, 3H), 2.28-2.23 (m, 3H), 1.57 (d, J=6.8 Hz, 3H).

### Example 31. Synthesis of N-[(1R)-1-[3-amino-5-(trifluoromethyl)phenyl]ethyl]-6-methoxy-2-methyl-pyrrolo[2,1-f]-[1,2,4]triazin-4-amine

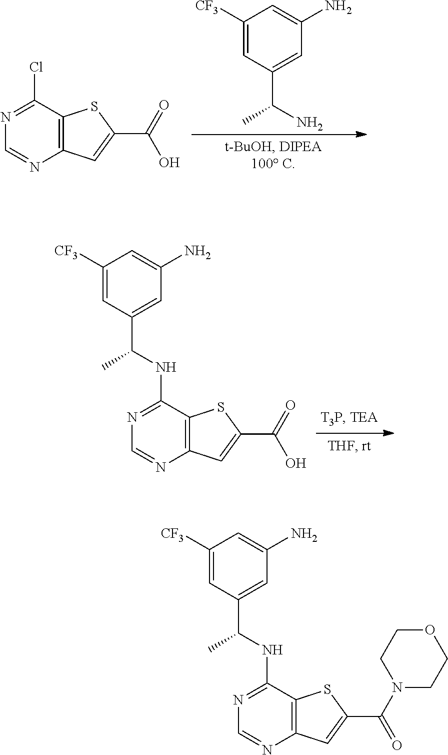 [SMILES: O=C(O)c1cc2ncnc(Cl)c2s1 ; C[C@@H](N)c1cc(N)cc(C(F)(F)F)c1 ; C[C@@H](Nc1ncnc2cc(C(=O)O)sc12)c1cc(N)cc(C(F)(F)F)c1 ; C[C@@H](Nc1ncnc2cc(C(=O)N3CCOCC3)sc12)c1cc(N)cc(C(F)(F)F)c1]

### Step 1.

To a mixture of 4-chlorothieno[3,2-d]pyrimidine-6-carboxylic acid (200 mg, 0.93 mmol) in t-BuOH (4 mL) was added 3-[(1R)-1-aminoethyl]-5-(trifluoromethyl)aniline (228 mg, 1.12 mmol) and DIPEA (1.62 mL, 9.32 mmol). The mixture was heated to 100° C. and stirred for 16 h in a crimped vial. The solvent was concentrated under reduced pressure and the residue was purified by prep-HPLC to give 4-[[(1R)-1-[3-amino-5-(trifluoromethyl)phenyl]ethyl]amino]thieno[3,2-d]pyrimidine-6-carboxylic acid (40 mg, 11% yield). LCMS (ESI): m/z: [M+H] calculated for C₁₆H₁₄F₃N₄O₂S: 383.1; found 383.1.

### Step 2.

To a mixture of 4-[[(1R)-1-[3-amino-5-(trifluoromethyl)phenyl]ethyl]amino]thieno[3,2-d] pyrimidine-6-carboxylic acid (30 mg, 78 μmol) in THF (2 mL) was added DIPEA (41 μL, 0.23 mmol), T3P (47 μL, 0.16 mmol) and morpholine (7.6 μL, 86 μmol). The mixture was stirred at rt for 6 h., the solvent was concentrated under reduced pressure and the residue was purified by prep-HPLC to give [4-[[(1R)-1-[3-amino-5-(trifluoromethyl)phenyl]ethyl]amino]thieno[3,2-d]pyrimidin-6-yl]-morpholino-methanone (10.6 mg, 30% yield). LCMS (ESI): m/z: [M+H] calculated for C₂₀H₂₁F₃N₅O₂S: 452.1; found 452.1; ¹H NMR (400 MHz, DMSO-d₆) δ ppm 8.42 (s, 1H), 8.39 (d, J=8.0 Hz, 1H), 7.63 (s, 1H), 6.86 (s, 1H), 6.82 (s, 1H), 6.69 (s, 1H), 5.55 (s, 2H), 5.44-5.40 (t, J=7.2 Hz, 1H), 3.66 (s, 8H), 1.50 (d, J=7.2 Hz, 3H).

### Example 32. Synthesis of N-[(1R)-1-[3-amino-5-(trifluoromethyl)phenyl]ethyl]-6-(tetrahydrofuran-3-ylmethyl)pyrrolo[3,4-d]pyrimidin-4-amine

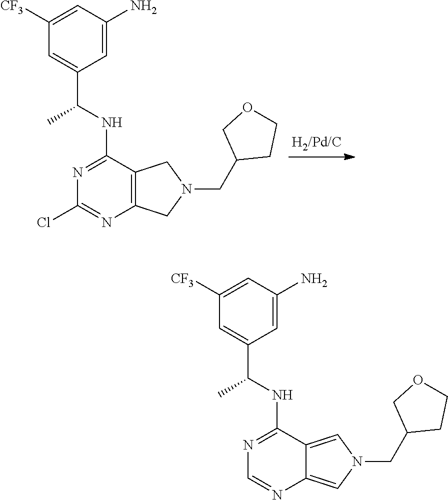 [SMILES: C[C@@H](Nc1nc(Cl)nc2c1CN(CC1CCOC1)C2)c1cc(N)cc(C(F)(F)F)c1 ; C[C@@H](Nc1ncnc2cn(CC3CCOC3)cc12)c1cc(N)cc(C(F)(F)F)c1]

### Step 1.

To a mixture of N-[(1R)-1-[3-amino-5-(trifluoromethyl)phenyl]ethyl]-2-chloro-6-(tetrahydrofuran-3-ylmethyl)-5,7-dihydropyrrolo[3,4-d]pyrimidin-4-amine (20 mg, 45 μmol) in THF (2 mL) and MeOH (4 mL) was added 10% wt. Pd on carbon (20 mg, 45 μmol). The mixture was heated to 40° C. and stirred under at atmosphere of H₂ for 48 h. The mixture was filtered, the solvent was concentrated under reduced pressure and the residue was purified by prep-HPLC to give N-[(1R)-1-[3-amino-5-(trifluoromethyl)phenyl]ethyl]-6-(tetrahydrofuran-3-ylmethyl)pyrrolo[3,4-d]pyrimidin-4-amine (5 mg, 27% yield). LCMS (ESI): m/z: [M+H] calculated for C₂₀H₂₃F₃N₅O: 406.2; found 406.1; ¹H NMR (400 MHz, DMSO-d₆) δ ppm 8.19 (s, 1H), 7.98 (d, J=7.6 Hz, 1H), 7.93 (s, 1H), 7.54 (s, 1H), 7.22 (d, J=1.6 Hz, 1H), 6.82 (d, J=5.2 Hz, 2H), 6.69 (s, 1H), 5.54 (br s, 2H), 5.41 (t, J=7.2 Hz, 1H), 4.15 (d, J=7.6 Hz, 2H), 3.86-3.74 (m, 1H), 3.72-3.59 (m, 2H), 3.45 (dd, J=8.4, 5.6 Hz, 1H), 2.83-2.70 (m, 1H), 2.00-1.87 (m, 1H), 1.66-1.53 (m, 1H), 1.48 (d, J=6.8 Hz, 3H).

### Example 33. Synthesis of [4-[[(1R)-1-[3-amino-5-(trifluoromethyl)phenyl]ethyl]amino]-2-methyl-pyrrolo[2,1-f][1,2,4]triazin-6-yl]-(3-hydroxyazetidin-1-yl)methanone

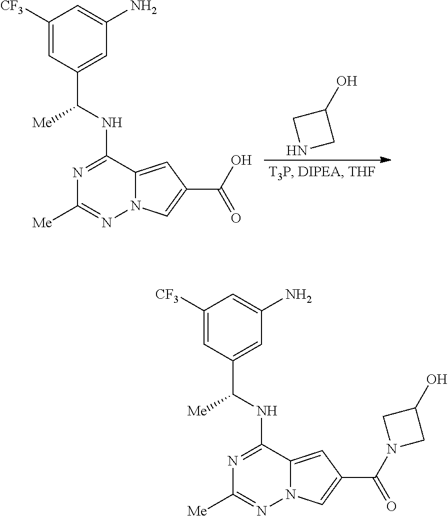 [SMILES: Cc1nc(N[C@H](C)c2cc(N)cc(C(F)(F)F)c2)c2cc(C(=O)O)cn2n1 ; OC1CNC1 ; Cc1nc(N[C@H](C)c2cc(N)cc(C(F)(F)F)c2)c2cc(C(=O)N3CC(O)C3)cn2n1]

### Step 1.

[4-[[(1R)-1-[3-amino-5-(trifluoromethyl)phenyl]ethyl]amino]-2-methyl-pyrrolo[2,1-f][1,2,4]triazin-6-yl]-(3-hydroxyazetidin-1-yl)methanone was synthesized in a manner similar to 4-[[(1R)-1-[3-amino-5-(trifluoromethyl)phenyl]ethyl]amino]-N-[(4-methoxyphenyl)methyl]-N2-dimethyl-pyrrolo[2,1-f][1,2,4]triazine-6-carboxamide except 1-(4-methoxyphenyl)-N-methyl-methanamine was substituted with azetidine-3-ol. LCMS (ESI): m/z: [M+H] calculated for C₂₀H₂₁F₃N₆O₂: 435.2; found 435.1; ¹H NMR (400 MHz, METHANOL-d₄) δ ppm 7.81 (d, J=2.0 Hz, 1H), 7.26 (d, J=2.0 Hz, 1H), 6.96 (d, J=7.8 Hz, 2H), 6.83 (s, 1H), 5.60-5.53 (m, 1H), 4.75 (d, J=8.3 Hz, 1H), 4.69 (tt, J=7.0, 3.5 Hz, 1H), 4.45-4.38 (m, 1H), 4.32 (d, J=5.9 Hz, 1H), 3.97 (d, J=11.2 Hz, 1H), 2.31 (s, 3H), 1.62 (d, J=6.8 Hz, 3H).

The following Examples 69-72 shown in Table 1 were synthesized in the manner similar to Example 33.

TABLE 1
Examples 69-72

| | | Mass |
|---|---|---|
| Example # | Structure | Found |
| Example 69. | 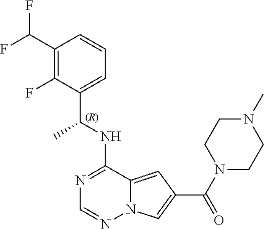 | 433.0 |
| Example 70. | 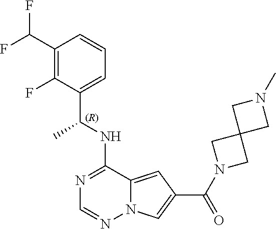 | 445.0 |
| Example 71. | 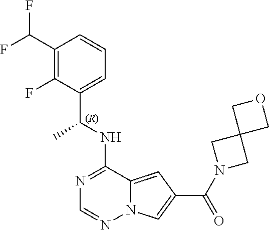 | 432.0 |
| Example 72. | 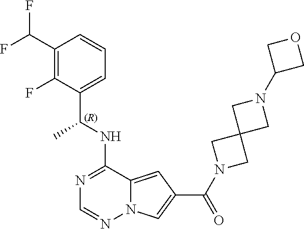 | 487.2 |

### Example 34. Synthesis of N-[(1R)-1-[3-amino-5-(trifluoromethyl)phenyl]ethyl]-2-methyl-6-(morpholinomethyl)pyrrolo[2,1-f][1,2,4]triazin-4-amine

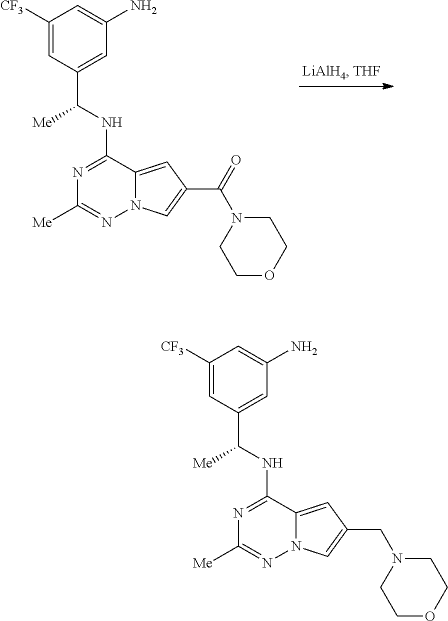 [SMILES: Cc1nc(N[C@H](C)c2cc(N)cc(C(F)(F)F)c2)c2cc(C(=O)N3CCOCC3)cn2n1 ; Cc1nc(N[C@H](C)c2cc(N)cc(C(F)(F)F)c2)c2cc(CN3CCOCC3)cn2n1]

### Step 1.

To a mixture of [4-[[(1 R)-1-[3-amino-5-(trifluoromethyl)phenyl]ethyl]amino]-2-methyl-pyrrolo[2,1-f][1,2,4]triazin-6-yl]-morpholino-methanone (20 mg, 45 μmol) in THF (1 mL) at 0° C. was added LiAlH₄ (1.7 mg, 45 μmol). The mixture was stirred at 0° C. for 2 h, then quenched by addition of H₂O (1 mL) at rt. The mixture was filtered, the solvent was concentrated under reduced pressure and the residue was purified by prep-HPLC to give N-[(1R)-1-[3-amino-5-(trifluoromethyl)phenyl]ethyl]-2-methyl-6-(morpholinomethyl)pyrrolo[2,1-f][1,2,4]triazin-4-amine (3.3 mg, 16% yield). LCMS (ESI): m/z: [M+H] calculated for C₂₁H₂₆F₃N₆O: 435.2; found 435.1; ¹H NMR (400 MHz, METHANOL-d₄) δ ppm 7.45 (d, J=1.5 Hz, 1H), 6.94 (d, J=8.3 Hz, 2H), 6.87 (s, 1H), 6.80 (s, 1H), 5.53 (q, J=6.8 Hz, 1H), 3.77-3.71 (m, 6H), 2.68 (s, 4H), 2.28 (s, 3H), 1.59 (d, J=6.8 Hz, 3H).

### Example 35. Synthesis of (R)-1-(4-((1-(3-amino-5-(trifluoromethyl)phenyl)ethyl)amino)-2-methylpyrrolo[2,1-f][1,2,4]triazin-6-yl)cyclobutane-1,3-diol

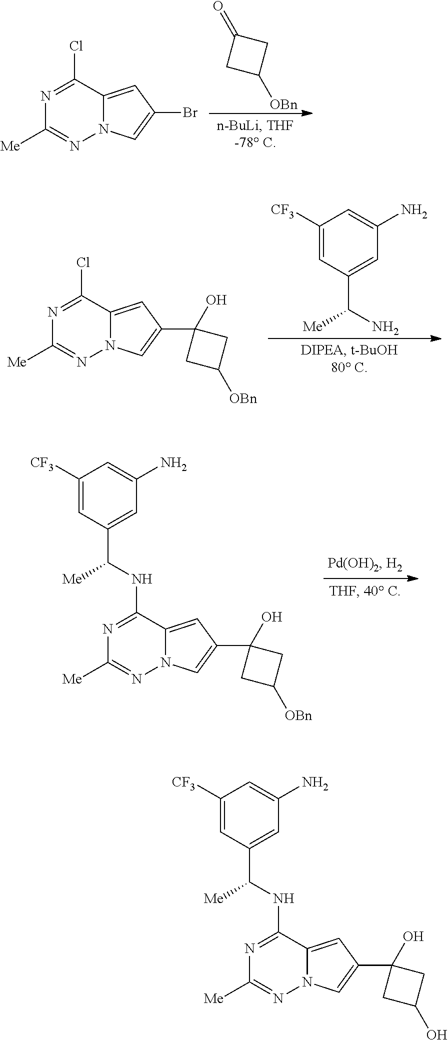 [SMILES: Cc1nc(Cl)c2cc(Br)cn2n1 ; O=C1CC(OCc2ccccc2)C1 ; Cc1nc(Cl)c2cc(C3(O)CC(OCc4ccccc4)C3)cn2n1 ; C[C@@H](N)c1cc(N)cc(C(F)(F)F)c1 ; Cc1nc(N[C@H](C)c2cc(N)cc(C(F)(F)F)c2)c2cc(C3(O)CC(OCc4ccccc4)C3)cn2n1 ; Cc1nc(N[C@H](C)c2cc(N)cc(C(F)(F)F)c2)c2cc(C3(O)CC(O)C3)cn2n1]

### Step 1.

To a mixture of 6-bromo-4-chloro-2-methylpyrrolo[2,1-f][1,2,4]triazine (700 mg, 2.84 mmol) in THF (10 mL) at −78° C. was added a 2.5 M solution of n-BuLi in n-hexanes (1.70 mL, 4.3 mmol). The mixture was stirred at −78° C. for 30 min, then 3-(benzyloxy)cyclobutanone (751 mg, 4.3 mmol) was added, and the mixture was stirred for a further 30 min at −78° C. The mixture was poured into ice-H₂O (30 mL), then extracted with EtOAc (40 mL×3), dried with anhydrous Na₂SO₄ and filtered. The solvent was concentrated under reduced pressure and the residue was purified by column chromatography to give 3-(benzyloxy)-1-(4-chloro-2-methylpyrrolo[2,1-f][1,2,4]triazin-6-yl)cyclobutanol (170 mg, 15% yield). LCMS (ESI): m/z: [M+H] calculated for C₁₈H₁₉ClN₃O₂ 344.11; found: 344.2; ¹H NMR (400 MHz, CDCl₃) δ ppm 7.40-7.28 (m, 5H), 6.90 (d, J=4.6 Hz, 1H), 6.79 (d, J=4.6 Hz, 1H), 4.57 (br s, 1H), 4.47 (s, 2H), 3.82 (quin, J=7.0 Hz, 1H), 2.97 (ddd, J=9.8, 6.8, 2.8 Hz, 2H), 2.65-2.55 (m, 5H).

### Step 2.

(R)-1-(4-((1-(3-amino-5-(trifluoromethyl)phenypethyDamino)-2-methylpyrrolo[2,1-f][1,2,4]triazin-6-yl)-3-(benzyloxy)cyclobutanol was synthesized in a manner similar to N-[(1R)-1-[3-amino-5-(trifluoromethyl)phenyl]ethyl]-6-bromo-2-methyl-pyrrolo[2,1-f][1,2,4]-triazin-4-amine except 6-bromo-4-chloro-2-methyl-pyrrolo[2,1-f][1,2,4]triazine was substituted with 3-(benzyloxy)-1-(4-chloro-2-methylpyrrolo[2,1-f][1,2,4]triazin-6-yl)cyclobutanol. LCMS (ESI): m/z: [M+H] calculated for C₂₇H₂₉F₃N₅O₂: 512.22; found: 512.2; ¹H NMR (400 MHz, CDCl₃) δ ppm 7.38-7.28 (m, 5H), 7.04 (s, 1H), 6.84 (d, J=17.0 Hz, 2H), 6.46-6.41 (m, 2H), 5.61-5.51 (m, 1H), 5.39-5.28 (m, 2H), 4.46 (s, 2H), 3.92-3.77 (m, 3H), 2.96-2.85 (m, 2H), 2.59-2.50 (m, 2H), 2.39 (s, 3H), 1.64 (d, J=6.8 Hz, 3H).

### Step 3.

A mixture of (R)-1-(4-((1-(3-amino-5-(trifluoromethyl)phenypethyl)amino)-2-methylpyrrolo[2,1-f][1,2,4]triazin-6-yl)-3-(benzyloxy)cyclobutanol (59 mg, 0.12 mmol) in THF (2 mL) was purged with N₂ and Pd(OH)₂ (32.4 mg, 0.23 mmol) was added. The suspension was degassed under vacuum and purged with H₂ several times, and the mixture was stirred under an atmosphere of H₂ at 40° C. for 12 h (40 psi). The mixture was filtered through a pad of Celite, and the filter cake was washed with MeOH (50 mL×10). The solvent was concentrated under reduced pressure and the residue was purified by prep-HPLC to give (R)-1-(4-((1-(3-amino-5-(trifluoromethyl)phenypethyDamino)-2-methylpyrrolo[2,1-f][1,2,4]triazin-6-yl)cyclobutane-1,3-diol (20 mg, 41% yield). LCMS (ESI): m/z: [M+H] calculated for C₂₀H₂₃F₃N₅O₂: 422.17; found 422.2; ¹H NMR (400 MHz, CDCl₃) δ ppm 7.04 (s, 1H), 6.86 (s, 1H), 6.82 (s, 1H), 6.47-6.41 (m, 2H), 5.56 (br t, J=6.9 Hz, 1H), 5.43 (s, 1H), 5.35 (br s, 1H), 4.14-4.04 (m, 1H), 3.87 (br s, 2H), 3.03-2.92 (m, 2H), 2.51-2.42 (m, 2H), 2.39 (s, 3H), 1.89 (br d, J=6.2 Hz, 1H), 1.64 (d, J=6.8 Hz, 3H).

### Example 36. Synthesis of (R)-(4-((1-(3-amino-5-(trifluoromethyl)phenyl)ethyl)amino)-2-methylpyrrolo[2,1-f][1,2,4]triazin-6-yl)(4-methylpiperazin-1-yl)methanone

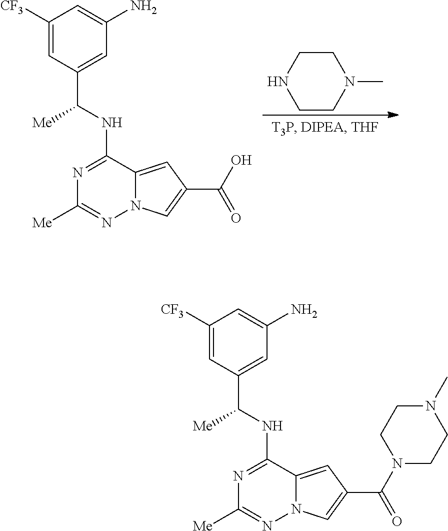

### Step 1.

(R)-(4-((1-(3-amino-5-(trifluoromethyl)phenyl)ethyl)amino)-2-methylpyrrolo[2,1-f][1,2,4]triazin-6-yl)(4-methylpiperazin-1-yl)methanone was synthesized in a manner similar to 4-[[(1R)-1-[3-amino-5-(trifluoromethyl)phenyl]ethyl]amino]-N-[(4-methoxyphenyl)methyl]-N2-dimethyl-pyrrolo[2,1-f][1,2,4]triazine-6-carboxamide except 1-(4-methoxyphenyl)-N-methyl-methanamine was substituted with N-methylpiperazine. LCMS (ESI): m/z: [M+H] calculated for C₂₂H₂₇F₃N₇O: 462.2; found 462.2; ¹H NMR (400 MHz, METHANOL-d₄) δ ppm 8.42-8.20 (m, 1H), 7.69 (d, J=1.7 Hz, 1H), 7.10 (s, 1H), 6.94 (d, J=6.6 Hz, 2H), 6.81 (s, 1H), 5.54 (d, J=7.2 Hz, 1H), 3.85 (s, 4H), 2.67 (s, 4H), 2.46 (s, 3H), 2.29 (s, 3H), 1.60 (d, J=7.1 Hz, 3H).

### Example 37. Synthesis of [4-[[(1R)-1-[3-amino-5-(trifluoromethyl)phenyl]ethyl]amino]-2,7-dimethyl-pyrrolo[2,3-d]pyrimidin-6-yl]-(4-methylpiperazin-1-yl)methanone

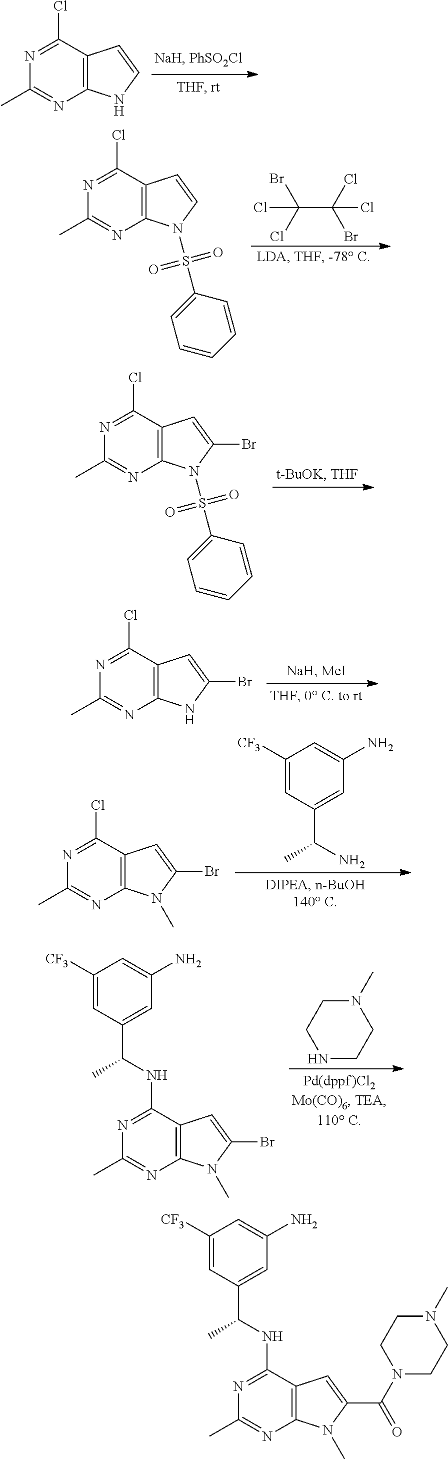

### Step 1.

To a mixture of 4-chloro-2-methyl-7H-pyrrolo[2,3-d]pyrimidine (4 g, 23.9 mmol) in THF (40 mL) was added NaH, 60% dispersion in oil (1.43 g, 35.8 mmol) at 0° C. The mixture was stirred for 30 min, then benzenesulfonyl chloride (3.97 mL, 31.0 mmol) was added at 0° C. The mixture was warmed to rt and stirred for 90 min. An aqueous solution of NH₄Cl (10 mL) and H₂O (20 mL) were added, then the mixture was extracted with EtOAc (10 mL×3). The combined organic layers were washed with brine (20 mL), dried over Na₂SO₄ and filtered. The solvent was concentrated under reduced pressure and the residue was purified by column chromatography to give 7-(benzenesulfonyl)-4-chloro-2-methyl-pyrrolo[2,3-d]pyrimidine (6.9 g, 94% yield). LCMS (ESI): m/z: [M+H] calculated for C₁₃H₁₁ClN₃O₂S: 308.02; found 308.1; ¹H NMR (400 MHz, DMSO-d₆) δ ppm 8.14-8.22 (m, 2H), 8.00 (d, J=4.0 Hz, 1H), 7.74-7.82 (m, 1H), 7.763-7.72 (m, 2H), 6.88 (d, J=4.0 Hz, 1H), 2.68 (s, 3H).

### Step 2.

To a 2M solution of LDA in heptane (4.06 mL, 8.1 mmol) was added to 7-(benzenesulfonyl)-4-chloro-2-methyl-pyrrolo[2,3-d]pyrimidine (1.0 g, 3.3 mmol) in THF (8mL) at −78° C. The mixture was stirred at −78° C. for 30 min, then 1,2-dibromo-1,1,2,2-tetrachloro-ethane (1.17 mL, 9.75 mmol) in THF (8 mL) was added and the mixture was stirred at −78° C. for 30 min. H₂O (20 mL) was added and the mixture was extracted with EtOAc (10 mL×3). The combined organic layers were washed with brine (20 mL), dried over Na₂SO₄, and filtered. The solvent was concentrated under reduced pressure and the residue was purified by column chromatography to give 7-(benzenesulfonyl)-6-bromo-4-chloro-2-methyl-pyrrolo[2,3-d]pyrimidine (1.1 g, 88% yield). LCMS (ESI): m/z: [M+H] calculated for C₁₃H₁₀BrClN₃O₂S: 385.93, 387.93; found 386.0, 387.9; ¹H NMR (400 MHz, METHANOL-d₄) δ ppm 8.12-8.26 (m, 2H), 7.68-7.78 (m, 1H), 7.57-7.66 (m, 2H), 6.96 (s, 1H), 2.73 (s, 3H).

### Step 3.

To a mixture of 7-(benzenesulfonyl)-6-bromo-4-chloro-2-methyl-pyrrolo[2,3-d]pyrimidine (850 mg, 2.2 mmol) in THF (10 mL) was added t-BuOK (1.23 g, 11.0 mmol). The mixture was stirred at rt for 2h and the solvent was concentrated under reduced pressure. The residue was diluted with H₂O (20 mL) and extracted with EtOAc (10 mL×3). The combined organic layers were washed with brine (20 mL), dried over Na₂SO₄, and filtered. The solvent was concentrated under reduced pressure and the residue was purified by column chromatography to give 6-bromo-4-chloro-2-methyl-7H-pyrrolo[2,3-d]pyrimidine (150 mg, 28% yield). LCMS (ESI): m/z: [M+H] calculated for C₇H₆BrClN₃: 245.94, 247.93; found 246.0, 248.0; ¹H NMR (400 MHz, METHANOL-d₄) δ ppm 6.63 (s, 1H), 2.65 (s, 3H).

### Step 4.

To a mixture of 6-bromo-4-chloro-2-methyl-7H-pyrrolo[2,3-d]pyrimidine (140 mg, 0.57 mmol) in THF (2 mL) was added NaH, 60% dispersion in oil (34 mg, 0.85 mmol) at 0° C. The mixture was stirred at 0° C. for 30 min, then iodomethane (106 μL, 1.70 mmol) was added. The mixture was warmed to rt and stirred for 30 min. A solution of NH₄Cl (10 mL) and H₂O (20 mL) were added and the mixture extracted with EtOAc (10 mL×3). The combined organic layers were washed with brine (20 mL), dried over Na₂SO₄, and filtered. The solvent was concentrated under reduced pressure and the residue was purified by prep-TLC to give 6-bromo-4-chloro-2,7-dimethyl-pyrrolo[2,3-d]pyrimidine (180 mg). LCMS (ESI): m/z: [M+H] calculated for C₈H₈BrClN₃: 259.95, 261.95; found 260.0, 262.0.

### Step 5.

To a mixture of 6-bromo-4-chloro-2,7-dimethyl-pyrrolo[2,3-d]pyrimidine (120 mg, 0.46 mmol) and 3-[(1R)-1-aminoethyl]-5-(trifluoromethyl)aniline (141 mg, 0.69 mmol) in n-BuOH (2 mL) was added DIPEA (802 μL, 4.61 mmol). The mixture was heated to 140° C. in a crimped vial and stirred for 12 h. The solvent was concentrated under reduced pressure and the residue was purified by column chromatography to give N-[(1R)-1-[3-amino-5-(trifluoromethyl)phenyl]ethyl]-6-bromo-2,7-dimethyl-pyrrolo[2,3-d]pyrimidin-4-amine (180 mg, 91% yield). LCMS (ESI): m/z: [M+H] calculated for C₁₇H₁₈BrF₃N₅: 428.06, 430.06; found 428.0, 430.0; ¹H NMR (400 MHz, METHANOL-d₄) δ ppm 6.94 (d, J=11.6 Hz, 2H), 6.78 (s, 1H), 6.58 (s, 1H), 5.45 (d, J=7.2 Hz, 1H), 3.68 (d, J=5.6 Hz, 3H), 2.44 (s, 3H), 1.56 (d, J=7.2 Hz, 3H).

### Step 6.

To a mixture of N-[(1R)-1-[3-amino-5-(trifluoromethyl)phenyl]ethyl]-6-bromo-2,7-dimethyl-pyrrolo[2,3-d]pyrimidin-4-amine (180 mg, 0.42 mmol) in 1-methylpiperazine (2 mL) under an atmosphere of N₂ was added Mo(CO)₆ (44 mg, 0.17 mmol), TEA (117 μL, 0.84 mmol), and Pd(dppf)Cl₂ ([1,1′-Bis(diphenylphosphino)ferrocene]dichloropalladium(II), 31 mg, 0.04 mmol). The mixture was heated to 110° C. under microwave irradiation and stirred for 1 h. The solvent was concentrated under reduced pressure and the residue was purified by prep-HPLC to give [4-[[(1R)-1-[3-amino-5-(trifluoromethyl)phenyl]ethyl]amino]-2,7-dimethyl-pyrrolo[2,3-d]pyrimidin-6-yl]-(4-methylpiperazin-1-yl)methanone (16 mg, 8% yield). LCMS (ESI): m/z: [M+H] calculated for C₂₃H₂₉F₃N₇O: 476.23; found 476.2; ¹H NMR (400 MHz, METHANOL-d₄) δ ppm 6.94 (d, J=6.8 Hz, 2H), 6.89-6.81 (m, 1H), 6.79 (s, 1H), 5.57-5.36 (m, 1H), 3.82 (d, J=1.6 Hz, 4H), 3.74 (s, 3H), 2.66 (s, 4H), 2.53-2.42 (m, 6H), 1.58 (d, J=7.2 Hz, 3H).

### Example 38. Synthesis of N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine and N-[(1R)-1-[3-amino-5-(trifluoromethyl)phenyl]ethyl]-7-methyl-6-(4-piperidyl)pyrrolo[2,3-d]pyrimidin-4-amine

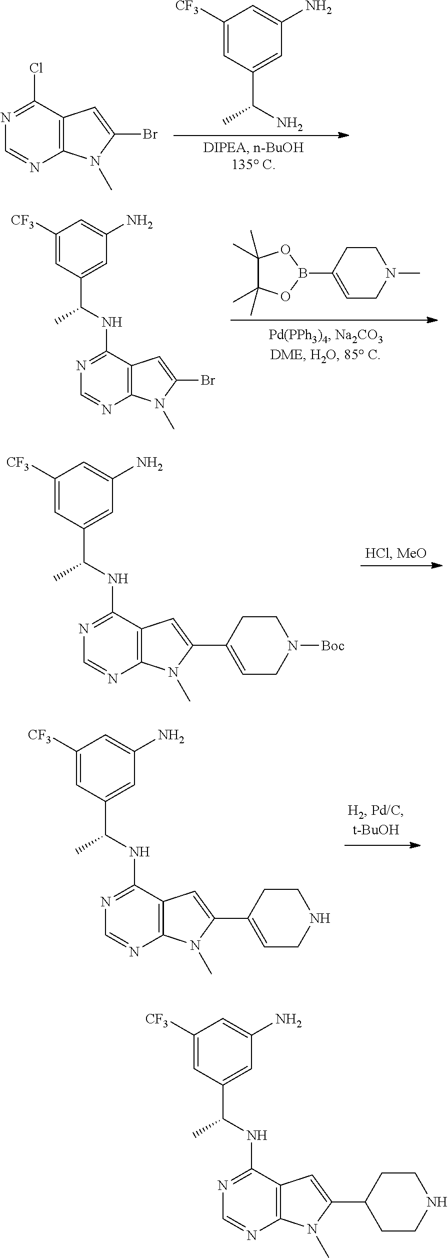

### Step 1.

To a mixture of 6-bromo-4-chloro-7-methyl-pyrrolo[2,3-d]pyrimidine (250 mg, 1.0 mmol) and 3-[(1R)-1-aminoethyl]-5-(trifluoromethyl)aniline (269 mg, 1.32 mmol) in n-BuOH (5 mL) was added DIPEA (883 μL, 5.1 mmol). The mixture was heated to 135° C. in a crimped vial and stirred for 15 h. The solvent was concentrated under reduced pressure and the residue was purified by column chromatography to give N-[(1R)-1-[3-amino-5-(trifluoromethyl)phenyl]ethyl]-6-bromo-7-methyl-pyrrolo[2,3-d]pyrimidin-4-amine (250 mg, 60% yield). LCMS (ESI): m/z: [M+H] calculated for C₁₆H₁₆BrF₃N₅: 414.0, 416.05; found 413.9, 415.9; ¹H NMR (400 MHz, CDCl₃) δ ppm 8.31 (s, 1H), 7.01 (s, 1H), 6.86 (s, 1H), 6.79 (s, 1H), 6.42 (s, 1H), 5.47-5.37 (m, 1H), 5.28-5.17 (m, 1H), 3.76 (s, 3H), 1.62 (d, J=6.8 Hz, 3H).

### Step 2.

A mixture of N-[(1R)-1-[3-amino-5-(trifluoromethyl)phenyl]ethyl]-6-bromo-7-methyl-pyrrolo[2,3-d]pyrimidin-4-amine (200 mg, 0.48 mmol), tert-butyl 4-(4,4,5,5-tetramethyl-1,3,2-dioxaborolan-2-yl)-3,6-dihydro-2H-pyridine-1-carboxylate (224 mg, 0.72 mmol), Pd(PPh₃)₄ (Palladium-tetrakis(triphenylphosphine, 56 mg, 0.05 mmol) and Na₂CO₃ (154 mg, 1.45 mmol) in H₂O (2 mL) and DME (10 mL) was degassed with N₂ (×3). The mixture was heated to 85° C. and stirred for 2 h, then the solvent concentrated under reduced pressure. The residue was diluted with H₂O (50mL) and extracted with EtOAc (50 mL×3). The combined organic layers were washed with brine (50 mL), dried over Na₂SO₄, and filtered. The solvent was concentrated under reduced pressure and the residue was purified by column chromatography to give tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyridine-1-carboxylate (240 mg, 96% yield). LCMS (ESI): m/z: [M+H] calculated for C₂₆H₃₂F₃N₆O₂: 517.2; found 517.1; ¹H NMR (400 MHz, CDCl₃) δ ppm 8.34 (s, 1H), 7.73-7.62 (m, 2H), 7.59-7.52 (m, 1H), 7.52-7.43 (m, 2H), 7.03 (s, 1H), 6.88 (s, 1H), 6.79 (s, 1H), 6.21 (s, 1H), 5.95 (s, 1H), 5.45 (t, J=6.9 Hz, 1H), 5.22-5.09 (m, 1H), 3.84 (s, 2H), 3.78 (s, 3H), 3.65 (t, J=5.4 Hz, 2H), 2.47 (s, 2H), 1.63 (d, J=6.8 Hz, 3H), 1.51 (s, 9H).

### Step 3.

A mixture of tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyridine-1-carboxylate (200 mg, 0.38 mmol) in 4M HCl in MeOH (5 mL) was stirred at rt for 1 h. The solvent was concentrated under reduced pressure and the residue was purified by prep-HPLC to give N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine (110 mg, 68% yield). LCMS (ESI): m/z: [M+H] calculated for C₂₁H₂₄F₃N₆: 417.2; found 417.0; ¹H NMR (400 MHz, CDCl₃) δ ppm 8.51 (s, 1H), 8.31 (s, 1H), 7.02 (s, 1H), 6.88 (s, 1H), 6.78 (s, 1H), 6.25 (s, 1H), 5.96 (s, 1H), 5.46-5.34 (m, 1H), 3.78 (s, 3H), 3.74 (s, 2H), 3.28 (t, J=5.7 Hz, 2H), 2.59 (s, 2H), 1.63 (d, J=6.8 Hz, 3H).

### Step 4.

A mixture of N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine (20 mg, 0.05 mmol) and 10% wt. Pd/C (10 mg) in t-BuOH (1 mL) was degassed and purged with H₂ (×3). The mixture was heated to 50° C. and stirred for 4 h under an atmosphere of H₂. The mixture was filtered, the solvent was concentrated under reduced pressure and the residue was purified by prep-HPLC to give N-[(1R)-1-[3-amino-5-(trifluoromethyl)phenyl]ethyl]-7-methyl-6-(4-piperidyl)pyrrolo[2,3-d]pyrimidin-4-amine (10 mg, 49% yield). LCMS (ESI): m/z: [M+H] calculated for C₂₁H₂₆F₃N₆: 419.2; found 419.2; ¹H NMR (400 MHz, CDCl₃) δ ppm 8.58 (s, 1H), 8.30 (s, 1H), 7.04 (s, 1H), 6.90 (s, 1H), 6.78 (s, 1H), 6.22-6.05 (m, 1H), 5.87-5.61 (m, 1H), 5.48-5.25 (m, 1H), 3.74 (s, 4H), 3.47-3.38 (m, 2H), 3.02-2.82 (m, 3H), 2.14-2.00 (m, 2H), 2.00-1.85 (m, 2H), 1.63 (d, J=6.7 Hz, 3H).

### Example 39. Synthesis of N-[(1R)-1-[3-amino-5-(trifluoromethyl)phenyl]ethyl]-2-chloro-7-methyl-6-(1,2,3,6-tetrahydropyridin-4-yl)pyrrolo[2,3-d]pyrimidin-4-amine

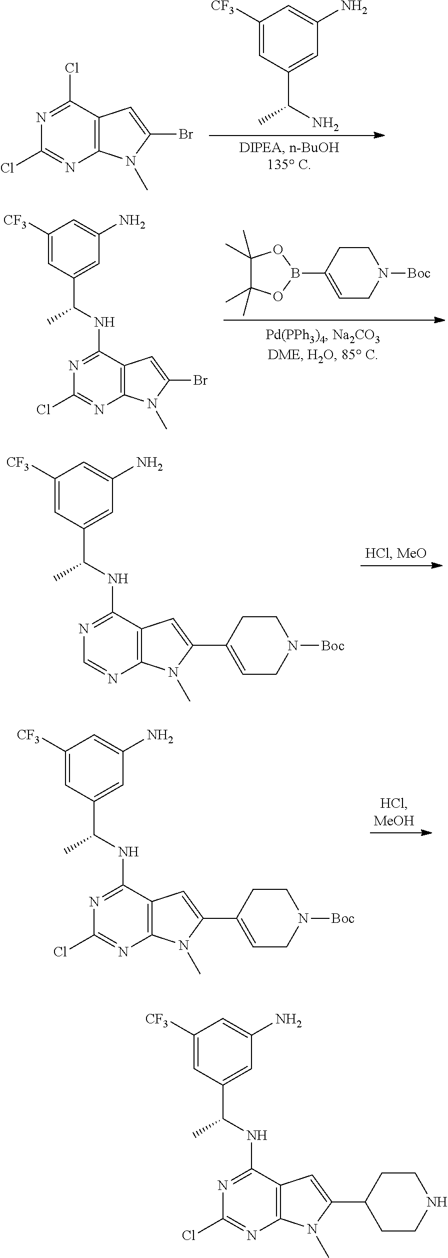

### Step 1.

N-[(1R)-1-[3-amino-5-(trifluoromethyl)phenyl]ethyl]-6-bromo-2-chloro-7-methyl-pyrrolo[2,3-d]pyrimidin-4-amine was synthesized in a manner similar to N-[(1R)-1-[3-amino-5-(trifluoromethyl)phenyl]ethyl]-6-bromo-7-methyl-pyrrolo[2,3-d]pyrimidin-4-amine except 6-bromo-4-chloro-7-methyl-pyrrolo[2,3-d]pyrimidine was substituted with 6-bromo-2,4-dichloro-7-methyl-pyrrolo[2,3-d]pyrimidine. LCMS (ESI): m/z: [M+H] calculated for C₁₆H₁₅BrClF₃N₅: 448.0; found 448.0; ¹H NMR (400 MHz, CDCl₃) δ ppm 7.02 (s, 1H), 6.88 (s, 1H), 6.82 (s, 1H), 6.38 (s, 1H), 5.47-5.36 (m, 1H), 5.32-5.19 (m, 1H), 3.73 (s, 3H), 1.63 (d, J=6.7 Hz, 3H).

### Step 2.

Ter t -butyl4-[4-[[(1R)-1-[3-amino-5-(trifluoromethyl)phenyl]ethyl]amino]-2-chloro-7-methyl-pyrrolo[2,3-d]pyrimidin-6-yl]-3,6-dihydro-2H-pyridine-1-carboxylate was synthesized in a manner similar to tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyri dine-1-carboxyl ate except N-[(1R)-1-[3 -amino-5-(trifluoromethyl)phenyl]ethyl]-6-bromo-7-methyl-pyrrolo[2,3-d]pyrimidin-4-amine was substituted with N-[(1R)-1-[3-amino-5-(trifluoromethyl)phenyl]ethyl]-6-bromo-2-chloro-7-methyl-pyrrolo[2,3-d]pyrimidin-4-amine. LCMS (ESI): m/z: [M+H] calculated for C₂₆H₃₁ClF₃N₆O₂: 551.2; found 551.1.

### Step 3.

N-[(1R)-1-[3-amino-5-(trifluoromethyl)phenyl]ethyl]-2-chloro-7-methyl-6-(1,2,3,6-tetrahydropyridin-4-yl)pyrrolo[2,3-d]pyrimidin-4-amine was synthesized in a manner similar to N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine except tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyridine-1-carboxylate was substituted with tert-butyl 4-[4-[[(1R)-1-[3-amino-5-(trifluoromethyl)phenyl]ethyl]amino]-2-chloro-7-methyl-pyrrolo[2,3-d]pyrimidin-6-yl]-3,6-dihydro-2H-pyridine-1-carboxylate. LCMS (ESI): m/z: [M+H] calculated for C₂₁H₂₂ClF₃N₆: 451.2; found 451.2; ¹H NMR (400 MHz, METHANOL-d₄) δ ppm 6.99 (s, 1H), 6.95 (s, 1H), 6.55 (s, 1H), 6.07 (s, 1H), 5.47-5.37 (m, 1H), 3.71 (s, 3H), 3.58-3.50 (m, 2H), 3.09 (t, J=5.8 Hz, 2H), 2.51-2.43 (m, 2H), 1.58 (d, J=7.0 Hz, 3H).

### Example 40. Synthesis of N-[(1R)-1-[3-amino-5-(trifluoromethyl)phenyl]ethyl]-2-chloro-7-methyl-6-(1,2,3,6-tetrahydropyridin-4-yl)pyrrolo[2,34]pyrimidin-4-amine

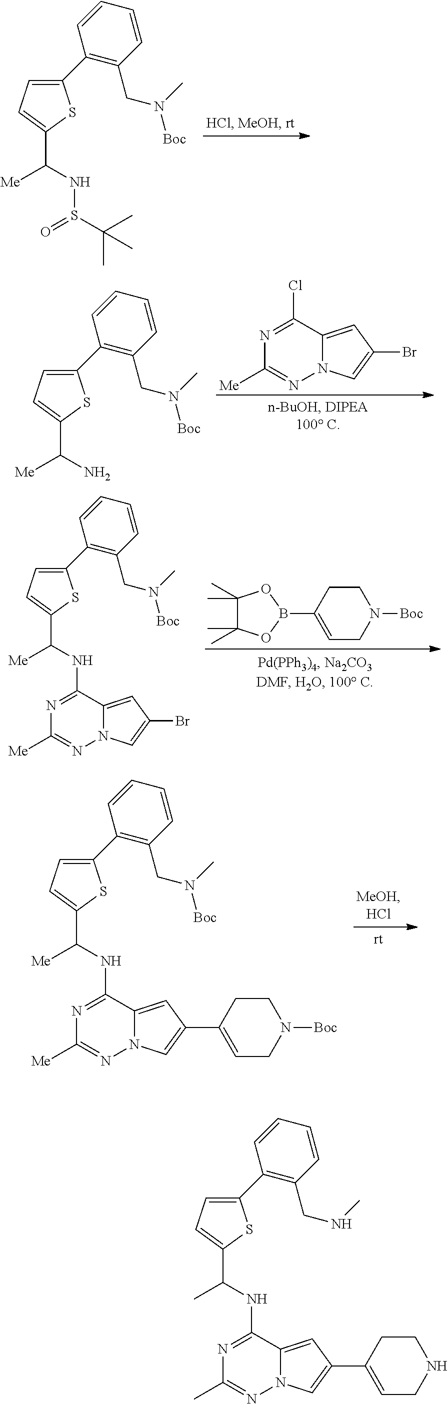

### Step 1.

To a mixture of tert-butyl N-[[2-[5-[1-(tert-butylsulfinylamino)ethyl]-2-thienyl]phenyl]methyl]-N-methyl-carbamate (0.5 g, 1.1 mmol) in MeOH (20 mL) at rt was added 4M HCl in MeOH (555 μL, 2.2 mmol). The mixture was stirred at rt for 1 h, then adjusted to pH ˜8 by dropwise addition of NaOH in MeOH. The solvent was concentrated under reduced pressure and MeOH: DCM (1: 5; 6 mL) was added and the mixture stirred at rt for 10 min. The mixture was filtered and the solvent concentrated under reduced pressure to give tert-butyl-N-[[2-[5-(1-aminoethyl)-2-thienyl]phenyl]methyl]-N-methyl-carbamate (0.5 g). ¹H NMR (400 MHz, CDCl₃) δ ppm 7.25-7.29 (m, 2H), 7.16-7.19 (m, 3H), 7.12 (d, J=2.8 Hz, 1H), 6.76 (d, J=3.2 Hz, 1H), 4.56-4.61 (m, 1H), 4.46 (d, J=14.4 Hz, 2H), 2.66 (s, 3H), 1.69 (d, J=6.4 Hz, 3H), 1.33-1.41 (m, 9H).

### Step 2.

To a mixture of 6-bromo-4-chloro-2-methyl-pyrrolo[2,1-f][1,2,4]triazine (0.2 g, 0.8 mmol) and tert-butyl N-[[2-[5-(1-aminoethyl)-2-thienyl]phenyl]methyl]-N-methyl-carbamate (337 mg, 0.97 mmol) in n-BuOH (2 mL) was added DIPEA (706 μL, 4.06 mmol). The mixture was heated to 100° C. and stirred for 2 h, the poured into H₂O (3 mL) and extracted with EtOAc (2 mL×3). The combined organic layers were washed with brine (2 mL), dried over Na₂SO₄, and filtered. The solvent was concentrated under reduced pressure and the residue was purified by column chromatography to give tert-butyl N-[[2-[5-[1-[(6-bromo-2-methyl-pyrrolo[2,1-f][1,2,4]triazin-4-yl)amino]ethyl]-2-thienyl]phenyl]methyl]-N-methyl-carbamate (0.35 g, 78% yield). ¹H NMR (400 MHz, CDCl₃) δ ppm 7.47 (d, J=1.6 Hz, 1H), 7.34-7.37 (m, 2H), 7.25-7.30 (m, 2H), 7.02 (s, 1H), 6.84 (d, J=3.2 Hz, 1H), 6.57 (s, 1H), 5.87-5.89 (m, 1H), 5.42 (s, 1H), 4.54-4.58 (m, 2H), 2.75 (m, 3H), 2.43 (s, 3H), 1.77 (d, J=1.6 Hz, 3H), 1.45 (m, 9H).

### Step 3.

Pd(PPh₃)₄ (Palladium-tetrakis(triphenylphosphine, 10 mg, 0.09 mmol) was added to a mixture of tert-butyl N-[[2-[5-[1-[(6-bromo-2-methyl-pyrrolo[2,1-f][1,2,4]triazin-4-yl)amino]ethyl]-2-thienyl]phenyl]methyl]-N-methyl-carbamate (0.1 g, 0.18 mmol), tert-butyl-4-(4,4,5,5-tetramethyl-1,3,2-dioxaborolan-2-yl)-3,6-dihydro-2H-pyridine-1-carboxylate (67 mg, 0.22 mmol), 2M Na₂CO₃ (180 μL, 0.36 mmol) and DMF (1 mL) under an atmosphere of N₂. The mixture was heated to 100° C. and stirred for 3 h, then poured into H₂O (2 mL) and extracted with EtOAc (2 mL×3). The combined organic layers were washed with brine (1 mL), dried over Na₂SO₄, and filtered. The solvent was concentrated under reduced pressure and the residue was purified by column chromatography to give tert-butyl 4-[4-[1-[5-[2-[[tert-butoxycarbonyl(methyl)amino]methyl]phenyl]-2-thienyl]ethylamino]-2-methyl-pyrrolo[2, 1-f][1,2,4]triazin-6-yl]-3,6-dihydro-2H-pyridine-1-carboxylate (0.08 g, 68% yield). ¹H NMR (400 MHz, CDCl₃) δ ppm 7.51-7.56 (m, 1H), 7.34-7.37 (m, 1H), 7.24-7.30 (m, 2H), 7.01-7.04 (m, 1H), 6.85 (s, 1H), 6.55 (s, 1H), 6.02 (s, 1H), 5.91 (s, 1H), 5.40-5.433 (m, 1H), 4.53-4.62 (m, 2H), 4.06-4.10 (m, 2H), 3.64 (t, J=5.2 Hz, 2H), 2.75 (d, J=23.2 Hz, 3H), 2.44-2.51 (m, 4H), 1.78 (d, J=4.0 Hz, 3H), 1.38-1.49 (m, 18H).

### Step 4.

2-methyl-N-[1-[5-[2-(methylaminomethyl)phenyl]-2-thienyl]ethyl]-6-(1,2,3,6-tetrahydropyridin-4-yl)pyrrolo[2,1-f][1,2,4]triazin-4-amine was synthesized in a manner similar to N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine except tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyridine-1-carboxylate was substituted with ter t-butyl 4-[4-[1-[5-[2-[[tert-butoxycarbonyl(methyl)amino]methyl]phenyl]-2-thienyl]ethylamino]-2-methyl-pyrrolo[2,1-f][1,2,4]triazin-6-yl]-3,6-dihydro-2H-pyridine-1-carboxylate. LCMS (ESI): m/z: [M+H] calculated for C₂₆H₃₁N₆S: 459.23; found 459.3; ¹H NMR (400 MHz, METHANOL-d₄) δ ppm 7.60 (s, 1H), 7.55-7.57 (m, 1H), 7.46-7.48 (m, 3H), 7.12 (d, J=3.6 Hz, 1H), 6.98-6.99 (m, 2H), 6.14 (s, 1H), 5.91-5.96 (m, 1H), 4.29 (s, 2H), 3.83 (s, 2H), 3.45 (t, J=6.0 Hz, 2H), 2.77 (s, 3H), 2.59 (s, 3H), 2.34 (s, 3H), 1.76 (d, J=6.8 Hz, 3H).

### Example 41. Synthesis of [4-[[(1R)-1-[3-amino-5-(trifluoromethyl) phenyl]ethyl]amino]-2-methyl-pyrrolo[2,1-f][1,2,4]triazin-6-yl]morpholino-methanone

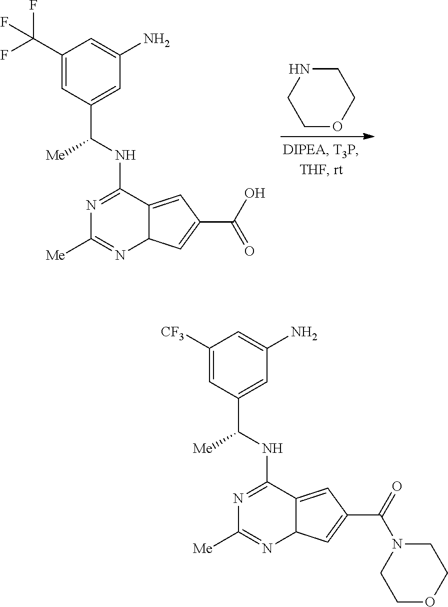 [SMILES: CC1=NC2C=C(C(=O)O)C=C2C(N[C@H](C)c2cc(N)cc(C(F)(F)F)c2)=N1 ; C1COCCN1 ; CC1=NC2C=C(C(=O)N3CCOCC3)C=C2C(N[C@H](C)c2cc(N)cc(C(F)(F)F)c2)=N1]

### Step 1.

[4-[[(1R)-1-[3-amino-5-(trifluoromethyl) phenyl]ethyl]amino]-2-methyl-pyrrolo[2,1-f][1,2,4]triazin-6-yl]morpholino-methanone was synthesized in a manner similar to 4-[[(1R)-1-[3-amino-5-(trifluoromethyl)phenyl]ethyl]amino]-N-[(4-methoxyphenyl)methyl]-N2-dimethyl-pyrrolo[2,1-f][1,2,4]triazine-6-carboxamide except 1-(4-methoxyphenyl)-N-methyl-methanamine was substituted with morpholine. LCMS (ESI): m/z: [M+H] calculated for C₂₁H₂₄F₃N₆O₂: 449.2; found 449.1; ¹H NMR (400 MHz, METHANOL-d₄) δ ppm 7.69 (d, J=1.5 Hz, 1H), 7.10 (d, J=1.0 Hz, 1H), 6.93 (d, J=7.3 Hz, 2H), 6.80 (s, 1H), 5.53 (q, J=6.8 Hz, 1H), 3.82-3.67 (m, 8H), 2.28 (s, 3H), 1.59 (d, J=6.8 Hz, 3H).

### Example 42. Synthesis of [7-[[(1R)-1-[3-amino-5-(trifluoromethyl)phenyl]ethyl]amino]thiazolo[5,4-d]pyrimidin-2-yl]-morpholino-methanone

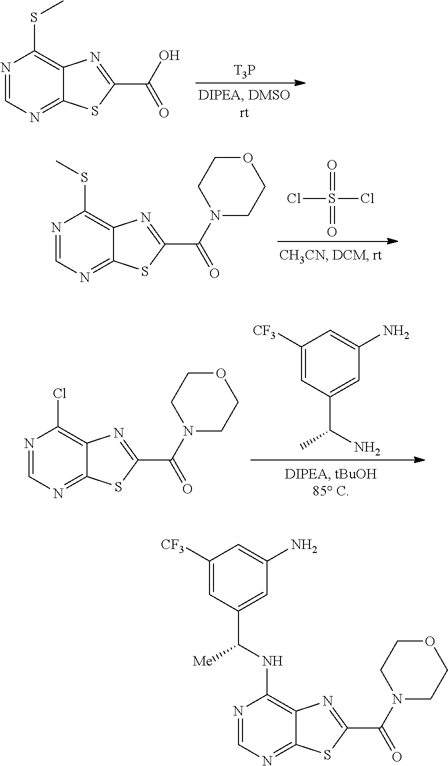

### Step 1.

To a mixture of 7-methylsulfanylthiazolo[5,4-d]pyrimidine-2-carboxylic acid (700 mg, 3.08 mmol) in DMSO (30 mL) was added DIPEA (1.61 mL, 9.24 mmol), morpholine (813 μL, 9.24 mmol), and T3P (5.5 mL, 18.5 mmol). The mixture was stirred at rt for 1 h, then poured into H₂O (60 mL) and the mixture extracted with EtOAc (30 mL×2). The combined organic layers were washed with brine (40 mL), dried with anhydrous Na₂SO₄, and filtered. The solvent was concentrated under reduced pressure and the residue was purified by column chromatography to give (7-methylsulfanylthiazolo[5,4-d]pyrimidin-2-yl)-morpholino-methanone (400 mg, 44% yield). LCMS (ESI): m/z: [M+H] calculated for C₁₁H₁₃N₄O₂S₂: 297.04; found 297.1; ¹H NMR (400 MHz, DMSO-d₆) δ ppm 9.01-8.99 (m, 1H), 4.27 (t, J=4.6 Hz, 2H), 3.76-3.68 (m, 6H), 2.69 (s, 3H).

### Step 2.

To a mixture of (7-methylsulfanylthiazolo[5,4-d]pyrimidin-2-yl)-morpholino-methanone (200 mg, 0.67 mmol) in MeCN (2 mL) at 0° C. was added a solution of sulfuryl chloride (337 μL, 3.37 mmol) in DCM (1 mL). The mixture was warmed to rt and stirred for 2 h, then poured into H₂O (5mL) then the pH adjusted to ˜7 with a saturated solution of Na₂CO₃. The mixture was extracted with EtOAc (10 mL+5 mL), the combined organic layers were washed with brine (5 mL), dried with anhydrous Na₂SO₄, and filtered. The solvent was concentrated under reduced pressure to give (7-chlorothiazolo[5,4-d]pyrimidin-2-yl)-morpholino-methanone (220 mg). LCMS (ESI): m/z: [M+H] calculated for C₁₀H₁₀ClN₄O₂S: 285.01; found 285.0; ¹H NMR (400 MHz, DMSO-d₆) δ ppm 9.13-9.11 (m, 1H), 4.28-4.23 (m, 2H), 3.77-3.71 (m, 7H).

### Step 3.

[7-[[(1R)-1-[3-amino-5-(trifluoromethyl)phenyl]ethyl]amino]thiazolo[5,4-d]pyrimidin-2-yl]-morpholino-methanone was synthesized in a manner similar to [1-[[(1R)-1-[3-amino-5-(trifluoromethyl)phenyl]ethyl]amino]-4-chloro-5,7-dihydropyrrolo[3,4-d]pyridazin-6-yl]-morpholino-methanone except (1,4-dichloro-5,7-dihydropyrrolo[3,4-d]pyridazin-6-yl)-morpholino-methanone was substituted with (7-chlorothiazolo[5,4-d]pyrimidin-2-yl)-morpholino-methanone. LCMS (ESI): m/z: [M+H] calculated for C₁₉H₂₀F₃N₆O₂S: 453.12; found 453.1; ¹H NMR (400 MHz, METHANOL-d₄) δ ppm 8.39 (s, 1H), 6.97 (s, 2H), 6.81 (s, 1H), 5.60-5.51 (m, 1H), 4.57-4.48 (m, 2H), 3.80 (s, 6H), 2.03 (s, 1H), 1.65 (d, J=7.0 Hz, 3H).

### Example 43. Synthesis of 1-[4-[4-[[(1R)-1-[3-amino-5-(trifluoromethyl)phenyl]ethyl]amino]pyrrolo[2,1-f][1,2,4]triazin-6-yl]piperazin-1-yl]ethanone

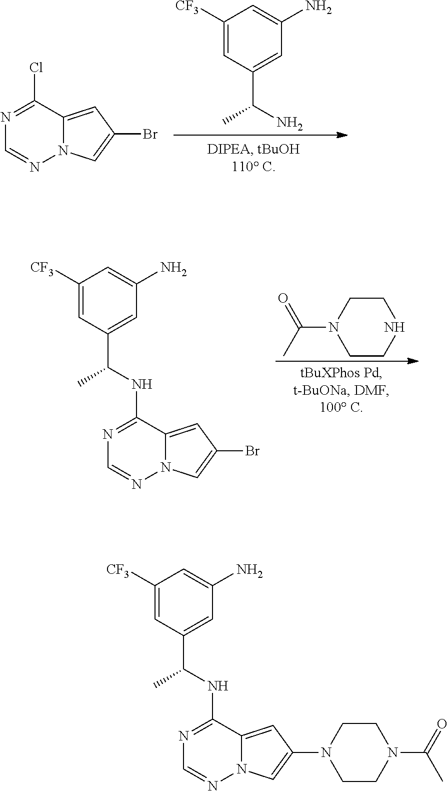

### Step 1.

To a mixture of 6-bromo-4-chloro-pyrrolo[2,1-f][1,2,4]triazine (100 mg, 0.43 mmol) and 3-[(1R)-1-aminoethyl]-5-(trifluoromethyl)aniline (88 mg, 0.43 mmol) in n-BuOH (2 mL) was added DIPEA (225 μL, 1.29 mmol). The mixture was heated to 110° C. and stirred for 1 h, then poured into ice-H₂O (5 mL) and extracted with EtOAc (5 mL×3). The combined organic layers were washed with brine (5 mL), dried with anhydrous Na₂SO₄, and filtered. The solvent was concentrated under reduced pressure and the residue was purified by column chromatography to give N-[(1R)-1-[3-amino-5-(trifluoromethyl)phenyl]ethyl]-6-bromo-pyrrolo[2,1-f][1,2,4]triazin-4-amine (100 mg, 58% yield). ¹H NMR (400 MHz, CDCl₃) δ ppm 7.88 (s, 1H), 7.51 (s, 1H), 6.95 (s, 1H), 6.79-6.78 (s, 2H), 6.57 (s, 1H), 5.46-5.44 (m, 1H), 5.44-5.42 (s, 1H), 5.32-5.30 (d, J=7.2 Hz, 1H), 3.85 (s, 2H), 1.59 (d, J=6.8 Hz, 3H).

### Step 2.

To a mixture of N-[(1R)-1-[3-amino-5-(trifluoromethyl)phenyl]ethyl]-6-bromo-pyrrolo[2,1-f][1,2,4]triazin-4-amine (70 mg, 0.18 mmol) and piperazin-1-ylethanone (90 mg, 0.7 mmol) in DMF (0.5 mL) under an atmosphere of N₂ at rt was added t-BuONa (34 mg, 0.35 mmol) and [2-(2-aminoethyl)phenyl]-chloro-palladium;di-tert-butyl-[2-(2,4,6-triisopropylphenyl)phenyl]phosphane (12 mg, 18 μmol). The mixture was heated to 110° C. and stirred for 10 h, then poured into ice-H₂O (5 mL) and extracted with EtOAc (5 mL×3). The combined organic layers were washed with brine (5 mL), dried with anhydrous Na₂SO₄, and filtered. The solvent was concentrated under reduced pressure and the residue was purified by prep-HPLC to give 1-[4-[4-[[(1R)-1-[3-amino-5-(trifluoromethyl)phenyl]ethyl]amino]pyrrolo[2,1-f][1,2,4]triazin-6-yl]piperazin-1-yl]ethanone (5 mg, 6% yield). LCMS (ESI): m/z: [M+H] calculated for C₂₁H₂₅F₃N₇O: 448.2; found 448.2; ¹H NMR (400 MHz, DMSO-d₆) δ ppm 7.85 (s, 1H), 7.49 (s, 1H), 6.92 (s, 1H), 6.87 (d, 1H), 6.79 (s, 1H), 5.38-5.31 (m, 1H), 3.60-3.59 (s, 4H), 3.05-3.03 (s, 2H), 2.99-2.98 (s, 2H), 2.04 (s, 3H), 1.53 (d, J=6.8 Hz, 3H).

### Example 44. Synthesis of N-[(1R)-1-[3-amino-5-(trifluoromethyl)phenyl]ethyl]-6-methoxy-pyrrolo[2,1-f][1,2,4]triazin-4-amine

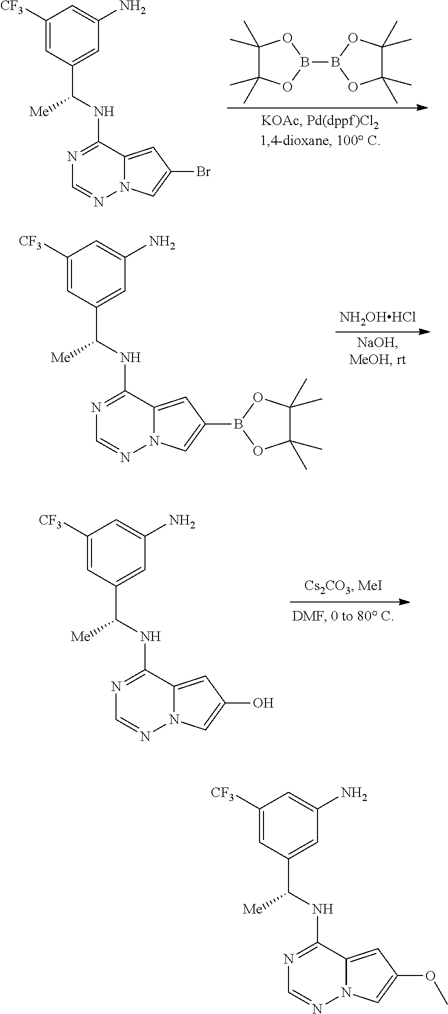

### Step 1.

N-[(1R)-1-[3-amino-5-(trifluoromethyl)phenyl]ethyl]-6-(4,4,5,5-tetramethyl-1,3,2-dioxaborolan-2-yl)pyrrolo[2,1-f][1,2,4]triazin-4-amine was synthesized in a manner similar to N-[(1R)-1-[3-amino-5-(trifluoromethyl)phenyl]ethyl]-2-methyl-6-(4,4,5,5-tetramethyl-1,3,2-dioxaborolan-2-yl)pyrrolo[2,1-f][1,2,4]triazin-4-amine except N-[(1R)-1-[3-amino-5-(trifluoromethyl)phenyl]ethyl]-6-bromo-2-methyl-pyrrolo[2,1-f][1,2,4]triazin-4-amine was substituted with N-[(1R)-1-[3-amino-5-(trifluoromethyl)phenyl]ethyl]-6-bromo-pyrrolo[2,1-f][1,2,4]triazin-4-amine. LCMS (ESI): m/z: [M+H] calculated for C₂₁H₂₆BF₃N₅O₂: 448.21; found 448.1.

### Step 2.

4-[[(1R)-1-[3-amino-5-(trifluoromethyl)phenyl]ethyl]amino]pyrrolo[2,1-f][1,2,4]triazin-6-ol was synthesized in a manner similar to 4-[[(1R)-1-[3-amino-5-(trifluoromethyl)phenyl]ethyl]amino]-2-methyl-pyrrolo[2,1-f][1,2,4]triazin-6-ol except N-[(1R)-1-[3-amino-5-(trifluoromethyl)phenyl]ethyl]-2-methyl-6-(4,4,5,5-tetramethyl-1,3,2-dioxaborolan-2-yl)pyrrolo[2,1-f][1,2,4]triazin-4-amine was substituted with N-[(1R)-1-[3-amino-5-(trifluoromethyl)phenyl]ethyl]-6-(4,4,5,5-tetramethyl-1,3,2-dioxaborolan-2-yl)pyrrolo[2,1-f][1,2,4]triazin-4-amine. LCMS (ESI): m/z: [M+H] calculated for C₁₅H₁₅F₃N₅O: 338.12; found 338.2; ¹H NMR (400 MHz, CDCl₃) δ ppm 7.93 (s, 1H), 7.27 (d, J=1.6 Hz, 1H), 7.01 (s, 1H), 6.85 (s, 1H), 6.82 (s, 1H), 6.12 (d, J=1.2 Hz, 1H), 5.53-5.44 (m, 1H), 5.20-5.13 (br m, 1H), 4.76-4.58 (br m, 1H), 3.96-3.82 (br m, 2H), 1.64 (d, J=7.2 Hz, 3H).

### Step 3.

N-[(1R)-1-[3-amino-5-(trifluoromethyl)phenyl]ethyl]-6-methoxy-pyrrolo[2,1-f][1,2,4]triazin-4-amine was synthesized in a manner similar to N-[(1R)-1-[3-amino-5-(trifluoromethyl)phenyl]ethyl]-6-methoxy-2-methyl-pyrrolo[2,1-f]-[1,2,4]triazin-4-amine except 4-[[(1R)-1-[3-amino-5-(trifluoromethyl)phenyl]ethyl]amino]-2-methyl-pyrrolo[2,1-f][1,2,4]triazin-6-ol was substituted with 4-[[(1R)-1-[3-amino-5-(trifluoromethyl)phenyl]ethyl]amino]pyrrolo[2,1-f][1,2,4]triazin-6-ol. LCMS (ESI): m/z: [M+H] calculated for C₁₆H₁₇F₃N₅O: 352.13; found 352.2; ¹H NMR (400 MHz, METHANOL-d₄) δ ppm 7.74 (s, 1H), 7.24 (d, J=2.0 Hz, 1H), 6.92 (d, J=7.8 Hz, 2H), 6.80 (s, 1H), 6.59 (d, J=1.6 Hz, 1H), 5.39 (q, J=7.2 Hz, 1H), 3.83 (s, 3H), 1.59 (d, J=7.2 Hz, 3H).

### Example 45. Synthesis of 1-[4-[4-[[(1R)-1-[3-(trifluoromethyl)phenyl]ethyl]amino]pyrrolo[2,1-f][1,2,4]triazin-6-yl]piperazin-1-yl]ethenone

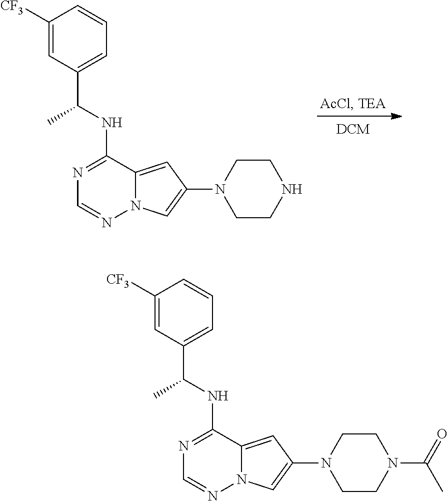 [SMILES: C[C@@H](Nc1ncnn2cc(N3CCNCC3)cc12)c1cccc(C(F)(F)F)c1 ; CC(=O)N1CCN(c2cc3c(N[C@H](C)c4cccc(C(F)(F)F)c4)ncnn3c2)CC1]

### Step 1.

To a mixture of 6-piperazin-1-yl-N-[(1R)-1-[3-(trifluoromethyl)phenyl]ethyl]pyrrolo[2,1-f][1,2,4]triazin-4-amine HCl salt (40 mg, 94 μmol) in DCM (10 mL) was added TEA (39 μL, 0.28 mmol) and acetyl chloride (7.4 μL, 0.1 mmol). The mixture was stirred at rt for 1 h, the solvent was concentrated under reduced pressure and the residue was purified by column chromatography, then re-purified by prep-HPLC to give 1-[4-[4-[[(1R)-1-[3-(trifluoromethyl)phenyl]ethyl]amino]pyrrolo[2,1-f][1,2,4]triazin-6-yl]piperazin-1-yl]ethenone (32 mg, 75% yield). LCMS (ESI): m/z: [M+H] calculated for C₂₁H₂₄F₃N₆O: 433.2; found 433.0; ¹H NMR (400 MHz, METHANOL-d₄) δ ppm 7.71-7.70 (m, 2H), 7.66 (d, J=6.8 Hz, 1H), 7.55-7.48 (m, 2H), 7.25 (d, J=1.6 Hz, 1H), 6.60 (s, 1H), 5.52 (q, J=6.8 Hz, 1H), 3.77-3.69 (m, 4H), 3.14-3.07 (m, 4H), 2.15 (s, 3H), 1.63 (d, J=6.8 Hz, 3H).

### Example 46. Synthesis of N-[(1R)-1-[3-amino-5-(trifluoromethyl)phenyl]ethyl]-2-methyl-6-(1,2,3,6-tetrahydropyridin-4-yl)thieno[2,3-d]pyrimidin-4-amine

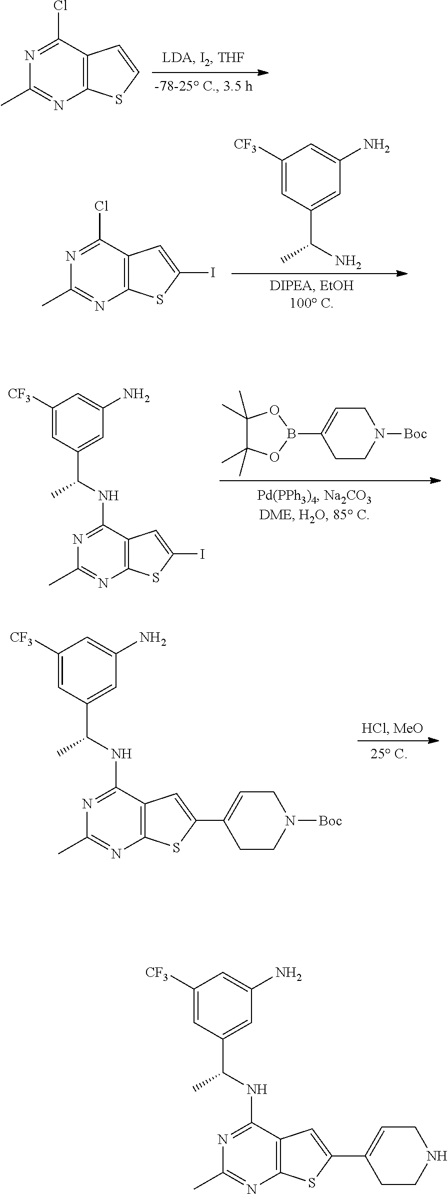

### Step 1.

To a mixture of 4-chloro-2-methyl-thieno[2,3-d]pyrimidine (300 mg, 1.62 mmol) in THF (10 mL) at −78° C. under an atmosphere of N₂ was added 2M LDA in hexanes (975 μL, 1.95 mmol). The mixture was stirred at −78° C. for 30 min, then a solution of I₂ (536 mg, 2.11 mmol) in THF (5 mL) was added at −78° C. The mixture was allowed to warm to rt and stirred for 3 h, then poured into ice-cooled H₂O (50 mL) and extracted with EtOAc (150 mL×3). The combined organic layers were washed with brine (50 mL), dried with anhydrous Na₂SO₄, and filtered. The solvent was concentrated under reduced pressure and the residue was purified by column chromatography to give 4-chloro-6-iodo-2-methyl-thieno[2,3-d]pyrimidine (110 mg, 22% yield). ¹H NMR (400 MHz, DMSO-d₆) δ ppm 7.90 (s, 1H), 2.67 (s, 3H).

### Step 2.

To a mixture of 4-chloro-6-iodo-2-methyl-thieno[2,3-d]pyrimidine (200 mg, 0.64 mmol) and 3-[(1R)-1-aminoethyl]-5-(trifluoromethyl)aniline (171 mg, 0.84 mmol) in EtOH (6 mL) was added DIPEA (337 μL, 1.93 mmol). The mixture was heated to 100° C. in a crimped vial and stirred for 6 hrs. The solvent was concentrated under reduced pressure and the residue was purified by column chromatography to give N-[(1R)-1-[3-amino-5-(trifluoromethyl)phenyl]ethyl]-6-iodo-2-methyl-thieno[2,3-d]pyrimidin-4-amine (152 mg, 49% yield). LCMS (ESI): m/z: [M+H] calculated for C₁₆H₁₅F₃IN₄S: 479.0; found 479.0; ¹H NMR (400 MHz, DMSO-d₆) δ ppm 8.12-8.01 (m, 2H), 6.83 (d, J=13.2 Hz, 2H), 6.69 (s, 1H), 5.54 (s, 2H), 5.41 (q, J=7.3 Hz, 1H), 2.36 (s, 3H), 1.48 (d, J=7.1 Hz, 3H).

### Step 3.

Tert-butyl4-[4-[[(1R)-1-[3-amino-5-(trifluoromethyl)phenyl]ethyl]amino]-2-methyl-thieno[2,3-d]pyrimidin-6-yl]-3,6-dihydro-2H-pyridine-1-carboxylate was synthesized in a manner similar to tert-butyl 4-[4-[[(1R)-1-[3-amino-5-(trifluoromethyl)phenyl]ethyl]amino]-7-methyl-pyrrolo[2,3-d]pyrimidin-6-yl]-3,6-dihydro-2H-pyridine-1-carboxylate except N-[(1R)-1-[3-amino-5-(trifluoromethyl)phenyl]ethyl]-6-bromo-7-methyl-pyrrolo[2,3-d]pyrimidin-4-amine was substituted with was substituted with N-[(1R)-1-[3-amino-5-(trifluoromethyl)phenyl]ethyl]-6-iodo-2-methyl-thieno[2,3-d]pyrimidin-4-amine.

### Step 4.

N-[(1R)-1-[3-amino-5-(trifluoromethyl)phenyl]ethyl]-2-methyl-6-(1,2,3,6-tetrahydropyridin-4-yl)thieno[2,3-d]pyrimidin-4-amine was synthesized in a manner similar to N-[(1R)-1-[3-amino-5-(trifluoromethyl)phenyl]ethyl]-7-methyl-6-(1,2,3,6-tetrahydropyridin-4-yl)pyrrolo[2,3-d]pyrimidin-4-amine except tert-butyl 4-[4-[[(1 R)-1-[3-amino-5-(trifluoromethyl)phenyl]ethyl]amino]-7-methyl-pyrrolo[2,3 -d]pyrimidin-6-yl]-3,6-dihydro-2H-pyridine-1-carboxylate was substituted with tert-butyl 4-[4-[[(1R)-1-[3-amino-5-(trifluoromethyl)phenyl]ethyl]amino]-2-methyl-thieno[2,3-d]pyrimidin-6-yl]-3,6-dihydro-2H-pyridine-1-carboxylate. LCMS (ESI): m/z: [M+H] calculated for C₂₁H₂₃F₃N₅ S : 434.2; found 434.2; ¹H NMR (400 MHz, DMSO-d₆) δ ppm 8.29 (s, 1H), 8.03 (d, J=8.1 Hz, 1H), 7.64 (s, 1H), 6.84 (d, J=10.6 Hz, 2H), 6.70 (s, 1H), 6.13 (s, 1H), 5.54 (s, 2H), 5.48-5.38 (m, 1H), 3.51 (s, 2H), 3.08 (t, J=5.4 Hz, 2H), 2.53-2.52 (m, 2H), 2.37 (s, 3H), 1.50 (d, J=7.1 Hz, 3H).

### Example 47. Synthesis of N-[(1R)-1-[3-amino-5-(trifluoromethyl)phenyl]ethyl]-6-morpholino-pyrrolo[2,1-f][1,2,4]triazin-4-amine

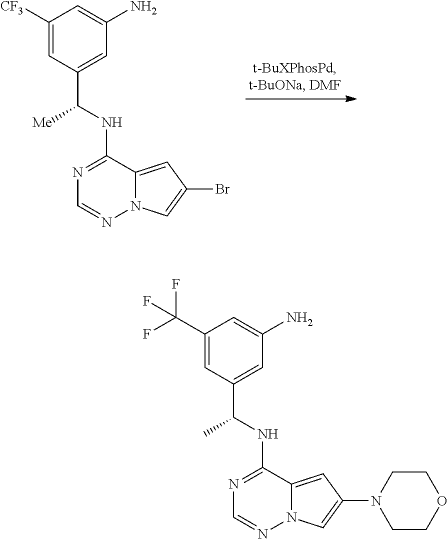 [SMILES: C[C@@H](Nc1ncnn2cc(Br)cc12)c1cc(N)cc(C(F)(F)F)c1 ; C[C@@H](Nc1ncnn2cc(N3CCOCC3)cc12)c1cc(N)cc(C(F)(F)F)c1]

### Step 1.

N-[(1R)-1-[3-amino-5-(trifluoromethyl)phenyl]ethyl]-6-morpholino-pyrrolo[2,1-f][1,2,4]triazine-4-amine was synthesized in a manner similar to 1-[4-[4-[[(1R)-1-[3-amino-5-(trifluoromethyl)phenyl]ethyl]amino]pyrrolo[2,1-f][1,2,4]triazin-6-yl]piperazin-1-yl]ethanone except piperazin-1-ylethanone was substituted with morpholine. LCMS (ESI): m/z: [M+H] calculated for C₁₉H₂₂F₃N₆O: 407.17; found 407.2; ¹H NMR (400 MHz, METHANOL-d₄) δ ppm 7.71 (s, 1H), 7.22 (d, J=1.6 Hz, 1H), 6.92 (m, 2H), 6.80 (s, 1H), 6.59 (d, J=1.6 Hz, 1H), 5.38 (m, 1H), 3.85 (m, 4H), 3.06 (m, 4H), 1.59 (d, J=6.8 Hz, 3H).

### Example 48. Synthesis of [5-[[(1R)-1-[3-amino-5-(trifluoromethyl)phenyl]ethyl]amino]-7-chloro-imidazo[1,2-a]pyrimidin-2-yl]-morpholino-methanone

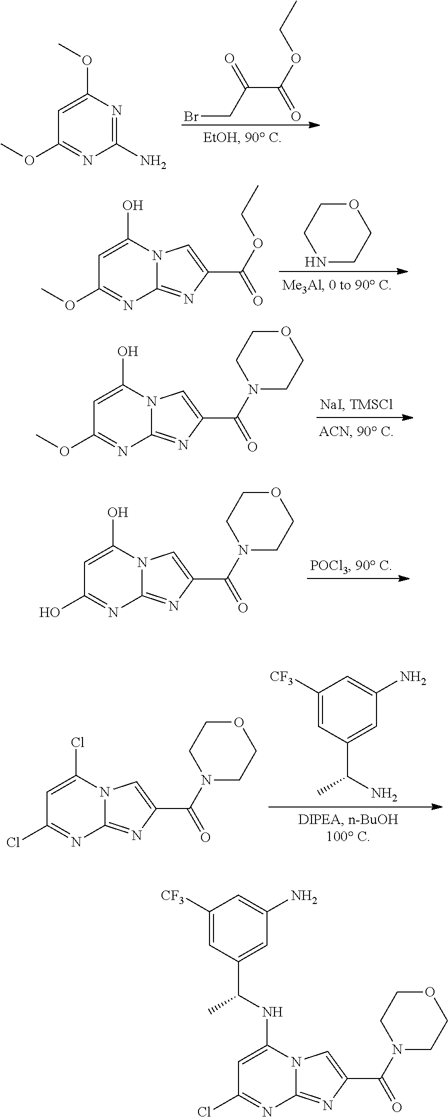

### Step 1.

A mixture of 4,6-dimethoxypyrimidin-2-amine (10 g, 64.5 mmol) and ethyl 3-bromo-2-oxo-propanoate (8.06 mL, 64.5 mmol) in EtOH (120 mL) was heated to 90° C. in a crimped vial and stirred for 16 h. The solvent was concentrated under reduced pressure, and the residue was washed with EtOAc (30 mL), then filtered and the solvent concentrated under reduced pressure to give ethyl 5-hydroxy-7-methoxy-imidazo[1,2-a]pyrimidine-2-carboxylate (2.7 g, 18% yield). LCMS (ESI): m/z: [M+H] calculated for C₁₀H₁₂N₃O₄: 238.07; found 238.0.

### Step 2.

To a mixture of 2M trimethylaluminum in toluene (3.16 mL, 6.3 mmol) and morpholine (20 mL) at 0° C. was added ethyl 5-hydroxy-7-methoxy-imidazo[1,2-a]pyrimidine-2-carboxylate (1.5 g, 6.3 mmol). The mixture was heated to 90° C. and stirred for 12 h, then H₂O (5 mL) was added and the mixture was filtered. The solvent was concentrated under reduced pressure and the residue was purified by column chromatography, then dissolved in DCM, filtered and the solvent concentrated under reduced pressure to give (5-hydroxy-7-methoxy-imidazo[1,2-a]pyrimidin-2-yl)-morpholino-methanone (400 mg, 23% yield). LCMS (ESI): m/z: [M+H] calculated for C₁₂H₁₅N₄O₄: 279.1; found 279.1; ¹H NMR (400 MHz, DMSO-d₆) δ ppm 7.86 (s, 1H), 5.12 (s, 1H), 3.86-3.60 (m, 11H).

### Step 3.

To a mixture of (5-hydroxy-7-methoxy-imidazo[1,2-a]pyrimidin-2-yl)-morpholino-methanone (400 mg, 1.44 mmol) in MeCN (4 mL) under an atmosphere of N₂ was added NaI (646 mg, 4.31 mmol) and TMSCl (547 μL, 4.31 mmol). The mixture was heated to 90° C. in a crimped vial and stirred for 2 h, then H₂O (10 mL) and NaHSO₃ (150 mg) were added, and the mixture was filtered. The filter cake was suspended in EtOH (5 mL), filtered, and the solvent was concentrated under reduced pressure to give (5,7-dihydroxyimidazo[1,2-a]pyrimidin-2-yl)-morpholino-methanone (360 mg, 95% yield). LCMS (ESI): m/z: [M+H] calculated for C₁₁H₁₃N₄O₄: 265.09; found 265.1; ¹H NMR (400 MHz, DMSO-d₆) δ ppm 7.82 (s, 1H), 5.00 (s, 1H), 3.75-3.59 (m, 8H).

### Step 4.

A mixture of (5,7-dihydroxyimidazo[1,2-a]pyrimidin-2-yl)-morpholino-methanone (310 mg, 1.17 mmol) in POCl₃ (3 mL) was heated to 90° C. and stirred for 4 h, then the mixture was concentrated under reduced pressure. Aqueous NaHCO₃ (pH 8) was added and the mixture was extracted with EtOAc (15 mL×2). The combined organic layers were washed with brine (10 mL), dried over Na₂SO₄, filtered and the solvent concentrated under reduced pressure to give (5,7-dichloroimidazo[1,2-a]pyrimidin-2-yl)-morpholino-methanone (70 mg, 20% yield). LCMS (ESI): m/z: [M+H] calculated for C₁₁H₁₁Cl₂N₄O₂: 301.13; found 301.1.

### Step 5.

To a mixture of (5,7-dichloroimidazo[1,2-a]pyrimidin-2-yl)-morpholino-methanone (70 mg, 0.23 mmol) and 3-[(1R)-1-aminoethyl]-5-(trifluoromethyl)aniline (52 mg, 0.26 mmol) in n-BuOH (1 mL) was added DIPEA (405 μL, 2.3 mmol). The mixture was heated to 100° C. and stirred for 8 h, then filtered. The solvent was concentrated under reduced pressure and the residue was purified by prep-HPLC to give [5-[[(1R)-1-[3-amino-5-(trifluoromethyl)phenyl]ethyl]amino]-7-chloro-imidazo[1,2-a]pyrimidin-2-yl]-morpholino-methanone (20 mg, 18% yield). LCMS (ESI): m/z: [M+H] calculated for C₂₀H₂₁ClF₃N₆O₂: 469.13; found 469.1; ¹H NMR (400 MHz, DMSO-d₆) δ ppm 8.73 (s, 1H), 8.52 (d, J=6.9 Hz, 1H), 6.90 (s, 1H), 6.82 (s, 1H), 6.74 (s, 1H), 6.09 (s, 1H), 5.62 (s, 2H), 4.95 (t, J=6.9 Hz, 1H), 4.35-4.21 (m, 2H), 3.65 (s, 6H), 1.56 (d, J=6.9 Hz, 3H).

### Example 49. Synthesis of N-[(1R)-1-[3-amino-5-(trifluoromethyl)phenyl]ethyl]-6-(1,2,3,6-tetrahydropyridin-4-yl)thieno[3,2-d]pyrimidin-4-amine

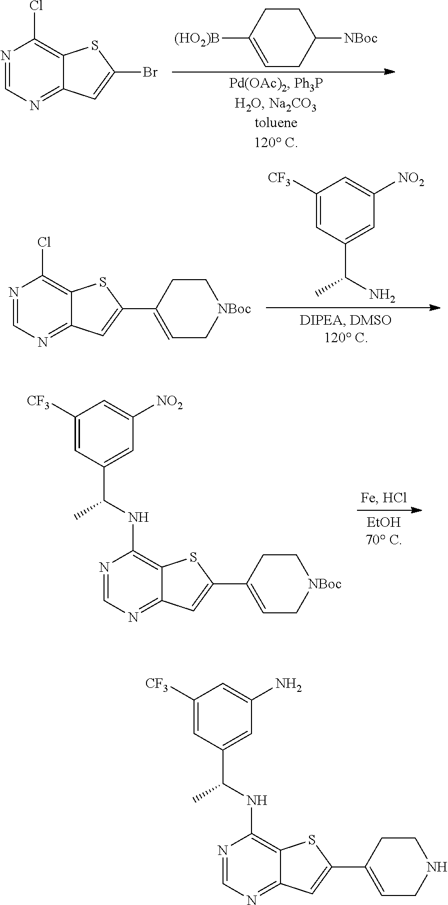

### Step 1.

To a mixture of 6-bromo-4-chlorothieno[3,2-d]pyrimidine (1.01 g, 4.1 mmol) and (1-(tert-butoxycarbonyl)-1,2,3,6-tetrahydropyridin-4-yl)boronic acid (1.06 g, 4.7 mmol) in toluene (20 mL) under an atmosphere of Ar was added Na₂CO₃ (1.47 g, 13.8 mmol) in H₂O (5.0 mL). The mixture was purged with Ar for 15 min, then Ph₃P (373 mg, 1.4 mmol) and Pd(OAc)₂ (110 mg, 0.5 mmol) were added. The mixture was heated to 110° C. and stirred overnight, then filtered through a short plug of Celite® and the filter cake washed with EtOAc. The filtrate was washed with H₂O and brine, then dried over anhydrous Na₂SO₄ and filtered. The solvent was concentrated under reduced pressure and the residue was purified by column chromatography to give tert-butyl 4-{4-chlorothieno[3,2-d]pyrimidin-6-yl}-1,2,3,6-tetrahydropyridine-1-carboxylate (1.14 g, 80% yield). LCMS (ESI): m/z: [M−H] calculated for C₁₆H₁₈ClN₃O₂S: 351.08; found 349.85; ¹H NMR (300 MHz, CDCl₃) δ ppm 8.91 (s, 1H), 7.37 (s, 1H), 6.47 (s, 1H), 4.20-4.15 (m, 2H), 3.69 (t, J=5.7 Hz, 2H), 2.63 (s, 2H), 1.50 (s, 9H).

### Step 2.

Tert-butyl 4-(4-{[(1R)-1-[3-nitro-5-(trifluoromethyl)phenyl]ethyl]amino}thieno[3,2-d]pyrimidin-6-yl)-1,2,3,6-tetrahydropyridine-1-carboxylate was synthesized in a manner similar to 2-chloro-N-[(1R)-1-[3-nitro-5-(trifluoromethyl)phenyl]ethyl]-7-(oxolan-3-ylmethyl)-5H,6H,7 H, 8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with tert-butyl 4-{4-chlorothieno[3,2-d]pyrimidin-6-yl}-1,2,3,6-tetrahydropyridine-1-carboxylate. The product was used directly in the next step.

### Step 3.

Tert-butyl 4-(4-{[(1R)-1-[3-amino-5-(trifluoromethyl)phenyl]ethyl]amino}thieno[3,2-d]pyrimidin-6-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with tert-butyl 4-(4-{[(1R)-1-[3-nitro-5-(trifluoromethyl)phenyl]ethyl]amino}thieno[3,2-d]pyrimidin-6-yl)-1,2,3,6-tetrahydropyridine-1-carboxylate. LCMS (ESI): m/z: [M+H] calculated for C₂₅H₂₈F₃N₅O₂S: 519.19; found 520.10.

### Step 4.

To a mixture of tert-butyl 4-(4-{[(1R)-1-[3-amino-5-(trifluoromethyl)phenyl]ethyl]amino}thieno[3,2-d]pyrimidin-6-yl)-1,2,3,6-tetrahydropyridine-1-carboxylate (386 mg, 0.74 mmol) in Et₂O (1.2 mL) at 0° C. was added 4M HCl in 1,4-dioxane (0.93 mL, 3.72 mmol). The mixture was stirred at rt overnight then poured into H₂O, and the pH adjusted to ˜7 with 10% aqueous NaHCO₃. The mixture was extracted with DCM, the combined organic layers were dried over anhydrous Na₂SO₄ and filtered. The solvent was concentrated under reduced pressure and the residue was purified by prep-HPLC to give N-[(1R)-1-[3-amino-5-(trifluoromethyl)phenyl]ethyl]-6-(1,2,3,6-tetrahydropyridin-4-yl)thieno[3,2-d]pyrimidin-4-amine (17 mg). LCMS (ESI): m/z: [M+H] calculated for C₂₀H₂₀F₃N₅S: 419.14; found 420.04; ¹H NMR (300 MHz, METHANOL-d₄) δ ppm 8.35 (s, 1H), 7.21 (s, 1H), 6.96 (d, J=5.9 Hz, 2H), 6.81 (s, 1H), 6.54 (s, 1H), 5.50 (q, J=7.0 Hz, 1H), 3.52 (d, J=3.2 Hz, 2H), 3.09 (t, J=5.7 Hz, 2H), 2.59 (s, 2H), 1.61 (d, J=7.1 Hz, 3H).

### Example 50. Synthesis of N-[(1R)-1-[3-amino-5-(trifluoromethyl)phenyl]ethyl]-6-(morpholine-4-carbonyl)pyrrolo[2,1-f][1,2,4]triazin-4-amine

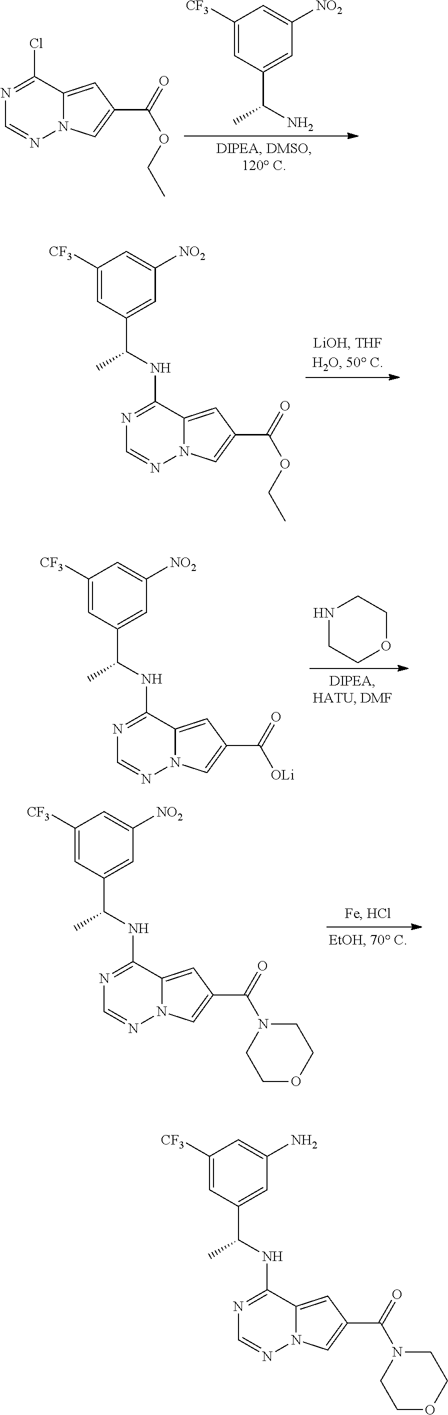

### Step 1.

Ethyl 4-{[(1R)-1-[3-nitro-5-(trifluoromethyl)phenyl]ethyl]amino}pyrrolo[2,1-f][1,2,4]triazine-6-carboxylateas was synthesized in a manner similar to 2-chloro-N-[(1R)-1-[3-nitro-5-(trifluoromethyl)phenyl]ethyl]-7-(oxolan-3-ylmethyl)-5H,6H,7 H, 8H,9H-pyrimido[4,5-d]azepin-4-amine except 2,4-dichloro-7-(oxolan-3-ylmethyl)-5H,6H,7H, 8H,9H-pyrimido[4,5-d]azepine was substituted with ethyl 4-chloropyrrolo[2,1-f][1,2,4]triazine-6-carboxylate. LCMS (ESI): m/z: [M+H] calculated for C₁₈H₁₆F₃N₅O₄: 423.12; found 424.20; ¹H NMR (300 MHz, DMSO-d₆) δ ppm 8.99 (d, J=7.7 Hz, 1H), 8.57 (s, 1H), 8.38 (s, 1H), 8.29 (s, 1H), 8.10 (d, J=1.7 Hz, 1H), 7.93 (s, 1H), 7.52 (d, J=1.6 Hz, 1H), 5.75-5.64 (m, 1H), 4.28 (q, J=7.1 Hz, 2H), 1.61 (d, J=7.0 Hz, 3H), 1.32 (t, J=7.1 Hz, 3H).

### Step 2.

To a mixture of 4-{[(1R)-1-[3-nitro-5-(trifluoromethyl)phenyl]ethyl]amino }pyrrolo[2,1-f][1,2,4]triazine-6-carboxylate (665 mg, 1.6 mmol) in THF (8.3 mL) and H₂O (8.3 mL) was added LiOH.H₂O (79 mg, 1.9 mmol). The mixture was heated to 50° C. and stirred overnight, then the solvent was concentrated under reduced pressure to give 4-{[(1R)-1-[3-nitro-5-(trifluoromethyl)phenyl]-ethyl]amino}pyrrolo[2,1-f][1,2,4]triazine-6-carboxylic acid as a lithium salt (640 mg, 100% yield). LCMS (ESI): m/z: [M+H] calculated for C₁₆H₁₂F₃N₅O₄: 395.08; found 396.15.

### Step 3.

To a mixture of 4-{[(1R)-1-[3-nitro-5-(trifluoromethyl)phenyl]ethyl]amino}pyrrolo[2,1-f][1,2,4]triazine-6-carboxylic acid lithium salt (265 mg, 0.7 mmol) in DMF (5.3 mL) was added DIPEA (345 μL, 2.0 mmol), morpholine (63 μL, 0.7 mmol) and HATU (502 mg, 1.3 mmol). The mixture was stirred at rt overnight, then H₂O was added and the mixture extracted with Et₂O/EtOAc (×2). The combined organic layers were dried over anhydrous Na₂SO₄, filtered and the solvent was concentrated under reduced pressure to give 6-(morpholine-4-carbonyl)-N-[(1R)-1-[3-nitro-5-(trifluoromethyl)phenyl]ethyl]pyrrolo[2,1-f][1,2,4]triazin-4-amine (320 mg, 100% yield). LCMS (ESI): m/z: [M+H] calculated for C₂₀H₁₉F₃N₆O₄: 464.14; found 465.15. Approximately half of the material was purified by prep-HPLC. LCMS (ESI): m/z: [M−H] calculated for C₂₀H₁₉F₃N₆O₄: 464.14; found 463.1; ¹H NMR (300 MHz, DMSO-d₆) δ ppm 8.88 (d, J=7.2 Hz, 1H), 8.56 (s, 1H), 8.38 (s, 1H), 8.29 (s, 1H), 7.94 (d, J=1.7 Hz, 1H), 7.90 (s, 1H), 7.27 (d, J=1.8 Hz, 1H), 5.79-5.60 (m, 1H), 3.73-3.55 (m, 8H), 1.62 (d, J=7.0 Hz, 3H).

### Step 4.

N-[(1R)-1-[3 -amino-5 -(trifluoromethyl)phenyl]ethyl]-6-(morpholine-4-carbonyl)pyrrolo[2,1-f][1,2,4]triazin-4-amine was synthesized in a manner similar to N-[(1R)-1-[3 -amino -5 -(trifluoromethyl)phenyl]ethyl]-2-chloro-7 -(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5 -d]azepin-4-amine was substituted with 6-(morpholine-4-carbonyl)-N-[(1R)-1-[3-nitro-5-(trifluoromethyl)phenyl]ethyl]pyrrolo[2,1-f][1,2,4]triazin-4-amine. LCMS (ESI): m/z: [M+H] calculated for C₂₀H₂₁F₃N₆O₂: 434.17; found 435.22; ¹H NMR (300 MHz, DMSO-d₆) δ ppm 8.70 (d, J=7.8 Hz, 1H), 7.90 (d, J=1.9 Hz, 2H), 7.28 (d, J=1.8 Hz, 1H), 6.81 (d, J=6.7 Hz, 2H), 6.71 (s, 1H), 5.59 (s, 2H), 5.41 (t, J=7.1 Hz, 1H), 3.74-3.54 (m, 8H), 1.52 (d, J=7.0 Hz, 3H).

### Example 51. Synthesis of N-[(1R)-1-[3-amino-5-(trifluoromethyl)phenyl]ethyl]-2-chloro-6-(morpholine-4-carbonyl)-5H-pyrrolo[3,2-d]pyrimidin-4-amine

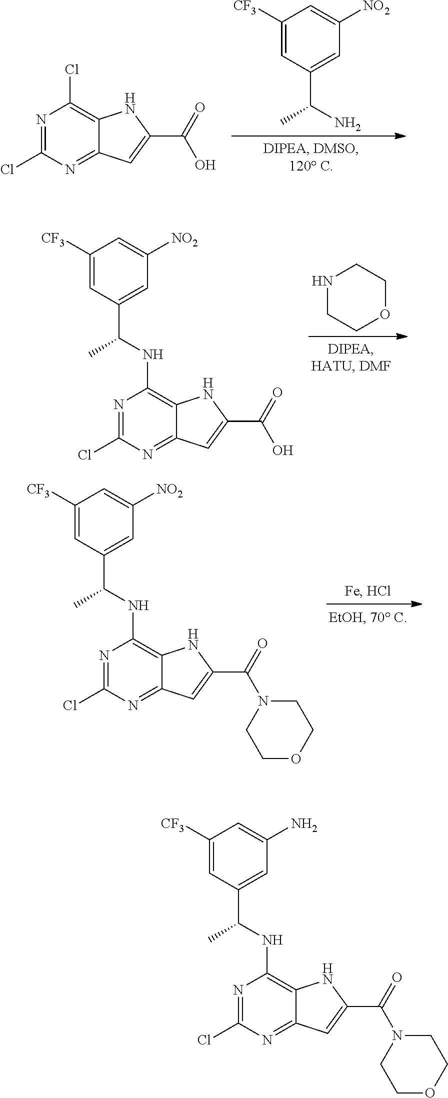

### Step 1.

2-Chloro-4-{[(1R)-1-[3-nitro-5-(trifluoromethyl)phenyl]ethyl]amino}-5H-pyrrolo[3,2-d]pyrimidine-6-carboxylic acid was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5 -d] azepin-4-amine except 2,4-dichloro-7-(oxolan-3-ylmethyl)-5H,6H,7H,8H,9H-pyrimido[4,5 -d] azepine was substituted with 2,4-dichloro-5H-pyrrolo[3,2-d]pyrimidine-6-carboxylic acid. LCMS (ESI): m/z: [M+H] calculated for C₁₆H₁₁ClF₃N₅O₄: 429.05; found 429.95; ¹H NMR (300 MHz, DMSO-d₆) δ ppm 12.02 (s, 1H), 8.56 (s, 1H), 8.43-8.29 (m, 3H), 6.88 (s, 1H), 5.60-5.45 (m, 1H), 1.64 (d, J=6.9 Hz, 3H).

### Step 2.

2-Chloro-6-(morpholine-4-carbonyl)-N-[(1R)-1-[3-nitro-5-(trifluoromethyl)phenyl]ethyl]-5H-pyrrolo[3,2-d]pyrimidin-4-amine was synthesized in a manner similar to 6-(morpholine-4-carbonyl)-N-[(1R)-1-[3-nitro-5-(trifluoromethyl)phenyl]ethyl]pyrrolo[2,1-f][1,2,4]triazin-4-amine except 4-{[(1R)-1-[3-nitro-5-(trifluoromethyl)phenyl]ethyl]amino}pyrrolo[2,1-f][1,2,4]triazine-6-carboxylic acid lithium salt was substituted with 2-chloro-4-{[(1R)-1-[3-nitro-5-(trifluoromethyl)phenyl]ethyl]amino}-5H-pyrrolo[3,2-d]pyrimidine-6-carboxylic acid. LCMS (ESI): m/z: [M+H] calculated for C₂₀H₁₈ClF₃N₆O₄: 498.10; found 499.12; ¹H NMR (300 MHz, DMSO-d₆) δ ppm 11.88 (s, 1H), 8.55 (s, 1H), 8.39 (m, 2H), 8.32 (s, 1H), 6.78 (s, 1H), 5.69-5.31 (m, 1H), 3.77 (s, 4H), 3.67 (d, J=4.5 Hz, 4H), 1.63 (d, J=6.9 Hz, 3H).

### Step 3.

N-[(1R)-1-[3-amino-5-(trifluoromethyl)phenyl]ethyl]-2-chloro-6-(morpholine-4-carbonyl)-5H-pyrrolo[3,2-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 2-chloro-6-(morpholine-4-carbonyl)-N-[(1R)-1-[3-nitro-5-(trifluoromethyl)phenyl]ethyl]-5H-pyrrolo[3,2-d]pyrimidin-4-amine. LCMS (ESI): m/z: [M+H] calculated for C₂₀H₂₀ClF₃N₆O₂: 468.13; found 469.13; ¹H NMR (300 MHz, DMSO-d₆) δ ppm 11.88 (s, 1H), 8.10 (s, 1H), 6.83 (d, J=4.6 Hz, 2H), 6.79-6.69 (m, 2H), 5.62 (s, 2H), 5.34-5.20 (m, 1H), 3.85-3.61 (m, 8H), 1.51 (d, J=6.9 Hz, 3H).

### Example 52. Synthesis of N-[(1R)-1-[3-amino-5-(trifluoromethyl)phenyl]ethyl]-2-(1,2,3,6-tetrahydropyridin-4-yl)pyrazolo[1,5-a]pyrimidin-7-amine

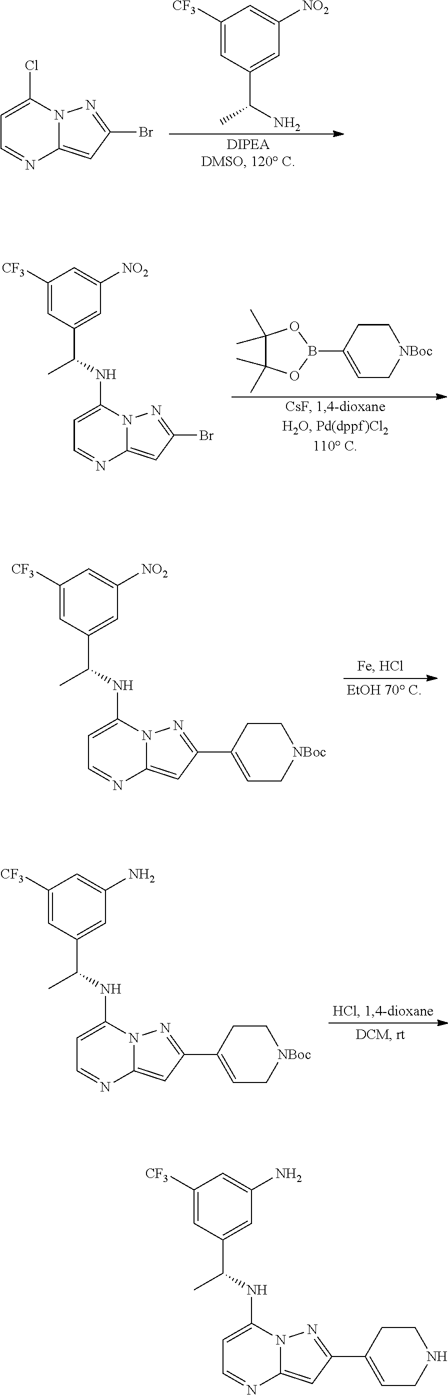

### Step 1.

2-Bromo-N-[(1R)-1-[3-nitro-5 -(trifluoromethyl)phenyl]ethyl]pyrazolo[1,5-a]pyrimidin-7-amine was synthesized in a manner similar to 2-chloro-N-[(1 R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H ,6H ,7 H,8H,9H-pyrimido[4,5-d]azepine was substituted with 2-bromo-7-chloropyrazolo[1,5-a]pyrimidine. LCMS (ESI): m/z: [M+H] calculated for C₁₅H₁₁BrF₃N₅O₂: 429.00; found 429.70; ¹H NMR (300 MHz, DMSO-d₆) δ ppm 8.88-8.78 (m, 2H), 8.52 (s, 1H), 8.38 (s, 1H), 8.12 (d, J=5.4 Hz, 1H), 6.62 (s, 1H), 6.29 (d, J=5.4 Hz, 1H), 5.36-5.23 (m, 1H), 1.69 (d, J=6.8 Hz, 3H).

### Step 2.

To an Ar-purged mixture of 2-bromo-N-[(1R)-1-[3-nitro-5-(trifluoromethyl)phenyl]ethyl]pyrazolo[1,5-a]pyrimidin-7-amine (707 mg, 1.64 mmol) and N-Boc-1,2,5,6-tetrahydropyridine-4-boronic acid pinacol ester (534 mg, 1.73 mmol) in 1,4-dioxane (25 mL) was added CsF (499 mg, 3.29 mmol) in H₂O (7 mL). The mixture was purged with Ar for a further 15 min, then Pd(dppf)Cl₂ ([1,1′-Bis(diphenylphosphino)ferrocene]dichloropalladium(II), 60 mg, 82 μmol) was added, the mixture heated to 110° C. and stirred overnight. The mixture was filtered through a pad of Celite® and the filter cake washed with EtOAc. The filtrate was washed with H₂O and brine, dried over anhydrous Na₂SO₄ and filtered. The solvent was concentrated under reduced pressure and the residue was purified by column chromatography to give tert-butyl 4 -(7 -{[(1R)-1-[3 -nitro-5 -(trifluoromethyl)phenyl]ethyl]amino}pyrazolo[1,5-a]pyrimidin-2-yl)-1,2,3,6-tetrahydropyridine-1 -carboxylate (602 mg, 69% yield). LCMS (ESI): m/z: [M+H] calculated for C₂₅H₂₇F₃N₆O₄: 532.20; found 533.00; ¹H NMR (300 MHz, DMSO-d₆) δ ppm 8.83 (s, 1H), 8.51 (s, 1H), 8.39 (s, 1H), 8.30 (d, J=8.5 Hz, 1H), 8.05 (d, J=5.2 Hz, 1H), 6.65-6.53 (m, 2H), 6.12 (d, J=5.4 Hz, 1H), 5.33-5.20 (m, 1H), 4.11-4.01 (m, 2H), 3.66-3.49 (m, 2H), 2.79-2.56 (m, 2H), 1.71 (d, J=6.8 Hz, 3H), 1.44 (s, 9H).

### Step 3.

Tert-butyl 4-(7-{[(1R)-1-[3-amino-5 -(trifluoromethyl)phenyl]ethyl]amino}pyrazolo[1,5-a]pyrimidin-2-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7 H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5 -d]azepin-4-amine was substituted with tert-butyl 4-(7-{[(1R)-1-[3-nitro-5-(trifluoromethyl)phenyl]ethyl]amino}pyrazolo[1,5-a]pyrimidin-2-yl)-1,2,3,6-tetrahydropyridine-1-carboxylate. LCMS (ESI): m/z: [M+H] calculated for C₂₅H₂₉F₃N₆O₂: 502.23; found 503.05; ¹H NMR (300 MHz, DMSO-d₆) δ ppm 8.03 (d, J=5.2 Hz, 1H), 7.86 (d, J=7.7 Hz, 1H), 6.93 (s, 1H), 6.85 (s, 1H), 6.72 (s, 1H), 6.60-6.52 (m, 2H), 5.91 (d, J=5.4 Hz, 1H), 5.63-5.56 (m, 2H), 4.92-4.80 (m, 1H), 4.11-4.01 (m, 2H), 3.64-3.53 (m, 2H), 2.72-2.59 (m, 2H), 2.08 (s, 3H), 1.62 (d, J=6.9 Hz, 3H), 1.44 (s, 9H).

### Step 4.

A mixture of tert-butyl 4-(7-{[(1R)-1-[3-amino-5-(trifluoromethyl)phenyl]ethyl]amino}pyrazolo[1,5-a]pyrimidin-2-yl)-1,2,3,6-tetrahydropyridine-1-carboxylate (564 mg, 1.12 mmol) in DCM (8.5 mL) and 4M HCl in 1,4-dioxane (3.7 mL, 14.6 mmol) was stirred at rt overnight. The solvent was concentrated under reduced pressure and the residue partitioned between H₂O and DCM mixture. Saturated NaHCO₃ was added and the aqueous layer was extracted with DCM (×2) and CHCl₃/MeOH (3:1, v/v). The combined organic layers were dried over anhydrous Na₂SO₄, filtered, then the solvent was concentrated under reduced pressure and the residue was purified by prep-HPLC to give N-[(1R)-1-[3-amino-5-(trifluoromethyl)phenyl]ethyl]-2-(1,2,3,6-tetrahydropyridin-4-yl)pyrazolo[1,5-a]pyrimidin-7-amine (80 mg, 18% yield). LCMS (ESI): m/z: [M+H] calculated for C₂₀H₂₁,F₃N₆: 402.18; found 403.24; ¹H NMR (300 MHz, DMSO-d₆) δ ppm 8.03 (d, J=5.2 Hz, 1H), 7.81 (d, J=7.7 Hz, 1H), 6.94 (s, 1H), 6.85 (s, 1H), 6.73 (s, 1H), 6.61 (s, 1H), 6.52 (s, 1H), 5.90 (d, J=5.3 Hz, 1H), 5.60 (d, J=5.2 Hz, 2H), 4.98-4.75 (m, 1H), 3.43 (m, 2H), 2.94 (m, 2H), 2.56-2.52 (m, 2H), 1.62 (d, J=6.8 Hz, 3H); ¹H NMR (300 MHz, METHANOL-d₄) δ ppm 8.00 (d, J=5.4 Hz, 1H), 6.98 (s, 1H), 6.94 (s, 1H), 6.85 (s, 1H), 6.68-6.62 (m, 1H), 6.51 (s, 1H), 5.89 (d, J=5.5 Hz, 1H), 4.88-4.83 (m, 1H), 3.59-3.51 (m, 2H), 3.12 (m, 2H), 2.78-2.63 (m, 2H), 1.72 (d, J=6.8 Hz, 3H).

### Example 53. Synthesis of N-[(1R)-1-[3-amino-5-(trifluoromethyl)phenyl]ethyl]-2-(1,2,3,6-tetrahydropyridin-4-yl)-[1,2,4]triazolo[1,5-a]pyrazin-8-amine

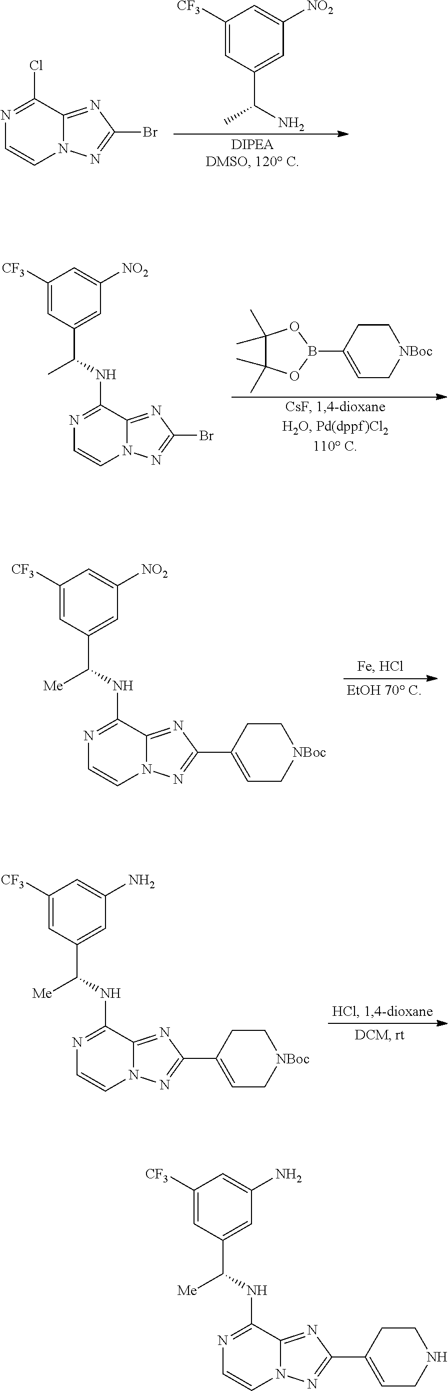

### Step 1.

2-Bromo-N-[(1R)-1-[3-nitro-5-(trifluoromethyl)phenyl]ethyl]-[1,2,4]triazolo[1,5-a]pyrazin-8-amine was synthesized in a manner similar to 2-chloro-N-[(1R)-1-[3-nitro-5-(trifluoromethyl)phenyl]ethyl]-7-(oxolan-3-ylmethyl)-5H,6H,7 H ,8H,9H-pyrimido[4,5 -d]azepin-4-amine except 2,4-dichloro-7-(oxolan-3-ylmethyl)-5H,6H,7H,8H,9H-pyrimido[4,5-d]azepine was substituted with 2-bromo-8-chloro-[1,2,4]triazolo[1,5-a]pyrazine. LCMS (ESI): m/z: [M+H] calculated for C₁₄H₁₀BrF₃N₆O₂: 430.00; found 430.80; ¹H NMR (300 MHz, DMSO-d₆) δ ppm 8.82 (d, J=8.1 Hz, 1H), 8.66 (s, 1H), 8.37-8.31 (m, 2H), 8.13 (d, J=4.6 Hz, 1H), 7.53 (d, J=4.6 Hz, 1H), 5.65-5.52 (m, 1H), 1.60 (d, J=7.0 Hz, 3H).

### Step 2.

Tert-butyl 4-(8-{[(1R)-1-[3 -nitro-5 -(trifluoromethyl)phenyl]ethyl]amino}-[1,2,4]triazolo[1,5-a]pyrazin-2-yl)-1,2,3,6-tetrahydropyridine-1-carboxylate was synthesized in a manner similar to tert-butyl 4-(7-{[(1R)-1-[3-nitro-5-(trifluoromethyl)phenyl]ethyl]amino}pyrazolo[1,5-a]pyrimidin-2-yl)-1,2,3,6-tetrahydropyridine-1-carboxylate except 2-bromo-N-[(1R)-1-[3-nitro-5-(trifluoromethyl)phenyl]ethyl]pyrazolo[1,5-a]pyrimidin-7-amine was substituted with 2-bromo-N-[(1R)-1-[3-nitro-5-(trifluoromethyl)phenyl]ethyl]-[1,2,4]triazolo[1,5-a]pyrazin-8-amine. LCMS (ESI): m/z: [M+H] calculated for C₂₄H₂₆F₃N₇O₄: 533.20; found 534.05; ¹H NMR (300 MHz, DMSO-d₆) δ ppm 8.68 (s, 1H), 8.61 (d, J=8.1 Hz, 1H), 8.37 (s, 1H), 8.33 (s, 1H), 8.10 (d, J=4.6 Hz, 1H), 7.45 (d, J=4.6 Hz, 1H), 6.95-6.90 (m, 1H), 5.64-5.55 (m, 1H), 4.13-4.07 (m, 2H), 3.61-3.53 (m, 2H), 2.69-2.60 (m, 2H), 1.63 (d, J=7.1 Hz, 3H), 1.44 (s, 9H).

### Step 3.

Tert-butyl 4-(8-{[(1R)-1-[3-amino-5-(trifluoromethyl)phenyl]ethyl]amino}-[1,2,4]triazolo[1,5-a]pyrazin-2-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7 H,8H,9H-pyrimido[4,5 -d]azepin-4-amine was substituted with tert-butyl 4-(8-{[(1R)-1-[3-nitro-5-(trifluoromethyl)phenyl]ethyl]amino}-[1,2,4]triazolo[1,5-a]pyrazin-2-yl)-1,2,3,6-tetrahydropyridine-1-carboxylate. LCMS (ESI): m/z: [M+H] calculated for C₂₄H₂₈F₃N₇O₂: 503.23; found 504.05. (note: crude product taken to the next step without purification).

### Step 4.

N-[(1R)-1-[3-amino-5-(trifluoromethyl)phenyl]ethyl]-2-(1,2,3,6-tetrahydropyridin-4-yl)-[1,2,4]triazolo[1,5-a]pyrazin-8-amine was synthesized in a manner similar to N-[(1R)-1-[3-amino-5-(trifluoromethyl)phenyl]ethyl]-2-(1,2,3,6-tetrahydropyridin-4-yl)pyrazolo[1,5 -a]pyrimidin-7-amine except tert-butyl 4-(7-{[(1R)-1-[3-amino-5-(trifluoromethyl)phenyl]ethyl]amino}pyrazolo[1,5-a]pyrimidin-2-yl)-1,2,3,6-tetrahydropyridine-1-carboxylate was substituted with tert-butyl 4-(8-{[(1R)-1-[3-amino-5-(trifluoromethyl)phenyl]ethyl]amino}-[1,2,4]triazolo[1,5-a]pyrazin-2-yl)-1,2,3,6-tetrahydropyridine-1-carboxylate. LCMS (ESI): m/z: [M+H] calculated for C₁₉H₂₀F₃N₇: 403.17; found 404.00; ¹H NMR (300 MHz, METHANOL-d₄) δ ppm 7.91 (d, J=4.7 Hz, 1H), 7.47 (d, J=4.7 Hz, 1H), 7.15-6.93 (m, 3H), 6.82 (s, 1H), 5.32 (q, J=7.0 Hz, 1H), 3.67-3.48 (m, 2H), 3.10 (t, J=5.8 Hz, 2H), 2.85-2.58 (m, 2H), 1.63 (d, J=7.0 Hz, 3H).

### Example 54. Synthesis of N-[(1R)-1-[3-amino-5-(trifluoromethyl)phenyl]ethyl]-2-(1,2,3,6-tetrahydropyridin-4-yl)-6-(trifluoromethyl)-[1,2,4]triazolo[1,5-a]pyridin-8-amine

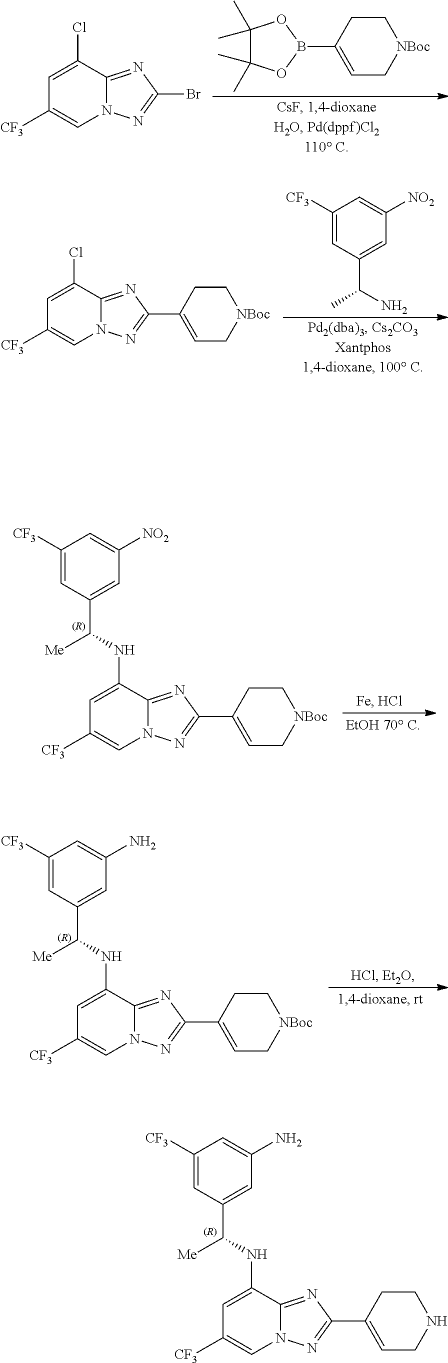

### Step 1.

Tert-butyl 4-[8-chloro-6-(trifluoromethyl)-[1,2,4]triazolo[1,5 -a]pyridin-2-yl]-1,2,3,6-tetrahydropyridine-1-carboxylate was synthesized in a manner similar to tert-butyl 4-(7-{[(1R)-1-[3-nitro-5-(trifluoromethyl)phenyl]ethyl]amino}pyrazolo[1,5-a]pyrimidin-2-yl)-1,2,3,6-tetrahydropyridine-1-carboxylate except 2-bromo-N-[(1R)-1-[3-nitro-5-(trifluoromethyl)phenyl]ethyl]pyrazolo[1,5-a]pyrimidin-7-amine was substituted with 2-bromo-8-chloro-6-(trifluoromethyl)-[1,2,4]triazolo[1,5-a]pyridine. LCMS (ESI): m/z: [M+H] calculated for C₁₇H₁₈ClF₃N₄O₂: 402.11; found 402.9; ¹H NMR (300 MHz, CDCl₃) δ ppm 7.40 (t, J=1.3 Hz, 1H), 7.70 (d, J=1.5 Hz, 1H), 7.12 (s, 1H), 4.18 (q, J=3.0 Hz, 2H), 3.67 (t, J=5.7 Hz, 2H), 2.73 (s, 2H), 1.50 (s, 9H).

### Step 2.

A mixture of tert-butyl 4-[8-chloro-6-(trifluoromethyl)-[1,2,4]triazolo[1,5-a]pyridin-2-yl]-1,2,3,6-tetrahydropyridine-1-carboxylate (0.49 g, 1.2 mmol), (1R)-1-[3-nitro-5-(trifluoromethyl)phenyl]ethan-1-amine HCl salt (0.395 g, 1.46 mmol), Cs₂CO₃ (0.99 g, 3 mmol), Pd₂(dba)₃ (56 mg, 0.06 mmol) and xantphos (106 mg, 0.18 mmol) in 1,4-dioxane (14.7 mL) under an atmosphere of Ar was heated to 100° C. (pre-heated block) and stirred overnight. The mixture was filtered through a pad of Celite®, the filter cake was washed with MeOH, the solvent was concentrated under reduced pressure and the residue was purified by column chromatography to give tert-butyl 4-(8-{[(1R)-1-[3-nitro-5-(trifluoromethyl)phenyl]ethyl]amino}-6-(trifluoromethyl)-[1,2,4]triazolo [1,5-a]pyridin-2-yl)-1,2,3,6-tetrahydropyridine-1-carboxylate (660 mg, 90% yield). LCMS (ESI): m/z: [M−H] calculated for C₂₆H₂₆F₆N₆O₄: 600.19; found 599.1 found; ¹H NMR (300 MHz, CDCl₃) δ ppm 8.45 (d, J=11.3, 2.0 Hz, 2H), 8.26 (s, 1H), 8.00 (s, 1H), 7.04 (s, 1H), 6.08 (s, 1H), 4.86-4.81 (m, 1H), 4.20 (d, J=3.2 Hz, 2H), 3.68 (t, J=5.7 Hz, 2H), 2.74 (s, 2H), 1.78 (d, J=6.8 Hz, 3H), 1.50 (s, 9H).

### Step 3.

Tert-butyl4-(8-{[(1R)-1-[3-amino-5-(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with ter t-butyl 4-(8-{[(1R)-1-[3-nitro-5-(trifluoromethyl)phenyl]ethyl]amino}-6-(trifluoromethyl)-[1,2,4] triazolo[1,5-a]pyridin-2-yl)-1,2,3,6-tetrahydropyridine-1-carboxylate. LCMS (ESI): m/z: [M+H] calculated for C₂₆H₂₈F₆N₆O₂: 570.22; found 571.10; ¹H NMR (300 MHz, DMSO-d₆) δ ppm 8.69 (d, J=1.4 Hz, 1H), 7.15 (d, J=7.2 Hz, 1H), 7.05-6.93 (m, 2H), 6.86 (s, 1H), 6.75-6.64 (m, 1H), 6.34 (d, J=1.5 Hz, 1H), 5.54 (s, 2H), 4.80 (t, J=7.0 Hz, 1H), 4.19-4.02 (m, 2H), 3.58 (t, J=5.6 Hz, 2H), 2.70-2.60 (m, 2H), 1.55 (d, J=6.7 Hz, 3H), 1.45 (s, 9H).

### Step 4.

To a mixture of tert-butyl 4-(8-{[(1R)-1-[3-amino-5-(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate (602 mg, 1.06 mmol) in Et₂O (9 mL) at 0° C. was added 4M HCl in 1,4-dioxane (3.43 mL). The mixture was warmed to rt and stirred overnight, then H₂O and 10% aqueous NaHCO₃ were added to adjust to pH ˜7. The mixture was extracted with DCM, the combined organic layers were dried over anhydrous Na₂SO₄ and filtered. The solvent was concentrated under reduced pressure and the residue was purified by prep-HPLC to give N-[(1R)-1-[3-amino-5-(trifluoromethyl)phenyl]ethyl]-2-(1,2,3,6-tetrahydropyridin-4-yl)-6-(trifluoromethyl)-[1,2,4]triazolo[1,5-a]pyridin-8-amine (163 mg). LCMS (ESI): m/z: [M+H] calculated for C₂₁H₂₀F₆N₆: 470.17; found 471.18; ¹H NMR (300 MHz, METHANOL-d₄) δ ppm 8.39-8.36 (m, 1H), 7.07-7.03 (m, 1H), 6.97 (d, J=6.3 Hz, 2H), 6.86-6.81 (m, 1H), 6.30 (d, J=1.5 Hz, 1H), 4.70 (q, J=6.7 Hz, 1H), 3.63-3.56 (m, 2H), 3.11 (t, J=5.8 Hz, 2H), 2.77-2.65 (m, 2H), 1.66 (d, J=6.8 Hz, 3H).

### Example 55. Synthesis of N-[(1R)-1-[3-amino-5-(trifluoromethyl)phenyl]ethyl]-2-(1,2,3,6-tetrahydropyridin-4-yl)-[1,2,4]triazolo[1,5-a]pyridin-5-amine

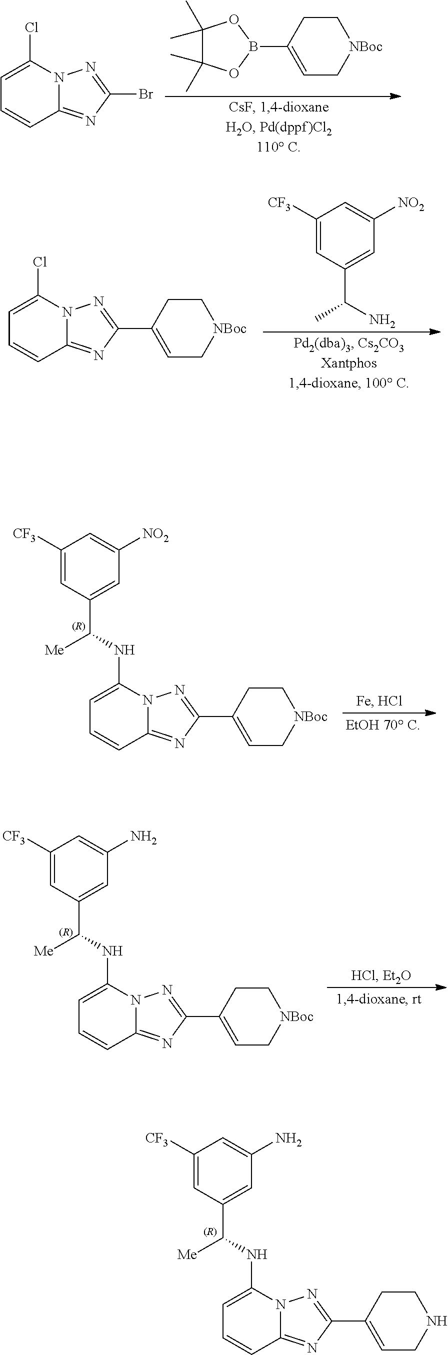

### Step 1.

Tert-butyl 4-{5-chloro-[1,2,4]triazolo[1,5-a]pyridin-2-yl}-1,2,3,6-tetrahydropyridine-1-carboxylate was synthesized in a manner similar to tert-butyl 4-(7-{[(1R)-1-[3-nitro-5-(trifluoromethyl)phenyl]ethyl]amino}pyrazolo[1,5-a]pyrimidin-2-yl)-1,2,3,6-tetrahydropyridine-1-carboxylate except 2-bromo-N-[(1R)-1-[3-nitro-5-(trifluoromethyl)phenyl]ethyl]pyrazolo[1,5-a]pyrimidin-7-amine was substituted with 2-bromo-5-chloro-[1,2,4]triazolo[1,5-a]pyridine. LCMS (ESI): m/z: [M+H] calculated for C₁₆H₁₉ClN₄O₂: 334.12; found 335.20; ¹H NMR (300 MHz, CDCl₃) δ ppm 7.65 (dd, J=9.0, 1.1 Hz, 1H), 7.46 (dd, J=8.9, 7.4 Hz, 1H), 7.08 (dd, J=7.4, 1.0 Hz, 1H), 7.01 (s, 1H), 4.25-4.05 (m, 2H), 3.67 (t, J=5.7 Hz, 2H), 2.79 (s, 2H), 1.50 (s, 9H).

### Step 2.

Tert-butyl 4-(5-{[(1R)-1-[3-nitro-5-(trifluoromethyl)phenyl]ethyl]amino}-[1,2,4]triazolo[1,5-a]pyridin-2-yl)-1,2,3,6-tetrahydropyridine-1-carboxylate was synthesized in a manner similar to tert-butyl 4-(8-{[(1R)-1-[3-nitro-5-(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate except ter t-butyl 4-[8-chloro-6-(trifluoromethyl)-[1,2,4]triazolo[1,5-a]pyridin-2-yl]-1,2,3,6-tetrahydropyridine-1-carboxylate was substituted with tert-butyl 4-{5-chloro-[1,2,4]triazolo[1,5-a]pyridin-2-yl}-1,2,3,6-tetrahydropyridine-1-carboxylate. LCMS (ESI): m/z: [M+H] calculated for C₂₅H₂₇F₃N₆O₄: 532.20; found 533.45; ¹H NMR (300 MHz, DMSO-d₆) δ ppm 8.79 (s, 1H), 8.48 (s, 1H), 8.37 (s, 1H), 7.66 (d, J=8.1 Hz, 1H), 7.36 (t, J=8.7 Hz, 1H), 6.98-6.86 (m, 1H), 6.10 (d, 1H), 5.22-5.10 (m, 1H), 4.09 (s, 2H), 3.58 (t, J=5.8 Hz, 2H), 2.80-2.63 (m, 2H), 1.69 (d, J=6.8 Hz, 3H), 1.44 (s, 9H).

### Step 3.

Tert-butyl4-(5-{[(1R)-1-[3-amino-5-(trifluoromethyl)phenyl]ethyl]amino}-[1,2,4]triazolo[1,5-a]pyridin-2-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7 H,8H,9H-pyrimido[4,5-d]azepin-4-amine was substituted with tert-butyl 4-(5-{[(1R)-1-[3-nitro-5-(trifluoromethyl)phenyl]ethyl]amino}-[1,2,4]triazolo[1,5-a]pyridin-2-yl)-1,2,3,6-tetrahydropyridine-1-carboxylate. LCMS (ESI): m/z: [M+H] calculated for C₂₅H₂₉F₃N₆O₂: 502.23; found 503.40.

### Step 4.

N-[(1R)-1-[3-amino-5-(trifluoromethyl)phenyl]ethyl]-2-(1,2,3,6-tetrahydropyridin-4-yl)-[1,2,4]triazolo[1,5-a]pyridin-5-amine was synthesized in a manner similar to N-[(1R)-1-[3-amino-5-(trifluoromethyl)phenyl]ethyl]-2-(1,2,3,6-tetrahydropyridin-4-yl)-6-(trifluoromethyl)-[1,2,4]triazolo[1,5 -a]pyridin-8-amine except tert-butyl 4-(8-{[(1R)-1-[3-amino-5-(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate was substituted with tert-butyl 4-(5-{[(1R)-1-[3-amino-5-(trifluoromethyl)phenyl]ethyl]amino}-[1,2,4]triazolo[1,5-a]pyridin-2-yl)-1,2,3,6-tetrahydropyridine-1-carboxylate. LCMS (ESI): m/z: [M+H] calculated for C₂₀H₂₁F₃N₆: 402.18; found 403.25; ¹H NMR (300 MHz, METHANOL-d₄) δ ppm 7.40 (dd, J=8.7, 7.9 Hz, 1H), 7.01-6.92 (m, 3H), 6.89 (dd, J=8.7, 1.0 Hz, 1H), 6.84-6.79 (m, 1H), 5.93 (dd, J=8.0, 1.0 Hz, 1H), 4.74 (q, J=6.8 Hz, 1H), 3.55 (q, J=2.9 Hz, 2H), 3.09 (t, J=5.7 Hz, 2H), 2.79-2.69 (m, 2H), 1.68 (d, J=6.8 Hz, 3H).

### Example 56. Synthesis of N-[(1R)-1-[3-amino-5-(trifluoromethyl)phenyl]ethyl]-2-(1,2,3,6-tetrahydropyridin-4-yl)thieno[3,2-c]pyridin-4-amine

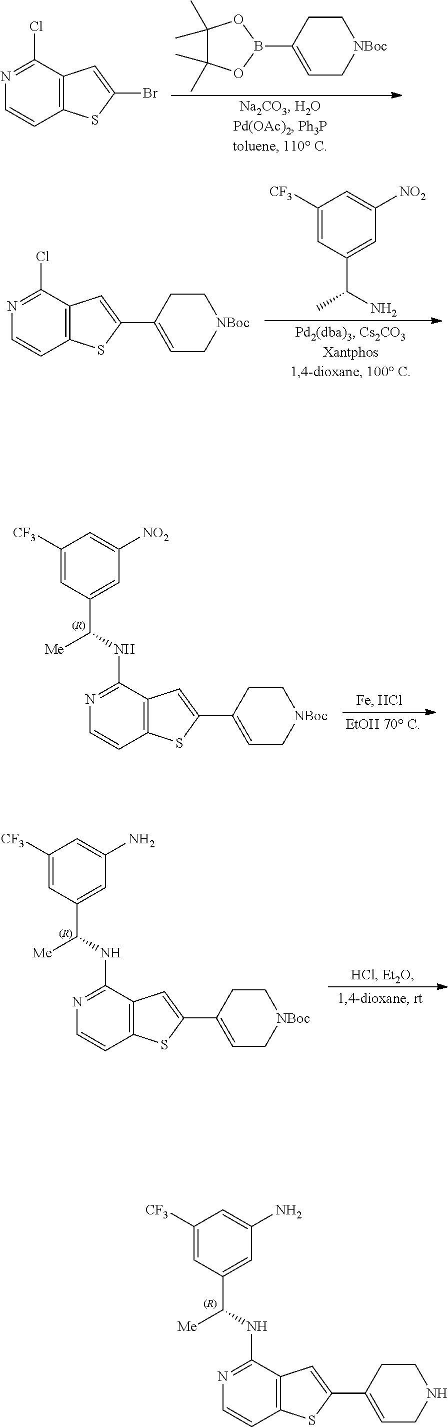

### Step 1.

To an Ar-purged mixture of 2-bromo-4-chlorothieno[3,2-c]pyridine (1.00 g, 4.02 mmol) and N-Boc-1,2,5,6-tetrahydropyridine-4-boronic acid pinacol ester (1.31 g, 4.23 mmol) in toluene (20 mL) was added Na₂CO₃ (1.45 g, 1.37 mmol) in H₂O (5.0 mL). The mixture was purged with Ar for a further 15 min, then Ph₃P (369 mg, 1.37 mmol) and Pd(OAc)₂ (108 mg, 0.48 mmol) were added. The mixture was heated to 110° C. and stirred overnight, then filtered through a short pad of Celite® and the filter cake washed with EtOAc. The filtrate was washed with H₂O and brine, then dried over anhydrous Na₂SO₄ and filtered. The solvent was concentrated under reduced pressure and the residue was purified by column chromatography to give tert-butyl 4-{4-chlorothieno[3,2-c]pyridin-2-yl}-1,2,3,6-tetrahydropyridine-1-carboxylate (1.41 g, 92% yield). LCMS (ESI): m/z: [M+H] calculated for C₁₇H₁₉ClN₂O₂S: 350.09; found 350.85; ¹H NMR (300 MHz, CDCl₃) δ ppm 8.18 (d, J=5.5 Hz, 1H), 7.61 (dd, J=5.5, 0.8 Hz, 1H), 7.30 (s, 1H), 6.25 (s, 1H), 4.17-4.10 (m, 2H), 3.68 (t, J=5.7 Hz, 2H), 2.64 (s, 2H), 1.50 (s, 9H).

### Step 2.

Tert-butyl 4-(4-{[(1R)-1-[3-nitro-5-(trifluoromethyl)phenyl]ethyl]amino}thieno[3,2-c]pyridin-2-yl)-1,2,3,6-tetrahydropyridine-1-carboxylate was synthesized in a manner similar to tert-butyl 4-(8-{[(1R)-1-[3-nitro-5-(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate except ter t-butyl 4-[8-chloro-6-(trifluoromethyl)-[1,2,4]triazolo[1,5-a]pyridin-2-yl]-1,2,3,6-tetrahydropyridine-1-carboxylate was substituted with tert-butyl 4-{4-chlorothieno[3,2-c]pyridin-2-yl}-1,2,3,6-tetrahydropyridine-1-carboxylate. LCMS (ESI): m/z: [M+H] calculated for C₂₆H₂₇F₃N₄O₄S: 548.17; found 549.40; ¹H NMR (300 MHz, DMSO-d₆) δ ppm 8.56 (s, 1H), 8.33 (s, 1H), 8.27 (s, 1H), 7.83 (s, 1H), 7.71 (d, J=5.7 Hz, 1H), 7.60 (d, J=7.3 Hz, 1H), 7.06 (d, J=5.7 Hz, 1H), 6.17 (s, 1H), 5.56 (t, J=7.1 Hz, 1H), 4.05 (s, 2H), 3.69-3.48 (m, 2H), 2.60 (s, 3H), 1.60 (d, J=7.1 Hz, 3H), 1.44 (s, 9H).

### Step 3.

Tert-butyl 4-(4-{[(1R)-1-[3-amino-5-(trifluoromethyl)phenyl]ethyl]amino}thieno[3,2-c]pyridin-2-yl)-1,2,3,6-tetrahydropyridine-1-carboxylate was synthesized in a manner similar to N-[(1R)-1-[3-amino-5-(trifluoromethyl)phenyl]ethyl]-2-chloro-7-(oxolan-3-ylmethyl)-5H,6H,7 H,8H,9H-pyrimido[4,5 -d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with tert-butyl 4-(4-{[(1R)-1-[3-nitro-5-(trifluoromethyl)phenyl]ethyl]amino}thieno[3,2-c]pyridin-2-yl)-1,2,3,6-tetrahydropyridine-1-carboxylate. LCMS (ESI): m/z: [M+H] calculated for C₂₆H₂₉F₃N₄O₂S: 518.20; found 519.05.

### Step 4.

N-[(1R)-1-[3-amino-5-(trifluoromethyl)phenyl]ethyl]-2-(1,2,3,6-tetrahydropyridin-4-yl)thieno[3,2-c]pyridin-4-amine was synthesized in a manner similar to N-[(1R)-1-[3-amino-5-(trifluoromethyl)phenyl]ethyl]-2-(1,2,3,6-tetrahydropyridin-4-yl)-6-(trifluoromethyl)-[1,2,4]triazolo[1,5-a]pyridin-8-amine except tert-butyl 4-(8-{[(1R)-1-[3-amino-5 -(trifluoromethyl)phenyl]ethyl]amino}-6-(trifluoromethyl)-[1,2,4]triazolo[1,5-a]pyridin-2-yl)-1,2,3,6-tetrahydropyridine-1-carboxylate was substituted with tert-butyl 4-(4-{[(1R)-1-[3-amino-5 -(trifluoromethyl)phenyl]ethyl]amino}thieno[3,2-c]pyridin-2-yl)-1,2,3,6-tetrahydropyridine-1-carboxylate. LCMS (ESI): m/z: [M+H] calculated for C₂₁H₂₁F₃N₄S: 418.14; found 419.21; ¹H NMR (300 MHz, METHANOL-d₄) δ ppm 7.71 (d, J=3.9 Hz, 1H), 7.68 (s, 1H), 7.05-6.91 (m, 3H), 6.79 (s, 1H), 6.26 (s, 1H), 5.30 (q, J=7.0 Hz, 1H), 3.52 (d, J=3.2 Hz, 2H), 3.11 (t, J=5.8 Hz, 2H), 2.72-2.59 (m, 2H), 1.60 (d, J=7.0 Hz, 3H).

### Example 57. Synthesis of N-[(1R)-1-[3-amino-5-(trifluoromethyl)phenyl]ethyl]-8-(morpholine-4-carbonyl)-9H-purin-6-amine

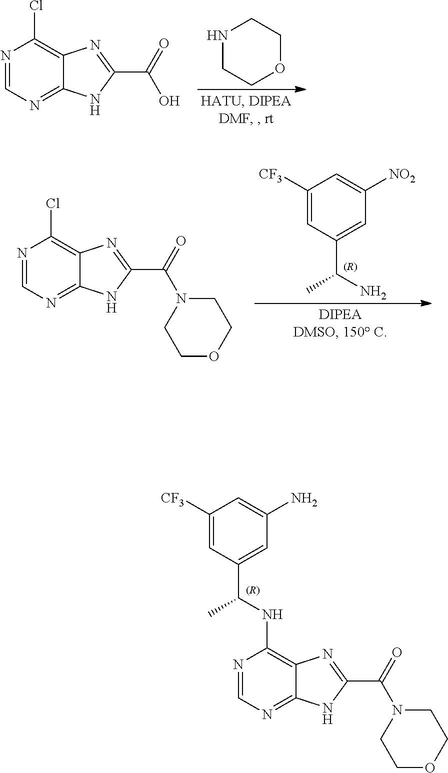

### Step 1.

To a mixture of 6-chloro-9H-purine-8-carboxylic acid (354 mg, 1.78 mmol) in DMF (8.9 mL) was added DIPEA (932 μL, 5.35 mmol) and HATU (1.02 g, 2.67 mmol). The mixture was stirred at rt for 4 h, then H₂O was added, an emerging precipitate was filtered and the filter cake was washed with H₂O. The filtrate was extracted with Et₂O and an emerging precipitate was filtered to give 6-chloro-8-(morpholine-4-carbonyl)-9H-purine (78 mg, 16% yield). LCMS (ESI): m/z: [M+H] calculated for C₁₀H₁₀ClN₅O₂: 267.05; found 267.95; ¹H NMR (300 MHz, DMSO-d₆) δ ppm 13.07 (s, 1H), 8.45 (s, 1H), 4.16-3.92 (m, 4H), 3.80-3.65 (m, 4H).

### Step 2.

A mixture of 6-chloro-8-(morpholine-4-carbonyl)-9H-purine (75 mg, 0.28 mmol) and 3-(1-aminoethyl)-5-(trifluoromethyl)aniline HCl salt (70 mg, 0.29 mmol) in DMSO (2.7 mL) was purged with Ar. DIPEA (0.19 mL, 1.1 mmol) was added and the mixture was heated to 150° C. and stirred for 1 h. H₂O and Et₂O were added and the aqueous layer was extracted with Et₂O (×2). The combined organic layers were dried, filtered, the solvent was concentrated under reduced pressure and the residue was purified by prep-HPLC to give N-[(1R)-1-[3-amino-5-(trifluoromethyl)phenyl]ethyl]-8-(morpholine-4-carbonyl)-9H-purin-6-amine (30 mg, 25% yield). LCMS (ESI): m/z: [M+H] calculated for C₁₉H₂₀F₃N₇O₂: 435.16; found 436.17.

### Example 73. (R)-(2-chloro-6-((1-(3-(1,1-difluoro-2-hydroxyethyl)phenyl)ethyl)amino)-9H-purin-8-yl)(morpholino)methanone

(R)-(2-chloro-6-((1-(3-(1,1-difluoro-2-hydroxyethyl)phenypethyDamino)-9H-purin-8-yl)(morpholino)methanonewas synthesized in the manner similar to Example 57.

| | | Mass |
|---|---|---|
| Example # | Structure | Found |
| Example 73. | 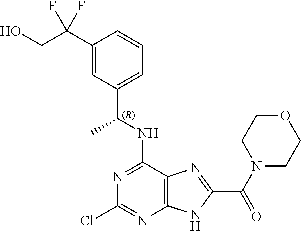 | 467.0 |

### Example 58. Synthesis of N-[(1R)-1-[3-amino-5-(trifluoromethyl)phenyl]ethyl]-6-methyl-2-(morpholine-4-carbonyl)pyrazolo[1,5-a]pyrazin-4-amine

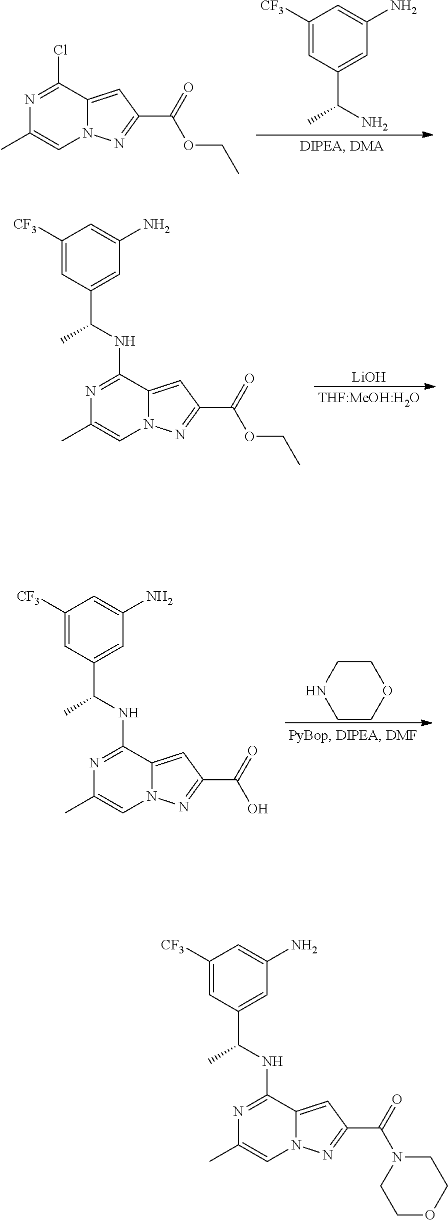

### Step 1.

To a mixture of 3-[(1R)-1-aminoethyl]-5-(trifluoromethyl)aniline (894 mg, 4.38 mmol) and ethyl 4-chloro-6-methylpyrazolo[1,5 -a] pyrazine-2-carboxylate (1.05 g, 4.38 mmol) in DMA (6.25 mL) was added DIPEA (1.52 mL, 8.76 mmol). The mixture was heated to 90° C. and stirred overnight. After cooling the mixture was diluted with H₂O and extracted with EtOAc. The combined organic layers were washed with brine and dried over MgSO₄, filtered, and the solvent was removed under reduced pressure to give ethyl 4-{[(1R)-1-[3-amino-5 -(trifluoromethyl)phenyl]ethyl]amino}-6-methylpyrazolo [1,5-a]pyrazine-2-carboxylate (1.79 g), which was used in the next step without further purification. LCMS (ESI): m/z: [M+H] calculated for C₁₉H₂₁F₃N₅O₂: 408.2; found 408.3.

### Step 2.

To a mixture of ethyl 4-{[(1R)-1-[3-amino-5-(trifluoromethyl)phenyl]ethyl]amino}-6-methylpyrazolo[1,5-a]pyrazine-2-carboxylate (1.79 g, 4.39 mmol) in THF, MeOH, H₂O (1:3:1; 21.9 mL) was added LiOH.H₂O (368 mg, 8.78 mmol). The mixture was stirred at rt for 45 min and the solvent was removed under reduced pressure to give 4-{[(1R)-1-[3-amino-5-(trifluoromethyl)phenyl]ethyl]amino}-6-methylpyrazolo[1,5-a]pyrazine-2-carboxylic acid (1.84 g), which was used in the next step without further purification. LCMS (ESI): m/z: [M+H] calculated for C₁₇H₁₇F₃N₅O₂: 380.1; found 380.4.

### Step 3.

To a mixture of 4-{[(1R)-1-[3-amino-5-(trifluoromethyl)phenyl]ethyl]amino}-6-methylpyrazolo[1,5-a]pyrazine-2-carboxylic acid (100 mg, 0.26 mmol) and morpholine (22.9 μL, 0.26 mmol) in DMF (1.75 mL) was added DIPEA (227 μL, 1.31 mmol) and PyBOP (150 mg, 0.29 mmol). The mixture was stirred at rt for 1 h, then the solvent was removed under reduced pressure and the crude product was purified by prep-HPLC to give N-[(1 R)-1-[3-amino-5-(trifluoromethyl)phenyl]ethyl]-6-methyl-2-(morpholine-4-carbonyl)pyrazolo[1,5-a]pyrazin-4-amine (11 mg, 9% yield). LCMS (ESI): m/z: [M+H] calculated for C₂₁H₂₄F₃N₆O₂: 449.2; found 449.5; ¹H NMR (500 MHz, METHANOL-d₄) δ ppm 7.62 (t, J=1.1 Hz, 1H), 7.28 (s, 1H), 7.00-6.93 (m, 2H), 6.79 (d, J=2.2 Hz, 1H), 5.41 (q, J=7.1 Hz, 1H), 4.01 (s, 2H), 3.75 (d, J=34.4 Hz, 7H), 2.78 (s, 2H), 2.24 (d, J=1.2 Hz, 3H), 1.59 (d, J=7.1 Hz, 3H).

The following examples 59-62 shown in Table 2 were synthesized in the manner similar to Example 58.

TABLE 2
Examples 59-62 and 74-82

| | | Mass |
|---|---|---|
| Example # | Structure | Found |
| Example 59. | 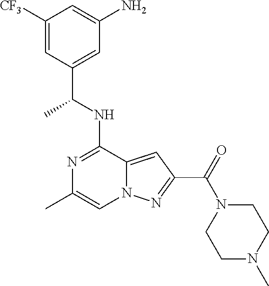 [SMILES: Cc1cn2nc(C(=O)N3CCN(C)CC3)cc2c(N[C@H](C)c2cc(N)cc(C(F)(F)F)c2)n1] | 462.5 |
| Example 60. | 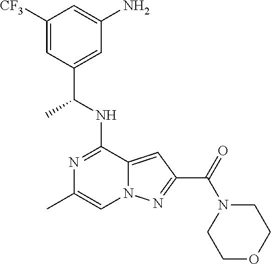 [SMILES: Cc1cn2nc(C(=O)N3CCOCC3)cc2c(N[C@H](C)c2cc(N)cc(C(F)(F)F)c2)n1] | 449.5 |
| Example 61. | 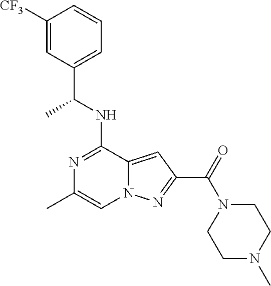 [SMILES: Cc1cn2nc(C(=O)N3CCN(C)CC3)cc2c(N[C@H](C)c2cccc(C(F)(F)F)c2)n1] | 447.6 |
| Example 62. | 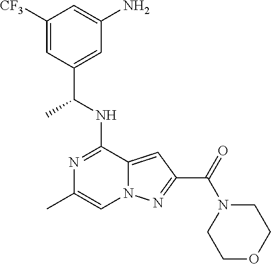 [SMILES: Cc1cn2nc(C(=O)N3CCOCC3)cc2c(N[C@H](C)c2cc(N)cc(C(F)(F)F)c2)n1] | 434.6 |
| Example 74. | 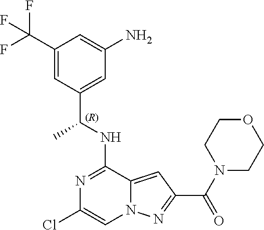 | 469.2 |
| Example 75. | 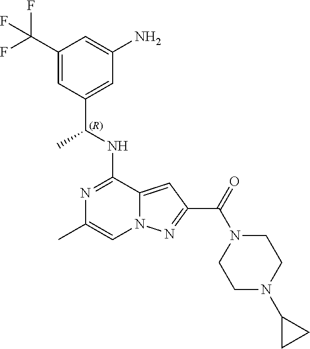 | 488.1 |
| Example 76. | 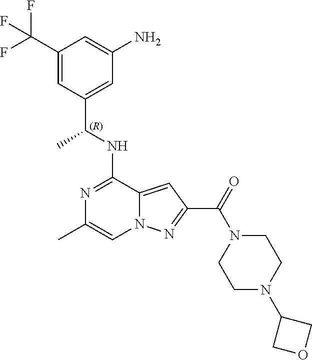 | 504.1 |
| Example 77. | 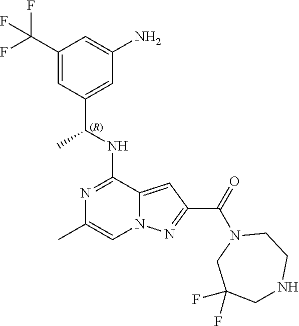 | 498.0 |
| Example 78. | 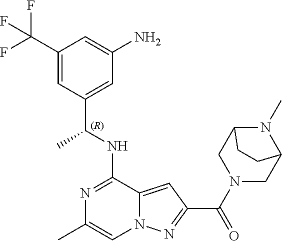 | 488.1 |
| Example 79. | 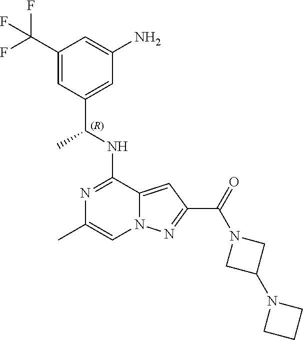 | 474.1 |
| Example 80. | 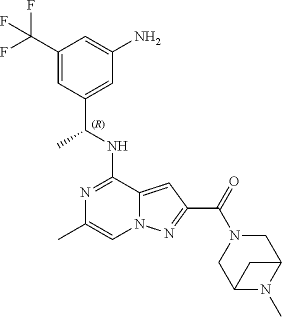 | 474.1 |
| Example 81. | 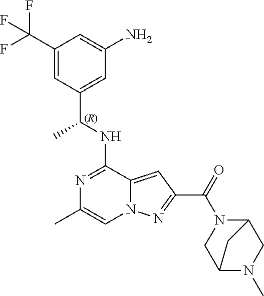 | 474.1 |
| Example 82. | 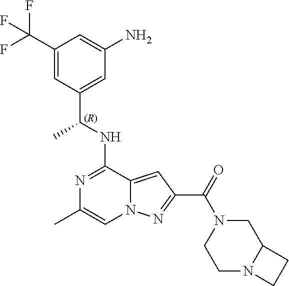 | 474.1 |

### Example 63. Synthesis of N-[(1R)-1-[3-amino-5-(trifluoromethyl)phenyl]ethyl]-2-methyl-6-[2-(morpholin-4-yl)pyridin-4-yl]pyrrolo[2,1-f][1,2,4]triazin-4-amine

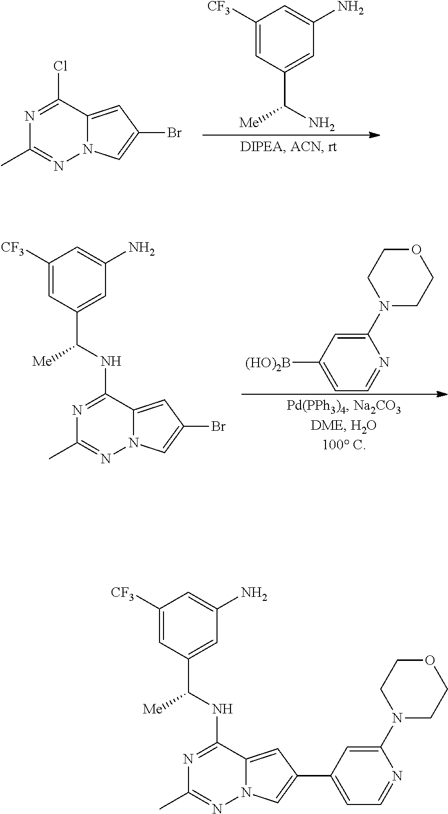 [SMILES: Cc1nc(Cl)c2cc(Br)cn2n1 ; C[C@@H](N)c1cc(N)cc(C(F)(F)F)c1 ; Cc1nc(N[C@H](C)c2cc(N)cc(C(F)(F)F)c2)c2cc(Br)cn2n1 ; OB(O)c1ccnc(N2CCOCC2)c1 ; Cc1nc(N[C@H](C)c2cc(N)cc(C(F)(F)F)c2)c2cc(-c3ccnc(N4CCOCC4)c3)cn2n1]

### Step 1.

To a mixture of 6-bromo-4-chloro-2-methylpyrrolo[2,1-f][1,2,4]triazine (2.0 g, 8.1 mmol) and 3-[(1R)-1-aminoethyl]-5-(trifluoromethyl)aniline (1.65 g, 8.1 mmol) in MeCN (8.1 mL) was added DIPEA (2.8 mL, 16.2 mmol). The mixture was stirred at rt for 5 h at rt and the solvent was removed under reduced pressure to give N-[(1R)-1-[3-amino-5-(trifluoromethyl)phenyl]ethyl]-6-bromo-2-methylpyrrolo[2,1-f][1,2,4]triazin-4-amine (3.8 g), which was used without further purification. LCMS (ESI): m/z: [M+H] calculated for C₁₆H₁₅BrF₃N₅: 413.0; found 414.2.

### Step 2.

A mixture of N-[(1R)-1-[3-amino-5-(trifluoromethyl)phenyl]ethyl]-6-bromo-2-methylpyrrolo[2,1-f][1,2,4]triazin-4-amine (100 mg, 0.24 mmol), [2-(morpholin-4-yl)pyridin-4-yl]boronic acid (50 mg, 0.24 mmol), (Ph₃P)₄Pd (28 mg, 24 μmol) and Na₂CO₃ (77 mg, 0.72 mmol) in DME (2.4 mL) and H₂O (0.6 mL) was purged with N₂ for 5 min. The mixture was heated to 100° C. and stirred for 2 h. After cooling, the mixture was filtered, the solvent was removed under reduced pressure and the crude residue was purified by prep-HPLC to give N-[(1R)-1-[3-amino-5-(trifluoromethyl)phenyl]ethyl]-2-methyl-6-[2-(morpholin-4-yl)pyridin-4-yl]pyrrolo[2,1-f][1,2,4]triazin-4-amine (6 mg, 5% yield). LCMS (ESI): m/z: [M+H] calculated for C₂₅H₂₆F₃N₇O: 497.2; found 498.6; ¹H NMR (500 MHz, METHANOL-d₄) δ ppm 8.08 (dd, J=5.3, 0.7 Hz, 1H), 7.94 (d, J=1.8 Hz, 1H), 7.30 (d, J=1.8 Hz, 1H), 7.06 (d, J=1.3 Hz, 1H), 7.03 (dd, J=5.3, 1.4 Hz, 1H), 6.98 (s, 1H), 6.95 (d, J=1.8 Hz, 1H), 6.82 (d, J=2.0 Hz, 1H), 5.55 (q, J=7.0 Hz, 1H), 3.87-3.76 (m, 4H), 3.55-3.47 (m, 4H), 2.30 (s, 3H), 1.62 (d, J=7.0 Hz, 3H).

### Example 64. Synthesis of N-[(1R)-1-[3-amino-5-(trifluoromethyl)phenyl]ethyl]-6-(2-methoxypyridin-3-yl)-2-methylpyrrolo[2,1-f][1,2,4]triazin-4-amine

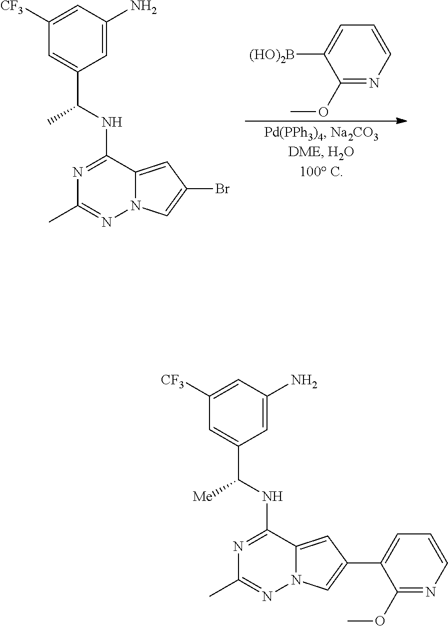 [SMILES: Cc1nc(N[C@H](C)c2cc(N)cc(C(F)(F)F)c2)c2cc(Br)cn2n1 ; COc1ncccc1B(O)O ; COc1ncccc1-c1cc2c(N[C@H](C)c3cc(N)cc(C(F)(F)F)c3)nc(C)nn2c1]

### Step 1.

N-[(1R)-1-[3 -amino-5 -(trifluoromethyl)phenyl]ethyl]-6-(2-methoxypyridin-3 -yl)-2-methylpyrrolo[2,1-f][1,2,4]triazin-4-amine was synthesized in a manner similar to N-[(1R)-1-[3-amino-5-(trifluoromethyl)phenyl]ethyl]-2-methyl-6-[2-(morpholin-4-yl)pyridin-4-yl]pyrrolo[2,1-f][1,2,4]triazin-4-amine except [2-(morpholin-4-yl)pyridin-4-yl]boronic acid was substituted with (2-methoxypyridin-3-yl)boronic acid. LCMS (ESI): m/z: [M+H] calculated for C₂₂H₂₁F₃N₆O: 442.2; found 443.3; ¹H NMR (500 MHz, METHANOL-d₄) δ ppm 8.03 (d, J=6.0 Hz, 1H), 7.97 (d, J=1.8 Hz, OH), 7.37 (d, J=1.8 Hz, OH), 7.04-6.94 (m, 1H), 6.81 (t, J=1.9 Hz, 1H), 5.56 (q, J=7.0 Hz, 1H), 4.07 (s, 2H), 2.30 (s, 1H), 1.62 (d, J=7.1 Hz, 1H).

### Example 65. Synthesis of N-[(1R)-1-[3-amino-5-(trifluoromethyl)phenyl]ethyl]-6-(morpholine-4-carbonyl)thieno[2,3-d]pyrimidin-4-amine

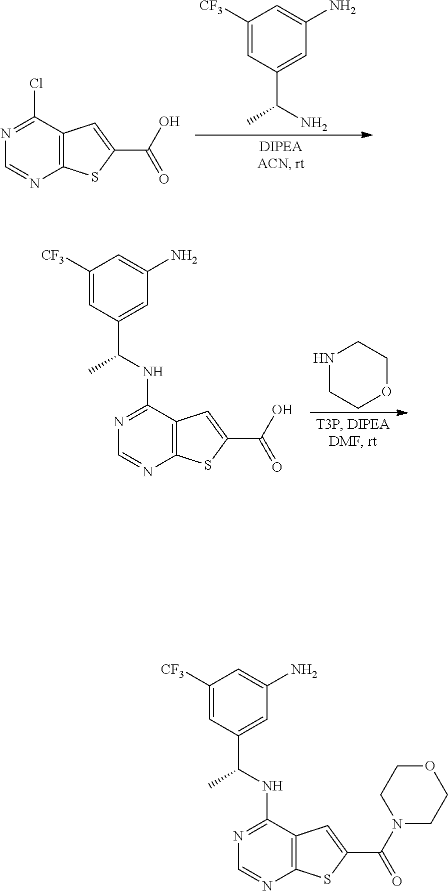 [SMILES: O=C(O)c1cc2c(Cl)ncnc2s1 ; C[C@@H](N)c1cc(N)cc(C(F)(F)F)c1 ; C[C@@H](Nc1ncnc2sc(C(=O)O)cc12)c1cc(N)cc(C(F)(F)F)c1 ; C1COCCN1 ; C[C@@H](Nc1ncnc2sc(C(=O)N3CCOCC3)cc12)c1cc(N)cc(C(F)(F)F)c1]

### Step 1.

To a mixture of 4-chlorothieno[2,3-d]pyrimidine-6-carboxylic acid (250 mg, 1.16 mmol) and 3-[(1R)-1-aminoethyl]-5-(trifluoromethyl)aniline (236 mg, 1.16 mmol) in MeCN (1.2 mL) was added DIPEA (411 μL, 2.32 mmol). The mixture was heated to 50° C. and stirred for 5 h, then the solvent was removed under reduced pressure to give 4-{[(1R)-1-[3-amino-5-(trifluoromethyl)phenyl]ethyl]amino}thieno [2,3-d] pyrimidine-6-carboxylic acid, which was used without further purification. LCMS (ESI): m/z: [M+H] calculated for C₁₆H₁₃F₃N₄O₂S: 382.1; found 383.4.

### Step 2.

To a mixture of 4-{[(1R)-1-[3-amino-5-(trifluoromethyl)phenyl]ethyl]amino}thieno[2,3-d]pyrimidine-6-carboxycacid (200 mg, 0.52 mmol) and morpholine (45 μL, 0.52 mmol) in DMF (2.6 mL) was added DIPEA (270 μL, 1.56 mmol) and T3P, 50 wt % in DMF (198 4, 0.33 mmol). The mixture was stirred at rt for 1 h and purified by prep-HPLC to give N-[(1R)-1-[3-amino-5-(trifluoromethyl)phenyl]ethyl]-6-(morpholine-4-carbonyl)thieno[2,3 -d]pyrimidin-4-amine (79 mg, 33% yield). LCMS (ESI): m/z: [M+H] calculated for C₂₀H₂₀F₃N₅O₂S: 451.1; found 452.4; ¹H NMR (500 MHz, METHANOL-d₄) δ ppm 8.32 (s, 1H), 7.96 (s, 1H), 6.94 (d, J=1.8 Hz, 2H), 6.80 (d, J=1.9 Hz, 1H), 5.48 (q, J=7.0 Hz, 1H), 3.80 (dd, J=6.8, 3.7 Hz, 4H), 3.77-3.68 (m, 4H), 1.61 (d, J=7.0 Hz, 3H).

### Example 83. (R)-(4-((1-(3-(1,1-difluoro-2-hydroxyethyl)phenyl)ethyl)amino)thieno[2,3-d]pyrimidin-6-yl)(morpholino)methanone

(R)-(4-((1-(3-(1,1-difluoro-2-hydroxyethyl)phenyl)ethyl)amino)thieno[2,3-d]pyrimidin-6-yl)(morpholino)methanone was synthesized in the manner similar to Example 65.

| | | Mass |
|---|---|---|
| Example # | Structure | Found |
| Example 83. | 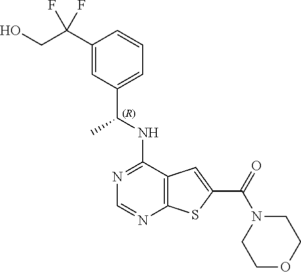 | 449.0 |

### Example 84. (R)-(4-((1-(3-(difluoromethyl)-2-fluorophenyl)ethyl)amino)thieno[2,3-d]pyrimidin-6-yl)(4-(oxetan-3-yl)piperazin-1-yl)methanone

(R)-(4-((1-(3-(difluoromethyl)-2-fluorophenyl)ethyl)amino)thieno[2,3-d]pyrimidin-6-yl)(4-(oxetan-3-yl)piperazin-1-yl)methanone was synthesized in the manner similar to Example 65.

| | | Mass |
|---|---|---|
| Example # | Structure | Found |
| Example 84. | 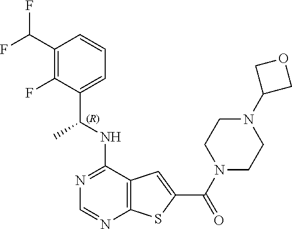 | 492.2 |

### Example 66. Synthesis of 4-[4-[[(1R)-1-[3-amino-5-(trifluoromethyl)phenyl]ethyl]amino]-2-methyl-pyrrolo[2,1-f][1,2,4]triazin-6-yl]-4-hydroxy-N,N-dimethylcyclohexanecarboxamide

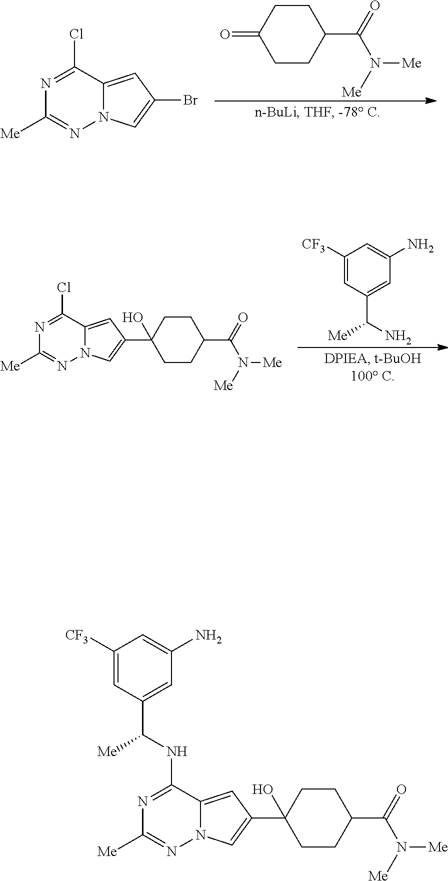 [SMILES: Cc1nc(Cl)c2cc(Br)cn2n1 ; CN(C)C(=O)C1CCC(=O)CC1 ; Cc1nc(Cl)c2cc(C3(O)CCC(C(=O)N(C)C)CC3)cn2n1 ; C[C@@H](N)c1cc(N)cc(C(F)(F)F)c1 ; Cc1nc(N[C@H](C)c2cc(N)cc(C(F)(F)F)c2)c2cc(C3(O)CCC(C(=O)N(C)C)CC3)cn2n1]

### Step 1.

4-(4-chloro-2-methyl-pyrrolo[2,1-f][1,2,4]triazin-6-yl)-4-hydroxy-N,N-dimethylcyclohexanecarboxamide was synthesized in a manner similar to 3-(benzyloxy)-1-(4-chloro-2-methylpyrrolo[2,1-f][1,2,4]triazin-6-yl)cyclobutanol except 3-(benzyloxy)cyclobutanone was substituted with N,N-dimethyl-4-oxo-cyclohexanecarboxamide. LCMS (ESI): m/z: [M+H] calculated for C₁₆H₂₂ClN₄O₂: 337.1; found 337.1.

### Step 2.

4-[4-[[(1R)-1-[3-amino-5 -(trifluoromethyl)phenyl]ethyl]amino]-2-methyl-pyrrolo[2,1-f][1,2,4]triazin-6-yl]-4-hydroxy -N,N-dimethylcyclohexanecarboxamide was synthesized in a manner similar to N-[(1R)-1-[3-amino-5-(trifluoromethyl)phenyl]ethyl]-6-bromo-2-methyl-pyrrolo[2,1-f][1,2,4]-triazin-4-amine except 6-bromo-4-chloro-2-methyl-pyrrolo[2,1-f][1,2,4]triazine was substituted with 4-(4-chloro-2-methyl-pyrrolo[2,1-f][1,2,4]triazin-6-yl)-4-hydroxy-N,N-dimethylcyclohexanecarboxamide. LCMS (ESI): m/z: [M+H] calculated for C₂₅H₃₂F₃N₆O₂: 505.2; found 505.1; ¹H NMR (400 MHz, METHANOL-d₄) δ ppm 6.95 (s, 1H), 6.93 (s, 1H), 6.86 (d, J=4.5 Hz, 1H), 6.83 (d, J=4.4 Hz, 1H), 6.80 (s, 1H), 6.62 (d, J=4.5 Hz, 1H), 6.46 (d, J=4.4 Hz, 1H), 5.56-5.49 (m, 1H), 3.14 (s, 3H), 3.12 (s, 1H), 2.95 (s, 3H), 2.89 (s, 1H), 2.85-2.72 (m, 2H), 2.31 (s, 3H), 2.25-2.19 (m, 2H), 2.16-2.05 (m, 4H), 1.88-1.77 (m, 1H), 1.65 (d, J=9.2 Hz, 2H), 1.58 (d, J=7.1 Hz, 4H).

### Example 85. Synthesis of [5-[[(1R)-1-[3-(difluoromethyl)-2-fluoro-phenyl]ethyl]amino]imidazo[1,2-c]pyrimidin-2-yl]-piperazin-1-yl-methanone hydrochloride

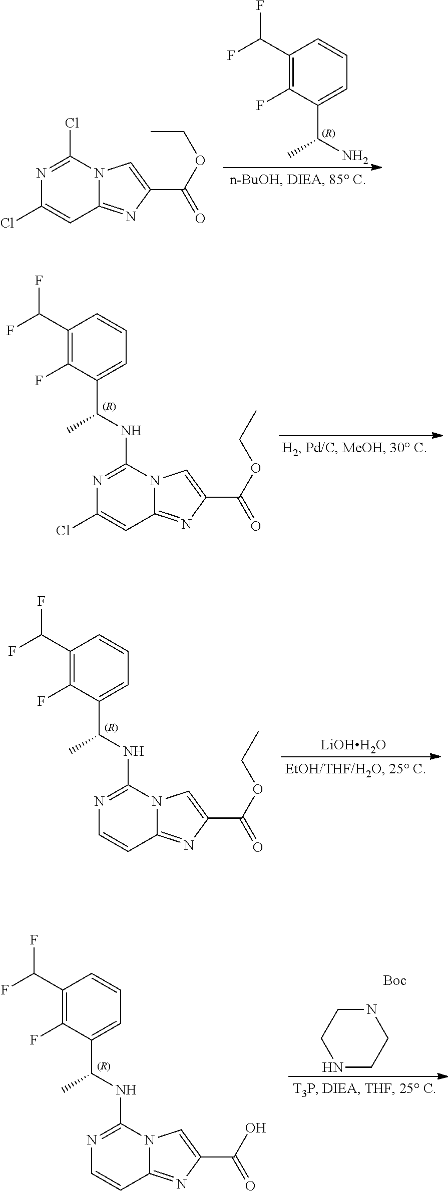

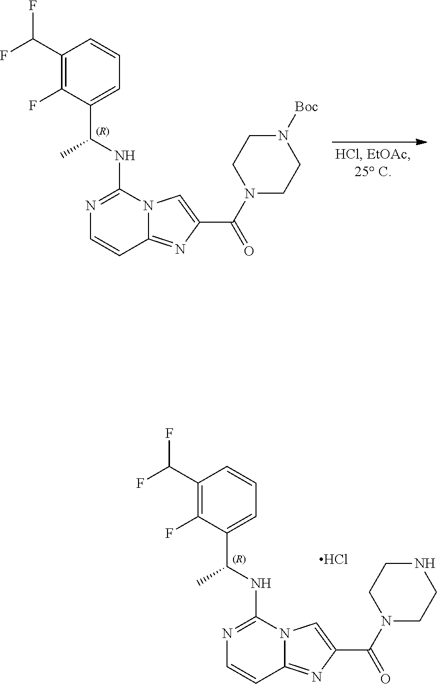

### Step 1.

To a mixture of ethyl 5,7-dichloroimidazo[1,2-c]pyrimidine-2-carboxylate (450 mg, 1.73 mmol) and (1R)-1-[3-(difluoromethyl)-2-fluoro-phenyl]ethanamine (327 mg, 1.73 mmol) in n-BuOH (1 mL) was added DIEA (1.12 g, 8.65 mmol). The reaction was stirred at 85° C. under N₂ for 3 h. The mixture was quenched by the addition of water (10 mL) and extracted with EtOAc (5 mL×3). The combined organic extracts were dried over Na₂SO₄, filtered, and concentrated under reduced pressure. The crude residue was purified by column chromatography to give ethyl 7-chloro-5-[[(1R)-1-[3-(difluoromethyl)-2-fluoro-phenyl]ethyl]amino]imidazo[1,2-c]pyrimidine-2-carboxylate (640 mg, 90% yield). LCMS (ESI): m/z: [M+H] calculated for C₁₈H₁₇ClF₃N₄O₂: 413.1; found: 413.1.

### Step 2.

To a solution of ethyl 7-chloro-5-[[(1R)-1-[3-(difluoromethyl)-2-fluoro-phenyl]ethyl]amino]imidazo[1,2-c]pyrimidine-2-carboxylate (640 mg, 1.55 mmol) in MeOH (1 mL) was added 10% Pd/C (43.6 mg, 31.01 _{N}mol) under N₂. The suspension was degassed under vacuum and purged with H₂ gas three times. The mixture was stirred under H₂ (15 psi) at 30° C. for 3 h. The reaction mixture was then filtered and the filtrate was concentrated to give ethyl 5-[[(1R)-1-[3-(difluoromethyl)-2-fluoro-phenyl]ethyl]amino]imidazo[1,2-c]pyrimidine-2-carboxylate (580 mg, 99% yield). LCMS (ESI): m/z: [M+H] calculated for C₁₈H₁₈F₃N₄O₂: 379.1; found: 379.1; ¹H NMR (400 MHz, METHANOL-d₄) δ ppm 9.01 (s, 1H) 8.10 (d, J=7.2 Hz, 1H) 7.67 (t, J=7.2 Hz, 1H) 7.52 (t, J=7.2 Hz, 1H) 7.27 (t, J=7.6 Hz, 1H) 6.86-7.14 (m, 2H) 5.73 (q, J=7.2 Hz, 1H) 4.50 (q, J=7.2 Hz, 2H) 1.74 (d, J=7.2 Hz, 3H) 1.45 (t, J=7.2 Hz, 3H).

### Step 3.

To a mixture of ethyl 5-[[(1R)-1 -[3-(difluoromethyl)-2-fluoro-phenyl]ethyl]amino]imidazo[1,2-c]pyrimidine-2-carboxylate (580 mg, 1.53 mmol) in EtOH (2 mL), THF (2 mL) and H₂O (2 mL) was added LiOH.H₂O (162 mg, 3.83 mmol). The mixture was stirred at 25° C. for 2 h under N₂. The reaction mixture was treated with a solution of HCl (2N in H₂O) until pH ˜4, then was extracted with CH₂Cl₂ (10 mL×3), dried over Na₂SO₄, filtered, and concentrated under reduced pressure to give 5-[[(1R)-1-[3-(difluoromethyl)-2-fluoro-phenyl]ethyl]amino]imidazo[1,2-c]pyrimidine-2-carboxylic acid (0.50 g, crude). LCMS (ESI): m/z: [M−H] calculated for C₁₆H₁₂F₃N₄O₂: 349.1; found 349.0; ¹H NMR (400 MHz, METHANOL-d₄) δ ppm 8.47 (s, 1H) 7.65-7.55 (m, 2H) 7.49 (t, J=6.8 Hz, 1H) 7.23 (t, J=7.6 Hz, 1H) 7.14-6.87 (m, 2H) 5.66 (q, J=6.4 Hz, 1H) 1.67 (d, J=7.2 Hz, 3H).

### Step 4.

To a mixture of 5-[[(1R)-1-[3-(difluoromethyl)-2-fluoro-phenyl]ethyl]amino]imidazo[1,2-c]pyrimidine-2-carboxylic acid (100 mg, 285 μmol) and tert-butyl piperazine-1-carboxylate (53.2 mg, 285 pmol) in THF (2 mL) were added T₃P (273 mg, 428 μmol) and DIEA (249 μL 1.43 mmol). The mixture was stirred at 25° C. for 10 h under N₂. The reaction mixture was quenched by water (10 mL), extracted with ethyl acetate (5 mL×3), dried over Na₂SO₄, filtered, and concentrated under reduced pressure. The crude residue was purified by column chromatography to give tert-butyl 4-[5-[[(1R)-1-[3-(difluoromethyl)-2-fluoro-phenyl]ethyl]amino]imidazo[1,2-c]pyrimidine-2-carbonyl]piperazine-1-carboxylate (120 mg, 81% yield). LCMS (ESI): m/z: [M+H] calculated for C₂₅H₃₀F₃N₆O₃: 519.2; found 519.2; ¹H NMR (400 MHz, METHANOL-d₄) δ ppm 8.44 (s, 1H) 7.63-7.59 (m, 2H) 7.50 (t, J=7.2 Hz, 1H) 7.24 (t, J=7.6 Hz, 1H) 7.00 (t, J=55.2 Hz, 1H) 6.81 (d, J=6.4 Hz, 1H) 5.66 (q, J=6.8 Hz, 1H) 3.98-3.77 (m, 4H) 3.53 (br s, 4H) 1.67 (d, J=6.8 Hz, 3H) 1.47 (s, 9H).

### Step 5.

tert-Butyl-4-[5-[[(1R)-1-[3-(difluoromethyl)-2-fluoro-phenyl]ethyl]amino]imidazo[1,2-c]pyrimidine-2-carbonyl]piperazine-1-carboxylate (120 mg, 231 μmol) was stirred in a 4M solution of HCl in EtOAc (578 μL, 2.31 mmol) at 25° C. for 1 h. The reaction mixture was then filtered to give [5-[[(1R)-1-[3-(difluoromethyl)-2-fluoro-phenyl]ethyl]amino]imidazo[1,2-c]pyrimidin-2-yl]-piperazin-1-yl-methanone hydrochloride (60 mg, 61% yield). LCMS (ESI): m/z: [M+H] calculated for C₂₀H₂₂F₃N₆O: 419.2; found: 419.2; ¹H NMR (400 MHz, METHANOL-d₄) δ ppm 9.12 (s, 1H) 8.12 (d, J=6.8 Hz, 1H) 7.77 (t, J=7.2 Hz, 1H) 7.52 (t, J=7.2 Hz, 1H) 7.27 (t, J=8.0 Hz, 1H) 7.14-7.00 (m, 2H) 5.76 (q, J=6.8 Hz, 1H) 4.14-4.08 (m, 4H) 3.42 (t, J=5.2 Hz, 4H) 1.76 (d, J=7.2 Hz, 3H).

The following examples 86-93 shown in Table 3 were synthesized in the manner similar to Example 85.

TABLE 3
Examples 86-93

| | | Mass |
|---|---|---|
| Example # | Structure | Found |
| Example 86. | 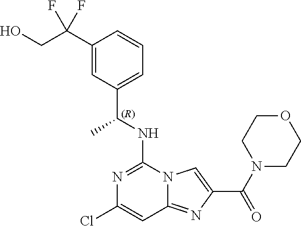 | 466.0 |
| Example 87. | 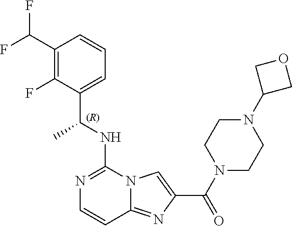 | 475.2 |
| Example 88. | 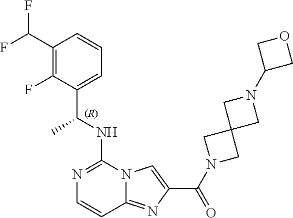 | 487.2 |
| Example 89. | 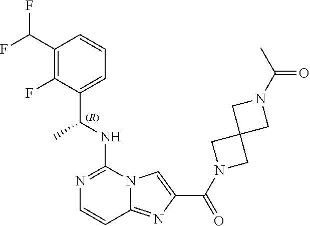 | 473.4 |
| Example 90. | 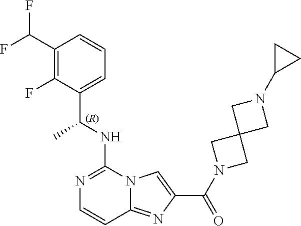 | 471.5 |
| Example 91. | 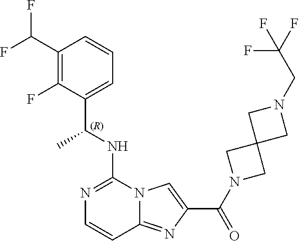 | 513.5 |
| Example 92. | 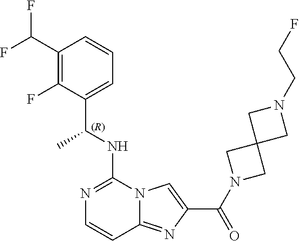 | 477.4 |
| Example 93. | 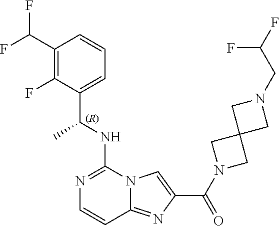 | 495.5 |

### Example 94. Synthesis of N-[(1R)-1-[3-amino-5-(trifluoromethyl)-phenyl]ethyl]-2-chloro-6-(4-methylpiperazine-1-carbonyl)pyrrolo[2,1-f][1,2,4]triazin-4-amine

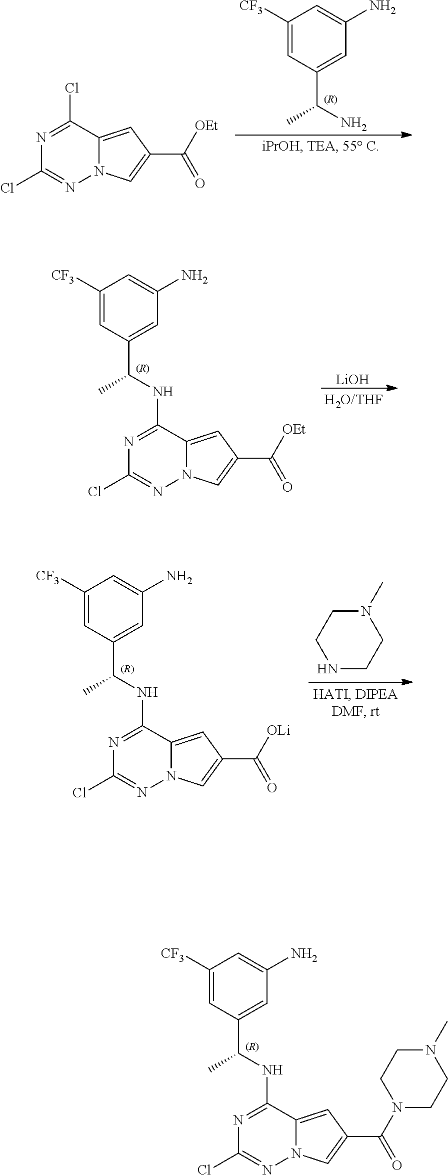

### Step 1.

Ethyl 2,4-dichloropyrrolo[2,1-f][1,2,4]triazine-6-carboxylate (371 mg, 1.43 mmol, 1.0 eq) and 3-[(1R)-1-aminoethyl]-5-(trifluoromethyl)aniline hydrochloride (446 mg, 1.86 mmol) were suspended in propan-2-ol (11.1 mL). After 3 min stirring trimethylamine (517 μL, 3.71 mmol) was added and the reaction was heated to 55° C. for 3 h. After cooling to rt the solvent was removed under reduced pressure. The residue was diluted with diethyl ether:EtOAc mixture (1:1) and washed with water. The water layer was extracted with EtOAc and the combined organic layers were washed with brine, dried over Na₂SO₄ and the solvent was removed under reduced pressure. The crude residue was purified by column chromatography column to give ethyl 4-{[(1R)-1-[3-amino-5-(trifluoromethyl)phenyl]-ethyl]amino}-2-chloropyrrolo[2,1-f][1,2,4]triazine-6-carboxylate (164 mg, yield=27%)¹H NMR (300 MHz, DMSO-d₆) δ 9.30 (d, J=8.1 Hz, 1H), 8.12 (d, J=1.7 Hz, 1H), 7.59 (d, J=1.8 Hz, 1H), 6.84 (s, 1H), 6.79 (s, 1H), 6.73 (s, 1H), 5.62 (s, 2H), 5.48-5.26 (m, 1H), 4.27 (q, J=7.1 Hz, 2H), 1.52 (d, J=7.0 Hz, 3H), 1.30 (t, J=7.1 Hz, 3H).

### Step 2.

Lithium hydroxide monohydrate (26 mg, 0.61 mmol) was added in one portion to a stirred suspension of ethyl 4-{[(1R)-1-[3-amino-5-(trifluoromethyl)phenyl]ethyl]amino}-2-chloropyrrolo[2,1-f][1,2,4]triazine-6-carboxylate (164 mg, 0.38 mmol) in a mixture of THF and water (8.3 mL, 10:7, v/v). The mixture was stirred for 72 h at rt. Solvents were removed under reduced pressure to give lithio 4-{[(1R)-1-[3-amino-5 -(trifluoromethyl)phenyl]-ethyl]amino}-2-chloropyrrolo[2,1-f][1,2,4]triazine-6-carboxylate (174 mg, 99%). ¹H NMR (300 MHz, DMSO-d₆) δ 8.27 (d, J=7.2 Hz, 1H), 7.81-7.71 (m, 1H), 7.71-7.62 (m, 1H), 7.45-7.31 (m, 1H), 5.56-5.42 (m, 1H), 4.74-4.37 (m, 4H), 3.62 (t, J=4.7 Hz, 4H), 3.24 (t, J=4.7 Hz, 4H), 1.51 (d, J=7.0 Hz, 3H).

### Step 3.

To a solution of lithio 4-{[(1R)-1-[3-amino-5-(trifluoromethyl)phenyl]ethyl]amino}-2-chloropyrrolo[2,1-f][1,2,4]triazine-6-carboxylate (173 mg, 0.43 mmol) in. DMF (5.2 mL) DIPEA (260 μL, 1.5 mmol) was added and N-methylpiperazine(66 μL, 0.6 mmol). After 10 min HATU (227 mg, 0.6 mmol) was added. The mixture was stirred at rt for 2 h. Water was added and the mixture extraceted with diethyl ether. The combined organic phases were dried over anhydrous Na₂SO₄ The solvent was removed under reduced pressure. The crude product was purified by prep-HPLC to give N-[(1R)-1-[3-amino-5-(trifluoromethyl)phenyl]ethyl]-2-chloro-6-(4-methylpiperazine-1-carbonyl)pyro-lo[2,1-f][1,2,4]triazin-4-amine (54 mg, yield=26%). LCMS ESI): exact mass for C21H23ClF3N7O: 481.16; [M+H]⁺=481.7 found; ¹H NMR (300 MHz, DMSO-d₆) δ 9.18 (d, J=8.1 Hz, 1H), 7.93 (d, J=1.7 Hz, 1H), 7.32 (d, J=1.8 Hz, 1H), 6.85 (s, 1H), 6.80 (s, 1H), 6.75 (s, 1H), 5.64 (s, 2H), 5.46-5.34 (m, 1H), 3.64 (s, 4H), 2.37 (s, 4H), 2.23 (s, 3H), 1.54 (d, J=7.0 Hz, 3H).

The following examples 95-100 shown in Table 4 were synthesized in the manner similar to Example 94.

TABLE 4
Examples 95-100

| | | Mass |
|---|---|---|
| Example # | Structure | Found |
| Example 95. | 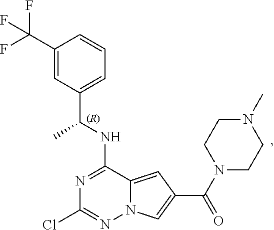 | 466.9 |
| Example 96. | 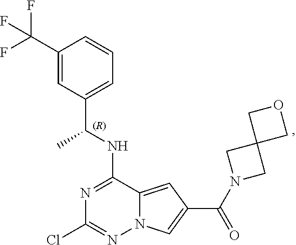 | 465.7 |
| Example 97. | 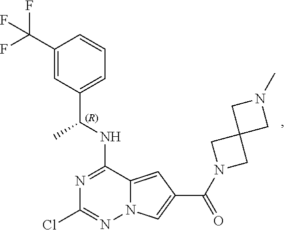 | 478.9 |
| Example 98. | 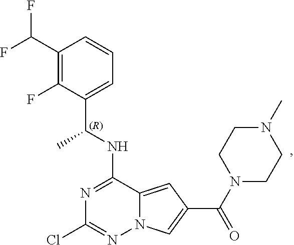 | 466.9 |
| Example 99. | 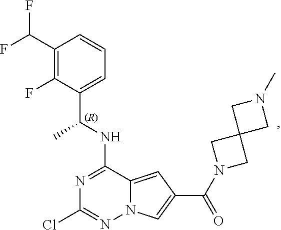 | 478.9 |
| Example 100. | 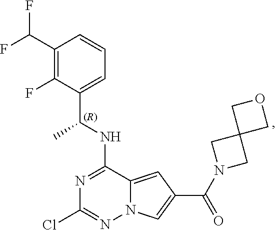 | 465.6 |

### Example 101. Synthesis of (2E)-3-(4-{[(1R)-1-[3-amino-5-(trifluoromethyl)phenyl]ethyl]amino}-2-chloropyrrolo[2,1-f][1,2,4]triazin-6-yl)prop-2-enoic acid

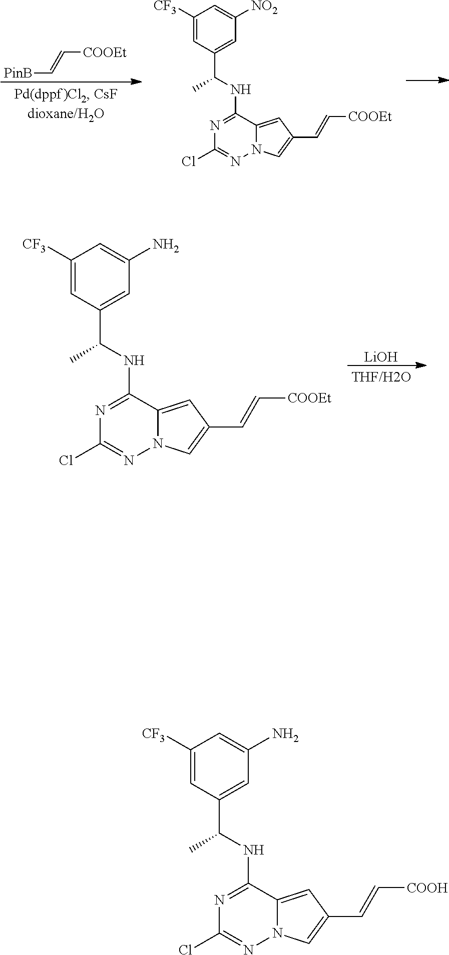

### Step 1.

A mixture of 6-bromo-2-chloro-N-[(1R)-1-[3-nitro-5-(trifluoromethyl)phenyl]ethyl]pyrrolo[2,1-f][1,2,4]triazin-4-amine (278 mg, 0.6 mmol) and 2-(ethoxycarbonyl)vinylboronic acid pinacol ester (142 mg, 0.63 mmol) in 1,4-dioxane (9.8 mL) was purged with Ar for 15 min. CsF (182 mg, 1.2 mmol) in H₂O (2.8 mL) and added and the mixture was purged with Ar for a further 15 min. Pd(dppf)Cl₂ (22 mg, 0.03 mmol) was added, the mixture heated to 110° C. and stirred overnight. The mixture was filtered through a pad of Celite® and the filter cake washed with EtOAc. The filtrate was washed with H₂O and brine, the organic layer was dried over Na₂SO₄ and filtered. The filtrate was concentrated under reduced pressure and the crude residue was purified by column chromatography to give ethyl (2E)-3-(2-chloro-4-{[(1R)-1-[3-nitro-5-(trifluoromethyl)phenyl]-ethyl]amino}pyrrolo[2,1-f][1,2,4]triazin-6-yl)prop-2-enoate (154 mg, 53% yield). LCMS (ESI): m/z: [M+H] calculated for C₂₀H₁₇ClF₃N₅O₄: 483.09; found 484.37; ¹H NMR (300 MHz, DMSO-d₆) δ ppm 9.36 (d, J=7.6 Hz, 1H), 8.58 (s, 1H), 8.40 (s, 1H), 8.31 (s, 1H), 8.19 (d, J=1.7 Hz, 1H), 7.66 (d, J=16.0 Hz, 1H), 7.32 (d, J=1.7 Hz, 1H), 6.40 (d, J=16.0 Hz, 1H), 5.72-5.56 (m, 1H), 4.18 (q, J=7.1 Hz, 2H), 1.63 (d, J=7.0 Hz, 2H), 1.25 (t, J=7.1 Hz, 3H).

### Step 2.

Ethyl (2E)-3-(4-{[(1R)-1-[3-amino-5-(trifluoromethyl)phenyl]ethyl]amino}-2-chloro-pyrrolo[2,1-f][1,2,4]triazin-6-yl)prop-2-enoate was synthesized in a manner similar to N-[(1R)-1-[3-amino-5-(trifluoromethyl)phenyl]ethyl]-2-chloro-7-(oxolan-3-ylmethyl)-5H,6H,7 H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with ethyl (2E)-3-(2-chloro-4-{[(1R)-1-[3-nitro-5-(trifluoromethyl)phenyl]-ethyl]amino}pyrrolo[2,1-f][1,2,4]triazin-6-yl)prop-2-enoate to give (119 mg, 83% yield). LCMS (ESI): m/z: [M+H] calculated for C₂₀H₁₉ClF₃N₅O₂: 453.12; found 454.20; ¹H NMR (300 MHz, DMSO-d₆) δ ppm 9.20 (d, J=8.1 Hz, 1H), 8.17 (d, J=1.7 Hz, 1H), 7.64 (d, J=16.0 Hz, 1H), 7.35 (d, J=1.8 Hz, 1H), 6.84 (s, 1H), 6.79 (s, 1H), 6.73 (s, 1H), 6.36 (d, J=15.9 Hz, 1H), 5.62 (s, 2H), 5.44-5.31 (m, 1H), 4.17 (q, J=7.1 Hz, 2H), 1.53 (d, J=7.0 Hz, 3H), 1.25 (t, J=7.1 Hz, 3H).

### Step 3.

To a mixture of ethyl (2E)-3-(4-{[(1R)-1-[3-amino-5-(trifluoromethyl)phenyl]ethyl]amino}-2-chloropyrrolo[2,1 -f][1,2,4]triazin-6-yl)prop-2-enoate (106 mg, 0.23 mmol) in THF (1.1 mL) and H₂O (0.7 mL) was added LiOH.H₂O (24 mg, 0.56 mmol). The mixture was stirred at rt overnight, then concentrated under reduced pressure and the crude residue was purified by preparative HPLC to give (2E)-3-(4-{[(1R)-1-[3-amino-5-(trifluoromethyl)phenyl]ethyl]amino}-2-chloropyrrolo[2,1-f][1,2,4]triazin-6-yl)prop-2-enoic acid. LCMS (ESI): m/z: [M+H] calculated for C₁₈H₁₅ClF₃N₅O₂: 425.09; found 425.96; ¹H NMR (300 MHz, DMSO-d₆) δ ppm 12.49-12.09 (br s, 1H), 9.18 (d, J=8.1 Hz, 1H), 8.12 (d, J=1.7 Hz, 1H), 7.57 (d, J=15.9 Hz, 1H), 7.34 (d, J=1.7 Hz, 1H), 6.84 (s, 1H), 6.79 (s, 1H), 6.73 (s, 1H), 6.28 (d, J=15.9 Hz, 1H), 5.62 (s, 2H), 5.36 (q, J=7.3 Hz, 1H), 1.53 (d, J=7.0 Hz, 3H).

### Example 102. Synthesis of N-[(1R)-1-[3-amino-5-(trifluoromethyl)phenyl]ethyl]-3-chloro-7-(morpholine-4-carbonyl)pyrrolo [1,2-a]pyrazin-1-amine

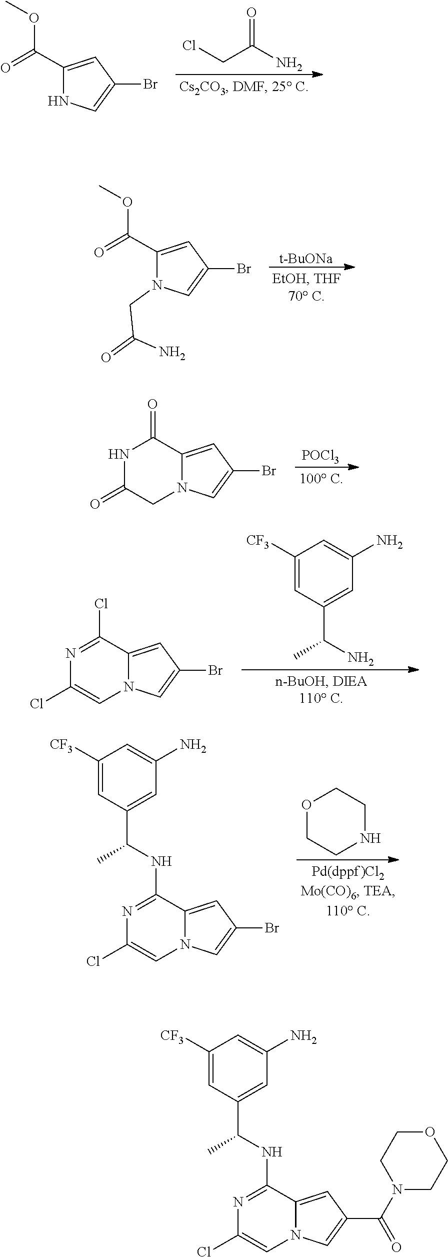

### Step 1.

To a mixture of methyl 4-bromo-1H-pyrrole-2-carboxylate (3 g, 14.70 mmol) and 2-chloroacetamide (1.65 g, 17.65 mmol) in DMF (30 mL) was added Cs₂CO₃ (6.23 g, 19.12 mmol). The mixture was stirred at 25° C. for 14 h under N₂. The mixture was poured into ice-water and the aqueous phase was extracted with EtOAc. The combined organic phases were washed with brine, dried over Na₂SO₄ and the solvent was removed under reduced pressure. The residue was purified by silica gel chromatography to afford methyl 1-(2-amino-2-oxo-ethyl)-4-bromo-pyrrole-2-carboxylate (3.1 g, 80.75% yield).¹H NMR (400 MHz, DMSO-d6) δ ppm 7.49 (br s, 1H) 7.26 (d, J=2.0 Hz, 1H) 7.10 (br s, 1H) 6.87 (d, J=2.0 Hz, 1H) 4.89 (s, 2H) 3.71 (s, 3H).

### Step 2.

A mixture of methyl 1-(2-amino-2-oxo-ethyl)-4-bromo-pyrrole-2-carboxylate (1.4 g, 5.36 mmol) and t-BuONa (1.29 g, 13.41 mmol) in THF (8 mL) and EtOH (60 mL) was heated to 70° C. and stirred for 14 h. The pH was adjusted to 6 using 2 N HCl, all solids were filtered off and the solvent was removed under reduced pressure. The crude product was triturated with EtOH and filtered to afford 7-bromo-4H-pyrrolo[1,2-a]pyrazine-1,3-dione (1 g, 81.42% yield).

### Step 3.

A mixture of 7-bromo-4H-pyrrolo[1,2-a]pyrazine-1,3-dione (1 g, 4.37 mmol) in POCl₃ (10 mL) with DIEA (564.30 mg, 4.37 mmol, 760.52 uL) was heated to 100° C. and stirred for 3 h. All volatiles were removed under reduced pressure and the residue was dissolved in EtOAc. The mixture was adjusted to pH=8 using sat. aq. NaHCO₃. The aqueous phase was extracted with EtOAc and the combined organic phases were washed with brine, dried over Na₂SO₄ and the solvent was removed under reduced pressure. The crude residue was purified by silica gel chromatography to afford 7-bromo-1,3-dichloro-pyrrolo[1,2-a]pyrazine (650 mg, 55.98% yield). LCMS (ESI): m/z: [M+H] calculated for C₇H₄BrCl₂N₂: 264.89; found: 264.9.

### Step 4.

To a mixture of 3-[(1R)-1-aminoethyl]-5-(trifluoromethyl)aniline (460.72 mg, 2.26 mmol) and 7-bromo-1,3-dichloro-pyrrolo[1,2-a]pyrazine (600 mg, 2.26 mmol) in n-BuOH (10 mL) was added DIEA (874.83 mg, 6.77 mmol, 1.18 mL). The mixture was heated to 110° C. and stirred for 2 h under N₂. Water was added and the mixture was filtered. The solvent was removed under reduced pressure and the crude residue was purified by prep-TLC to give N-[(1R)-1-[3-amino-5-(trifluoromethyl)phenyl]ethyl]-7-bromo-3-chloro-pyrrolo[1,2-a]pyrazin-1-amine (400 mg, 40.88% yield). ¹H NMR (400 MHz, DMSO-d6) δ ppm 7.91 (br d, J=8.0 Hz, 1H) 7.70 (d, J=0.8 Hz, 1H) 7.63 (d, J=1.6 Hz, 1H) 7.21 (s, 1H) 6.82 (s, 1H) 6.78(s, 1H) 6.69 (s, 1H) 5.54 (br s, 2H) 5.24 (q, J=7.2 Hz, 1H) 1.48 (d, J=7.2 Hz, 3H)

### Step 5.

To a mixture of N-[(1R)-1-[3-amino-5-(trifluoromethyl)phenyl]ethyl]-7-bromo-3-chloro-pyrrolo[1,2-a]pyrazin-1-amine (200 mg, 461.20 umol) and TEA (93.34 mg, 922.40 umol, 128.39 uL) in morpholine (2 mL) was added Mo(CO)₆ (36.53 mg, 138.36 umol, 18.64 uL). Then Pd(dppf)Cl₂ (33.75 mg, 46.12 umol) was added under N₂. The mixture was heated to 100° C. and stirred for 3 h under N₂. After cooling to rt the mixture was filtered and the solvent was removed under reduced pressure. The crude residue was purified by prep-TLC to give [1-[[(JR)-1-[3-amino-5-(trifluoromethyl)phenyl]ethyl]amino]-3-chloro-pyrrolo[1,2-a]pyrazin-7-yl]-morpholino-methanone (10 mg, 4.63% yield). LCMS (ESI): m/z: [M+H] calculated for C₂₁H₂₂ClF₃N₅O₂: 468.13; found: 468.1; ¹H NMR (400 MHz, DMSO-d6) δ ppm 8.05 (br d, J=8.0 Hz, 1H) 7.76 (s, 1H) 7.71 (s, 1H) 7.33 (s, 1H) 6.84 (s, 1H) 6.80 (s, 1H) 6.70 (s, 1H) 5.55 (br s, 2H) 5.26-5.30 (m, 1H) 3.64 (d, J=6.0 Hz, 8H) 1.49 (d, J=6.80 Hz, 3 H).

### Example 103. Synthesis of (R)-(2-chloro-6-((1-(3-(1,1-difluoro-2-hydroxyethyl)phenyl)ethyl)amino)-9-methyl-9H-purin-8-yl)(morpholino)methanone

(R)-(2-chloro-6-((1-(3-(1,1-difluoro-2-hydroxyethyl)phenyl)ethyl)amino)-9-methyl-9H-purin-8-yl)(morpholino)methanone was synthesized in the manner similar to Example 57.

| Example | | Mass |
|---|---|---|
| # | Structure | Found |
| Example 103. | 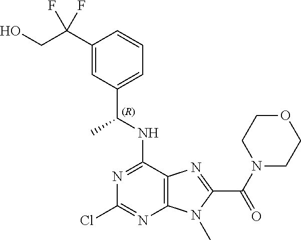 | 481.2 |

### Biological Examples

### Bodipy-FL-GTP Association Assay.

This assay was used to examine the potency with which compounds inhibit the SOS1-mediated exchange of KRAS-4B:GDP to KRAS-4B:GTP in a defined biochemical setting. A low IC₅₀ value for a given compound is indicative of high potency of said compound in inhibiting the guanine nucleotide exchange factor (GEF) activity of SOS1 on KRAS-4B in this assay setting.

Reagents: BODIPY FL GTP (ThermoFisher Scientific, Cat. G12411); KRAS4-B (Cytoskeleton Inc., Cat. CSRS03); SOS1 (Cytoskeleton Inc., Cat. CS-GE02) ; 2× Assay Buffer: 40 mM Tris-HCl, pH 7.5; 100 mM NaCl; 20 mM MgCl₂; 0.1 mg/mL BSA; 0.02% NP-40

Assay Procedure: Test compounds were dissolved in DMSO to create 20 mM master stocks. The stocks were diluted in a 3× dilution series in 100% DMSO to achieve 100× compound stocks. A 1 μl spot of each test compound stock was delivered to two adjacent wells of a 96-well assay plate prior to running the assay. Reaction Mix preparation: The following were mixed in order at room temperature to obtain the “Reaction Mix” (5.75 mL 2× Exchange Buffer; 3.22 mL MilliQ ddH2O; 3 μL 5 mM BODIPY FL GTP; 230 μL 50 μM KRAS-4B; 9.203 mL Total volume. Reaction initiation: 80 μL of Reaction Mix was pipetted into each well of a half-area black 96-well plate (Corning, Cat. 3686) containing either a 1 μL spot of DMSO or a 1 μL spot test compound at the concentrations listed above. 20 μL of 1 μM SOS1 was then added to each well to initiate the reaction. For the no GEF control wells this was replaced with 1× exchange buffer. Kinetic measurement: The reaction was monitored in a SpectraMax M2 Microplate Reader (Molecular Devices) under the following protocol: 5 second rapid circular mixing before first read; 61 readings, 30 seconds apart; Assay temperature: 22° C.; Excitation wavelength: 485 nm; Emission wavelength: 513 nm. Data Analysis: The V max values for the SOS1-mediated BODIPY FL GTP exchange curves in the presence of test compounds were normalized to the most dilute test sample columns or DMSO only control wells to give the % Activity for each concentration of test compound. Plots of % Activity vs. the Log10 of the compound concentration were fit by non-linear regression to a 4-parameter logistic model.

Bodipy-FL-GTP Association Assay results are shown in the Table 5 below. Potency Table Key: <1 μM+; 1-5 μM++; >5 μM+++.

TABLE 5

| | Bodipy-FL-GTP |
|---|---|
| Example # | Association Assay |
| Example 1. | ++ |
| Example 2. | |
| Example 3. | ++ |
| Example 4. | ++ |
| Example 5. | +++ |
| Example 6. | ++ |
| Example 7. | + |
| Example 8. | |
| Example 9. | + |
| Example 10. | ++ |
| Example 11. | + |
| Example 12. | + |
| Example 13. | ++ |
| Example 14. | ++ |
| Example 15. | +++ |
| Example 16. | +++ |
| Example 17. | +++ |
| Example 18. | +++ |
| Example 19. | +++ |
| Example 20. | +++ |
| Example 21. | ++ |
| Example 22. | +++ |
| Example 23. | + |
| Example 24. | +++ |
| Example 25. | +++ |
| Example 26. | ++ |
| Example 27. | + |

Blank = Not Determined

### Mode of Action Assay: Inhibition of SOS1 Nucleotide Exchange Activity

The purpose of this assay was to characterize the inhibitory activity of compounds on SOS1 nucleotide exchange of KRAS. Data was reported as IC₅₀ values based on the TR-FRET signal.

Note—the following protocol describes a procedure for monitoring the inhibition of SOS1 nucleotide exchange activity of wild-type KRAS in response to a compound of the invention. Other KRAS mutants and RAS isoforms maybe employed.

In assay buffer containing 20 mM HEPES, pH 7.5, 150 mM NaCl, 5 mM MgCl₂, 0.05% Tween-20, 0.1% BSA, 1 mM DTT, concentration series of test compounds were generated spanning 100 μM to 1.7 nM over eleven 3-fold serial dilutions in a 384-well assay plate at a volume of 20 μL. The purified tagless catalytic domain of SOS1 (residues 564-1049) was first diluted in assay buffer at a concentration of 100 nM, and then 20 μL of the SOS1 containing solution was directly dispensed into compound plates. The SOS1/compound mixture was incubated at room temperature with constant mixing on an orbital shaker for 20 minutes to allow the reaction to reach equilibrium. A KRAS mixture was prepared by diluting 66.7 nM avi-tagged KRAS (residue 1-169), 3.33 nM Streptavidin-Tb and 333 nM EDA-GTP-DY-647P1 in assay buffer. This mixture was prepared immediately before addition to the SOS1/compound mixture to prevent intrinsic nucleotide exchange. Then 5 μL of the pre-incubated SOS1/compound mixture and 7.5 μL of the KRAS mixture were added sequentially in a 384-well low volume black round bottom plate and incubated at room temperature with constant shaking for 30 minutes. Time-resolved fluorescence was measured on a PerkinElmer Envision plate reader. DMSO and 10 μM of compound (i) were used as negative and positive controls, respectively.

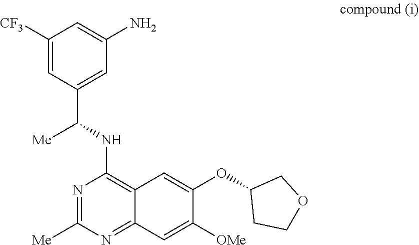 [SMILES: COc1cc2nc(C)nc(N[C@H](C)c3cc(N)cc(C(F)(F)F)c3)c2cc1O[C@H]1CCOC1]

Three replicates were performed for each compound. Data were normalized by the following: (Positive control—Sample signal)/(Positive control—negative control)*100. The data were fit using a four-parameter logistic fit.

SOS1 TR-FRET IC50 Assay results are shown in the Table 6 below: Table 3 Key: ≤1 μM+; >1 μM++.

TABLE 6

| Example # | TR-FRET IC50 |
|---|---|
| Example 28. | |
| Example 29. | ++ |
| Example 30. | |
| Example 31. | ++ |
| Example 32. | |
| Example 33. | + |
| Example 34. | ++ |
| Example 35. | ++ |
| Example 36. | + |
| Example 37. | ++ |
| Example 38. | |
| Example 39. | ++ |
| Example 40. | ++ |
| Example 41. | + |
| Example 42. | + |
| Example 43. | ++ |
| Example 44. | ++ |
| Example 45. | ++ |
| Example 46. | ++ |
| Example 47. | ++ |
| Example 48. | |
| Example 49. | ++ |
| Example 50. | + |
| Example 51. | ++ |
| Example 52. | ++ |
| Example 53. | ++ |
| Example 54. | ++ |
| Example 55. | ++ |
| Example 56. | ++ |
| Example 57. | + |
| Example 58., 60. | ++ |
| Example 59. | ++ |
| Example 61. | ++ |
| Example 62. | ++ |
| Example 63. | ++ |
| Example 64. | ++ |
| Example 65. | + |
| Example 66. | |
| Example 67. | |
| Example 68. | |
| Example 69. | + |
| Example 70. | + |
| Example 71. | + |
| Example 72. | + |
| Example 73. | + |
| Example 74. | ++ |
| Example 75. | ++ |
| Example 76. | + |
| Example 77. | ++ |
| Example 78. | + |
| Example 79. | ++ |
| Example 80. | ++ |
| Example 81. | + |
| Example 82. | + |
| Example 83. | + |
| Example 84. | + |
| Example 85. | + |
| Example 86. | + |
| Example 87. | + |
| Example 88. | + |
| Example 89. | + |
| Example 90. | + |
| Example 91. | + |
| Example 92. | + |
| Example 93. | + |
| Example 94. | + |
| Example 95. | ++ |
| Example 96. | ++ |
| Example 97. | + |
| Example 98. | + |
| Example 99. | + |
| Example 100. | + |
| Example 101. | |
| Example 102. | ++ |
| Example 103. | + |

Blank = Not Determined

Potency Assay: Measurement of the Binding Affinity of Compounds of the Invention to SOS! using Surface Plasmon Resonance (SPR)

The purpose of the SPR assay was to measure the direct binding of compounds to SOS1 catalytic domain (residues 564-1049) immobilized on a sensor chip. Data was reported as equilibrium dissociation constant (K_{d}) values.

Using a GE Biacore 8K SPR instrument, avi-tagged SOS1 catalytic domain protein was immobilized to a level of approximately 6000 response units (RU) on a streptavidin-coated SPR sensor chip in assay buffer containing 0.01 M HEPES, 0.15 M NaCl and 0.05% v/v Surfactant P20. In assay buffer containing 2% DMSO, concentration series of test compounds were generated spanning 5 μM to 4.9 nM over ten 2-fold dilutions. For each test compound, a separate 0 μM sample was generated for use during subsequent double reference subtraction. Serially for each test compound, individual dilution samples were flowed over the immobilized SOS1 protein at a flow rate of 50 μL/minute to monitor the association with SOS1. Dissociation of bound test compound from the SOS1 protein was immediately monitored by flowing assay buffer over the sensor surface and monitoring the decrease in binding signal back to the baseline level seen in the absence of compound. This was repeated for all compound dilutions in each series. The binding level response for each test compound concentration was noted immediately prior to the end of the association phase, and a secondary plot generated showing binding response level versus test compound concentration generated for each compound dilution series. This data was fitted to a model describing reversible equilibrium 1:1 binding between test compound and SOS1, yielding an estimate of the K_{d} value for the interaction.

SOS1 using Surface Plasmon Resonance (SPR) results are shown in the Table 7 below: Table 4 Key: ≤0.4 μM+; >0.4 μM++.

TABLE 7

| | SOS1 SPR |
|---|---|
| Example # | Equilibrium Kd |
| Example 28. | ++ |
| Example 29. | ++ |
| Example 30. | ++ |
| Example 31. | + |
| Example 32. | + |
| Example 33. | ++ |
| Example 34. | ++ |
| Example 35. | + |
| Example 36. | + |
| Example 37. | ++ |
| Example 38. | |
| Example 39. | + |
| Example 40. | + |
| Example 41. | + |
| Example 42. | + |
| Example 43. | ++ |
| Example 44. | ++ |
| Example 45. | + |
| Example 46. | |
| Example 47. | ++ |
| Example 48. | ++ |
| Example 49. | |
| Example 50. | ++ |
| Example 51. | ++ |
| Example 52. | + |
| Example 53. | + |
| Example 54. | |
| Example 55. | |
| Example 56. | |
| Example 57. | + |
| Example 58., 60. | ++ |
| Example 59. | + |
| Example 61. | |
| Example 62. | |
| Example 63. | + |
| Example 64. | ++ |
| Example 65. | + |
| Example 66. | + |
| Example 67. | + |
| Example 68. | |
| Example 69. | + |
| Example 70. | + |
| Example 71. | + |
| Example 72. | + |
| Example 73. | + |
| Example 74. | ++ |
| Example 75. | |
| Example 76. | + |
| Example 77. | |
| Example 78. | |
| Example 79. | |
| Example 80. | |
| Example 81. | + |
| Example 82. | + |
| Example 83. | + |
| Example 84. | + |
| Example 85. | + |
| Example 86. | + |
| Example 87. | + |
| Example 88. | + |
| Example 89. | + |
| Example 90. | + |
| Example 91. | + |
| Example 92. | + |
| Example 93. | + |
| Example 94. | |
| Example 95. | |
| Example 96. | |
| Example 97. | + |
| Example 98. | |
| Example 99. | + |
| Example 100. | + |
| Example 101. | |
| Example 102. | |
| Example 103. | ++ |

Blank = Not Determined

Potency Assay: pERK

The purpose of this assay is to measure the ability of test compounds to inhibit SOS1 function in cells. SOS1 activates RAS proteins by catalyzing the conversion of RASGDP to RASGTP in response to receptor tyrosine kinase activation. Activation of RAS induces a sequence of cellular signaling events that results in increased phosphorylation of ERK at Threonine 202 and Tyrosine 204 (pERK). The procedure described below measures the level of cellular pERK in response to test compounds in PC-9 cells (EGFR Ex19Del).

PC-9 cells were grown and maintained using media and procedures recommended by the ATCC. On the day prior to compound addition, cells were plated in 384-well cell culture plates (40 μL/well) and grown overnight in a 37° C., 5% CO₂ incubator. Test compounds were prepared in 10, 3-fold dilutions in DMSO, with a top concentration of 10 mM. On the day of the assay, 40 nL of test compound was added to each well of cell culture plate using an Echo550 liquid handler (LabCyte). Concentrations of test compound were tested in duplicate with highest test concentration being 10 μM. After compound addition, cells were incubated for 1 hour at 37° C., 5% CO₂. Following incubation, culture medium was removed and cells were washed once with phosphate buffered saline.

Cellular pERK level was determined using the AlphaLISA SureFire Ultra p-ERK1/2 Assay Kit (PerkinElmer). Cells were lysed in 25 μL lysis buffer, with shaking at 600 RPM at room temperature for 15 minutes. Lysate (10 μL) was transferred to a 384-well Opti-plate (PerkinElmer) and 5 μL acceptor mix was added. The plate was centrifuged at 1000 RPM for 1 minute, and incubated in the dark for 2 hours. Following this incubation, 5 μL of donor mix was added, the plate was sealed and centrifuged at 1000 RPM for 1 minute, and the mixture was incubated for 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

SOS1 pERK IC50 Assay results are shown in the Table 8 below. Table 5 Key: ≤1 μM+; >1 μM++.

TABLE 8

| Example # | SOS1 pERK IC50 |
|---|---|
| Example 28. | + |
| Example 29. | ++ |
| Example 30. | ++ |
| Example 31. | ++ |
| Example 32. | |
| Example 33. | + |
| Example 34. | |
| Example 35. | ++ |
| Example 36. | + |
| Example 37. | ++ |
| Example 38. | ++ |
| Example 39. | ++ |
| Example 40. | ++ |
| Example 41. | + |
| Example 42. | + |
| Example 43. | |
| Example 44. | |
| Example 45. | |
| Example 46. | |
| Example 47. | ++ |
| Example 48. | + |
| Example 49. | ++ |
| Example 50. | + |
| Example 51. | ++ |
| Example 52. | |
| Example 53. | ++ |
| Example 54. | |
| Example 55. | |
| Example 56. | ++ |
| Example 57. | + |
| Example 58., 60. | |
| Example 59. | |
| Example 61. | ++ |
| Example 62. | |
| Example 63. | |
| Example 64. | |
| Example 65. | + |
| Example 66. | ++ |
| Example 67. | |
| Example 68. | + |
| Example 69. | + |
| Example 70. | + |
| Example 71. | + |
| Example 72. | + |
| Example 73. | ++ |
| Example 74. | + |
| Example 75. | + |
| Example 76. | + |
| Example 77. | |
| Example 78. | + |
| Example 79. | |
| Example 80. | |
| Example 81. | + |
| Example 82. | + |
| Example 83. | + |
| Example 84. | + |
| Example 85. | + |
| Example 86. | |
| Example 87. | ++ |
| Example 88. | + |
| Example 89. | + |
| Example 90. | + |
| Example 91. | + |
| Example 92. | |
| Example 93. | |
| Example 94. | + |
| Example 95. | ++ |
| Example 96. | + |
| Example 97. | + |
| Example 98. | + |
| Example 99. | + |
| Example 100. | + |
| Example 101. | |
| Example 102. | ++ |
| Example 103. | + |

Blank = Not Determined

### Equivalents

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound having the structure of Formula (I), 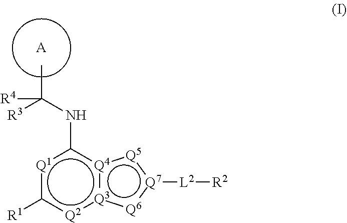
or a pharmaceutically acceptable salt, solvate, stereoisomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N-C₁₋₆ alkyl, N-C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}; wherein lea is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—, 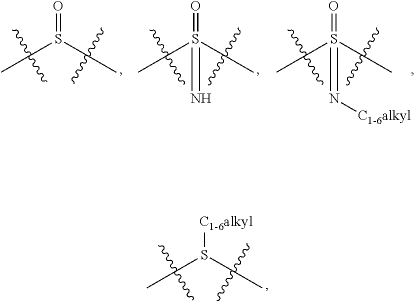
—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, -NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

2. The compound of claim 1, or a pharmaceutically acceptable salt, solvate, stereoisomer, prodrug, or tautomer thereof, wherein no more than five of Q¹, Q², Q³, Q⁴, Q⁵, Q⁶, and Q⁷ is N, NH, NCH3, O, or S.

3. The compound of claim 1 having the structure of Formula (I-a), 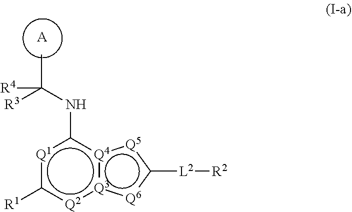
or a pharmaceutically acceptable salt, solvate, stereoisomer, prodrug, or tautomer thereof, wherein:
Q¹, Q², Q⁵ and A are as defined in claim 1;
Q³ and Q⁴ are independently C or N, wherein at least one of Q³ and Q⁴ is C;
Q⁶ is CH, N, NH, O, or S;
wherein at least one of Q¹, Q², Q³, Q⁴, Q⁵, and Q⁶ is N, NH, O, or S;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}, wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl; and
R³ and R⁴ are independently H or C₁₋₆ alkyl; wherein at least one of R³ and R⁴ is not H; or R³ and R⁴ together with the atom to which they are attached combine to form a 3-6 membered cycloalkyl.

4. The compound of claim 1, or a pharmaceutically acceptable salt, solvate, stereoisomer, prodrug, or tautomer thereof, wherein A is an optionally substituted 6-membered aryl.

5. The compound of claim 1, or a pharmaceutically acceptable salt, solvate, stereoisomer, prodrug, or tautomer thereof, wherein A is an optionally substituted 5-6 membered heteroaryl.

6. The compound of claim 1 having the structure of Formula (II), 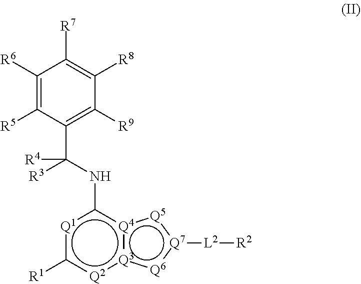
or a pharmaceutically acceptable salt, solvate, stereoisomer, prodrug, or tautomer thereof, wherein:
L², Q¹, Q², Q³, Q⁴, Q⁵, Q⁶, Q⁷, R¹, R², R³ and R⁴ are as defined in claim 1;
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

7. The compound of claim 6, or a pharmaceutically acceptable salt, solvate, stereoisomer, prodrug, or tautomer thereof, wherein no more than five of Q¹, Q², Q³, Q⁴, Q⁵, Q⁶, and Q⁷ is N, NH, NCH₃, O, or S.

8. The compound of claim 6 having the structure of Formula (II-a), 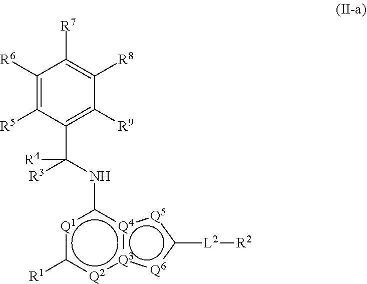
or a pharmaceutically acceptable salt, solvate, stereoisomer, prodrug, or tautomer thereof, wherein:
Q¹, Q², Q⁵, R², R³, R⁴, R⁵, R⁶, R⁷, R⁸, R⁹, R¹⁰, R¹¹, R¹², R¹³ and R¹⁴ are as defined in claim 6;
Q³ and Q⁴ are independently C or N, wherein at least one of Q³ and Q⁴ is C;
Q⁶ is CH, N, NH, O, or S;
wherein at least one of Q¹, Q², Q³, Q⁴, Q⁵, and Q⁶ is N, NH, O, or S;
R¹ is selected from the group consisting of H, halogen, C₁₋₆ alkyl, cyclopropyl, —CN, and —OR^{1a}; wherein R^{1a} is H or C₁₋₆ alkyl; and
L² is selected from the group consisting of a bond, —C(O)—, —C(O)O—, —C(O)NH(CH₂)ₒ—, —S(O)₂—, —C(O)(CH₂)ₚ—, —(CH₂)ₚ, and —O—; wherein o is 0, 1, or 2; and wherein p is a number from 1 to 6.

9. The compound of claim 1 having the structure of Formula (III), 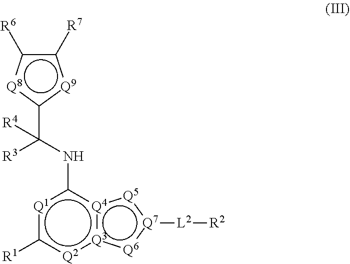
or a pharmaceutically acceptable salt, solvate, stereoisomer, prodrug, or tautomer thereof, wherein:
L², Q¹, Q², Q³, Q⁴, Q⁵, Q⁶, Q⁷, R¹, R², R³ and R⁴ are as defined in claim 1;
Q⁸ and Q⁹ are independently CH, N, NH, O, or S, provided at least one of Q¹ and Q⁹ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰ S(O)₂NR¹¹R¹², —NR¹⁰ S(O)₂R¹¹, —S(O)NR¹¹R¹², —S(O)R¹⁰, —NR¹⁰ S(O)NR¹¹R¹², —NR¹⁰ S(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

10. The compound of claim 9 or a pharmaceutically acceptable salt, solvate, stereoisomer, prodrug, or tautomer thereof, having the structure of Formula (III-a), 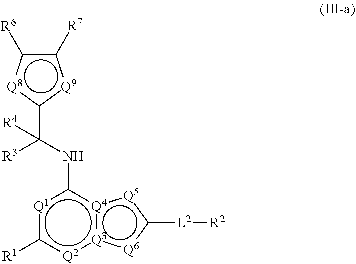
wherein L², Q¹, Q², Q³, Q⁴, Q⁵, Q⁶, Q⁸, Q⁹, R¹, R², R³, R⁴, R⁶, and R⁷ are as defined in claim 9.

11. The compound of claim 9, or a pharmaceutically acceptable salt, solvate, stereoisomer, prodrug, or tautomer thereof, wherein no more than five of Q¹, Q², Q³, Q⁴, Q⁵, Q⁶, and Q⁷ is N, NH, NCH₃, O, or S.

12. The compound of claim 1, or a pharmaceutically acceptable salt, solvate, stereoisomer, prodrug, or tautomer thereof, wherein: 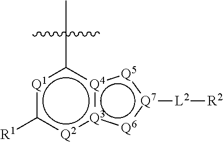
is selected from the group consisting of:

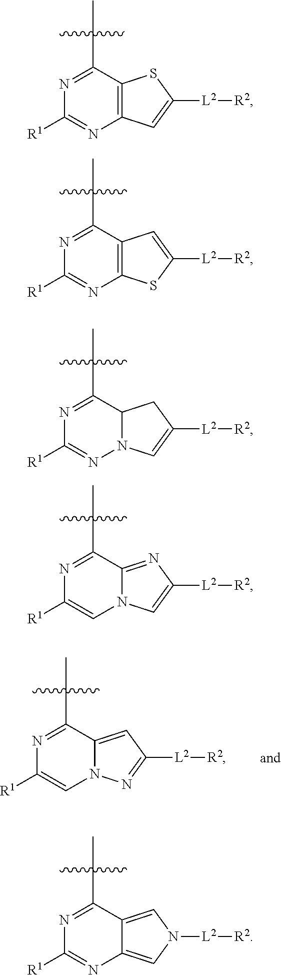

13. The compound of claim 1, or a pharmaceutically acceptable salt, solvate, stereoisomer, prodrug, or tautomer thereof, wherein: 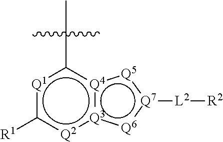
is selected from the group consisting of: of:

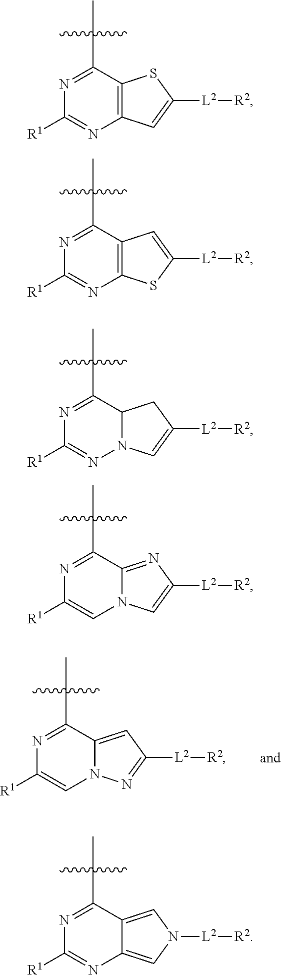

14. The compound of claim 1, or a pharmaceutically acceptable salt, solvate, stereoisomer, prodrug, or tautomer thereof, wherein: 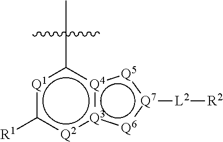
is selected from the group consisting of:

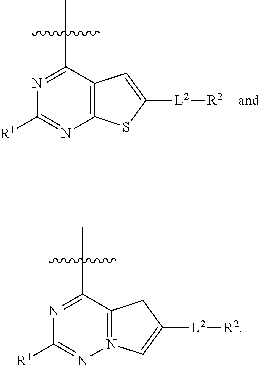

15. The compound of claim 1, or a pharmaceutically acceptable salt, solvate, stereoisomer, prodrug, or tautomer thereof, wherein R¹ is H, halogen, C₁₋₆ alkyl, cyclopropyl, —CN, or —OR^{1a}; wherein R^{1a} is H or C₁₋₆ alkyl.

16. The compound of claim 1, or a pharmaceutically acceptable salt, solvate, stereoisomer, prodrug, or tautomer thereof, wherein L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6.

17. The compound of claim 1, or a pharmaceutically acceptable salt, solvate, stereoisomer, prodrug, or tautomer thereof, wherein L² is selected from the group consisting of

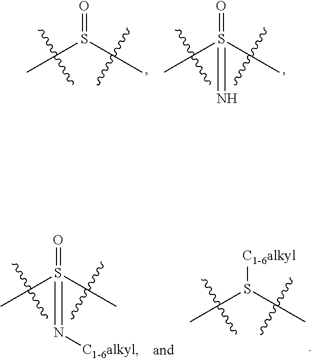

18. The compound of claim 1, or a pharmaceutically acceptable salt, solvate, stereoisomer, prodrug, or tautomer thereof, wherein R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, —OH, —OR^{2a}, oxo, halogen, —C(O)R^{2a}, —C(O)OR^{2a}, —C(O)NR^{2b}R^{2c}; —CN; —NR^{2b}R^{2c}, 3-6 membered cycloalkyl, 3-7 membered heterocyclyl, 6-10 membered aryl, or 5-10 membered heteroaryl.

19. The compound of claim 1, or a pharmaceutically acceptable salt, solvate, stereoisomer, prodrug, or tautomer thereof, wherein R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, and 5-10 membered heteroaryl is independently optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

20. The compound of claim 3, or a pharmaceutically acceptable salt, solvate, stereoisomer, prodrug, or tautomer thereof, wherein R³ is H and R⁴ is —CH₃ and the compound is of the following formula: 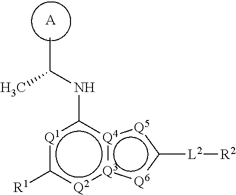
wherein A, L², Q¹, Q², Q³, Q⁴, Q⁵, Q⁶, R¹, R², m and n are as defined in claim 3.

21. The compound of claim 8, or a pharmaceutically acceptable salt, solvate, stereoisomer, prodrug, or tautomer thereof, wherein R³ is H and R⁴ is —CH₃ and the compound is of the following formula: 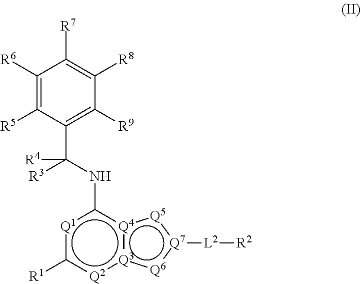
wherein L², Q¹, Q², Q³, Q⁴, Q⁵, Q⁶, R¹, R², R⁴, R⁵, R⁶, R⁷, R⁸, R⁹, m and n are as defined in claim 8.

22. A compound, or a pharmaceutically acceptable salt, solvate, hydrate, tautomer, or stereoisomer thereof, selected from the group consisting of compounds of Collection 1.

23. The compound of claim 1, or a pharmaceutically acceptable salt, solvate, hydrate, tautomer, or stereoisomer thereof, selected from the group consisting of compounds of Collection 3.

24. The compound of claim 1, or a pharmaceutically acceptable salt, solvate, hydrate, tautomer, or stereoisomer thereof, selected from the group consisting of compounds of Collection 4.

25. A compound, or a pharmaceutically acceptable salt, solvate, hydrate, tautomer, or stereoisomer thereof, selected from the group consisting of compounds of Table A.

26. A pharmaceutical composition comprising a compound of claim 1, or a pharmaceutically acceptable salt, solvate, hydrate, tautomer, or stereoisomer thereof, and a pharmaceutically acceptable carrier.

27. A method of inhibiting SOS1 in a subject, comprising administering to the subject a compound of claim 1, or a pharmaceutically acceptable salt, solvate, hydrate, tautomer, or stereoisomer thereof.

28. A method of inhibiting the interaction of SOS1 and a RAS-family protein in a cell or inhibiting the interaction of SOS1 and RAC1 in a cell, comprising administering to the cell a compound of claim 1, or a pharmaceutically acceptable salt, solvate, hydrate, tautomer, or stereoisomer thereof.

29. A method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof an effective amount of a compound of claim 1, or a pharmaceutically acceptable salt, solvate, hydrate, tautomer, or stereoisomer thereof.

30. A method of treating or preventing cancer in a subject in need thereof, comprising administering to the subject an effective amount of a compound of claim 1, or a pharmaceutically acceptable salt, solvate, hydrate, tautomer, or stereoisomer thereof.

31. The method of claim 30, wherein the cancer is selected from the group consisting of pancreatic cancer, lung cancer, colorectal cancer, hematological cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

32. The method of claim 29, wherein the disease is a RASopathy.

33. The method of claim 32, wherein the RASopathy is selected from the group consisting of Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.
